SEQUENCE LISTING (240 sequences)

SEQ ID NO: 1 - protein, 32 aa, Synechococcus sp.
MKTNQLLTSVSRSTALAFLALTLGLGGEKALA

SEQ ID NO: 2 - protein, 37 aa, Synechococcus sp.
MKSQNVFSTKSAKLIVGGTIFVSAITAANFTMLSAYA

SEQ ID NO: 3 - protein, 38 aa, Synechococcus sp.
MLRLLFLHRKKAAQDFQGFTVIELMIVMIITGILTAIA

SEQ ID NO: 4 - protein, 40 aa, Synechococcus sp.
MKNFTFKLLQQLNKKKADKGFTLIELLVVIIIIGILSAIA

SEQ ID NO: 5 - protein, 39 aa, Synechococcus sp.
MSSYKAICVWLIHYSKRNNQGFTLIELLVVMIIIGILSA

SEQ ID NO: 6 - protein, 34 aa, Synechococcus sp.
MINQPCIVPAEKGFTLIELLTGMLIVGILASISA

SEQ ID NO: 7 - protein, 25 aa, Synechococcus sp.
MQLKKLFVPLLAGMLFLGGTSGAIA

SEQ ID NO: 8 - protein, 43 aa, Synechococcus sp.
MQLKKLFVPLLAGMLFLGGTSGAIAEELLRTITVTGRGEEAIA

SEQ ID NO: 9 - protein, 202 aa, Synechococcus sp.
LESTVAQFTDISGDIYRNEIAQAVNVGFIAGFNDNTFRPTDVLTREQLVSMAIEGLQALPNASLAVPTQVANAPYPDVAADRWSAAKITWAQANNIVSGYPDGTFQPTQPVTRAELLAVLRRTAEYAKAAQGQPMTLVATNGPIAFSDTAGHWANDLAAQMSTYCRVASPLNESGDRFFPDTASQRNYAAAATLRTLQCSVR

SEQ ID NO: 10 - protein, 202 aa, Synechococcus sp.
LEVLAAPGLVDPLPYLPTFTDVQNHWAKPFIQAIANLGYIHGSAQGQFFPDQPLNRAQFALWIQAIFHPSPRRPRKQFFDVPSHLPAAEAIQQGYQGCFFSGFPDHTFQPQQPLRRVHLLVAIAQGLRLPPGDIALTEHYADQEEIPPYAQAAVATALQAKIGVLPQEKLMLKPQAIASRAEGLVYCHQALVYGQRLLPLTE

SEQ ID NO: 11 - protein, 202 aa, Synechococcus sp.
LELFSQGQVQALNSPYIVTQDVVAVDYRITAGTIIPISYTADKILLTQDEILPVTLTVDANIVNTQGIVLIPQGSEIQGEFRPSGNGTRFVAQRLELPNGQMYNINAASQVITDTESVRRGTDVGNLLRNAALGTGAAAAIAAITGDRAIATEELLIGAGAGILATLIPQFLGLDRVDLLVVETNTDLDLTLANDLILQVNP

SEQ ID NO: 12 - protein, 202 aa, Synechococcus sp.
LESLGYLADEAADSTESNGLFNGEYGALAQIAFNLGDRAELGVTYVNSYHDSGAIYDFGGGSAVNGTAWANALGLFGTEANSYGVQGKFDITDRISLAAYGMYTDAKVSGSSDEFDIWSYGLGVAFNDLGKEGNVLGLFAGAPPYLAEGDLKTPLQVEGFYKYQLTDGISITPGVIWLKDAAQGVLGEEDAIIGTLRTTFTF

SEQ ID NO: 13 - DNA, 96 bp, Synechococcus sp.
atgaaaaccaatcagcttttaacatccgtaagtcgctctactgccctggcctttctcgcactcaccctaggacttgggggcgaaaaagcactggcc

SEQ ID NO: 14 - DNA, 111 bp, Synechococcus sp.
atgaaatcccagaacgtttttagcaccaaatctgccaagcttattgttggtggtacgatctttgtttcggccattaccgctgccaacttcacaatgctgtcagcctacgca

SEQ ID NO: 15 - DNA, 114 bp, Synechococcus sp.
atgttgcgtcttctctttctccatcgtaagaaagcagcccaagatttccaaggtttcaccgtgattgaactcatgattgtaatgataatcacgggcatcttaacggcgatcgcc

SEQ ID NO: 16 - DNA, 120 bp, Synechococcus sp.
atgaaaaatttcacttttaagcttctgcaacaactcaacaagaagaaagctgacaaaggttttaccctgattgaactgctcgttgtaatcatcatcatcggtattctgtctgctatcgcc

SEQ ID NO: 17 - DNA, 117 bp, Synechococcus sp.
atgtccagttacaaagcgatttgtgtttggttaatacactatagtaagagaaataatcaaggatttaccttgattgaattactcgtcgttatgattatcattggcatcttatcagca

SEQ ID NO: 18 - DNA, 102 bp, Synechococcus sp.
atgattaatcaaccatgcattgttcccgctgaaaaaggctttacgctaattgaactccttacagggatgttgattgtggggattctagcttcaatttcagcc

SEQ ID NO: 19 - DNA, 75 bp, Synechococcus sp.
atgcaactgaaaaaactgtttgtgccactgttggcgggaatgttgttcctggggggaacctctggggcgatcgcc

SEQ ID NO: 20 - DNA, 129 bp, Synechococcus sp.
atgcaactgaaaaaactgtttgtgccactgttggcgggaatgttgttcctggggggaacctctggggcgatcgccgaagaactattgcgcacgatcactgtcacggggcgcggcgaagaagccattgcc

SEQ ID NO: 21 - DNA, 609 bp, Synechococcus sp.
ctcgagtctaccgtggcccaatttaccgatattagtggggatatctaccgcaatgaaattgcccaggcggttaacgtgggttttatcgccgggtttaatgataacacctttcgccccaccgatgtgctcacccgggaacaactcgtcagtatggccattgaaggcctccaggcgctgcccaatgccagcctcgcggtccccacccaagttgccaacgcgccctatcccgatgtggccgcggatcgttggtctgccgcgaaaattacctgggcccaggcgaataacatcgtcagtggctaccccgatggtacctttcaacccacccagcccgtcacccgcgccgaactgttggcggttctgcgtcggaccgccgaatatgcgaaagccgcgcagggtcagcccatgaccttggtcgccaccaacggtcccattgcgttttccgataccgccgggcattgggcgaatgatttggccgcgcaaatgagcacctattgtcgcgttgcctcccccctcaacgaaagcggcgatcgctttttccccgataccgcctctcaacgtaattacgccgcggccgcgaccttgcgtaccctccagtgcagtgtgcggtaa

SEQ ID NO: 22 - DNA, 609 bp, Synechococcus sp.
ctcgaggtcctcgccgcgcccggtctcgttgatcccctgccctacttgcccacctttaccgatgttcaaaatcactgggccaaaccctttattcaggccatcgcgaacctcggctatattcatggttccgcgcaagggcagtttttccccgatcaacccttgaatcgggcccagtttgcgctctggattcaagccatctttcacccctccccccgtcgtccccgcaaacaatttttcgatgtgcccagccatctgcccgccgcggaagccattcaacagggttaccaagggtgtttctttagtggctttcccgatcacacctttcagccccaacagcccctgcgtcgtgttcatctgttggtggccattgcgcaaggtctgcgtttgccccccggtgatatcgccttgaccgaacactatgcggatcaggaagaaattcccccctacgcccaagccgcggtcgccaccgcgctgcaggcgaaaattggtgttttgccccaagaaaaactcatgctgaaaccccaggccatcgcgtcccgggccgaaggtctcgtgtattgccatcaggcgctggtctacggtcaacgtctcctgcccctcaccgaataa

SEQ ID NO: 23 - DNA, 609 bp, Synechococcus sp.
ctcgagctcttttctcagggtcaggtgcaagccctcaatagtccctatatcgtgacccaagatgttgtggccgtcgattaccggattaccgcggggaccattatccccatttcttacaccgccgataaaatcctgttgacccaggatgaaattctgcccgtgaccttgaccgtcgatgcgaatatcgttaacacccaaggcattgtgctcatcccccagggttccgaaattcaaggggaatttcgccccagcggtaatgggacccgctttgtcgcccagcggctggaattgcccaacggtcaaatgtacaatatcaacgccgcgtcccaagttattaccgataccgaaagcgtccgtcgtggtaccgatgttggtaatctcctgcgtaacgccgcgctcggtaccggtgccgcggccgcgattgccgcgatcaccggtgatcgtgccatcgcgaccgaagaattgctcattggtgccggtgcgggtattctcgccaccctgatcccccagtttctcggtctggatcgcgtggatctgttggtcgttgaaaccaataccgatttggatctcaccctggccaatgatttgattctccaagtcaacccctaa

SEQ ID NO: 24 - DNA, 609 bp, Synechococcus sp.
ctcgagtccttgggctacctcgccgatgaagccgcggattctaccgaaagtaatggtctctttaacggcgaatatggtgccctggcgcaaattgcgtttaatctcggggatcgggccgaactgggcgttacctatgtgaactcctaccatgatagcggtgcgatctatgattttggtggtgggagcgccgtcaatggtaccgcctgggcgaacgccctcggtctgtttggtaccgaagccaattcctacggtgttcaggggaaatttgatattaccgatcgcatcagcctcgccgcgtatggcatgtacaccgatgcgaaagtttctggtagttccgatgaatttgatatttggagttatggtctgggggttgcctttaatgatttgggcaaagagggtaacgtgttgggtctctttgcgggtgcccccccctacctggccgaaggtgatctcaaaacccccctgcaagtggaaggcttttataaataccagttgaccgatggtattagtatcacccccggggtgatttggttgaaagatgccgcgcaaggggtcctcggcgaagaagatgccattatcggcaccctccgcaccacctttaccttttaa

SEQ ID NO: 25 - DNA, 591 bp, Synechococcus sp.
gttataaaataaacttaacaaatctatacccacctgtagagaagagtccctgaatatcaaaatggtgggataaaaagctcaaaaaggaaagtaggctgtggttccctaggcaacagtcttccctaccccactggaaactaaaaaaacgagaaaagttcgcaccgaacatcaattgcataattttagccctaaaacataagctgaacgaaactggttgtcttcccttcccaatccaggacaatctgagaatcccctgcaacattacttaacaaaaaagcaggaataaaattaacaagatgtaacagacataagtcccatcaccgttgtataaagttaactgtgggattgcaaaagcattcaagcctaggcgctgagctgtttgagcatcccggtggcccttgtcgctgcctccgtgtttctccctggatttatttaggtaatatctctcataaatccccgggtagttaacgaaagttaatggagatcagtaacaataactctagggtcattactttggactccctcagtttatccgggggaattgtgtttaagaaaatcccaactcataaagtcaagtaggagattaattcc

SEQ ID NO: 26 - DNA, 1000 bp, Synechococcus sp.
ctggccacgaatttttgtaattccacgatgatctttcaacaatccagacacagccgttgcccccgccagcagaataatgcggggattgaccaagcgaatctgctctaataagtagggtatgcaggccgctgcttcaatgggtgttgggacacggttgccagggggacggcacttcacaatgttgcagatataggcatcccgctcgctgtcgagattgaccgaagccaggattttatcgaggagttgccccgctttaccgacaaaggggcggccactttcatcctctgcttggcctggcccttccccaataatcatcagcttggcagcaggattaccgcggctgaccaccacatgggtacgggtggccgctaaaccacagcgttgacactgctgacaatgtaccgcaagggcctccaagttgcggtaggtgccggcgggaatgggcacctcagcccgtagcggaatttgatcgtaggtggcaggatccaggggcgtcgctggtgcaggttcagcttctgtggggctgtcaaataagctaaattgcaacggctcactcatacaaatcgtaacttcctgagaacaatgttaaagaaacttcacaaaaattaggaaaaacttaggacaaactagaccaattttatggcgatcgctagaagcttaatttatctcacaaaagtattttacaaattaataactacggcgaaacaggtttcccaccgcattgtataagaaatacctgaagggtttaacaacacggctgttgtttcccaggcccctctgcggaacaagccatcagcaatcgttaggcctttccggcacgccaagagcgttgcacgtttcttaaaagacacaccaaggatcagcttggtcgctctcgggttgcttggcacagcctttagggagttgctgattagcctccctaaaaatcctgccttatctctgtgggtaggaatgtcaagaaggtctcacttctttaataacctttaaggagaattgatcc

SEQ ID NO: 27 - DNA, 1000 bp, Synechococcus sp.
ttggctaggaaatactgctcaaaggcggacttgagccaactgacccatggcccttgcacggccagagctatagctgctcttgcctgtcttccgaagccgccacctactgctactcgtggctgggtctaggaggggatcccatcctggcaatccagtagccgcactggtgttctccgttcacctgccaggtgaggcgctctacccggcagtcgggcagggcggcggcaaacatctccagctcgtggccgcagacgctgggaaaacgctgggcaatttgggcaatggcgcagtggtgctccaccaatacgtattgctcggcatactgctcagcgccttctgttgccctggccgcagccaggtggttggggtaggggtagtactccgccatgtagccttctgcctggcgcaactgcaccaagcgctccagccgcttggccagggatccgcatcccatctgggcttggtactcttgggctttgcgctgccactgctgctggagcagggatcccatctgttcggatcccagggtttcggccagcgtattgagcaggcccagggcaaactcgtcgtagcttgtggggaattgcgcttccccttgggggctaagctgatagaagtgctgggggcggcccaggccactggcttgagcgacgtggtggatcagcccctccgcctctagatccttgaggtggcggcgaatggcttggggagaaatgcccaagtgctctgccagagtctgggcggtggcctgccccgccttgcgcaaatagactaggatggcctgcttgctggaaaggggctgcgtgaaccgcccggtgatcttgtgcgcgctctccaccctcgccctcctggttagactgggttcactcggctgccgctgccagcacagctactttgacaacgggatcattgctacagtaacctgagggttagttaagcaacaaccgtgttgttttagttggccgtctgtcttgaccctgtccctaaccaagagccatcc

SEQ ID NO: 28 - DNA, 1000 bp, Synechococcus sp.
gtcatcgcaaatggccagttttaccgccgcctcttgattcttgaaattcatgggcaacacccctggggcatccaagagttcaaggacgggggacaagcgcacccagcgcagttgccgcgtcacccccggacgcgctgcactctccaccacccgctgctgcaagaggcgattaatgagggctgatttgcccacattgggaaaccctaaaaccacagctcgtacggctcttacttgcatgccccgctgttgacggcgttgattaatggccgctcccgcccgtaccgccgcctgttcgagtcggcgaatcccttcaccccgctgtgcattggtgaaaaaaaccgtttccccttgggtctcaaaccatgtcagccactgctggcgatcgcgatcgctaatcctatccattccattgagcactaagaggcgctgcttgccactcgcccactgacgaatttggggatggcaactggcaaggggaatgcgtgcatcccgcacctcaaagatcacatccacctgtttcaattgttccttgagggcacgttctgccttggcaatgtggccaggataccattgaatgatcggactcatagaaattccaccccctttttttcaaaaacaatagagcgatcgccccccatgaggaccattaaattgatttcaggcaatcatcagtgcctgttgaatagaagggagactatagttgtacacctctaattagaaacctataaacaactctaaagattgataacccaaacctatagaataattaagaatctccaaagcaacagttattgcattgggtatcaactaagctgatcctaagcataagacctacgacaatatcacaggcttgcggatggttgcatcatctcgctgagttttgcactgctcaattttgccccatttgcggggaatcgtagcgttcacactacccattgcaaaggttgcccgtagagattgctctattcggcacagtcactgttaaagaggaacaacgtct

SEQ ID NO: 29 - DNA, 1000 bp, Synechococcus sp.
tagcgcaaggcgccctggtagcgagcaatttggccttccaggccgtggtggaacatgagcaccacccgctcagggccagggggcaaccggcgaatggccgccgccaactgctcaatcgacttgggggccgccgacccataccactgggatccgatcacccgcaccccacagggcagatccagatagcccccccgcccacctgaccaggggcgcagctctaaaccctcctcgcctgcttccggctccagcaggatcaggtcgccgtgatcggccaggtagcgcagccaactggtcttgacaccgtaggggcggctgtcgtggttgccctcaatggccaagacggggatcccggcctgcttcagctcccgcaagacaatctgggcttggttgaggacaccgggctgaatttgccggtgctcaaacagatccccggcaatgaggacaaaatccaccgggttctggatggcatggcggcgcaccacatcccgaaaggccaaaaagaaatctttgctccgctccgggctgttgtagcggtcgtagcccagatgcacatcggccaggtgcaaaaaggtgcaggtggaagtggccatcgcccgaacctgccagcaaattgcccacagtttagcagacaaacctcagtgctcaactagatatagggatcccttcccggccatttgcggctttgctcgggttgccacccctagagctaggcgccgccccgccgcgctgcctgttgcccagttccaacctttcatgggcacagcttgggttggtgaaagttcgttacatatatttacatctttattggagaaacgattgccaagcaaccctaactccgatagggcaagggatccctggtttaattattgtgaagcgacgggggggttacaaaggctgacctataatgccaggtaaatcccgccttgggagagatccccgagcttcacggctgcagacaggcgggagcgcttcgttccttgtcgcaagagaggagttcttg

SEQ ID NO: 30 - DNA, 238 bp, Synechococcus sp.
gcagtcgtcatgatgttttgagtccagtgaatttttatgtatgtctaaggcgtaatgccttatgagctaataataacaaaactttgcgaattgtgaagcacttctcagatcaaacttggcgatcgccccaacaatcagctgtgatcacctacagtccggcctataccctcgttcccacctacgagtgcttcaatcgctgcacctactgcaacttccggcgcgatcccggaatggacga

SEQ ID NO: 31 - DNA, 1000 bp, Synechococcus sp.
gtggttttgtagctgggtctttctgcactttaccatcaactccatattctgcgccatgacatgggcagacaaatttcttcgcttgggcttgccatgctacggtacagcctttgtggctacaagtaggattgacagcaatcagatttgcgtccttagatgtacccacgaccaacaccgggccaattggtgaattttcgttgagtaattgaccagttttatctagttcagcaacagtcccgatcgcttgcccctctgtagatgttgtgggctgggaagaacaagcagcgatcgctacaggtaagctacttgctatccaacccaaacctacccaattgatgaaatcccgacgtttcatagccactgaagttatgtattagttgtaaacaaaagtctagccttgttttaccaacatttttagctactcattagttaagtgtaatgcagaaaacgcatattctctattaaacttacgcattaatacgagaattttgtagctacttatactattttacctgagatcccgacataaccttagaagtatcgaaatcgttacataaacattcacacaaaccacttgacaaatttagccaatgtaaaagactacagtttctccccggtttagttctagagttaccttcagtgaaacatcggcggcgtgtcagtcattgaagtagcataaatcaattcaaaataccctgcgggaaggctgcgccaacaaaattaaatatttggtttttcactattagagcatcgattcattaatcaaaaaccttaccccccagcccccttcccttgtagggaagtgggagccaaactcccctctccgcgtcggagcgaaaagtctgagcggaggtttcctccgaacagaacttttaaagagagaggggttgggggagaggttctttcaagattactaaattgctatcactagacctcgtagaactagcaaagactacgggtggattgatcttgagcaaaaaaactttatgagaaccagctc

SEQ ID NO: 32 - DNA, 84 bp, Synechococcus sp.
aattctcgagtaacaccgtgcgtgttgactattttacctctggcggtgataatggttgcaggatccttttgctggaggaaaacc

SEQ ID NO: 33 - DNA, 84 bp, Synechococcus sp.
aattctcgagtaacaccgtgcgtgttgactattttacctctggcggtgataatggttgcaggatccttttagtggaggtaaacc

SEQ ID NO: 34 - DNA, 80 bp, Synechococcus sp.
aattcttgacaattaatcatccggctcgtataatgtgtggaattgtgagcggataacaatttcacacaggaaacagaccc

SEQ ID NO: 35 - DNA, 80 bp, Synechococcus sp.
aattcttgacaattaatcatccggctcgtataatgtgtggaattgtgagcggataacaatttcatagtggaggtagaccc

SEQ ID NO: 36 - DNA, 504 bp, Synechococcus sp.
aattcgtgcaggttcagcttctgtggggctgtcaaataagctaaattgcaacggctcactcatacaaatcgtaacttcctgagaacaatgttaaagaaacttcacaaaaattaggaaaaacttaggacaaactagaccaattttatggcgatcgctagaagcttaatttatctcacaaaagtattttacaaattaataactacggcgaaacaggtttcccaccgcattgtataagaaatacctgaagggtttaacaacacggctgttgtttcccaggcccctctgcggaacaagccatcagcaatcgttaggcctttccggcacgccaagagcgttgcacgtttcttaaaagacacaccaaggatcagcttggtcgctctcgggttgcttggcacagcctttagggagttgctgattagcctccctaaaaatcctgccttatctctgtgggtaggaatgtcaagaaggtctcacttctttaataaccttagtggaggtttgacc

SEQ ID NO: 37 - DNA, 365 bp, Synechococcus sp.
aattcgtgcaggttcagcttctgtggggctgtcaaataagctaaattgcaacggctcactcatacaaatcgtaacttcctgagaacaatgttaaagaaacttcacaaaaattaggaaaaacttaggacaaactagaccaattttatggcgatcgctagaagcttaatttatctcacaaaagtattttacaaattaataactacggcgaaacaggtttcccaccgcattgtataagaaatacctgaagggtttaacaacacggctgttgtttcccaggcccctctgcggaacaagccatcagcaatcgttaggcctttccggcacgcctttaataacctttaaggagaattgatccatggccatca

SEQ ID NO: 38 - DNA, 415 bp, Synechococcus sp.
aattcgtgcaggttcagcttctgtggggctgtcaaataagctaaattgcaacggctcactcatacaaatcgtaacttcctgagaacaatgttaaagaaacttcacaaaaattaggaaaaacttaggacaaactagaccaattttatggcgatcgctagaagcttaatttatctcacaaaagtattttacaaattaataactacggcgaaacaggtttcccaccgcattgtataagaaatacctgaagggtttaacaacacggctgttgtttcccaggcccctctgcggaacaagccatcagcaatcgttaggcctttccggcacgccaagagcgttgcacgtttcttaaaagacacaccaaggatcagcttggtcgctttaataacctttaaggagaattgatccatggccatca

SEQ ID NO: 39 - DNA, 420 bp, Synechococcus sp.
ttgggcgatcgccaaaaatcagcatatatacaccaattctaaataagatcttttacaccgctactgcaatcaacctcatcaacaaaattcccctctagcatccctggaggcaaatcctcacctggccatgggttcaaccctgcttaacatttcttaataattttagttgctataaattctcatttatgcccctataataattcgggagtaagtgctaaagattctcaactgctccatcagtggtttgagcttagtcctagggaaagattggcgatcgccgttgtggttaagccagaataggtctcgggtggacagagaacgctttattctttgcctccatggcggcatcccacctaggtttctcggcacttattgccataatttattatttgtcgtctcaattaaggaggcaattctgtg

SEQ ID NO: 40 - DNA, 86 bp, Synechococcus sp.
ctaaatgcgtaaactgcatatgccttcgctgagtgtaatttacgttacaaattttaacgaaacgggaaccctatattgatctctac

SEQ ID NO: 41 - DNA, 124 bp, Synechococcus sp.
catcgcctctgccttttttataacggtctgatcttagcgggggaaggagattttcacctgaatttcataccccctttggcagactgggaaaatcttggacaaattcccaatttgaggtggtgtg

SEQ ID NO: 42 - DNA, 80 bp, Synechococcus sp.
atcgcctttttgggcacggagtagggcgttaccccggcccgttcaaccacaagtccctatagatacaatcgccaagaagt

SEQ ID NO: 43 - DNA, 1020 bp, Synechococcus sp.
atgaattctcaagccgttccatctcccaagtggtggtttcagatcatcttcctctctctgtttttggggggactccaaacaaagcaagcctccgcccaaaccccaggatgctttacgacgaatgtcccctcttcccctctcagctacgatgtcaccagcacaacccaaaccgaaagctacgccgtgacatttcgctgtaccgatgatggcacgaccggaggaagcaacctcagcaatgttgatctagatgtgacgctactgccgctcactgcaccaaccgctggcccggctaatctggatctcggttctccgaatggtgttactcatacgatttcgattggttcgggtggttcctttacgaatctggtcgatactcaaaccaccgtaaataacagtggctcaaccaatttagtcgtgtcaactgctggtggtaaaggcgaaaatctcttcctcgatggtaccggaacgatcacggtgaatatccaatcccgctttgcactccaggggagcacctccgaatttgccgctggcacctacaccacccagtttgaagttgatgtaaccccagttggtgggggcaccactgctgatgaaactaccacaatcagtagtacggtcagccccagttgcgtcctcgataatgtgattcgcttccgggaaacagccaccccctatatcaaaacaggcagtgaacccaatgtttcccagcttcaagccagcgatacagcgaagtttgactgtaatgccaccaccgtcgatatcaactttagtgcagacagcgctacctacacgccgccaacagggggggcaaccaacctgaccgcaacccatcaattcgcctatgaactcaatggcaacggcttcaacaattacagtggcccagagcttattgaaaaccaaaatacagatgacaatggtgatgcaaccttaacgattcgctccacctggacgccgaatagtgatcaactgttcgcttcagaatacaacgcccaaaccactgtcaccattacggctaaataa

SEQ ID NO: 44 - DNA, 804 bp, Synechococcus sp.
atggcttattctgttgtgtcttggcgcaaaaaccttagctgggcgctctgttctttggctttacttttgccactccccctcaacgcccaggtgcaagtctctcccatggtgatcaaaacagaaaccagccaggggatggcgaatggggtgatcagtctaacaaaccagggaacccaatcccagcgggtacgcctctcggcggaatcttttacctatactcgaactggttttgccaccgcagagtccgatccctatgacctcagtccttatttgatgttttcccctagggagttggttctagaacccggccaaacgagacgagtgcgactgattacgcgaatgttgccttcgacggcaaatggtgaatatcggtcggtgatctttgctgaacccctgcgagaacgagatgaagcggggggcggtttgagtattcgggcccgtgtgggggtgacagtttacgtgaaacatggccaggtcaattttgccttgactcccgttgaggcgagctacgatccaacgaaacaagaatttcaacttttggtgagtaaccccagcaatggtacggtgcaatcaaaaggcacctggacattgtcacaaaatgatcagcctttgctccaggcagatattgatcaacgtactgtaattgccggaggcgatcgccttttccccctagagctgcccccagaccggactaacttaccagcgggaacctatcaagtagcagggcagttgcaatggagcgaatctggagcagtgaccacaacaccattttcctttgatgtcacggtgcctgccgcacggtag

SEQ ID NO: 45 - DNA, 2853 bp, Synechococcus sp.
gtggcgcactctaacctgaaaaagtctcacatttttccccgtcgtttagagtatttacccttgacctttcggctactactattcagctttttcatgcttttcctattgggtgctgaggttgttgatgcccaacaggacagcgagcctgctgataatggtgcaacggaaaccacgtcggagactttccctgcatcctttgatttgattccagtggggattaagcttggcgatcgcacggccaatcctggcaccttggttcggggttcagaaaatggcattcaagctattgatttttccaactgggcgatcgcctacaatgatgtcctcaaagcgctccaatttacggcaaccccccttgccgatggcaccatagagttgcggtctccggcggcagtcatcaggctcgatcccagccttctcgatacagatccacaattgggcttggtattcaccgtcacccagatccgcgatctgctacaaattccggtggagtttgatatttctgaatatgctattgttctgacccctgagtggctgagggcatcaggttctttgggattaactgggcgatattccctcccggagcggcccattgtgttggagggtttaccccgcattgaagctccgaatttgtcttttagtgccattggtcaagaagttcgtgtgacaggaggaggcgatcgccccacagaatacgaaggcgatctggttggcatcgggacattttttgggggaagttggtacagcaagattgatcaacgcgatttaaccgatccccgcagttggcaactcgaagaatttcaatacctacgccaaacccctagcaccgactatgtcatcggcgatcagcgtaccttttggccagagggcagtggtcgctatacaggtgtcagtatcgtgcgccgctttggctttcaacctcccaccgaatttaccaatgccagcgatggctttaatccccaacaacgtctcaatagcgatcgcctagagcgtgatatccgaggccgcgccgaaccagggaccctcgttcaactggtcaataaaaatggcaatttaattgttggggaacaactcgttgatcagtctggcatctatcgctttgaaaatattcccagtgcttctaccaataaaggcagaggcggcatagccggtaatcgctacgaacttcgactttatcccaatggtcaactgagtgccttcccagaaattcgggccgctgaattttcttctctgcctgggcagctgagtaaaggtacctcagccctcctcctctctgctggctttgaacggcttcgacaagcggatactttttttggctccctctcgaatgatctccagggaggattcgcctaccgttggggcgccacggacaatctcaccctcggtacgggtcttttttacgacggtcaacttaaaggtctaggggaatttttctttcagcccggtcgattgcccctgcgaattactggggcagcaacctttaatagcgacgaacaacggggagaacaacaatctgatttccgctacgatctaaatgtccgcttcaatccaggccagaggtttgattttgagtttgacaaagatgaactgtctgagcgcattcgcacccgctgggatgtcagtgacaaatttcgtcttgccttcaacagtaacagcagcgatcaaatcgcccaggccacttggcggctttttccgggttttagtacgcgggttggttggagctttaacaataaagccctggaaggtggattcgacctcagtggtgcccttggggatcttttaattcgcaatagcgtaacctttagtgccgaccaaagccttgattggcgattgttttcccgctatcaaaacctcaccctagaccaccggctgcgtgaccgtcagattgcaacggaagtagagtattttttccgtaatcctgaagccctggtggatacgggtcactcggtctttgcgcgctaccaaagtagccccaacgaggacaacgaggcccggacgaacgagctgctcgtggcagggtggcgctatgaggcaaattcgacggtgggcgatcgcctttccgactggatcgtcgatcttggctatggcgttggcacccagggagcaggatggcaaattgctgtaaccaccaatcagctcttgggcctgaatctaaccgcgcggtaccaagatatttctcttacgggtaatgagtcaagcttcagcctcctcattggttccgatgcaatcctttcacctaatttcagcctaaaacccagtcgctttgaacgtttacgaacagagggtggcattgtggtgatcccttttatcgatgccaatcggaatggtgtccaagatgaaacagaaacggcctatttgcaagggattgaggcggaaaccgcagactttttattcttgattaacgaacagcccattaaccgctttagtgaatatgagccggatttgcgacggagaggaatctttgtgcgactgccaccggatacctatcgcttcgatgtagatccggcgggcttgcccctgggctggcagacaacgcagtcggcctttgcagtagaagtgagtgctggtagttacacgcctatttatgtgccccttacccgtgcctacattgtcgcgggcacggtggtcaatgcccaagggaaaccactgggtggggtaagggtcgaagcagtcaaccaaaacaacccccaggagcgatcattgtcggtgaccaatggcgcaggtatctactatctagaatccgtaggaactggtgtctatgacctattcatcgatggcaaacccgctaaaccgggccagctccgcattgagatagatgctgaagaatttacagaattggatttgcgcctgtaa

SEQ ID NO: 46 - DNA, 1224 bp, Synechococcus sp.
atgttagatctaatcaaacttgcgggacaactgccagacatgggggcgcacctccaggaacaggctgtcacgggacgagaacgaatcgagcggggaatttctctgctccgggaagcccaggcggatttccagaccctccaggcccaccaaaatacctggggcgatcgcctcatttttaaccatggcattcccctcgaacccctggagactcgcgttcccatttcgcccccttcccaagcccacaccgtttttgccacggatggctcccaaattgctccgtctcaccatgaaattgcctattgttatttgattaatattggtcgagtgatgctccactacggccaaagcttgcacccattgctggatcatctgccggagattttctatcgcagcgaagatctgtacacctcccgcaaatggggcatccgcaccgatgaatggctcggttatcgccgcaccgcctccgaagctgaagtgctcgctgagatggcctgtaaatgggtgttaccccccggtgcccacggtcatattcccaatgtggcgatggtggatggctctctggtctattggtttttagaaaatttgcccgccgaagcccgccaacaaattctcgaacccctcctaggggcctggcaacaactccgagaaacccgtattccgctgattggctacattagttccacccgcagtgtagaggcggttcatttcctgcggctccaggcttgcccccacgacaaacccgattgtcaaagccattgcctcgacggcgaaaccaaggaacgtaaagcagaatttcgcgaaactcttccctgccaaaccattgaaccgttgcgggatagcactctttttgagcaactgttgcaaccgggcgatcgcagtgggctttggctcagtcaggcacgcattttaaatcattatccagaagcggatcaggtttgtttttgttatctccatgtggggacggaggtggcgcggatcgagatgccccgctgggtcgcggcagatcctcaactcctcgatcaaaccctaggcattgtcctcggccaagtgcaaaaggggtttgggtatcccgtggcgatcgccgaagcccataatcaagctgtgatccggggtggcgatcgcgcccgattttttgcgctcctcgaacaacaactcctcaaagcagggttaaccaacgtaggtatctcttacaaagaaacccgcaaacggggttccgtggcttaa

SEQ ID NO: 47 - DNA, 636 bp, Synechococcus sp.
atgcccgaaatgcccgaaaactctcaatttcccgttgaaccgccccagaaacccagtggcacggagcaacagcatgaagaaaatccctgggtagagaccatcaagacccttgtgaccgctggtattttggccattgggatccgcactttcgtcgccgaggcccgctacattccctccgagtcgatgctgccgaccctagaagtgaacgatcgcctaatcattgaaaaaatcagctatcacttcaaaaatccccaacggggagatgtggtggtctttaacccgacagaaattctccagcagcaaaactatcgggatgcttttattaagcgggtgatcgggattcccggggataccgtacaagtcagcggcggcaccgtttttatcaatggggaagccctcgaagaagactatatcaacgaagccccagaatatgactacggccccgtgacgattccagaagatcactacctcgtccttggcgataaccgcaacaatagctatgattcccactattggggttttgtcccccgtgaaaagcttgtggggaaagcctttattcgtttttggccctttaatcgcgtgggcatcctcaacgaagagccgcaatttgccgacgaagaaccgattacaccctag

SEQ ID NO: 48 - DNA, 765 bp, Synechococcus sp.
atgagtgaaccatcccctttgctgcaagcatcaggtctccataaaagtttcggtggcatccgtgcggtgcaaaatgcttcgattacggtgccccgcggacagattacggggttgattggccccaatggggcgggcaaaacgacgttgtttaatttgctctcgaattttattacgccggatcgggggacagttatttttaacggccaggaagtgcagcatttaccgtctcaccagattgcggcacggggttttgtgcgtaccttccaagtggcacgggtgttatcgcggttatcggtactagacaatatgttgctggcggcccaacagcaaacgggggaaaacttcctgcgggtgtggcaacaggggaaaattcgtcgccaagaaaaggcaaatcgggaaaaggcgatcgccatcttagaatccgtcggtctagggaaaaaagcccaggattacgctggtgccctgtcggggggacaacgcaaactcctggaaatggccagggctttgatgagcgatccccagttaattttgttggatgagcctgcggcgggcgtgaatcccactttgatcaaccaaatttgtgaacacattgtccgctggaaccagcagggaatttcttttttgatcattgagcacaatatggatgtgatcatgtccttgtgtaaccacatctgggtactggcagaggggagcaatttggcggacggaacccccgaagatatccagtgtaatgaacaggttttagaggcttatttgggatcgtaa

SEQ ID NO: 49 - DNA, 684 bp, Synechococcus sp.
atgcgcgttttattaacaaatgacgacgggattgatgcccctgggattgcaaccttacaaaaggcgatctccccccatgcgagagaagtagtgacggtggccccccaaacacagatgtcggaatgtggccatcggtttacggtttatgctcccattccggtggagcaacggacgaaaaatgcctatgcggtggcaggtacgccagcagattgtacacgcttgggtctcacgcagtttgcggcagatgttgattgggtgctgtcgggggtaaatgcagggggaaacctcggcgtggatatttacacttcaggaacggtggcggcggtgcgggaagcgacaatcctcggtaagcgggcgatcgccttttcccatttcatccagcggcctttagagattgactgggatcttgtcacccactggacggggaaacttttggcgcaattattgacccaggaactaccggaaaagcatttttggaatgtgaattttccccatttaacgggagactctgacccggaaattattttctgtgagcgcagcaccgacccgatgcaagtgcgctatgaagcacgggatcaacagttccattatgtcggttcctaccctgagcgcccccgggccgctggtaccgatgtggatgtctgtttttcagggaatattgccgtaacccaaatttcgatctag

SEQ ID NO: 50 - protein, 339 aa, Synechococcus sp.
MNSQAVPSPKWWFQIIFLSLFLGGLQTKQASAQTPGCFTTNVPSSPLSYDVTSTTQTESYAVTFRCTDDGTTGGSNLSNVDLDVTLLPLTAPTAGPANLDLGSPNGVTHTISIGSGGSFTNLVDTQTTVNNSGSTNLVVSTAGGKGENLFLDGTGTITVNIQSRFALQGSTSEFAAGTYTTQFEVDVTPVGGGTTADETTTISSTVSPSCVLDNVIRFRETATPYIKTGSEPNVSQLQASDTAKFDCNATTVDINFSADSATYTPPTGGATNLTATHQFAYELNGNGFNNYSGPELIENQNTDDNGDATLTIRSTWTPNSDQLFASEYNAQTTVTITAK

SEQ ID NO: 51 - protein, 267 aa, Synechococcus sp.
MAYSVVSWRKNLSWALCSLALLLPLPLNAQVQVSPMVIKTETSQGMANGVISLTNQGTQSQRVRLSAESFTYTRTGFATAESDPYDLSPYLMFSPRELVLEPGQTRRVRLITRMLPSTANGEYRSVIFAEPLRERDEAGGGLSIRARVGVTVYVKHGQVNFALTPVEASYDPTKQEFQLLVSNPSNGTVQSKGTWTLSQNDQPLLQADIDQRTVIAGGDRLFPLELPPDRTNLPAGTYQVAGQLQWSESGAVTTTPFSFDVTVPAAR

SEQ ID NO: 52 - protein, 950 aa, Synechococcus sp.
VAHSNLKKSHIFPRRLEYLPLTFRLLLFSFFMLFLLGAEVVDAQQDSEPADNGATETTSETFPASFDLIPVGIKLGDRTANPGTLVRGSENGIQAIDFSNWAIAYNDVLKALQFTATPLADGTIELRSPAAVIRLDPSLLDTDPQLGLVFTVTQIRDLLQIPVEFDISEYAIVLTPEWLRASGSLGLTGRYSLPERPIVLEGLPRIEAPNLSFSAIGQEVRVTGGGDRPTEYEGDLVGIGTFFGGSWYSKIDQRDLTDPRSWQLEEFQYLRQTPSTDYVIGDQRTFWPEGSGRYTGVSIVRRFGFQPPTEFTNASDGFNPQQRLNSDRLERDIRGRAEPGTLVQLVNKNGNLIVGEQLVDQSGIYRFENIPSASTNKGRGGIAGNRYELRLYPNGQLSAFPEIRAAEFSSLPGQLSKGTSALLLSAGFERLRQADTFFGSLSNDLQGGFAYRWGATDNLTLGTGLFYDGQLKGLGEFFFQPGRLPLRITGAATFNSDEQRGEQQSDFRYDLNVRFNPGQRFDFEFDKDELSERIRTRWDVSDKFRLAFNSNSSDQIAQATWRLFPGFSTRVGWSFNNKALEGGFDLSGALGDLLIRNSVTFSADQSLDWRLFSRYQNLTLDHRLRDRQIATEVEYFFRNPEALVDTGHSVFARYQSSPNEDNEARTNELLVAGWRYEANSTVGDRLSDWIVDLGYGVGTQGAGWQIAVTTNQLLGLNLTARYQDISLTGNESSFSLLIGSDAILSPNFSLKPSRFERLRTEGGIVVIPFIDANRNGVQDETETAYLQGIEAETADFLFLINEQPINRFSEYEPDLRRRGIFVRLPPDTYRFDVDPAGLPLGWQTTQSAFAVEVSAGSYTPIYVPLTRAYIVAGTVVNAQGKPLGGVRVEAVNQNNPQERSLSVTNGAGIYYLESVGTGVYDLFIDGKPAKPGQLRIEIDAEEFTELDLRL

SEQ ID NO: 53 - protein, 407 aa, Synechococcus sp.
MLDLIKLAGQLPDMGAHLQEQAVTGRERIERGISLLREAQADFQTLQAHQNTWGDRLIFNHGIPLEPLETRVPISPPSQAHTVFATDGSQIAPSHHEIAYCYLINIGRVMLHYGQSLHPLLDHLPEIFYRSEDLYTSRKWGIRTDEWLGYRRTASEAEVLAEMACKWVLPPGAHGHIPNVAMVDGSLVYWFLENLPAEARQQILEPLLGAWQQLRETRIPLIGYISSTRSVEAVHFLRLQACPHDKPDCQSHCLDGETKERKAEFRETLPCQTIEPLRDSTLFEQLLQPGDRSGLWLSQARILNHYPEADQVCFCYLHVGTEVARIEMPRWVAADPQLLDQTLGIVLGQVQKGFGYPVAIAEAHNQAVIRGGDRARFFALLEQQLLKAGLTNVGISYKETRKRGSVA

SEQ ID NO: 54 - protein, 211 aa, Synechococcus sp.
MPEMPENSQFPVEPPQKPSGTEQQHEENPWVETIKTLVTAGILAIGIRTFVAEARYIPSESMLPTLEVNDRLIIEKISYHFKNPQRGDVVVFNPTEILQQQNYRDAFIKRVIGIPGDTVQVSGGTVFINGEALEEDYINEAPEYDYGPVTIPEDHYLVLGDNRNNSYDSHYWGFVPREKLVGKAFIRFWPFNRVGILNEEPQFADEEPITP

SEQ ID NO: 55 - protein, 254 aa, Synechococcus sp.
MSEPSPLLQASGLHKSFGGIRAVQNASITVPRGQITGLIGPNGAGKTTLFNLLSNFITPDRGTVIFNGQEVQHLPSHQIAARGFVRTFQVARVLSRLSVLDNMLLAAQQQTGENFLRVWQQGKIRRQEKANREKAIAILESVGLGKKAQDYAGALSGGQRKLLEMARALMSDPQLILLDEPAAGVNPTLINQICEHIVRWNQQGISFLIIEHNMDVIMSLCNHIWVLAEGSNLADGTPEDIQCNEQVLEAYLGS

SEQ ID NO: 56 - protein, 227 aa, Synechococcus sp.
MRVLLTNDDGIDAPGIATLQKAISPHAREVVTVAPQTQMSECGHRFTVYAPIPVEQRTKNAYAVAGTPADCTRLGLTQFAADVDWVLSGVNAGGNLGVDIYTSGTVAAVREATILGKRAIAFSHFIQRPLEIDWDLVTHWTGKLLAQLLTQELPEKHFWNVNFPHLTGDSDPEIIFCERSTDPMQVRYEARDQQFHYVGSYPERPRAAGTDVDVCFSGNIAVTQISI

SEQ ID NO: 57 - protein, 261 aa, Synechococcus sp.
MKTNQLLTSVSRSTALAFLALTLGLGGEKALAQWQPTISVPEFKNETNGSYWWWNSSTSQELADALSNELTATGNFRVVERQNLGAVLSEQELAELGIVRPETGAQRGQVTGAQYIVLGQITSYEEGVKEESTGFGLSGIRIGGVRLGGGGRGSSEEAYVAVDLRVVDSTTGEVLYARTVEGKAKSDSTSGGATASFAGINLGGDRTETNRAPVGQALRAALIEATDYLSCVMVEQNGCMAEYEAKDERRRENTRSVLDLF

SEQ ID NO: 58 - protein, 194 aa, Synechococcus sp.
MKSQNVFSTKSAKLIVGGTIFVSAITAANFTMLSAYAVDDTASFSGTVAPACALSNDDGAVAFDAGDRTYTATGSGVDVTELSETQYVDFECNTDTATVAIAAPVTSKPMAPTNASGLVATHVAKYAVDDTDTLVNPDPTSGTIINEATGVAGFSQAVNATGLFRVGVESKWSGANGMLAGDYSADITVTVTPN

SEQ ID NO: 59 - protein, 180 aa, Synechococcus sp.
MLRLLFLHRKKAAQDFQGFTVIELMIVMIITGILTAIALPAFLNQVDKSRYAKARLQMRCMLQELKVYRLNHGSYPPDQNRNVPYYPGSECFKVHTGYVRDRPDINRNNNTDIPFHSVYDYERWDYNSGCYIAVTFFGKNGLRRFTQAAINEISTTGFHFYDGTDDDLVLVVDITDSPCD

SEQ ID NO: 60 - protein, 144 aa, Synechococcus sp.
MSESLRLRYLQYLAQRKDEQGEEEKGFTLVELLVVIIIVGILAAVALPNLLAQTDKAYASEGKSAVGAALRTLSAATLDPNYVTNASCTQLGIGSSAGNFDLTCGNASQVTAAGSGKAANINVTGTIGTDGKFTVIATKGSATL

SEQ ID NO: 61 - protein, 144 aa, Synechococcus sp.
MSDSLRLRYLQYLAQRKDEQGEEEKGFTLVELLVVIIIVGILAAVALPNLLDQTDKAYASEGKSAVGAALRTLSAATLDPNYVTNASCTQLGIGSSAGNFNITCGNASQVTAAGSGKAANINVTGTIGTDGKFTVIATKGSATL

SEQ ID NO: 62 - protein, 144 aa, Synechococcus sp.; identical to SEQ ID NO: 60

SEQ ID NO: 63 - protein, 369 aa, Synechococcus sp.
MALEYMIEDLMEQLVEMGGSDMHIQAGAPVYFRVSGKLEPINEEVLTPQESQKLIFSMLNNSQRKELEQNWELDCSYGVKGLARFRINVYKERGCYAACLRALSSKIPNFEQLGLPNIVREMAERPRGLILVTGQTGSGKTTTLAAILDLINRTRAEHILTIEDPIEYVFPNVRSLFHQRQRGEDTKSFSNALRAALREDPDIVLVGELRDLETIALAITAAETGHLVFGTLHTNSAAGTIDRMLDVFPANQQAQIRAMLSNSLLAVFAQNLVKKKSPKPGEFGRALVQEIMVITPAIANLIREGKAAQIYSAIQTGAKLGMQTMEQGLATLVVSGVISLEEGLAKSGKPDELQRLIGGMTPQVAAKRR

SEQ ID NO: 64 - protein, 234 aa, Synechococcus sp.
MQLKKLFVPLLAGMLFLGGTSGAIAEELLRTITVTGRGEEAIATSLSEVRLGVEVRGATATQVQADIAKRSNQVVDFLKSKNVAKLTTTGINLQPEYDYNNGDRRLIGYLATNTVSFEVPTAQAGSLMDEAVKAGATRIDGISFRATEAALTEAEKTALAEAAQDARTQAQTVLGALGLSPQEIVQIQVNGATPPTPIFKTMDTARIALESAAPSPVEGGEQTVNASVTLTIRY

SEQ ID NO: 65 - protein, 261 aa, Synechococcus sp.
MKTNQLLTSVSRSTALAFLALTLGLGGEKALAQWQPTISVPEFKNETNGSYWWWNSSTSQELADALSNELTATGNFRVVERQNLGAVLSEQELAELGIVRPETGAQRGQVTGAQYIVLGQITSYEEGVKEESTGFGLSGIRIGGVRLGGGGRGSSEEAYVAVDLRVVDSTTGEVLYARTIEGQAKSDSTSGGATASFAGINLGGDRTETNRAPVGQALRAALIEATDYLSCVMVEQNGCMAEYEAKDERRRENTQSVLDLF

SEQ ID NO: 66 - DNA, 786 bp, Synechococcus sp.
atgaaaaccaatcagcttttaacatccgtaagtcgctctactgccctggcctttctcgcactcaccctaggacttgggggcgaaaaagcactggcccagtggcaaccgactatttctgtcccagaatttaaaaacgaaaccaatggcagctattggtggtggaacagcagcacctcccaagaactggccgatgccctcagcaatgagcttactgccactggcaacttccgcgttgttgaacggcaaaacctaggggccgtcctgtcagaacaggaattagctgaattgggaattgttcgcccagaaacgggagcccaacggggccaagtcacaggggcgcaatacatcgtgctcggtcagatcacctcctacgaagaaggggtcaaggaagaatcgactggctttgggctcagtggtattcggatcggtggcgtccggctcggcggtggtggccgtggctctagtgaagaagcctacgttgccgtggatctacgggttgttgactcaaccactggggaagtgctctatgcgcgtaccgttgaaggaaaggcaaagtctgattcgacttccggaggtgcaacggctagttttgctgggattaatcttggtggcgatcgcaccgaaacaaatcgcgctcccgttggccaagcgctccgggcggccttgattgaagccactgattatctcagttgtgtgatggtcgaacaaaatggctgcatggctgaatatgaagcgaaggacgagcgccgtcgggaaaatacccggagtgtccttgatcttttctag

SEQ ID NO: 67 - DNA, 585 bp, Synechococcus sp.
atgaaatcccagaacgtttttagcaccaaatctgccaagcttattgttggtggtacgatctttgtttcggccattaccgctgccaacttcacaatgctgtcagcctacgcagttgatgacaccgcttctttttcgggtacggtcgctccagcttgtgcactctccaacgatgatggtgcagtagcatttgatgccggcgacagaacttatacagccacaggtagtggcgtagatgtcactgagctttctgaaactcagtatgttgattttgaatgtaataccgacactgctactgttgcgatcgctgcacctgttacttcaaaaccaatggctcctacaaatgcaagtggcttagttgccactcatgttgctaaatatgcggtagacgatactgatactcttgtaaatccagatccaacgtctggtacgatcattaatgaggctactggcgttgctggattttctcaagcagtaaatgcaactggcttatttagagtgggtgttgaatctaaatggagcggagctaatggaatgttagccggggactattctgctgatatcactgtaacagtgactcctaactaa

SEQ ID NO: 68 - DNA, 543 bp, Synechococcus sp.
atgttgcgtcttctctttctccatcgtaagaaagcagcccaagatttccaaggtttcaccgtgattgaactcatgattgtaatgataatcacgggcatcttaacggcgatcgccttgcctgcctttttaaatcaagtggacaagtcccgatatgctaaagcgcggctgcaaatgcgctgtatgcttcaagagctcaaagtttatcgcctgaatcacggcagttaccccccggatcaaaatcgaaatgttccttactatcctgggtctgagtgttttaaggtacatacagggtatgttagggatagaccggatatcaatcgaaataataatacagatattccatttcattctgtctatgattatgaacgctgggattacaattctggctgttatattgcggtgacattttttggcaaaaatggtctgagaagatttactcaagctgccattaatgaaatatccaccactggatttcatttctatgatggaactgatgatgatttggtcttggttgtggatattactgatagtccctgtgattaa

SEQ ID NO: 69 - DNA, 435 bp, Synechococcus sp.
atgtctgaatcgctccgtctacgttatctgcaatatcttgcccagcgtaaagacgaacaaggtgaagaagaaaaaggtttcacccttgtcgagttgctggtcgttatcatcatcgttggcatcttggcagcagttgcattaccgaacctgttggctcaaacagataaagcctacgcctctgaaggtaaatcagcagtcggtgctgctcttcgtacccttagtgcggcgacactagaccctaactacgtcaccaatgcgtcttgtacacagcttggtattggtagcagtgcaggtaactttgacctaacttgtggcaatgctagccaagtaacggctgcgggaagtggtaaagcagcgaatattaacgtgactggcacaatcgggacagacggtaagtttaccgttattgcaaccaaaggcagcgcaactctttaa

SEQ ID NO: 70 - DNA, 435 bp, Synechococcus sp.
atgtctgactccctccgtcttcgttatctacaatatcttgcccagcgtaaagacgaacaaggtgaagaagaaaaaggttttacccttgtcgagttgctggtcgttatcattatcgttggcatcttggcagcagttgcattaccgaacctgttggatcaaacagataaagcctatgcctctgaaggcaaatcagcagtcggtgctgctcttcgtacccttagtgcggcgacactagatcctaactacgtcaccaatgcgtcttgtacacagcttggtattggtagcagtgcaggtaactttaacataacttgcggcaatgctagccaagtaacggctgctggaagtggtaaagcagcgaatattaacgtgactggcacaatcgggacagacggtaaatttaccgttattgcaaccaaaggcagcgcaactctttaa

SEQ ID NO: 71 - DNA, 435 bp, Synechococcus sp.; identical to SEQ ID NO: 69

SEQ ID NO: 72 - DNA, 1110 bp, Synechococcus sp.
atggctttggaatacatgatcgaagacctcatggagcagttggtggaaatgggcggctccgatatgcacattcaagcgggggcaccggtttatttccgggtgagcggcaaattagaaccgattaacgaggaagttttaactccccaggaaagccaaaagttaatcttcagcatgctgaacaattcccaacggaaagaactagaacaaaattgggaattggactgttcctatggcgtgaaaggtttagctcgtttccggattaacgtttacaaagaacggggttgttatgccgcctgtttacgggccctttcttctaaaattcccaactttgaacaattgggactgcccaacattgtgcgggaaatggcggaacgcccccggggactaattctagtgacgggacaaactggctccggtaaaaccaccactttggcagcaattttagacttaattaaccgcaccagggccgaacatattctcaccatcgaagatccgatcgagtatgtgtttcccaacgtgcgcagtctttttcaccagcggcaacggggggaagatacgaaaagtttctccaatgctctgcgggcagcgttacgggaagatccggacattgtactggtgggagaattgcgggatttggaaaccattgcccttgccatcactgcggcagaaaccggacacttggtttttggcactctccacaccaactcagcagcgggcaccattgaccggatgttggatgtgtttccggctaaccaacaggcccaaattagagccatgttatccaactctttactagcggtatttgcccaaaacttagtcaagaaaaagtcccccaaacccggggagtttggccgggccctagtgcaggaaattatggtcattaccccggcgatcgccaacctaattcgggaaggcaaagcggcccagatttattccgccattcaaaccggagcaaaactaggtatgcagaccatggaacagggcctggccacgttggtggtgtcgggggtaatttccctggaagaaggtttagctaagagtggtaagccggacgagctacagcgcttaatcggtggcatgaccccccaggttgccgctaaacgtcgttag

SEQ ID NO: 73 - DNA, 705 bp, Synechococcus sp.
atgcaactgaaaaaactgtttgtgccactgttggcgggaatgttgttcctggggggaacctctggggcgatcgccgaagaactattgcgcacgatcactgtcacggggcgcggcgaagaagccattgccacgagtctttctgaagtacgccttggggtcgaggtgcggggggcgacggcaacccaagtccaggcagatatcgccaagcgcagtaaccaagtggtggattttctcaagtccaaaaatgtggccaagctcaccaccacgggcattaacctccagccggaatatgactacaacaatggcgatcgccgcctcatcggttatctcgctaccaatacagtgagctttgaggtgcccaccgcccaagccgggagcctgatggatgaagctgtcaaagccggagcaacccgcattgatgggatttctttccgagccaccgaagccgccctcactgaagcagaaaaaactgccctcgctgaagccgcccaggatgcgcgcacccaggcccaaactgtcctcggtgccttgggtttgagtccccaagaaattgtccaaatccaggtcaatggggcgacgccgccaacccccatttttaaaaccatggatacggcacgaatcgcccttgaaagtgcagcaccttctccggtagaagggggtgaacagacggtgaatgcttccgtaaccctgacgatccgttactaa

SEQ ID NO: 74 - DNA, 794 bp, Synechococcus sp.
atgaaaaccaatcagcttttaacatccgtaagtcgctctactgccctggcctttctcgcacttaccctaggacttgggggcgaaaaagcactggcccagtggcaaccgactatttctgtcccagaatttaaaaacgaaaccaatggcagctattggtggtggaacagcagcacctcccaagaactagccgatgccctcagcaatgagcttactgccactggcaacttccgcgtcgttgaacggcaaaacctaggggccgtcctgtcagaacaggaattagctgaattgggaattgttcgcccagaaacgggagcccaacggggccaagtcacaggggcgcaatacatcgtgctcggtcagatcacctcctacgaagaaggggtcaaggaagaatcgactggctttgggctcagtggtattcggatcggtggcgtccggctcggcggtggtggccgtggctctagtgaagaagcctacgttgccgtggatctacgggttgttgactctaccactggggaagttctctatgcgcgtaccattgaaggacaggcaaaatctgattcgacttccggaggtgcaacagcgagttttgctggcattaatcttggtggcgatcgcaccgaaacaaatcgcgctcccgttggccaagcgctccgggcggccttgattgaagccactgattatctcagttgtgtgatggtcgaacaaaatggctgcatggccgaatatgaagcgaaggacgagcgccgccgggaaaatacccagagtgtccttgatcttttctagaccgttga

SEQ ID NO: 75 - DNA, 12002 bp, Artificial Sequence
  Description of Artificial Sequence Synthetic polynucleotide
ccagccgcttgccagccgttatcagtaaagttaatctctgtgccagctcccagatcaattaaagggacaaaagcaaattcgtcagggttatcaaaattaaaaccgatgatagcaatatcacctgcggacaaaatcgtagccgtcacgttaaattctccctttgaatttgaattaagttaaatcgggtaaatcggaaatgttttgttaatttgagaaaagccatgattaatgaaaaatcctcatgcatcattgcatttgttaatttgcaagtctgagggaatcgcctcacaatacaaggacttcaaagttgcatgccgattcttctaatcactcatctgccaaaatgttcatcgaagttaggaggtagttttaggtatcctaaattcgacgattatatttggggtcaatatgatctatgctgatgaaactaataacatcgagctctcaaaaaatgccgctaatagaagcggttcgaaatagtgacgctatctttcgtaccatcaacattgttcaaattgacacctaaattgaaattttgccttaaagaaagctacagattttgagctgagttaggtttagttgtcatacactttaaaaacttatccaacaaaataatctcctagtttcctaaagaaacagtaaagcctgccttaaatcttggagaaaatacgcggaaatcccaggaaaatacttaggttttctcagcgactgcttataaggtaaagataaagagacaggatgtcttgcttctatatttagtaaggtcaagcaaaagtatgacgagaacacaataggttacaattttactggatggcaaagtatattttggtttgtgagaaataatttagttttctctaaatgtatagtcaatcacattagattactgtggaattttatccatcaccataattcattatcaatccccccctttagtattatcttaaataaagttttacttttaattttttaatcccacagcatttttatggtgattagggtgcaatttggggtttgcatgacttatagctaattcagggatattgccaaaagtctattctgttgactccaaacaaaagtttgttcagtctcgatggaagtaatttaaacttgtgcgatcaccttcaaaagctaaatttttcccgaagcatgacgatcaacaaaatagactgtatttctcagaaatggttttacagtggagagtgtccaacttttcaaagattactctatgattaccgttcctgggggaactatcaaaccccattggtttgtccgtcgtgatctcgatggtttttttggcctcgcactgaataattttgtccaaatcctagttattgttagtctgactcaaggggtgctgcaatttccggtcgaactgctttatggccgcattttgccaggtagtgcccttagtttaatcgtcggcaatgcttattacagttggctcgcctataagcagggttgcgcagaacagcgggacgatattgctgcccttccctatggcatcaataccatcagtttatttgcttatatttttctcgtgatgttgcctgtccgtctccaggcgatcgccaccgtcgccgacatcctgaaaaaaacaaaaattctgctgattaactttccaaccctttctttatcaacctcaatccccttaaaaatcatgattcaagagattgccactagcattcgtactacagtctttctttggttacttactgctttgatttaccctttgctgattttcatgatcggtcagggcctttttccaatccaagccaacggcagtctaattgtgaataaccaagggcaagtcattggctctagcctcatcggtcaagcctttaatagcgaagactatttctggggtcgccctagcgcagtcaattacagtatcggcgaagatgccgcccctaccggactatccggagcgacgaacctagctcccagtaatcctgacctgttggcattggttaaggaaagagccgaaatcctgcgggctgccgatcttgagccgactgctgacctgctctacagttctggctctggcctcgacccccatatttctcctactagtgcgatcgctcaaatcgaccggattgccgccgctcgtaatctttcccctgatgacttaaaaatcttaattgaacagaataccgaaggacgattcttaggaatttttggtgaacctgctgtcaacacagtcacccttaatttggcccttgatcagctctaaagctttgaaaacaggaactttctagcctgcatattctctgactatcctcttgctcttttagatgtacaaccctactgcgatcgccgctactaactaccaaatactcccccgtcgaggcaaacacaaaatttttattggcatggcccctggagttggtaaaacataccgaatgctcgaagaaggacatgcccttaagcaagatggtgttgatgtggctattggcttattagaagcccatgggcgggaggaaacaaccctgaaagctgaaggcttagagctaattcctcgtaaacaagtttattgtcgaaatgttttattgcaagaaatggataccgaggaaatcttaaggcgatcgccccagttagtcctgatagatgaactagcccacacgaatatacccggctctaaacaagaaaaacgttatcaagatgttgaaaaaattctaaatgccggaatcgatgtctattcaacggttaatattcagcatctagaaagcttaaatgatctcgtatatcgcatttctggggttgtagtaagagaacgtattcccgaccgaatcattgatgatgctgatgaaattgttgtagttgatgtaaccccagaaacattacaagaacgacttcaggaaggaaaaatttatgcgcctgaaaaaattgatcaagctctacaaaacttttttcgtcgcagtaatttaattgctttaagagaattagcgctacgtgaaattgccgataacattgaagaagaatccatttctgaaactaaaaatctccattacaatattcgagaacgggttttagtatgtatttcaacttacccaaattctattcagcttttacgccggggcgctcgaattgccaatcatatgaatggacaattgtttgtgttatttgtagcacctgcgggtaaatttttatcaaaatcagaagcattacatatcgagacatgcaaacgattgtgtcaagaatttacaggggaattcttaaggatagaatcaccgaatattgttaccacaatcgtcaaaattgcaacccaagaacgtattactcaaattgttttaggggaaactcgtcgctcccgatttcaattattattcaaaggctctattgtacagcagctaatgagagcattaccccaagttgatatacatatcattgccaataattaaaattcaactaactggacaagagaacgcacttaaacagcaagcatcgattataaaaagtatcgtctcaatcctctattcagaagatatcctgatgaaagcaaagccgtaaagattgctcgaaaattcaagcatctccccccaaaggcggcgtttttttatgtttttatgagagtccaagataagggacaagataaatttgaattccggggacggacgggggcgatcgcataacctctactgcttctaatgattttgatgatcaaaaaaatgaagaagatcaataaccatgaccttgcctccaggatccgttctccaatttttttaacctgcggaatgtaggaattagggcagcgcgaaagtacgggtatgtaaattattttgtgcaagcggtcaaaatcaataaatcagtaacggtgtttatttagcaatggacccgttttggtcgctaaggctaaaaccattgatatataaacttttcaagcatgagtataataactcattaaaagttcgactttgacaaaccgtattcttcagcaaatatataactgtcccatttatgatgcttagagcaataacccaaaaagtctagaagtcttataaatagtgggtttggcaagaataattgtcccatttaaacatagaagtgggacagtattctgactttctaggaactcaaaagttgacctccagaattgcttagtgagcaaaacggatccattgctaaataaacaccaaatagtgatgaccttcaagaattggctaagggttgcgacctactcatcttgccaacgaccccggatgtagtcagtctagaaccgatgctggcgatcgccaatgatgtgggagatgcaaagtatcgagcattactcacaattgtgcccccataccctagcaaggaaggggaaacgatgcgtaatgagttaatcgcaaacggtatccctacttttcaaagcatgattcgccgtagcgctgcttttcaaaaagcggctttagctggtaaaccagtaaaccagatgtctggtagagatagaatcccttggaatgactttgaggcactgggtaaagaaattatggaggtactaagaaaatgagcggcaaatttggagacatcatcggcagagcaaaacaaaccagtaaaccagataaccagatatctgaccaacaaaataatcagcaatctgctcagtccaccgagaccgaaaaaatggtgaatctttgtgcgaaggttcccaagtctctcaggcagcattgggcggcagaggctaaacgaaacggcatcaccatgacagaagtaatcattgatgctcttaaccagaagtttggcaaaccataaaaccagatttaatgctatggtcgcttttcttttgacgaaatgaccccataaaaccccagatttttcgttgggattgtagttcaaagagctgggtgaatctccttttattcaggcaggatgcagtcgaagcggctctatcccgccgtttccttttaaaccagtgggaaaaggcatcggtgggcaacctcaccacagtcatcagggcgatgcaggatttagaggaaattatcgatgatgtcccggcggcgatcgcctcttgctagaagctaaatgaccgggttagaggctgtttcattttgactctgctgtccgaaatggaggtaaaggggttgaagtaataggcgtttgcttaagttcagtcattgataggcttgagctaatttttctgtaacctctgaaaatagcgtggctcgataaaaccgcttacttgtcgattgacaagtaaaaaaatagcacctatcttggaatcaagacatgaatcgaaatcgacatcccaataaagaaatcgaagcggcgctggagtacgcagaagctaacggctggagagtggaaataggcggcgggcattgttgggggagactcctatgccccgaaaacaagggctgtcgcaatcgtctgttttgcgctaattcgatttggtctacgccgaaaaatcctcaaggtcatgccagaaatattcgtaaatgggttgatggctgcgatgagaatagggaatgaataaaggactaaaaccatgaaatactataactttgagctatctttcaggctccctggtgtcaatacaaaccctgagcaatacttagacgcactatttgaggctggctgcgacgatgcaatgatcggtatgggccgtaatgggtttataggagctgactttagtcgggaatctgaatctttagagttagctgtcgagtccgcaattaaagatatcgagagcgctattcccggagctgtactgattgaggctgggccagatttagtcggggcttcagatattgctgctattctcggatgtagtcgccagaatattagacagcacctgacaatggctgatgaaaacggccctattcctgtataccaaggtaaacgcgacctgtggcatcttgcagaagttctgatctggttacgagatgctaaagggttaaaaattgaacctgaactaatagaagtcgcggcttacgtgatgacattcaactcgaattgccagtctaaaaaaaccaaaaaagtagcggccttggcttaatcatctatggtcaactacaagccttttttacgaatccgattcacgcctgagagcgatttataaagaaggctcctcggcgtaataatttttcttatggtcgccgcccgctttggttgaacttaccctcaaaagtttcaacacattcaaaaatcctctcaattttccgacctaaggaaatcagtctccgatagcggcgattttgcctccgtcagaaaaaaacctccagaggtgcgtcaggtgtggggaaattttttctttatcaccaggaggtaattgattatcattgctcagctctttttggtcatctgctggagatttggtgatactctactggctttttgctggtcttttgtctcttgtttgctggatattctcaataaatctggataatctagcagttattgcaaatacaatctttcttgagttgttcaggctttagcaaagcaccatctaatccccaccagataactagcatttcaccatcaagactaattacaaacaattgtactgattaatcttgcgtccattatttaatttattcgtccgtccgccacttaaaggagagtgattaaaaacgggggaatcaagcagcattaggcactccagtttccttcagtggcggagaataccatcggtcaaccacttctttccagtcttgatttaggactttagttctcacctgtagtaatagatgagcacccttcggagtccatcgcatttgctgcttcttctccattcgtttagccacgacttgattgactgtagattccacaaatcccgttgagatgcgctccccacagcggtaacgttctccatagttgggaataaaatggccattgttctgaatatacgtgtagaactcagcaatagcccgccgcatctttttgagtttcggataattactttcgagaaagtcagcatcctcctccagccattccagctcttgtaatgccctgaatacattcccatgccagagataccatttgacactctttagcctctctaccatagacttgccattgtctaaatcatagtgcttcaaccccctgagatattgactgagctgagtgatccgcatggtgacatgaaaccagtccagaagatattccgcttgaggattgagaaatcgctgcaagtctcggactgtatcgcctccatcagaaaagaaggtaacctgctaattcatttgcattccttgggattttaggagttcaaataaccgtcgctttggtttggtgtcataggtctggacaaaaccaaagcttttgccaggtccttcatctggaatactcttgcccacaatcagttcaaaatggcgctgttgtttactccctttagaacagtgagcatggatataggcaccatcaattcctacattgaggggaagctcaggtcgagggagttgctcccattggcgagggctaccttctacatacataatttgttcttctcccaattcatcatcgagcttttgccccatttgatgcaggtgatatctcacggtagaaggattaagggtgccgttcaatggcaacacctcatcaagtaactggacactcagaccataggaaaccagggaggcaaattttgtctgcaggtaaaggtattcaggagactgccgttcagtcaggagtaaggctaatgggctaaagctttttgttgcttgggactggcagggacaatgaaaaaatcgaggactagttaacgtcagcttcccaaatacagaacgataaattagcttatgtttccctttgcatttacgcggttgaccacagtctggacaaaacgcctgttgctctacatattgactgacctgtgacgaaaccatttcttgttggatcccttgcagaatctgttttgattcggccaaagtcaagcctagattagtaggagtcaatgggcctcgctccaattgagcgacatcctcaatgatttttatgctgccatcatcggcttcaatccttaagctgattttgatattcataagcctagaaaataatgtcctctcgaaaagctgggtcgtcactcaagggattttgctgtaagcgatgattgagttttggtaactcaaggttgtctagtagcagccaataactcagttgccgaagttcactcagatgtggcttaagctcatcatctccttggaaaagaatttcagccatgcggtggagtaccattcctgtagagaattcctgtctaagttcctggaaagcccaagctcttccacaatcttcaatggggccatttcgtttacccttcaatacaaatcgggtaagttttttcgggctcaagttcagttatcttttcaactcgcagttgtacctgccagttatcgtaatagttgtactcgtagcgaaaccgttctcgcacccgcaaacccagacttaagagagtcacagaatggggatcatcgctgaagcattccccaccaatatgatggatgccgtaggagttgccgtggatgacgaagcgatgtaaatatgtgtcgctccagccaaaagcgatctgtagaataaaatgtagttgagcaatcgttgtgtcgcctctcactaggaaccgacgccagatcattgggctgacaccgacaataacagctttgatctgaaatacatgtgagcctgagccactatccataccactacatcaacgaatagactcaatagtcttataaagtgtactttgcaagactgtgcaaaacagtgttttatttaatgtctcattatggctttatcatcgtgtctaagacgttataaaacattacggtgagtcatcatggcgatggttggctacgcgcgagtcagttctgttgggcagagtctggaagttcaaatcgagaagctcaagcattgcgacaagattttcaaagaaaagtgcagtggtgtctcttacaaacgccctaggcttaaagcctgtcttgaatatgtcagagaaggcgacacattagttgtgactcgtttagatcgcctggctcgctcaaccctgcacctgtgcgaaattgcggaacaattggaatctaagcaggtgaatcttcaagtgcttgaacagagtattgatactggagatgcgaccggacggcttttattcaatatgttgggggcgatctcccaatttgaaacggaaatacgtgcggaacgtcaaatggatggcatccaaaatgcgaaagctcgtggggttcagtttggccgtaaaaagcagctcacgccaggtcaatgccaagaactacgtaaaagacgctcacaaggggtattaatcaaaacgttgatggaagtctaaggcaactatctatcgctatttgaaggaagcggaaacggtaaaaagttgatgagaatttagatccctgcgatccactctgagtatactttcccccgtttttaatcactctcgatctcccacagtattcaaaccgctcctgggctgccatgagactctcttctaggcggtcttttgtgacatattcgtccaagggcagcagaatccctaggtctatggttggtgattcctgaagctgggtctggatatgggataggactcccagaattttgtacagagccttgatgtatttcaggtcattgttgcgcaccgcggaaccctgaaacttcttggcaccggcacccaggagatagcaggcatcgccatgggatacccaagcattctccacatgggtataccccatcatccacccagaggagcgtgggtagtctgggggcactgccgcgcagtagggggacatcacaaaacattgggtagaagtaccctctaggcaataggccacttttgttgcactactaccggggtcaatcacaattttgagtttagacataatttattcctcctactgatgaagagaaattgccctgaacagcacggatagaagtgatttatttgttacacaacttcataatggttcgtctgtaaaagctgtgattgtcttgggctggtcttggttcgggggggcgaatttttgctgagaattttctcttattgatgcttaggctgaaaagtaaacttgtattactgtttactctgatggttaattgctggttatccgctgggggttgggtggtattttgctaacatctggataggtcaatgcaagttctattcgcaatagttgttgaatgatcccggtttcttgagaattatcagcaaacaacaggcaatgagccacctagccatgggcaaagcgccatcaacttctgactgtctagccacggaatcagaagcacgctctgccgtttatcctctagtcaaaccaaggaaatttccccacaccttatacacctccggaggttttttctggcggtggcaaaatcgctgctatcggaagcagatttccttgggtcggaaatttgaggaaattttccgagattttggcgatcgcccatacaactttgaaaaaagtgagtaatcaaaacagatattttgttcgtttcagtcgtaatgcctattgaaataggaggcccgtattctatatccttgccatgggcatcgggtcgttcaccagcgaggccatctaatgtcctatcagccgcccttcaccattaccccccaaatcattaaccagatcagtattatctctcagtagatcggggcactcgaccattcgcctcttacctcctccccagcgaaagcctgatcaaatagcaccccattcatttgctttatgctggatatcattaatacaaccctgcgccaaaacctgacatcctccagtccactatcaacaacaaaaaataacccaggcgatcaagtaaatgatcaagccgatcaagtaagcgatcaagtaaaacaactccttgccattatggatgaccaattttggagcactaatgctctaatggaatctttttctctcacccgtaaacccaccttccgcaaaaactatctctatcccgcgatccaggctgggctagtggtgatgaaatatccggataatccccgtcatccccagcaaaagtacaaaaaggtagatgggtaagtttgtcgctatgccaggaatagcatcacctccaacgaaattacaagcatcaatcgaactcctggcgctagacgcagcgtatctctttgcccctggtgcaagatgcgagtggttgaaaagtgtacccagttgtaaggatggatcgctaacagtgggacgtttcagtcccgtagtcgggatttagtggttggaaagttgagttcccaggagcattaaaatttaacattcatgaatcgtaattgtattttatccgctactacccatactctaactatgactcaacaacagacagcctgttcttcagtaataacatcagaacaagttcttgaaacgctgaggaactatcccaatctctttgaaaaatttagtattaaaactctggcattatttgggtcaactgctcataatcaagccacagcgaccagtgacttagactttgttgttgaatttcaaaatgaaacaacccttagcttgtacatggacttaaagtttttcctggaagaattatttaataaaccggttgacttagcaaccaaaaagtctttaaaagaaatcattcgtgaacaagtattgaatgaggctaaatatgtctaggagccttaagctttacctcaatgatattctaacaagcattgacaaaatccaggaatatagtgagggtctggaaaaagaagcattcttaaagcactcacttatctttgatgcagtgacccttaacctacaaatcattggagaagccagcaagaaaatcccagaacaaatccgaaatcagtatccacatattccatggcgaaacatcattggcctgagaaacattattgcccacacatacttttacttggacgaagacattctttggcacactatccagcatgaactggaaccactacaaaagtgcatccaggaactctgggataaagaagcataactaaccactaagtacaggacaaaaattgtatggggtgaaaccctgacccaagtagaggttgggtagaaacggtaaaaaagcgacgtaataagtcaaaaccatagcgaaacaaactccgcgct

SEQ ID NO: 76 - protein, 6 aa, Artificial Sequence
  Description of Artificial Sequence Synthetic 6xHis tag
HHHHHH

SEQ ID NO: 77 - protein, 4 aa, Artificial Sequence
  Description of Artificial Sequence Synthetic peptide
GGSG

SEQ ID NO: 78 - DNA, 2847 bp, Synechococcus elongatus
atgctgaatttgctgctgggcgatcccaacgtccgcaaagtcaaaaagtacaaacccctcgtcactgaaatcaatctgttggaagaggacattgagccactgtccgacaaggatttaattgccaaaacggctgagtttcgccagaagctcgacaaggtttcccactcgccagctgcagagaaggaattgctggcggagttgctgcccgaagcctttgcggtcatgcgcgaagccagtaaacgagtgctggggctgcgccactttgatgtgcagatgatcggcggcatgattctgcacgacggtcagattgccgagatgaagacgggtgaagggaaaaccctcgtcgctacgctgccgtcctatctcaatgcactgtcgggtaaaggtgcgcacgtcgtcaccgtcaacgactacttggctcgccgcgacgcggaatggatgggacaagtccaccgcttcctaggcttgagtgttggcctaatccagcagggaatgtcgccggaagagcgtcgccgcaactacaactgcgacattacctacgctaccaacagcgaactgggctttgattacctgcgcgacaacatggccgcagtgattgaagaggtagtccagcgtcccttcaactacgccgtgatcgacgaggtggactcgattctgatcgacgaagcccggacacccttgatcatttccggtcaggtcgatcgcccgagcgaaaaatacatgcgggcatcggaagtcgcggcgctcttgcagcgatcgacgaatacggacagtgaagaagagccggatggcgattacgaagttgacgaaaaaggccgtaatgtcctgctgacggatcaaggctttatcaacgctgagcaattgttaggtgtcagcgatctgtttgactccaatgacccttgggctcactacatctttaatgcgattaaggccaaggagctgttcattaaagatgtgaactacatcgtgcgcggtggcgagattgtcatcgtcgatgagttcacagggcgcgtgatgcctgggcgtcgctggagtgatggtctgcatcaggccgtggagtcgaaggaaggcgttgagattcaacccgaaacccaaacccttgcttcgattacttaccaaaacttcttcctgctctaccccaaactgtcgggcatgaccggtacggcgaagacagaagagttggagtttgagaagacttacaagctagaagtaaccgttgttccgaccaaccgagtcagccgtcgtcgggatcagcctgatgtcgtctacaaaactgagatcggcaagtggcgtgcgatcgcagcggactgtgctgaactgcacgcggaaggtcgtcctgttctggtcggtactaccagtgttgagaagtcggagttcctgtcacaactgctgaatgagcagggcatcccccacaacctgctcaacgccaaacccgaaaacgtagaacgcgaggcggaaatcgttgcacaggcaggccgtcggggtgccgtcacgatttcgaccaacatggcaggtcgcgggaccgacatcatcttgggcggtaatgcggactacatggcgcggctgaagctgcgcgagtattggatgccgcaactggtcagcttcgaagaggatggctttggcattgctggggttgctggtttagagggcggtcgcccggcagcgcaaggttttgggtcgcccaacggccagaagccacgcaagacttggaaagcgtcgtcggatattttcccagcagaactgagtactgaggccgaaaagctgctgaaagcagcggtagacctcggggtgaaaacctacggcggtaacagcctctcggagctggtagcggaagacaagatcgctacggcggctgagaaggcgccgacggatgatccggtgattcaaaaactgcgggaagcctaccagcaagtccgcaaagaatacgaagcagtcacgaagcaggagcaagccgaggtcgttgaactgggcggcctgcatgtgattggtacggaacgccacgagtcacgccgagtggataaccagttgcgcggtcgtgccggtcggcaaggggacccaggatccacgcgtttcttcctgagcttggaagataacctgctgcggatttttggtggcgatcgcgtggccaaactgatgaatgccttccgcgtcgaagaagatatgccgatcgagtcgggcatgctgacgcgatcgctcgagggtgctcagaagaaggtcgagacctactactacgacatccgcaagcaggtgtttgagtacgacgaggtgatgaacaaccagcgtcgtgccatctatgcagaacgccgccgtgttctcgaaggacgagagctaaaagaacaagtgattcagtacggcgaacggacgatggatgaaatcgtcgatgcccacatcaatgtggatttgccgtcggaagagtgggatctggaaaagctggtcaataaggtcaagcagttcgtctatctgcttgaagacctagaggccaagcaactggaagacctgtctcctgaggcgatcaagatcttcctgcacgagcaattgcggattgcctacgacctcaaagaagcccagatcgatcaaatccagccaggcttgatgcggcaggccgaacgctacttcatccttcagcagatcgacacgctctggcgtgagcatttgcaggcgatggaagccttgcgcgaatccgtcggtctgcggggctatgggcaaaaagatccactgctggagtataagagtgagggctacgagctgttcctcgagatgatgacggcgattcgccgcaacgtgatctactcgatgttcatgttcgatccgcagcctcaagcccgtccacaagctgaggtggtttag

SEQ ID NO: 79 - protein, 948 aa, Synechococcus elongatus
MLNLLLGDPNVRKVKKYKPLVTEINLLEEDIEPLSDKDLIAKTAEFRQKLDKVSHSPAAEKELLAELLPEAFAVMREASKRVLGLRHFDVQMIGGMILHDGQIAEMKTGEGKTLVATLPSYLNALSGKGAHVVTVNDYLARRDAEWMGQVHRFLGLSVGLIQQGMSPEERRRNYNCDITYATNSELGFDYLRDNMAAVIEEVVQRPFNYAVIDEVDSILIDEARTPLIISGQVDRPSEKYMRASEVAALLQRSTNTDSEEEPDGDYEVDEKGRNVLLTDQGFINAEQLLGVSDLFDSNDPWAHYIFNAIKAKELFIKDVNYIVRGGEIVIVDEFTGRVMPGRRWSDGLHQAVESKEGVEIQPETQTLASITYQNFFLLYPKLSGMTGTAKTEELEFEKTYKLEVTVVPTNRVSRRRDQPDVVYKTEIGKWRAIAADCAELHAEGRPVLVGTTSVEKSEFLSQLLNEQGIPHNLLNAKPENVEREAEIVAQAGRRGAVTISTNMAGRGTDIILGGNADYMARLKLREYWMPQLVSFEEDGFGIAGVAGLEGGRPAAQGFGSPNGQKPRKTWKASSDIFPAELSTEAEKLLKAAVDLGVKTYGGNSLSELVAEDKIATAAEKAPTDDPVIQKLREAYQQVRKEYEAVTKQEQAEVVELGGLHVIGTERHESRRVDNQLRGRAGRQGDPGSTRFFLSLEDNLLRIFGGDRVAKLMNAFRVEEDMPIESGMLTRSLEGAQKKVETYYYDIRKQVFEYDEVMNNQRRAIYAERRRVLEGRELKEQVIQYGERTMDEIVDAHINVDLPSEEWDLEKLVNKVKQFVYLLEDLEAKQLEDLSPEAIKIFLHEQLRIAYDLKEAQIDQIQPGLMRQAERYFILQQIDTLWREHLQAMEALRESVGLRGYGQKDPLLEYKSEGYELFLEMMTAIRRNVIYSMFMFDPQPQARPQAEVV

SEQ ID NO: 80 - DNA, 354 bp, Synechococcus elongatus
ttggaggtgcatcccatagaaactattaccttcgacaagtttctgaaggttgagcttcgtgtcggcaagattgttgatgcaactgagtttgtgggtgcgcggaggccagcctacatcctgcatatcgacttcggtgaagagattggtgtcaagaaatcaagtgcgcagatcaccgcactctacaagccggaagaactgatcggtgggcttgtcgtagcagtggtcaactttccatgtaagcaaatcggtctgcttatgtctgattgccttgtcacgggattccagagcgagaacagagaagtagcgctctgcatccttgacaagtccgttctgctgggctcaaaattgctttaa

SEQ ID NO: 81 - protein, 117 aa, Synechococcus elongatus
MEVHPIETITFDKFLKVELRVGKIVDATEFVGARRPAYILHIDFGEEIGVKKSSAQITALYKPEELIGGLVVAVVNFPCKQIGLLMSDCLVTGFQSENREVALCILDKSVLLGSKLL

SEQ ID NO: 82 - DNA, 1905 bp, Synechococcus elongatus
atggccaaagttgtcggaatcgacctcggaaccaccaactcttgcgtggctgtcatggagggcggcaagcccactgtgatcgctaatgcggaaggttttcgcaccactccttcagtcgttgcttttgcgaaaaaccaagaccgcctcgtgggtcaaatcgccaaacgccaggcggtgatgaaccccgagaacaccttctactcggttaagcgcttcatcggccgtcgtccggatgaagtcacgaacgaactgaccgaagtggcctacaaagtcgatacttcgggcaatgccgtcaagctggatagctccaatgctggcaagcagttcgctcctgaagaaatttcggcgcaggtgctgcgcaaactggccgaagacgccagcaaatacctgggtgaaaccgtcacccaagccgtgatcacggttccggcctacttcaatgactcccagcgccaagcgaccaaagacgctggcaaaatcgccggcctagaagtgctgcggatcatcaacgagccgacggcagccgcgctggcctacggtcttgataagaagagcaacgaacgcatccttgtctttgacttgggcggcggtactttcgacgtctcggtcttggaagtgggcgacggcgtttttgaagtgctggcgacctcgggtgatacccacctcggtggcgacgacttcgacaaaaaaatcgttgacttcctggctggtgaattccagaagaacgaaggcatcgatctgcgcaaagacaagcaggctctgcagcgtctgacggaagccgctgagaaagccaaaatcgagctgtccagcgccactcaaactgaaatcaacctgcccttcatcacggcaacccaagacgggccgaagcacctcgacctgaccttaacccgcgccaagtttgaagaattggcttcggatctgatcgatcgctgccggattccggtggagcaagcgatcaaagatgccaagttggccctgagcgaaattgacgaaatcgtcttggtcggtggttcgacccggattcctgcggtgcaggcgatcgtcaagcaaatgacgggcaaagagcccaaccaaagtgtcaaccccgatgaggtggtggcgatcggtgcggcgattcaaggtggcgtcttggctggggaagtcaaagacatcctgctgctcgacgtgacgccactatccttgggggtagaaacccttggtggcgtgatgactaagttgatcccacgcaacaccactatccccaccaagaagtcggaaaccttctcgacggcggcggacggtcaaaccaacgtcgaaatccacgtgctccaaggcgagcgcgaaatggccagcgacaacaagagcttgggaaccttccggctggatggcattccgccggctccccgtggcgtgccccaaatcgaagtgatcttcgacatcgacgctaacggcatcctcaatgtcacggccaaagacaaagggtcgggcaaagagcagtcgatcagcatcaccggcgcttcgaccttgtctgacaacgaagtcgatcgcatggtcaaagacgccgaagcgaatgcagcagcggacaaagaacggcgcgaacgtatcgacctgaagaaccaagccgacacgctggtctatcagtctgagaaacaactcagcgagctgggtgacaagatctcggctgatgagaaaagcaaagtcgaaggctttatccaagagctgaaagatgccttggctgccgaagactacgacaagatcaagtcgatcatcgagcaactgcagcaagctctctacgccgctggcagcagcgtctaccagcaggctagcgctgaagcttcggccaacgcccaagccggtccttcctcgtcctcgagcagcagctctggcgatgatgatgtgattgacgcagagttctctgagtcgaagtaa

SEQ ID NO: 83 - protein, 655 aa, Synechococcus elongatus
MGKVIGIDLGTTNSCVAVLEGGKPIIVTNREGDRTTPSIVAVGRKGDRIVGRMAKRQAVTNAENTVYSIKRFIGRRWEDTEAERSRVTYTCVPGKDDTVNVTIRDRVCTPQEISAMVLQKLRQDAETFLGEPVTQAVITVPAYFTDAQRQATKDAGAIAGLEVLRIVNEPTAAALSYGLDKLHENSRILVFDLGGSTLDVSILQLGDSVFEVKATAGNNHLGGDDFDAVIVDWLADNFLKAESIDLRQDKMAIQRLREASEQAKIDLSTLPTTTINLPFIATATVDGAPEPKHIEVELQREQFEVLASNLVQATIEPIQQALKDSNLTIDQIDRILLVGGSSRIPAIQQAVQKFFGGKTPDLTINPDEAIALGAAIQAGVLGGEVKDVLLLDVIPLSLGLETLGGVFTKIIERNTTIPTSRTQVFTTATDGQVMVEVHVLQGERALVKDNKSLGRFQLTGIPPAPRGVPQIELAFDIDADGILNVSARDRGTGRAQGIRITSTGGLTSDEIEAMRRDAELYQEADQINLQMIELRTQFENLRYSFESTLQNNRELLTAEQQEPLEASLNALASGLESVSNEAELNQLRQQLEALKQQLYAIGAAAYRQDGSVTTIPVQPTFADLIGDNDNGSNETVAIERNDDDATVTADYEAIE

SEQ ID NO: 84 - DNA, 1131 bp, Synechococcus elongatus
atggctgctgactactaccaactgcttggcgttgctcgcgacgcagacaaggacgaaattaaacgtgcttatcggcgtttggctcgcaagtaccatccagatgtgaacaaggagccaggcgctgaagacaagttcaaagaaatcaaccgcgcctacgaggtgctgtcggagcctgaaacccgcgctcgctacgaccaatttggggaagcgggtgtctctggtgccggagccgctggtttccaagattttggcgacatgggtggattcgctgacatctttgaaaccttcttcagcgggtttggaggcatgggcgggcaacaagcctccgctcgccggcgcggacccactcggggtgaagacctacggctggatttgaaactggatttccgagatgccatctttggtggcgagaaagaaattcgggtcacccatgaagaaacttgcggcacctgtcaggggagtggggctaaggccggaacccggccgcaaacttgtacgacctgtggtggtgcaggccaagtccgacgagcaacccggacgcccttcggcagctttacccaagtttcagtctgtcccacctgcgagggcagcgggcagatgatcgttgataagtgcgatgactgtggcggagcagggcgtctacggcggccgaagaaactgaagatcaatattccagctggggtggatagcggtacgcggctgcgagtagccaatgaaggcgatgcggggctgcgcggtgggccgccgggcgacctttacgtctatttgttcgtcagtgaggacacccagttccggcgggaaggcatcaatctcttctccaccgtgaccatcagctacctgcaagccattttgggctgcagcctagaagttgcgactgtagacggccccaccgagctgatcattccgcccggaacacaacccaatgccgtactgacggtggagggcaagggcgtgccacgactggggaatccggtcgctcggggcaatcttttggtcacaattaaggtggaaattcccaccaaaattagcgctgaagaacgcgaactgttggaaaaagtggtgcaaattcgcggcgatcgcgctggaaaaggagggattgaaggcttcttcaaaggagtctttggcggatga

SEQ ID NO: 85 - protein, 214 aa, Synechococcus elongatus
MAILEQGNITIHTDNIFPIIKKSLYSEHEIFLRELISNAVDAIQKLKMVSYAGELEGEIGDPQITLSIDRDRKQLKIADNGIGMTADEIKRYINQVAFSSAEDFIEKYKGGADQPIIGHFGLGFYSAFIVADRVEIETLSYQKGATPVHWTCDGSPSFELSEGSRTERGTTIILNLSEEELEYLEPARIRQLVKTYCDFMPVPIALEGEVLNKQ

SEQ ID NO: 86 - DNA, 312 bp, Synechococcus elongatus
atggcagctgtatctctgagtgtttcgaccgtgacgcccctgggcgatcgcgtttttgtgaaagtcgctgaagccgaagaaaaaactgctggcggcatcatcctgcccgataacgctaaagagaagccccaagtcggcgaaatcgtggcagttggccctggcaaacgcaacgacgacggcagccgccaagcgcctgaagtcaaaatcggcgacaaagttctctactccaagtacgccggtactgacatcaaactcggcaacgacgactacgtgttgctgtccgagaaagacatcttggccgttgttgcctag

SEQ ID NO: 87 - protein, 103 aa, Synechococcus elongatus
MAAVSLSVSTVTPLGDRVFVKVAEAEEKTAGGIILPDNAKEKPQVGEIVAVGPGKRNDDGSRQAPEVKIGDKVLYSKYAGTDIKLGNDDYVLLSEKDILAVVA

SEQ ID NO: 88 - DNA, 1635 bp, Synechococcus elongatus
atggctaaacggatcatttacaacgaaaacgcccgtcgcgcccttgaaaaaggcatcgacattctggcggaagccgttgcagtcaccctcggccccaaaggtcgcaacgttgttcttgagaaaaagttcggcgcaccgcaaatcatcaatgacggtgtgacgatcgccaaagaaatcgaactggaagaccacatcgaaaacaccggtgtggcgctgattcgtcaagccgcttccaaaaccaacgacgcagccggtgacggcaccaccaccgcaaccgtcttggcgcacgctgtggtcaaagaaggtctgcgtaacgtggctgctggcgctaacgccattttgctgaagcgcgggatcgacaaagccaccaacttcttggttgagcaaatcaagtcccacgctcgtccggtcgaagactccaagtcgatcgcccaagtcggtgcaatctcggctggcaacgactttgaagtcggccaaatgatcgccgatgctatggacaaagtcggcaaagaaggcgtcatctcgctggaagaaggcaaatcgatgaccaccgaactggaggtcaccgaagggatgcgtttcgacaagggctacatctcgccctactttgccaccgacaccgagcggatggaagccgtctttgacgagcccttcatcttgatcaccgacaagaaaatcggtttggttcaagacttggtgcccgtgctggagcaagtggctcgcgctggccgtccgctggtgatcatcgccgaggacatcgagaaagaagccctcgccaccttggtcgtcaaccgtctgcgtggcgtgctcaacgttgctgcagtcaaagcgcctggtttcggcgatcgccgcaaagccatgctggaagacattgctgtcctgactggtggtcaactgatcactgaagacgcaggtctgaagctggataccaccaagcttgatcagctgggtaaagcccgccggatcacgatcaccaaagacaacaccacgatcgtggctgaaggcaacgaagcggctgtgaaggcccgcgttgaccaaatccgtcgccaaatcgaagaaactgagtcgtcctacgacaaagagaagctgcaagagcgcttggctaagctctccggtggcgttgcagtcgtcaaagttggcgcggcaaccgaaactgaaatgaaagaccgcaaactgcgtctggaagatgcgatcaacgccaccaaagcggcggttgaagaaggcatcgtccctggtggcggcaccaccttggcgcacctcgctcctcagctggaagagtgggcaaccgctaacctcagcggtgaagagctgaccggcgctcaaatcgtggctcgtgccttgacggctccgctgaagcggattgctgaaaacgctggcctcaacggtgctgtgatctccgagcgcgtcaaagaactgcccttcgacgaaggctacgacgcctccaacaaccagttcgtgaatatgttcacggctggcatcgttgacccggccaaagtgactcgtagtgccctgcaaaacgcagcttcgatcgcagccatggtgctgacgaccgagtgcattgtggtcgacaaaccggaaccgaaagaaaaagccccggctggtgctggcggcggcatgggcgacttcgactactaa

SEQ ID NO: 89 - protein, 544 aa, Synechococcus elongatus
MAKRIIYNENARRALEKGIDILAEAVAVTLGPKGRNVVLEKKFGAPQIINDGVTIAKEIELEDHIENTGVALIRQAASKTNDAAGDGTTTATVLAHAVVKEGLRNVAAGANAILLKRGIDKATNFLVEQIKSHARPVEDSKSIAQVGAISAGNDFEVGQMIADAMDKVGKEGVISLEEGKSMTTELEVTEGMRFDKGYISPYFATDTERMEAVFDEPFILITDKKIGLVQDLVPVLEQVARAGRPLVIIAEDIEKEALATLVVNRLRGVLNVAAVKAPGFGDRRKAMLEDIAVLTGGQLITEDAGLKLDTTKLDQLGKARRITITKDNTTIVAEGNEAAVKARVDQIRRQIEETESSYDKEKLQERLAKLSGGVAVVKVGAATETEMKDRKLRLEDAINATKAAVEEGIVPGGGTTLAHLAPQLEEWATANLSGEELTGAQIVARALTAPLKRIAENAGLNGAVISERVKELPFDEGYDASNNQFVNMFTAGIVDPAKVTRSALQNAASIAAMVLTTECIVVDKPEPKEKAPAGAGGGMGDFDY

SEQ ID NO: 90 - protein, 65 aa, Artificial Sequence
  Description of Artificial Sequence Synthetic polypeptide
DTKKKVDDDLGTIENLEEAKKKLLKDVEVLSQRLEEKALAYDKLEKTKTRLQQELDDLLVDLDHQ

SEQ ID NO: 91 - protein, 73 aa, Artificial Sequence
  Description of Artificial Sequence Synthetic polypeptide
LVLGALLDTSHKFRNLDKDLCEKCAKCISMIGVLDVTKHEFKRTTYSENEVYDLNDSVQTIKFLIWVINDILV

SEQ ID NO: 92 - protein, 90 aa, Artificial Sequence
  Description of Artificial Sequence Synthetic polypeptide
LVLGALLDTSHKFRNLDKDLCEKCAKCISMIGVLDVTKHEFKRTTYSENEVYDLNDSVQTIKFLIWVINDILVPAFWQSENPSKQLFVAL

SEQ ID NO: 93 - protein, 70 aa, Artificial Sequence
  Description of Artificial Sequence Synthetic polypeptide
LVLGALLDTSHKFRNLDKDLCEKCAKCISMIGVLDVTKHEFKRTTYSENEVYDLNDSVQTIKFLIWVIND

SEQ ID NO: 94 - protein, 75 aa, Artificial Sequence
  Description of Artificial Sequence Synthetic polypeptide
TLVLGALLDTSHKFRNLDKDLCEKCAKCISMIGVLDVTKHEFKRTTYSENEVYDLNDSVQTIKFLIWVINDILVP

SEQ ID NO: 95 - protein, 76 aa, Artificial Sequence
  Description of Artificial Sequence Synthetic polypeptide
LVLGALLDTSHKFRNLDKDLCEKCAKCISMIGVLDVTKHEFKRTTYSENEVYDLNDSVQTIKFLIWVINDILVPAF

SEQ ID NO: 96 - protein, 74 aa, Artificial Sequence
  Description of Artificial Sequence Synthetic polypeptide
ITLVLGALLDTSHKFRNLDKDLCEKCAKCISMIGVLDVTKHEFKRTTYSENEVYDLNDSVQTIKFLIWVINDIL

SEQ ID NO: 97 - protein, 76 aa, Artificial Sequence
  Description of Artificial Sequence Synthetic polypeptide
SDITLVLGALLDTSHKFRNLDKDLCEKCAKCISMIGVLDVTKHEFKRTTYSENEVYDLNDSVQTIKFLIWVINDIL

SEQ ID NO: 98 - protein, 13 aa, Artificial Sequence
  Description of Artificial Sequence Synthetic peptide
MINQPCIVPAEKG

SEQ ID NO: 99 - protein, 29 aa, Artificial Sequence
  Description of Artificial Sequence Synthetic peptide
MELKKLFVPLLAGMLFLGGTSGAIAEELL

SEQ ID NO: 100 - protein, 21 aa, Artificial Sequence
  Description of Artificial Sequence Synthetic peptide
MASNFKFKLLSQLSKKRAEGG

SEQ ID NO: 101 - protein, 20 aa, Artificial Sequence
  Description of Artificial Sequence Synthetic peptide
MEIDGFGGILYTSDEAILGG

SEQ ID NO: 102 - DNA, 876 bp, Synechococcus sp.
ctcaactttttagcaagtggtggtgatggctatccatttcccacaggtgactcagtcaaccgagttgatctcactgaccttgatggcgatggtcaagacgacaatcagctaaccggggatgccaccttcgcagcagatggaactgaacaggatgctctagctgagtatttactagataacttcagtactccagaaacagcatttgctcaagaggatgtaggccgtactctggatgaacgtatccaaaacctcaacttccgtgatgatagtgtacttggtgaatctactaatggccaggttatctttagagcgataaatttaatttcctctatttttcaaagagttttggggccatttagcaacaatgtgaatctgggtaacatcctcttcagtgatgaagaaggtgttgaagatagctttgatatcttcaacacaaatttacggcagcgtatcctggggaatcgcaacaacaccacttccttaaataacctggataaccagatgtggggccgcaataactcggatgatgtgatgaacgccttgggcggtgacgattccgggtacggccagagtgatgatgacatcctgcgcggcgatcgcggcaacggcatcctgaatggtggcataggtgatgacattctcacgggtggcaagggtctaggaacctttgtcctcaactccggcggggcaggcgttaataccatcactgactttgaactcggcattgaccgtattgtcttaggcaacttaagcgttaacgaggttcagttggctgacacatctattaacactatgatgtcggctagtcccagtgatctactaggcatctttaccggtgtacagctcagtggttttgaaagcgaggtttttgcataa

SEQ ID NO: 103 - protein, 291 aa, Synechococcus sp.
LNFLASGGDGYPFPTGDSVNRVDLTDLDGDGQDDNQLTGDATFAADGTEQDALAEYLLDNFSTPETAFAQEDVGRTLDERIQNLNFRDDSVLGESTNGQVIFRAINLISSIFQRVLGPFSNNVNLGNILFSDEEGVEDSFDIFNTNLRQRILGNRNNTTSLNNLDNQMWGRNNSDDVMNALGGDDSGYGQSDDDILRGDRGNGILNGGIGDDILTGGKGLGTFVLNSGGAGVNTITDFELGIDRIVLGNLSVNEVQLADTSINTMMSASPSDLLGIFTGVQLSGFESEVFA

SEQ ID NO: 104 - DNA, 303 bp, Synechococcus sp.
gatgatgacatcctgcgcggcgatcgcggcaacggcatcctgaatggtggcataggtgatgacattctcacgggtggcaagggtctaggaacctttgtcctcaactccggcggggcaggcgttaataccatcactgactttgaactcggcattgaccgtattgtcttaggcaacttaagcgttaacgaggttcagttggctgacacatctattaacactatgatgtcggctagtcccagtgatctactaggcatctttaccggtgtacagctcagtggttttgaaagcgaggtttttgcataa

SEQ ID NO: 105 - protein, 100 aa, Synechococcus sp.
DDDILRGDRGNGILNGGIGDDILTGGKGLGTFVLNSGGAGVNTITDFELGIDRIVLGNLSVNEVQLADTSINTMMSASPSDLLGIFTGVQLSGFESEVFA

SEQ ID NO: 106 - DNA, 603 bp, Synechococcus sp.
gtacttggtgaatctactaatggccaggttatctttagagcgataaatttaatttcctctatttttcaaagagttttggggccatttagcaacaatgtgaatctgggtaacatcctcttcagtgatgaagaaggtgttgaagatagctttgatatcttcaacacaaatttacggcagcgtatcctggggaatcgcaacaacaccacttccttaaataacctggataaccagatgtggggccgcaataactcggatgatgtgatgaacgccttgggcggtgacgattccgggtacggccagagtgatgatgacatcctgcgcggcgatcgcggcaacggcatcctgaatggtggcataggtgatgacattctcacgggtggcaagggtctaggaacctttgtcctcaactccggcggggcaggcgttaataccatcactgactttgaactcggcattgaccgtattgtcttaggcaacttaagcgttaacgaggttcagttggctgacacatctattaacactatgatgtcggctagtcccagtgatctactaggcatctttaccggtgtacagctcagtggttttgaaagcgaggtttttgca

SEQ ID NO: 107 - protein, 201 aa, Synechococcus sp.
VLGESTNGQVIFRAINLISSIFQRVLGPFSNNVNLGNILFSDEEGVEDSFDIFNTNLRQRILGNRNNTTSLNNLDNQMWGRNNSDDVMNALGGDDSGYGQSDDDILRGDRGNGILNGGIGDDILTGGKGLGTFVLNSGGAGVNTITDFELGIDRIVLGNLSVNEVQLADTSINTMMSASPSDLLGIFTGVQLSGFESEVFA

SEQ ID NO: 108 - DNA, 900 bp, Synechococcus sp.
gcaacagggatacagttaatcgatcagttgcctgatggtctttattatgtttcgggcacaggcacagattggacttgtccgcttgtgagctttaccgccccaggcccgccaacccccgaagatctgagggatattgaatgcagttacaacggaacccttaccccaggagccacggcaccaaccttgacgattacggtgtatgtccaagacaccgcccccagtactctcgaaaattttgttacagtctttggcgatcaacccgatcccaatgacgacaataatacggatttagaccggacaacgattactgatggtgttgctaacgctcctgatttaattcttgtaaaacgtattactgcggttatcagcgaaaacaatactacaaattacactgtgtatagggatgatacgagtagtgacagtaccgcagctaatgataatgcgccgttttggcctggttatagtgcgggcaatcaaagtaacaccttcacagtgggagagttaggccttgaggctaaaccgaatgatacagttgaatacactatttatttcctcaatcaaggcaatgccccggccagcaatatcaaaatttgcgatcgcctatcccaatacttagattattcgccagatgcttacggttcatctatgggtattaaactgaactttaacaacagtgaaaccaatttaacgggcgttgctgatgttgatgcgggacaatttttcggccctgaccttacccctagcggatgtattcgtccagacaacctacaacccatgaccgccgccgataatccaaatggaaccctgagagttgagctagctaatgttgaccccgcgactagccctgcgactccagctaattcctatggctatatccgcttccgggctcgtgtgaaataa

SEQ ID NO: 109 - protein, 299 aa, Synechococcus sp.
ATGIQLIDQLPDGLYYVSGTGTDWTCPLVSFTAPGPPTPEDLRDIECSYNGTLTPGATAPTLTITVYVQDTAPSTLENFVTVFGDQPDPNDDNNTDLDRTTITDGVANAPDLILVKRITAVISENNTTNYTVYRDDTSSDSTAANDNAPFWPGYSAGNQSNTFTVGELGLEAKPNDTVEYTIYFLNQGNAPASNIKICDRLSQYLDYSPDAYGSSMGIKLNFNNSETNLTGVADVDAGQFFGPDLTPSGCIRPDNLQPMTAADNPNGTLRVELANVDPATSPATPANSYGYIRFRARVK

SEQ ID NO: 110 - DNA, 900 bp, Synechococcus sp.
gctggtcgcaacgttagcgcaacaaacaatgtcaatgctggcaataacatcaatgccgccaacaatgttgaagcgggtcaagatgtcaatgctgtccgtaacgtcagcgctggtaacaatgtcaatgttggcaataacgctaacgttgggaataatctgcaagtcggtcaagacgccttcattaacagaaacgcggtcgtgggaggcgttctagacgttaccggaaacgcacaattcgatagtaatgttaatgttactggcgaaacaactctcaacggtttaaccacaaccaatggcatcaacaacaccggagctatcaatactgatactctaaatgcagccggtgctgtggatattcagggtttaacgacaactaatggcatcgacaataccggtgcgattacaactgatactctcgacgtggcaggcaccctggaagtagatggtacaactactctcaatggtcccacgactatcaataatgatctaactgttcaaaacaatacaacacttggcgatgctgccggtgatactctagacgtcaatgctggcaatgttttcttcaataaccttcccagcagcagctccactgacctcctcgttatcgaaagtgacggtcgagtcggtgtaaacaacaatatcattgatgatctcagatctggtattgctgccaccattgcgatggataacgcagaagctgaacttcgtcctggtcatcgctttgccatcggtattggtctcggggtctacgaagacgaaactgcaattggtacttctggtaagttcctctttaccgatcccaacagcactggaaccgctgttactttcaaagcaagtgctggtttcggtcttactaccgatagcttcgctgccggtgcaggtctcggcctaagcttctaa

SEQ ID NO: 111 - protein, 299 aa, Synechococcus sp.
AGRNVSATNNVNAGNNINAANNVEAGQDVNAVRNVSAGNNVNVGNNANVGNNLQVGQDAFINRNAVVGGVLDVTGNAQFDSNVNVTGETTLNGLTTTNGINNTGAINTDTLNAAGAVDIQGLTTTNGIDNTGAITTDTLDVAGTLEVDGTTTLNGPTTINNDLTVQNNTTLGDAAGDTLDVNAGNVFFNNLPSSSSTDLLVIESDGRVGVNNNIIDDLRSGIAATIAMDNAEAELRPGHRFAIGIGLGVYEDETAIGTSGKFLFTDPNSTGTAVTFKASAGFGLTTDSFAAGAGLGLSF

SEQ ID NO: 112 - DNA, 900 bp, Anabaena sp.
atcgttacggaaaacgctaacgaaggtatagacacagttcagtcatctgttacttatactctgggcgcgaatgtagaaaatttgactctgactggtacgggtgcaatcaacggtacaggtaacagtctcaacaatacgatcactggcaacagtggcaataataccctcaatggcgatgctggtaatgatttcctgattgctggcaatggtaatgacattctcaatggtggtacaggcaatgatacgatgcttggtggcggaggtaacgacacctacattgttgatagtataggcgactacgttttggaaaatgccaaccaaggtacagacttagttcagtcatctatcagctatacattaggcaatagtttagagaatttgactctcacaggtacatctgcaatcaatggtacaggtaaccgtcttaacaacgtcattacaggtaacagtggcaacaataccctaaatggtggagatggcaatgatactcttaatggtagtgcaggtgttgatactctccttggtggtaacggtaatgacatcctcgttggtggtactggtaacgatacactaacagggggtgtaggacgcgatcgctttacattcaattctcgtagtgaaggtatcgacagaattaccgattttaacgtggttgatgacactattgttgtctctgcggctggctttggtggcgggttggttgtaggtgcggcgatcgcatctagtcagtttttactaggttcagccgccactactgctagccaccgattcctctacgaccgaaacaacggcgctctcttctttgatcaggatggcacgggtgcgatcgctaaagttcaatttgctaccctcaatactggactgtccttgaccaatgcagatattctcgttgttgcttag

SEQ ID NO: 113 - protein, 299 aa, Anabaena sp.
MVTENANEGIDTVQSSVTYTLGANVENLTLTGTGAINGTGNSLNNTITGNSGNNTLNGDAGNDFLIAGNGNDILNGGTGNDTMLGGGGNDTYIVDSIGDYVLENANQGTDLVQSSISYTLGNSLENLTLTGTSAINGTGNRLNNVITGNSGNNTLNGGDGNDTLNGSAGVDTLLGGNGNDILVGGTGNDTLTGGVGRDRFTFNSRSEGIDRITDFNVVDDTIVVSAAGFGGGLVVGAAIASSQFLLGSAATTASHRFLYDRNNGALFFDQDGTGAIAKVQFATLNTGLSLTNADILVVA

SEQ ID NO: 114 - DNA, 900 bp, Anabaena sp.
ttgcgagtctttgatgcagaaggtaatgaactggcgaagaccgattttgatgactttcaagccgcaccggatgaggtgttctcagcctttaatgacccttacttagagttcaccgctgaaacaactggtacttactatgttggcatcagtcagattggtaatgactattatgatccgaatgtggttggtagtggttctggttggctattcgctgatttcggaattgaaaatggtgagtacacagttagttttaatctgactccagaacaacccactaaccccgttgggacttcaggtgatgataccctgattgggactgacgaggaagagagcctgtttggtaatggtggtaatgacatactctatgctagaggcggtgatgacaagctatttggcggtgctggtgacgacctcttagatggtggcgagggtaatgacgcgttgtttggtggtgctggtacagataccttgcttggtggtgctggtaatgattacttaactggtggtactggcgacaatctattagatgggggtgacggtaatgatctcctctatggtaatggtggtcaagatactttactgggcggtgctggtgatgacattatctacagtggctctggtgatgacttgattaatggtggtcttggtaatgacatcatcttcttgaatggtggtcaagatactatagttgtggctcaaggtgcgggtattgacactatcaacaatttccaagtcagtttgggtcaaaaggttggtttgagtggtggtatcacttttgagcaactaactttcagtcaaagtggtttggatacgctgattcaggtcggtgatgaggctctggctgtgttgaagtttgttcaatctagtagtctgagttctgcggcgtttactgttgtttaa

SEQ ID NO: 115 - protein, 299 aa, Anabaena sp.
MRVFDAEGNELAKTDFDDFQAAPDEVFSAFNDPYLEFTAETTGTYYVGISQIGNDYYDPNVVGSGSGWLFADFGIENGEYTVSFNLTPEQPTNPVGTSGDDTLIGTDEEESLFGNGGNDILYARGGDDKLFGGAGDDLLDGGEGNDALFGGAGTDTLLGGAGNDYLTGGTGDNLLDGGDGNDLLYGNGGQDTLLGGAGDDIIYSGSGDDLINGGLGNDIIFLNGGQDTIVVAQGAGIDTINNFQVSLGQKVGLSGGITFEQLTFSQSGLDTLIQVGDEALAVLKFVQSSSLSSAAFTVV

SEQ ID NO: 116 - DNA, 900 bp, Anabaena sp.
accagagcgtcattaggtgagtttgttatctttaatgaagatggtacacctgctgtcacctgggaaggtattgctggcttccctgaaccagatggcactggtggcggtttcttcgtcactttaaccgaaccgacagcatccctcagcctgaaggtgtttgatgatggtgctaatgaaggtattgaaagcttaaccttcaatttggtggatggagaacagtatcaagtcagccctgatgctggtagtattgctctgactatcagtgataccccaaccaatcctgttggtgatgctggtgacaacatcctagttggtgatggcaataacaacagtttgtttggtaatgctggcaatgaccgcatctttggtggtctgggtaatgactacctgtttggcggtgctgacgacgacctcttaaatggtggcgacggtaacgacgcgctgtttggtggtgctggtaataacaccctattaggtggtgctggtaatgactacttaactggtggtgctggcaataacctcttagatggaggtgacggtaacgatatcctctatggtggtaatggtaataatactttactaggtggtgctggtaatgacatcatctacagtggctctggtgatgacctgattaacggtggtcttggtaatgacaccattttcttgaatggtggacaagatactgtggttgtggctcaaggtgcaggtattgacactatcaacaatttccaagtcagtttgggtcaaaaggttggtttgagtggtggacttacctttgagcaattgactttgactcaaagcggtttggatacgttggtgaaagttggtgatgaaactctggctgtgttgaagtttgttcaatctagtgatttgagttcttcagcttttacaacggtctaa

SEQ ID NO: 117 - protein, 299 aa, Anabaena sp.
TRASLGEFVIFNEDGTPAVTWEGIAGFPEPDGTGGGFFVTLTEPTASLSLKVFDDGANEGIESLTFNLVDGEQYQVSPDAGSIALTISDTPTNPVGDAGDNILVGDGNNNSLFGNAGNDRIFGGLGNDYLFGGADDDLLNGGDGNDALFGGAGNNTLLGGAGNDYLTGGAGNNLLDGGDGNDILYGGNGNNTLLGGAGNDIIYSGSGDDLINGGLGNDTIFLNGGQDTVVVAQGAGIDTINNFQVSLGQKVGLSGGLTFEQLTLTQSGLDTLVKVGDETLAVLKFVQSSDLSSSAFTTV

SEQ ID NO: 118 - DNA, 900 bp, Anabaena sp.
acggcgaatcctgatagcaatatctatccagttaaagtcaaccgtggcgatcgcactattgaggtagaagggtttcagggagtaggacggggaagcaatccctcgctggaagtgcgggaaacctttgatgaactcatatttacaggagagggtttagttgccaaaaacttgctccttacccaaactggtgatgatttagttgtcagttttgaaggggttgatgatacccaagtgattctcaaggactttgctttagaaaacctggataacttgccgattcctggtggtcagcatggtcagattggtaacatcatgtttgatggtgatgaaaccctgcaagatagttttgatgtctttgacgcagactccacgcaaaacagaatttggaatcgcaacaccgtcaccttcctgaatgatttagataatcatgtacgtggctttgacaactccgatgatgtcatcaacggtcaaggtggtaatgacattattgggggtttgagtggcgatgatattttgcgcggtggtgaaggtaatgatatcctttatgctggaacaggtactgatattctcgtaggtgggctaggaaacgataccctgtatttgggaagtgatcgccacattgatacagtaatatatcgtcaaggtgatggcagtgatgtgatccatcagttccagcgtggtgcaggcggagatttattgcaatttgaaggtatcgaggcgatcgatgtagtggtgcatggccgcaatacctatttccatttaggtgacggggtgactggaaatacaggatttggttcaggtgagttattagccgagttacgcggtgtcgggggatttacctcagataacatcgggttaaatctggcatctggcaatactgcacagttcttgtttgcataa

SEQ ID NO: 119 - protein, 299 aa, Anabaena sp.
TANPDSNIYPVKVNRGDRTIEVEGFQGVGRGSNPSLEVRETFDELIFTGEGLVAKNLLLTQTGDDLVVSFEGVDDTQVILKDFALENLDNLPIPGGQHGQIGNIMFDGDETLQDSFDVFDADSTQNRIWNRNTVTFLNDLDNHVRGFDNSDDVINGQGGNDIIGGLSGDDILRGGEGNDILYAGTGTDILVGGLGNDTLYLGSDRHIDTVIYRQGDGSDVIHQFQRGAGGDLLQFEGIEAIDVVVHGRNTYFHLGDGVTGNTGFGSGELLAELRGVGGFTSDNIGLNLASGNTAQFLFA

SEQ ID NO: 120 - DNA, 900 bp, Anabaena sp.
tccctttctggtacatctagtgcagatgttctcaacggctttggtggtgatgattatatagaaggtttagctgggaatgacacaatagatggtgggattggaagatttgatcggttgtttggcggtgatggagatgatgcaattaccgatccagatggaatcttaggagcgcatggtggtttaggcaacgatacaatcaacgttacttttgctgccaactgggataatgatagtaatcccaacaactccccacgttctgatggcaagattactggaggctacggcgacgataacattacagtaacgatgaataatagcaagttcttcatcaacatgaagggtgatgagccagttaataacgctcaaggcggtaatgatgtaattacactattaggaagctaccaaaatgcaattgttgacctgggaggtggcgacgatacttttataggtggcaatggcagtgataatgtctctggtggtgctggcaacgataccatttttggtttcggaggtaatgacaacttaactggcaatgacggtgatgatattctcgtcggtggtagcggtaacgatcgcttaactggtggtagtgggaaagatatttttagcttctcttctcttgctgatggcattgacaccattacagactttagcgttgctgatgacaaaattcgtgtcaatgctgctgggttcggtagtgggcttgtagctggtaatctggacgcatcacaatttgtcttgggttcatctgcacaagatggaagcgatcgctttatctacaatcaagcaactggcgctctgttgtttgatgttgacggtataggggcgaatactgccgttcaaattgccactctgtcaaataaaattgcgattaactctacaagtattgtaattgtctaa

SEQ ID NO: 121 - protein, 299 aa, Anabaena sp.
SLSGTSSADVLNGFGGDDYIEGLAGNDTIDGGIGRFDRLFGGDGDDAITDPDGILGAHGGLGNDTINVTFAANWDNDSNPNNSPRSDGKITGGYGDDNITVTMNNSKFFINMKGDEPVNNAQGGNDVITLLGSYQNAIVDLGGGDDTFIGGNGSDNVSGGAGNDTIFGFGGNDNLTGNDGDDILVGGSGNDRLTGGSGKDIFSFSSLADGIDTITDFSVADDKIRVNAAGFGSGLVAGNLDASQFVLGSSAQDGSDRFIYNQATGALLFDVDGIGANTAVQIATLSNKIAINSTSIVIV

SEQ ID NO: 122 - DNA, 900 bp, Anabaena sp.
aacaatgccgtcaatcgcttagaaggcggtgacggcaatgactggttaatcggtaaagatggtaacgatatcctgattggcggtaatggtaatgaccgactcaatggcgagactggtgaggatacattagaaggtggtttaggtaacgacgtttatgaaattgatagtgtaggcgacgtaattattgaagccgcagatgcaggaatagatacagtcatctcatcggtagattggactttaggggtgaatctggaaaacttgactttggtgggtaatcaagccacattaggcataggcaatgatctggataaccgcattactggtaataatgctgataatgtcttgtttggtgaagctggtaatgacatcctgaatggtggtgctggtaacgatgagttgtttggtagtgatggtaatgacatcctgaatggcggtgctggcaacgatgagttgtttggtagtgatggtaatgacatcctgaatggtggtgctggcaacgatgagttgtttggtggtgctggtaatgacatcctgaatggtggtactggtgctgattccttcagttttggtaatccgggtaatcccttcaacaatagtgattttggtatagatactgttgctgattttgcagttggtgtggatgacattaagttagataaggtcagcttctccgctctaactagtgtggttggcaatggttttagtgtaggtggtgagtttgccagtgtcagtaacgatacattggcggcaattagcaatgggttgattgtttacagtttaggtagtggtcgcttgttctataaccaaaatggtagtgctgatggtttgggttctggcgctcactttgctacactctccggcgctcccactctcactgctaataatttcgtgattttttag

SEQ ID NO: 123 - protein, 299 aa, Anabaena sp.
NNAVNRLEGGDGNDWLIGKDGNDILIGGNGNDRLNGETGEDTLEGGLGNDVYEIDSVGDVIIEAADAGIDTVISSVDWTLGVNLENLTLVGNQATLGIGNDLDNRITGNNADNVLFGEAGNDILNGGAGNDELFGSDGNDILNGGAGNDELFGSDGNDILNGGAGNDELFGGAGNDILNGGTGADSFSFGNPGNPFNNSDFGIDTVADFAVGVDDIKLDKVSFSALTSVVGNGFSVGGEFASVSNDTLAAISNGLIVYSLGSGRLFYNQNGSADGLGSGAHFATLSGAPTLTANNFVIF

SEQ ID NO: 124 - DNA, 900 bp, Anabaena sp.
gacaccgttgtttatgacggtaattatgcagattatggtatctctttcctgagcaatggtgatttgcaagtcattgacaagaacctcaccaatggaaatgacggtactgacaccatcaggggtgtagaagtcatcaactttagacaaggcggaagttatggagtggtcacaggtactacaggtaatgatgtattgaccgcatcaaatatgtggtcattcgtcttcggtggtggcggtaacgacattattactggtgggactggcaacgataccttggatggtagtactggcaatgatacgttgattggtggcgctggcaatgatacgttgattggtggtgctggtgttgatactgccgtttatgcgggaaattatgcagattatggtatctctttcctgagcaatggtgatttgcaagtcattgacaagaacctcaccaatggaaatgacggtactgacatcctcaagggtgtagaagtcatcaactttacacaaggcggaagttatggagtggtcacaggtactactggtaataatgtattgaccgcatcaaatatgtggtcatttgtcttcggtggtaatggtaacgacactattactggcggcactggcaatgatactttagtcggagggcttggtgctgataccctcacaggtggacttggggctgataaatttgtctttaactctctttctgaaggaattgatgtgatcaaagacttttcttggcaacaaggagataagattcaaattctcggctctagttttggtgcaacttccactagtcagttcagctttgaccagaatacaggtggtttattctttaacgcccagcaatttgccactcttgagaacaaacctgctggtttcttgacaaatgctgacatccaaattgtttag

SEQ ID NO: 125 - protein, 299 aa, Anabaena sp.
DTVVYDGNYADYGISFLSNGDLQVIDKNLTNGNDGTDTIRGVEVINFRQGGSYGVVTGTTGNDVLTASNMWSFVFGGGGNDIITGGTGNDTLDGSTGNDTLIGGAGNDTLIGGAGVDTAVYAGNYADYGISFLSNGDLQVIDKNLTNGNDGTDILKGVEVINFTQGGSYGVVTGTTGNNVLTASNMWSFVFGGNGNDTITGGTGNDTLVGGLGADTLTGGLGADKFVFNSLSEGIDVIKDFSWQQGDKIQILGSSFGATSTSQFSFDQNTGGLFFNAQQFATLENKPAGFLTNADIQIV

SEQ ID NO: 126 - DNA, 900 bp, Anabaena sp.
aatgatttcggtgtcacgggaactaccaccaatcctgatgggacaattagcattagagtttccccactagctgaaagactggctctcttggaactccccgataatttaccagtcacacaaccattagatattcagttcggctcctctggtagtgataatattacggcggaacctggtcaaatattattcacaggtgatggtgccgatacggtagattctcctgggaataatactatctccacgggcaacggtgatgatacggtatttgtgggcagtgatgcttctgtctctactggtaatggtaacgatcaaatcttcatcggtgtcgagagtccagccagcaataccacagctaatggtggtaatggtgacgacgaaatcaccgtgattgaagcaggtggaagtaataacctttttggcgcagcaggtaatgatactctgcaagtcattgaaggttctcgtcaatttgcctttggtggttctggtaacgacaccctcacaagtaacggtagctataaccgtctcaatggtggttcaggagatgacaaattattctccagtgtgaatgactctttgttcggtggtgatggtgatgatgtgctatttgcaggtcaagctggtagtaaccgcctcactggtggcgctggtgctgaccagttttggattgctaatggtagtttaccaactagcaagaatactgtgactgattttgcagtcggtgttgacaaaatcggactgggtggaattggtgtgacacaatttagcgctttgagtctggtacagcaaggcgctgatactttggtgaaactaggggcgactgagttagttgcattacaaggaattacttcaactagtctgagtgtgactgactttgtttttgctgtaagtttggtgggttag

SEQ ID NO: 127 - protein, 299 aa, Anabaena sp.
NDFGVTGTTTNPDGTISIRVSPLAERLALLELPDNLPVTQPLDIQFGSSGSDNITAEPGQILFTGDGADTVDSPGNNTISTGNGDDTVFVGSDASVSTGNGNDQIFIGVESPASNTTANGGNGDDEITVIEAGGSNNLFGAAGNDTLQVIEGSRQFAFGGSGNDTLTSNGSYNRLNGGSGDDKLFSSVNDSLFGGDGDDVLFAGQAGSNRLTGGAGADQFWIANGSLPTSKNTVTDFAVGVDKIGLGGIGVTQFSALSLVQQGADTLVKLGATELVALQGITSTSLSVTDFVFAVSLVG

SEQ ID NO: 128 - DNA, 900 bp, Anabaena sp.
agttggacattagatgataatttagaaaatctcactctcacaggcagcaatgctattaatgggactggtaatgcgctgagaaataccatcacaggtaacagtgctgataatatcctgtctggtggtgataacgatgacactctcagaggaaatgctggcaacgatattctcaatggaggtgctggtaacgattccttagatggtggacttggtgacgatgtaatgacaggtggcgctagtaatgatacttatttcgttgatagcagcaatgacaccatcatagaagaagctgatgggggaactgatactgttcgtgccagtattacgctaactttaggcgaccacttagaaaatctcatcttgatcggtaatagcccaattgatggtactggtaatgctttaagaaataatattactggtaatgtcgcaaacaacatcttatctggtggtgctgataatgacaccataatcagtggagatggagatgatacgctttatggcgatagtggtaatgatactttaactggcgggaacggcaacgatatactcgtgggtggtatgggtagtgatcgcttgactggcggtaatggtaaagatacttttgctttctctgctccaattaccgatggcatcgacacgattacagactttaatccccttgacgatctccttcgtgttgacgctgctggatttggtggtgggcttgtagctggtactctgcttgcaagtcagtttgttttgggtacagcagccaagactacaagcgatcgctttatttataatcaatccacaggtgcgttattctttgatgttgacggcacaggttctagcagtcaagttcagattgctactctatcgaataaacctgtgattaatgcgacgaatatctcggtaatttaa

SEQ ID NO: 129 - protein, 299 aa, Anabaena sp.
SWTLDDNLENLTLTGSNAINGTGNALRNTITGNSADNILSGGDNDDTLRGNAGNDILNGGAGNDSLDGGLGDDVMTGGASNDTYFVDSSNDTIIEEADGGTDTVRASITLTLGDHLENLILIGNSPIDGTGNALRNNITGNVANNILSGGADNDTIISGDGDDTLYGDSGNDTLTGGNGNDILVGGMGSDRLTGGNGKDTFAFSAPITDGIDTITDFNPLDDLLRVDAAGFGGGLVAGTLLASQFVLGTAAKTTSDRFIYNQSTGALFFDVDGTGSSSQVQIATLSNKPVINATNISVI

SEQ ID NO: 130 - DNA, 900 bp, Synechocystis sp.
gtggatttggttctgccagcggatgctccccgcaccggcctggccacctttgcccccgatggttccgagcaagatgtcctagcggagtatttagcagccaacttcaatagcctggagactgcatttaatcaggcagacacttccccggaatttgatgtccgaatccaaaatctagccttccgtgtggatactgttattgattccactgggcccgttgacccaatcgccaatgagattggagtagtggccgaaaacggcttcttctttgtcctacttcctgggggcgatgaagtacagcttaaatttaacaatcaaccctttgccagtggcacctttggcaattggcaaattttggaagcagaaacggtcaacggcatcaatcaagtgctttggcaaaatcccaaccttggtcagattggtgtttggaatgccgactccaactggaactggatttcttcgcaaacttggcctaccaattccttcaatactctggaagcagaggttaccttccagattgacatcaacaacgatgacctccttggcgatcgcctgacgaccgtggaaaaccagggcaacgtcagtctgctggaaggcatcttgggtaattactacgtccaatctggggatgatttaaccacaccaatcaaatacctaggggaggcttttgacaacaacctcggtaactggcaagccctagcggcggaaactgtacaaggggttaatcaagtgctgtggcaaaatctcgacaccaaccaaatcggtgtttggaactctagtgctgattggaactggatttcctccaatgtatttgaagctggttctccccaggcgatcgcccaagctgaaatttttggtatcccaactaccgtcctaaccacggctgactccgttttagtctaa

SEQ ID NO: 131 - protein, 299 aa, Synechocystis sp.
MDLVLPADAPRTGLATFAPDGSEQDVLAEYLAANFNSLETAFNQADTSPEFDVRIQNLAFRVDTVIDSTGPVDPIANEIGVVAENGFFFVLLPGGDEVQLKFNNQPFASGTFGNWQILEAETVNGINQVLWQNPNLGQIGVWNADSNWNWISSQTWPTNSFNTLEAEVTFQIDINNDDLLGDRLTTVENQGNVSLLEGILGNYYVQSGDDLTTPIKYLGEAFDNNLGNWQALAAETVQGVNQVLWQNLDTNQIGVWNSSADWNWISSNVFEAGSPQAIAQAEIFGIPTTVLTTADSVLV

SEQ ID NO: 132 - DNA, 900 bp, Synechocystis sp.
aatacgtcctatgtctttgatggtcaaaccggtaccctggactatgcctttgccagtgctagcttggcagcacaggtaactggcgcaacagaatgggggatcaacgccgatgaagcagatgccctggactacaacctcgactttgggcgggatgtcaatatttttgatggtacggttccctatcgctcctcagaccatgaccccataattgtcggccttaaccttgcttcccccgttgagccgatcgccaacgaaattggcgtaatggccgaaaatggcttcttctttgtcctacttcctgggggtgatgaagtacagcttaaatttaacaatcaaccctttgccagtggcacctttggcaattggcaaattttggaagcagaaacggtcaatggcatcaatcaagtgctttggcaaaatcccaaccttggtcagattggtgtttggaatgccgactccaactggaactggatttcttcgcaaacttggcctaccaattccttcaatactctggaagcagaagttaccttccagattgacatcaacaacgatgacctccttggcgatcgcctgacgaccgtggaaaaccaaggttctacaactctcctggaaggcatcttgggtaattactacgtccaatctggggatgatttaaccacaccaatcaaataccttggggaagcctttgacaacaacctcggtaactggcaagccctagcggcggaaactgtacaaggggttaaccaagtgctgtggcaaaacctcaacactaatcaaattggtgtttggaactctagtgctgactggaactggatttcctccagtgtgtttgaagctggttctccccaggcgatcgcccaggctggcatttttggtgttgatctgaatgctgtaatttaa

SEQ ID NO: 133 - protein, 299 aa, Synechocystis sp.
NTSYVFDGQTGTLDYAFASASLAAQVTGATEWGINADEADALDYNLDFGRDVNIFDGTVPYRSSDHDPIIVGLNLASPVEPIANEIGVMAENGFFFVLLPGGDEVQLKFNNQPFASGTFGNWQILEAETVNGINQVLWQNPNLGQIGVWNADSNWNWISSQTWPTNSFNTLEAEVTFQIDINNDDLLGDRLTTVENQGSTTLLEGILGNYYVQSGDDLTTPIKYLGEAFDNNLGNWQALAAETVQGVNQVLWQNLNTNQIGVWNSSADWNWISSSVFEAGSPQAIAQAGIFGVDLNAVI

SEQ ID NO: 134 - DNA, 900 bp, Synechocystis sp.
gatggtggtaaaggattccagcttggcaaagacggtactaccagtttcatcggtggtgacgattctatttctggtggcgacggcaatgatttcttagccggtgactttgtcctggtagaccaattgtcagcgccatttgatcccttggatcccaacgattggacatttgtcaatccctacgccactctccaaggccaggcgggtgatagtaaagctcaagctgctcaagctgctatcaatttggctcaactccgccttgagttccgtgccgttggcggcgatgacgagctcgtgggtggtcgtggcaacgatactttctatggtggtcttggtgcagacactattgatatcggtaatgatgtcactgtcggcggtgttggcgttaacggtgccaatgaaatctggtacatgaatggtgcctttgaaaacgcagcggtcaatggagccaacgtcgataacattactggtttcaacgtaaacaacgacaaatttgtcttcgcggctggagccaataacttcttgtctggtgatgctacatccggccttgccgtccaacgtgtccttaatttacaggcggggaatacggtcttcaatctaaacgatccgatccttaatgcctctgctaataacatcaacgatgtgttcttagctgtaaatgcagacaacagtgtcggtgcgtctctctccttctccttgctacccggcttgccttctctggttgagatgcaacagatcaatgtctcttctggtgctctggctggtcgcgaattcctgttcatcaacaacggtgttgcggctgtcagctcccaagacgacttcctcgtagaacttacaggtattagcggtacctttggtctggacttgactcctaacttcgaggttcgtgagttctacgcctaa

SEQ ID NO: 135 - protein, 299 aa, Synechocystis sp.
DGGKGFQLGKDGTTSFIGGDDSISGGDGNDFLAGDFVLVDQLSAPFDPLDPNDWTFVNPYATLQGQAGDSKAQAAQAAINLAQLRLEFRAVGGDDELVGGRGNDTFYGGLGADTIDIGNDVTVGGVGVNGANEIWYMNGAFENAAVNGANVDNITGFNVNNDKFVFAAGANNFLSGDATSGLAVQRVLNLQAGNTVFNLNDPILNASANNINDVFLAVNADNSVGASLSFSLLPGLPSLVEMQQINVSSGALAGREFLFINNGVAAVSSQDDFLVELTGISGTFGLDLTPNFEVREFYA

SEQ ID NO: 136 - DNA, 900 bp, Synechococcus elongatus
agctatgtggtgtttggcaacgcagcaccggtgcttgatttggatggcaccacatcaccagagctgaactttggcgctgtctttactggtacgccagtctcagttgtgggttcaggactcaccattaccgatctcaactctccaaccctcgccgcagcgaccgtgaccttggtcaaccggcccgatggcattgctgaaagtttgagtgcaatcacggatggcactgcaattaaggccagctatgacagcaataccggggtgctgctgctcgtgggtctggctactgtggcggattatgagaaagtcctgcgcaccgtcacctataccaacacctctaatgcagccgatctggatgtaagccgtcgcacgattgagtttgtcctcgacgatggagcagattttgccaacaccagtgcggtagtcactaccacgctgagcttcaagaatgaagtcaatacaatcactggaacccccagactcgacttcctccgaggcagcaagggagatgacttgattacggggctcggggggaatgacttcctgtttggcagggctggtaatgacaccttgattggcggactcggctctgacgtcctttctggtggagccggcaaggaccgctttgtctacaccgctgttactgaggctcgcgacttaatcatcgactttaatgccaagcaggatgttctggatctaagcgggttgttggatagtctgggctatcaaggctctaatcctgttgcggatcaggtcctgcgcttgaacagtcagtctttcttgggcacgacggtctctgtcaatgtagcgggactcggtggagtgcccgactttgtctccctagtgaccctgcttggtgtctcttcttctgccctcgtcattggtgaaaacatcatcatttag

SEQ ID NO: 137 - protein, 299 aa, Synechococcus elongatus
SYVVFGNAAPVLDLDGTTSPELNFGAVFTGTPVSVVGSGLTITDLNSPTLAAATVTLVNRPDGIAESLSAITDGTAIKASYDSNTGVLLLVGLATVADYEKVLRTVTYTNTSNAADLDVSRRTIEFVLDDGADFANTSAVVTTTLSFKNEVNTITGTPRLDFLRGSKGDDLITGLGGNDFLFGRAGNDTLIGGLGSDVLSGGAGKDRFVYTAVTEARDLIIDFNAKQDVLDLSGLLDSLGYQGSNPVADQVLRLNSQSFLGTTVSVNVAGLGGVPDFVSLVTLLGVSSSALVIGENIII

SEQ ID NO: 138 - DNA, 900 bp, Synechococcus elongatus
aaaggtcctgagcctgaaggtgtcgtgattggccagattaacgatcgcacctatgcctttgtcggtcttgagcggaccggtggcgtcatagtctacgacgtgactacccctaacaatcccacctttgttcagtacctcaacaatcgtaatttcaacgctgatgttgaaagtgccgaagcgggtgatttaggccctgagggtcttgctttcatctctgcagaggacagccccaacggcaaacctctgttggttgtcgccaacgagatcagtggaactacaacgctctatgagattaatgtcggttctaatcctgacttgatcaagttagacaacagcgcccagattgcttacatcacttatctaggacggcctggcgatcgcggtggactgaccttttggaatgaggttctgagagatgccgaaatcagctacgaccctcaaactggtgatttaattactggtgaagaagttcttcccttcaacgccttcatcaacgggtttggagattcttctgaagctgatcaaatctacggtggtaaatctgcagccgatcaggtgaacttaatttataactttgccttcaatcgtaatgctgagagtgctggccaagccttctgggtcaaccagctgaatagtcgccagctcagcttggcggaactggctctagaaattggtctgaacgcgacaggcaatgattcagtagttcttaacaacaagattagaagtgccactctgttcaccgattcgattgacacgaatgttgaactagctgcttatcaaggtagtaaggggaccagctttggtcagacctggctagatcagtttgactttagccaaagtagccaagctctggttgatagtgctcttaacgctttagtcaatgacctacctcttggatag

SEQ ID NO: 139 - protein, 299 aa, Synechococcus elongatus
KGPEPEGVVIGQINDRTYAFVGLERTGGVIVYDVTTPNNPTFVQYLNNRNFNADVESAEAGDLGPEGLAFISAEDSPNGKPLLVVANEISGTTTLYEINVGSNPDLIKLDNSAQIAYITYLGRPGDRGGLTFWNEVLRDAEISYDPQTGDLITGEEVLPFNAFINGFGDSSEADQIYGGKSAADQVNLIYNFAFNRNAESAGQAFWVNQLNSRQLSLAELALEIGLNATGNDSVVLNNKIRSATLFTDSIDTNVELAAYQGSKGTSFGQTWLDQFDFSQSSQALVDSALNALVNDLPLG

SEQ ID NO: 140 - protein, 180 aa, Synechococcus sp.
MINQPCIVPAEKGFTLIELLTGMLIVGILASISAPSFLGLVNRGRVNEALNRTRGALQEAQREVIKKSNTCNLTFSPSGQTVNITGGCLVTGPRVMSRVTYRHTLANNDPANVIELDFKGVPVEDNFNDGQEVFVFRGNGNYERCLVISRALGLIRVGTYNTSGTSDTSTDATKCITGQV

SEQ ID NO: 141 - protein, 245 aa, Synechococcus sp.
MINQPCIVPAEKGFTLIELLTGMLIVGILASISAPSFLGLVNRGRVNEALNRTRGALQEAQREVIKKSNTCNLTFSPSGQTVNITGGCLVTGPRVMSRVTYRHTLANNDPANVIELDFKGVPVEDNFNDGQEVFVFRGNGNYERCLVISRALGLIRVGTYNTSGTSDTSTDATKCITGQVDTKKKVDDDLGTIENLEEAKKKLLKDVEVLSQRLEEKALAYDKLEKTKTRLQQELDDLLVDLDHQ

SEQ ID NO: 142 - protein, 174 aa, Synechococcus sp.
MKIANFISRKNINLNYGFTLFELLAGLVIVGILAGISVPSFLAFVERGRVNEAANILRGVIQSSQREAIKKSTDCTIQLPAKQTKNPTISSTCSIDGPRRLKNVVIQYNQTDQISIDYQGRFNRKRTIVLYSENTNYKRCLVVSSFIGMTRTGIYTDQDLNTVSADYCQKTNVG

SEQ ID NO: 143 - protein, 239 aa, Synechococcus sp.
MKIANFISRKNINLNYGFTLFELLAGLVIVGILAGISVPSFLAFVERGRVNEAANILRGVIQSSQREAIKKSTDCTIQLPAKQTKNPTISSTCSIDGPRRLKNVVIQYNQTDQISIDYQGRFNRKRTIVLYSENTNYKRCLVVSSFIGMTRTGIYTDQDLNTVSADYCQKTNVGDTKKKVDDDLGTIENLEEAKKKLLKDVEVLSQRLEEKALAYDKLEKTKTRLQQELDDLLVDLDHQ

SEQ ID NO: 144 - protein, 180 aa, Synechococcus sp.; identical to SEQ ID NO: 59

SEQ ID NO: 145 - protein, 245 aa, Synechococcus sp.
MLRLLFLHRKKAAQDFQGFTVIELMIVMIITGILTAIALPAFLNQVDKSRYAKARLQMRCMLQELKVYRLNHGSYPPDQNRNVPYYPGSECFKVHTGYVRDRPDINRNNNTDIPFHSVYDYERWDYNSGCYIAVTFFGKNGLRRFTQAAINEISTTGFHFYDGTDDDLVLVVDITDSPCDDTKKKVDDDLGTIENLEEAKKKLLKDVEVLSQRLEEKALAYDKLEKTKTRLQQELDDLLVDLDHQ

SEQ ID NO: 146 - protein, 175 aa, Synechococcus sp.
MSSYKAICVWLIHYSKRNNQGFTLIELLVVMIIIGILSAISLPVMFSMAAKARQSEAKTTLSVLNRGQQAYYAEKSTFSPDILNLGVTTIIETNNFSYGNAGSLVNYQTGAAYGATPKDPATVKDYSAGVTSLAIARVPLIICEEEDPTVVGPFPPLLDSGAGTLSCPVGYIKLR

SEQ ID NO: 147 - protein, 240 aa, Synechococcus sp.
MSSYKAICVWLIHYSKRNNQGFTLIELLVVMIIIGILSAISLPVMFSMAAKARQSEAKTTLSVLNRGQQAYYAEKSTFSPDILNLGVTTIIETNNFSYGNAGSLVNYQTGAAYGATPKDPATVKDYSAGVTSLAIARVPLIICEEEDPTVVGPFPPLLDSGAGTLSCPVGYIKLRDTKKKVDDDLGTIENLEEAKKKLLKDVEVLSQRLEEKALAYDKLEKTKTRLQQELDDLLVDLDHQ

SEQ ID NO: 148 - protein, 190 aa, Synechococcus sp.
MKNFTFKLLQQLNKKKADKGFTLIELLVVIIIIGILSAIALPAFLNQAAKAKQSEAKQTLGALNRGQQAYRLESPEFAPEVDLLALGVEIDTTNYAYGDDGSATTGNGEFAFNFNNLEGTDFTETAGIGARAKDTAAVRDYDGATGATEDSEGNATTVTVICEETAPQDDDQDMSYSFADGLGCDAGNQL

SEQ ID NO: 149 - protein, 255 aa, Synechococcus sp.
MKNFTFKLLQQLNKKKADKGFTLIELLVVIIIIGILSAIALPAFLNQAAKAKQSEAKQTLGALNRGQQAYRLESPEFAPEVDLLALGVEIDTTNYAYGDDGSATTGNGEFAFNFNNLEGTDFTETAGIGARAKDTAAVRDYDGATGATEDSEGNATTVTVICEETAPQDDDQDMSYSFADGLGCDAGNQLDTKKKVDDDLGTIENLEEAKKKLLKDVEVLSQRLEEKALAYDKLEKTKTRLQQELDDLLVDLDHQ

SEQ ID NO: 150 - protein, 34 aa, Synechococcus sp.
MKKFSFALAAASALSLSLASTAQAGQGGIAAGAA

SEQ ID NO: 151 - protein, 37 aa, Synechococcus sp.
MHYWYRLLGFTGGVALFWAAQELSAVAASPQPSDATA

SEQ ID NO: 152 - protein, 44 aa, Synechococcus sp.
MTTFTFSRPQSLKLATAGAFLALGVLSIAQPAKADNVSSSTMIS

SEQ ID NO: 153 - protein, 34 aa, Synechococcus sp.
MLSRFLILCLALCLWAVSPLPSFAASPFAGERPT

SEQ ID NO: 154 - protein, 36 aa, Synechococcus sp.
MNFAKIAAVAAGAAALSLGFASSAKAEFAASVSFVD

SEQ ID NO: 155 - protein, 44 aa, Synechococcus sp.
MTTFAFSRPQSLKLATAGAFLALGVLSIAQPAKADNVSSSTMIS

SEQ ID NO: 156 - protein, 33 aa, Synechococcus sp.
MRKSNLSLKTLAIATLLSSSLFACGSPNQSITS

SEQ ID NO: 157 - protein, 35 aa, Synechococcus sp.
MKQSATRLRTLSLGLAGLTLTAALAACNTTQTPTE

SEQ ID NO: 158 - protein, 33 aa, Synechococcus sp.
MTMKIRYAATLVSISLLSLGAIAGCSGVKNPCA

SEQ ID NO: 159 - protein, 39 aa, Synechococcus sp.
MAYSVVSWRKNLSWALCSLALLLPLPLNAQVQVSPMVIK

SEQ ID NO: 160 - protein, 38 aa, Synechococcus sp.
MKNLSVKLLSGTATMTAVSLMAINPATADTVSGSVTFT

SEQ ID NO: 161 - protein, 38 aa, Synechococcus sp.
MKITRHTIGKGLMLGTMILMGSSFSANAAPLSSTGPLP

SEQ ID NO: 162 - protein, 35 aa, Synechococcus sp.
MKTSLSLWKSLSIASAAVGVSVATAGTAQAQANNS

SEQ ID NO: 163 - protein, 33 aa, Synechococcus sp.
MTSLKTVSLAATAFVTMASQAIAADNQGLLEQI

SEQ ID NO: 164 - protein, 34 aa, Synechococcus sp.
MFKPITLLNVALLGLLGFTPLLQASTPNASQIAS

SEQ ID NO: 165 - protein, 37 aa, Synechococcus sp.
MTKFLNYCLSVALAIAVCFGVTQPASALPQPSFTLAS

SEQ ID NO: 166 - protein, 36 aa, Synechococcus sp.
MTLKRKHLLALSAVFTTFAPLSLTTAPTLANTDTSP

SEQ ID NO: 167 - protein, 38 aa, Synechococcus sp.
MKFKLPHFVLGLSIALVISLHGCTFGNSGQTLVVAIAA

SEQ ID NO: 168 - protein, 39 aa, Synechococcus sp.
MKSQTKRLKRACSYLVLALSAMVPSVALAGHTNTILHTM

SEQ ID NO: 169 - protein, 32 aa, Synechococcus sp.
MKKIIALLSLGSVMLTAGAAQAQITPTNQYSY

SEQ ID NO: 170 - protein, 29 aa, Synechococcus sp.
MSKSTMIHSRQFYSAAAIALCFGSLLVSC

SEQ ID NO: 171 - protein, 28 aa, Synechococcus sp.
MKSRSLSLCGLFLGLAIATGCTPATNNN

SEQ ID NO: 172 - protein, 33 aa, Synechococcus sp.
MKPPKIALLSSLCCLGFTSLAVATLPQASQIVS

SEQ ID NO: 173 - protein, 30 aa, Synechococcus sp.
MGKTQFQPVSQILALASLATLAFSSQSLAQ

SEQ ID NO: 174 - protein, 32 aa, Synechococcus sp.
MKQQARDSFALAVGSMMPVLIATQPAQAQTSA

SEQ ID NO: 175 - protein, 36 aa, Synechococcus sp.
MLSPSKKFLILVLASLLILPMPAAIATPIDPCLLRE

SEQ ID NO: 176 - protein, 36 aa, Synechococcus sp.
MTNCYRKLLLFLSLSLMMGAGQVSAASLVGPIQDPL

SEQ ID NO: 177 - protein, 37 aa, Synechococcus sp.
MSTTSISPGKTGTITCLSALLLSTAIAPFAALNPAQA

SEQ ID NO: 178 - protein, 28 aa, Synechococcus sp.
MQTSKFNLAIALSLAAIATFTGACQDTT

SEQ ID NO: 179 - protein, 32 aa, Synechococcus sp.
MLRRVILAIAIALWWGLWVVWAAPQSQFLTIA

SEQ ID NO: 180 - protein, 36 aa, Synechococcus sp.
MKSGLKLSLTLAFAAGIVVPAGSVNAQVCSDVGGGA

SEQ ID NO: 181 - protein, 34 aa, Synechococcus sp.
MAFYKQISAFCSATSLLTIPLAIAPAQAQQSYPL

SEQ ID NO: 182 - protein, 35 aa, Synechococcus sp.
MRFTKTLALSLALGSTLGFSTVAQAGDYGSYGDKT

SEQ ID NO: 183 - protein, 31 aa, Synechococcus sp.
MFKTLIKNSAAIAFVLLGSIAVIPGASSQIS

SEQ ID NO: 184 - protein, 36 aa, Synechococcus sp.
MNHFLPRPLLRSLFAVCLAVMTWAIAPAAFAVNNPE

SEQ ID NO: 185 - protein, 29 aa, Synechococcus sp.
MKALILALGISCLAIPVAAQGTCLRISDF

SEQ ID NO: 186 - protein, 38 aa, Synechococcus sp.
MSKTVRTFLSGASVALGATVAFSGTAQANTELLDQINS

SEQ ID NO: 187 - protein, 33 aa, Synechococcus sp.
MKTLTFLMIPAMALSLMPQSVLAWNAYHLYNKD

SEQ ID NO: 188 - protein, 37 aa, Synechococcus sp.
MKSTPHFSRTRMLVMGGFMSLSSVALAAPALAHHPFG

SEQ ID NO: 189 - protein, 29 aa, Synechococcus sp.
MKRMLGLAMALFIASPASAGNLLQGEPYY

SEQ ID NO: 190 - protein, 38 aa, Synechococcus sp.
MNLKFLKSLWATAAIAFAISVNPSLVFAETEPPSETKT

SEQ ID NO: 191 - protein, 39 aa, Synechococcus sp.
MKFNLFNPYLLAASAIISACFILPKPTQAASWLECNGDS

SEQ ID NO: 192 - protein, 33 aa, Synechococcus sp.
MTRFFLVIAPILAGLAVAAGAFASHGLKETLDA

SEQ ID NO: 193 - protein, 36 aa, Synechococcus sp.
MKLPLLWVSLVLILLLSFGWGSRSAATSAPTVDLET

SEQ ID NO: 194 - protein, 39 aa, Synechococcus sp.
MVKMFQFKRTLSVGAIATSLTMITGGVWAAEKPTIQIAI

SEQ ID NO: 195 - protein, 35 aa, Synechococcus sp.
MDYLNFVYFFTTMIALAALPSTSVALVVTRSATAG

SEQ ID NO: 196 - protein, 37 aa, Synechococcus sp.
MGIKKAIATFFISTALFPLGFSNSAQAEVATLEFDYE

SEQ ID NO: 197 - protein, 41 aa, Synechococcus sp.
MNRLKTAATYLLLGAIALVMLFPLLWLLSTALKSPTENVFS

SEQ ID NO: 198 - protein, 611 aa, Synechococcus sp.
MIHDDGRSNYSNNRPFQDIFKARFSRRSMLQKSMMLSAAGFIGAIAGNSVLKPSTAATQVAQRRTSPLLGFNAVTLAQGNGPVPSISSDYQYQVLIPWGTPIQPGGPEYNGDPNTRPTADEQAQQIGIGHDGMWFFPLGNNNDHGLLAINHEFGINEHVLGKADPASLEDVRLSQHAHGASVVEIKKNNRGVWEVVRSNYARRIHANTPMAFSGPAANHPLLKTAAGNAPKGTINNCSNGHTPWGTYLTCEENFNTYFGATGEWTPTEAQTRYGLASSSRYGWANYDERFDLSKAAYKNEENRFGWVVEIDPMDPNQTPVKRTALGRFKHEGAEIVVGRGGRVVCYMGDDERFDYIYKFVSANNWQSMRARGISPFDEGQLYVAKFNDDGSGEWLPLSMDNPALQGKFQDQAEILVYTRLAADAAGATPMDRPEWITVGTEENVYCALTNNSRRTEADAANPLAPNPDGHIIRWQDSDRHVGTTFTWDIFAIAQDTHGTEESFASPDGLWADPDGRLFIQTDGAQKDGLNDQLLVADTNTKEIRRLFTGVTDCEVTGITVTPERRTMFINVQHPGDGNPATTNFPAPQGSGMVPRDSTVVITRKDGGIVGS

SEQ ID NO: 199 - DNA, 1836 bp, Synechococcus sp.
atgattcacgacgacggcagaagtaattattcaaataatcgtcctttccaagatattttcaaggcgcgattctcccgccggagtatgctccaaaaaagcatgatgctctccgccgctggttttatcggggcgatcgccggcaatagcgtcctcaaacccagcaccgccgccacccaagttgcccaacggcgcaccagtccccttttgggattcaatgctgtaaccctagcccaaggcaatggccccgtccccagtatttccagtgactaccaataccaagtgttgatcccctggggtacccccatccaacccggtggccccgaatacaatggcgaccccaacacccgacccaccgccgacgaacaggcccagcagattggcatcggccacgatgggatgtggtttttccccctcggcaacaacaatgaccatggtttgttggcaattaaccacgaatttggcatcaacgaacacgtcctgggtaaagcagatcccgccagccttgaggatgtgcgattgtctcaacatgcccatggtgcctccgtcgttgaaattaagaaaaataatcgtggcgtttgggaagtggttcgcagtaactatgcccgccggatccatgccaatacccccatggccttcagtggccctgcagcaaatcatcctctcctaaaaacggcagcgggcaatgcgccgaaagggactatcaataactgttctaacggtcacactccctggggcacctacctcacctgtgaggaaaacttcaacacctactttggggcaaccggagaatggacgcccaccgaagcccagacccgctatggactcgccagcagttctcgctatggttgggcaaactatgacgagcgattcgacttgtcaaaggcggcctacaaaaatgaagaaaaccgctttggttgggtcgtcgaaattgatccgatggatcccaaccagacccctgtgaagcgcacagcccttggtcgttttaagcatgaaggggcagaaattgtcgttggtcgtggcggtcgtgtggtctgctatatgggtgacgatgaacgctttgactacatttacaagttcgtttcggcaaacaattggcagtcaatgcgggcgcgggggatcagtcccttcgatgaaggccagttgtatgttgccaagttcaacgatgatggctctggagagtggttacccctcagcatggataacccagccttacaaggaaaattccaagaccaggctgaaatccttgtgtatactcgcttagcggcagatgcggctggggcaacgccgatggatcgtccggaatggatcactgtcggcaccgaggaaaacgtttattgtgccctcactaacaatagccgtcgcacggaagctgatgcggcgaaccccctggcaccgaatcctgatggccacattattcgctggcaggatagcgatcgccacgtggggacaaccttcacctgggatatttttgcgatcgcccaagatacccatggcaccgaagaatcttttgcctctcccgatggactatgggctgaccccgatggccgtctctttatccaaaccgacggtgcccagaaggacggcttgaatgaccaactgctcgtagcggataccaataccaaggaaattcggcgtctctttactggggtgacagattgcgaagtaacggggattacggtgaccccagagcgtcgcacgatgtttattaacgtgcagcacccaggcgatggcaacccagccaccaccaatttcccggctccccaggggagtgggatggtgccccgggatagcaccgtggtcatcacccgtaaagatggcggcatcgttggctcatag

SEQ ID NO: 200 - protein, 493 aa, Synechococcus sp.
MNLNSGVKSLVASMVKPKLKASFKLALLSTLAGLPLGTLIFPPQAIAQNATIRGEVVFTLTDLAGAEMLAVTKDGRHALVVGAKTATLVAIEDNALTVEGTWTLTDEFLPAGSADAELTGVSISPDGAFALIGVKDADDANLDTFDEMPGKVVALSLPDLEPLGHVTVGRGPDSVAIAPNGQFAAVANEDEENEEDLTNLENGAGTVSIIDLRRGPNRMTQVEVPIPPDNIPFFPHDPQPETVRIAADSSFIVATLQENNAVARIEIPSPLPKRLTPDIFSVQNFDVGVRTGFGLVQDKVGEGSCRSGSYDLSLRQEFTSAREPDGIAITPDGRYFVTADEDNLTNVNNQSYEGILLSPHGTRSISVFDATTGELLGDSGNSIEESIIALGLPQRCNSKGPEPEVVSVGVVNGRTLAFVAIERSDAITIHDISNPRNVQLLDTVVLNPDVVRANQEAGFEPEGIEFIPATNQVIVSNPEGNAMSLVNINVMPR

SEQ ID NO: 201 - DNA, 1482 bp, Synechococcus sp.
atgaacttaaatagtggtgtgaaaagcttagtggcatcaatggtgaagcccaagctaaaagctagtttcaagttagctctcttatcgactcttgccggccttccattgggcacgctaatctttccgccccaagcgatcgcccaaaacgcaactattcgaggtgaagttgttttcacattaacggatctcgccggcgcagaaatgctcgctgtcacaaaagatggtcgccacgcccttgtggtcggcgcaaaaacagcgaccttagtggcgatcgaagataatgccttaaccgtcgaagggacttggaccctaacggatgaatttttgcccgcaggttctgcggacgctgaactcactggagtttccattagcccagacggggccttcgcactcatcggggtcaaagacgcagatgacgcaaatctggatacctttgacgaaatgccaggcaaggtcgtggccctctctctccccgatctagaaccccttgggcacgtaactgtaggtcgcggcccagactccgtggcgatcgccccgaatggtcagtttgctgccgtcgccaatgaagatgaagaaaacgaagaagatctgacgaacctagaaaacggcgctggaaccgtttcgatcattgatctccgacgtggccccaatcgcatgacccaggtcgaggtgcccattccccccgacaatattccctttttcccccacgacccacagcctgagacggttcgcatcgcggctgatagctcttttattgtcgccacactacaagaaaataatgctgtcgctcgcattgaaattccctctcctttgcccaaacgtctaacccctgatatcttttcggtgcaaaactttgatgtcggcgttcgtacgggtttcggtttagttcaagataaagttggagaaggaagctgtcgttctggcagctatgacctatccctcagacaagaattcacctctgcccgtgaacccgatggcattgccattaccccagatggtcgctactttgtcaccgccgatgaagataatttgaccaatgtcaataaccagtcctacgaaggaattctcttaagtccccatggtacccgcagtattagtgtctttgacgcaaccacgggtgaacttttgggagatagcggcaattccatcgaagaaagcatcatcgccctcggcttgccccagcgctgtaacagcaaaggcccagaacctgaggttgtttccgttggtgttgtaaatggtcgtaccctagcattcgtggcgatcgagcgttcagatgcgatcacaatccatgacatttccaaccctagaaatgttcagctgctcgatactgtcgttctcaaccctgatgttgttcgggccaatcaagaggctgggtttgagccagaagggattgaatttattcctgcaacgaatcaagtgattgtctccaacccagaaggcaacgccatgagcttggtaaacatcaatgtgatgccacgctag

SEQ ID NO: 202 - protein, 483 aa, Synechococcus sp.
MVSLAIAPLSLWAETVELQLLHLNDVYEITPLGGGATGGLARLATLRKELLAENPHTFTVLAGDLFSPSALGTAVVDGDRLAGKQIVAVMNQVGLDLATFGNHEFDISESQFKQRLAESDFQWFSGNVLTAAGEPWDNVPPYVIETIYGEAGTPVRVGFVGVVIPSNPVDYVTYLDPLEQMEILVAELEAQTDIIVAVTHLAMQDDHHLAENIPEIDLILGGHDHENIQQWRGADFTPIFKADANARTVYLHNLSYDTETEQLTVQSHLQPITGAIAADPETEQEVNYWQQLAFDGFRADGFEPEQIITESPIALDGLESSVRNQATALTDIIAQSMLTATPAAELAIFNGGSIRVDDVLPPGPLSQYDVIRILPFGGNLATVEIKGTTLERILNQGLANRGTGGYLQTARVTFVPESQTWQIGDRPLDPERIYRVAATEFLISGRETGLDFFTPDHPDVTLLETGEDVRFAFIQQLQQEWID

SEQ ID NO: 203 - DNA, 1452 bp, Synechococcus sp.
atggtcagtttggcaatcgcccccctatctctctgggctgaaacggtagaattgcaactgcttcacctcaatgatgtctatgaaattacgcccctgggtggtggggcaacggggggcctggcgcggttggcgaccctacgcaaggaactgctcgccgaaaatccccacactttcaccgttttagctggggatttatttagtccgtcggccttggggactgcggtggttgatggcgatcgcctcgcaggaaaacaaattgtggcggtgatgaaccaagtgggcttggatcttgccaccttcggtaaccacgaatttgacatcagcgaatcccagttcaagcaacgcttagcagaatcagatttccagtggttttcggggaatgtcctgacggcggcgggggaaccctgggataatgtacctccctacgtgattgaaaccatttatggtgaggcgggcaccccggtgcgtgttggttttgtgggggtggtaattccgagcaatcccgtagattacgtcacctatctcgacccgctagaacagatggaaatcctcgtcgcagaattagaggcacaaacggatattattgtggcggtcactcacctggcgatgcaggatgaccatcatcttgctgaaaatatcccggaaattgacctaatcctggggggccacgaccatgaaaatattcaacagtggcgtggtgcggattttacgccgattttcaaggccgatgccaatgctcgcacggtttatctccataatctcagctacgacacagaaacggagcagcttacagttcaatcacatttgcaaccgattaccggggcgatcgccgcagatccagaaacagaacaggaggttaattattggcagcaactggcctttgatggttttcgggctgatggttttgaaccagagcaaatcattaccgaaagtccaatcgccctagatggtttggaaagttccgtgcgcaaccaagccacagcgttaacggacatcattgcccagtcgatgttaacggcgacacccgctgccgaattagccatttttaatggcggctcgatccgtgttgatgatgtgctgcctcccggcccgttgtcccagtatgatgtgattcggattttgcccttcggcggaaatttggccaccgtcgagatcaagggcacaaccttggaacgcattctcaatcaaggtttagccaatcgcggcaccgggggatatttgcaaacggcgagggtgacctttgtcccggaaagtcaaacctggcaaattggcgatcgccctttagatcccgaacgcatttatcgggtcgcagcgacggaatttctcatctccgggcgagaaacgggcctcgatttcttcacgcctgaccatcccgatgtgaccttgctcgaaacgggagaagatgtacgttttgcctttattcaacagctccaacaggaatggatcgattag

SEQ ID NO: 204 - protein, 351 aa, Synechococcus sp.
MHGNRRQFLTYGGLALGSVLISRGIIAKSQAIANSAPTALNAPAPGETRLVVISDLNSAYGSTDYLSQVKRAIALIPDWQPDLVLCAGDMVAGQKSSLTPAQLTSMWQAFERYIAQPLRQANIPFAFTLGNHDASGSLRNGQYAFAADRQAASQYWRNPAHTPTLDFVDRRHFPFYYSFTQDNIFYSVWDASTARISPAQLAWIEASLASDQAQRSRLRFALGHLSLYPVASGSRSEPGNYLHDGDRLQALLEKYNVHTYISGHQHAYYPAHRGQLELLHTGALGDGPRSLVQGNLSPYRSLTMIDIPRGGTNLRYTTYNMDRLTVVDHGTLPGSLNTPRGYLQRRDLRAT

SEQ ID NO: 205 - DNA, 1056 bp, Synechococcus sp.
atgcacgggaatcgacgacagtttttaacctatgggggcttggccctagggagtgtacttatttcgcgtgggattattgcaaaatctcaggcgatcgctaattctgcaccgactgcacttaatgccccagccccaggggagacgcgcctggttgtgattagcgacctgaacagtgcctatggttccacggattatctgtcccaagtgaaacgggcgatcgccttgattcccgattggcaaccggatctagtgctctgtgcgggcgatatggtcgcaggccaaaaaagcagcctcaccccagcccagctcacctccatgtggcaagcctttgaacgatacattgcccaacccctgcgccaagcaaacattcccttcgccttcaccctcgggaaccacgatgcttccggctccctgcgcaatggacaatacgcctttgccgcagatcgtcaggcggccagtcaatattggcgcaaccctgcccataccccgaccctagactttgttgaccgtcgtcattttcctttctattacagctttacccaagacaatattttttactctgtgtgggatgcttccaccgcccgcattagtccagcacagttggcttggatcgaagccagtctcgccagtgaccaagctcaacggagtcgtttacggttcgccctagggcatttatccctctatcctgtcgcttcgggcagccgctcagagccaggaaattatctccatgatggcgatcgcctccaggctctgctcgaaaaatacaacgtccacacctacatcagcggtcaccaacacgcttactatcccgctcaccgggggcaattggaactgctccatacaggtgctttaggagatgggccgcgttctctagttcaaggcaatctttccccttaccggagcctcacgatgattgatattcccaggggcggcacaaacttgcgctacaccacctacaacatggatcgcctgactgtggttgatcacggcactttacccggcagtttgaatactccgaggggatatttgcaacgccgcgatctgcgggccacttga

SEQ ID NO: 206 - protein, 157 aa, Synechococcus sp.
MAYKLLFVCLGNICRSPSAENIMRHLLEQEGLSNKILCDSAGTSSYHIGAAPDRRMQAAAQKRDIRLMGSARQFSRADFEAFDLILAMDRANYRDILSLDRADIYGEKVKMMCDYATNFPDSEVPDPYYGGQSGFDYVIDLLLDACQGLLTEIKQEM

SEQ ID NO: 207 - DNA, 474 bp, Synechococcus sp.
atggcctataaattattattcgtttgcctcggtaacatctgccgttccccctccgccgaaaatattatgcggcatcttttggagcaagaaggtttaagcaataaaattctctgcgattcggccgggacttctagctatcacataggagccgccccagaccgacggatgcaggcagcggcccaaaagcgcgatattcgtctgatgggtagcgcccggcaattttcccgcgctgattttgaagcatttgacctgatcctggcaatggatcgcgctaattatcgtgacattttgtccctagaccgggcggatatctatggcgaaaaagttaaaatgatgtgtgactacgccacgaattttcccgatagcgaagtgccagatccctactacggcggccaatcgggttttgactatgtgattgatttgctcctcgatgcctgccaaggactcctcacagaaattaaacaggaaatgtga

SEQ ID NO: 208 - protein, 600 aa, Synechococcus sp.
MSITLPYLRASGSLALTFQAADLVGDRYWVVAPQIWQDTKPEAPPDCTAPNDLAQRYGKLYSRQLHLPRIYDILSLPEGEILLLDNIPINNQGELLPALGSVWADASPLQQLNWLWQMLDLWEDLAAVAMGTSLLPLENIRVDGWRLRLMELLADPPGAPVTLGALVTPWRSLLAESTPPVQAMLTELIESFSEPDADLEIILPRLNQLLLEQSSQQHLQMAIASATDQGKLPTSNQDAHYPTTQDLAAPPTATLALSDHLLMVCDGVEGHGQGDVASQLAIQSLKLQLTGFFQGLFDTDEVVPPAVIEQQLAAYIRITNNLIAERNDQEGRTGGDRMATTLTLALQVPQRPKADKLQDSHSHELYIAQVGDSRAYWITKDQCVCLTVDDDLLSREVQAGRAIYRQGLQRPDHMALTQALGIKGGDRLHPVIRRFVFAEDGVLVVCSDGLSDQQFLESHWQTFAPVIIQGHLPPAALLQGLIEKAIAKNPEDNITAAIAFYRFTTDTFTQAPDIETAPAPEDFEPEFVPPDLALDTTLEAELESEPETENSLSQFTLILVSLVAILLMLVLAAFGLNWLLNRGPEPTQPGEPNLETPTNA

SEQ ID NO: 209 - DNA, 1806 bp, Synechococcus sp.
atgtctattactcttccttatctccgagcatcgggttccttggcgttaacctttcaggcagcggatcttgttggcgatcgctactgggtggttgcaccgcaaatttggcaagacaccaagcccgaagcaccaccggactgcacagcccccaatgacctggcccaacgctatggcaaattatattcccgtcaactgcacttgccccgcatttacgatattttgtctctcccggaaggggaaattttactcctcgacaacatcccaattaacaatcaaggggaactgctgcctgccctaggatcggtctgggccgatgcttctcccctgcaacagttaaattggctgtggcaaatgctcgatctttgggaagatttggcagccgtggccatgggcaccagtcttttgccgttagaaaatatccgggtcgatggttggcgactccgactgatggaattattggccgatccccctggtgcccctgtcaccttaggggccttagtaacgccctggcgatcgctcctggccgaaagcacgccgccagtccaagcaatgctgacggaactaattgaaagctttagcgaaccggatgcagatctagagattattttgccccggttaaatcagctccttttagagcagtctagccagcaacatctgcaaatggcgatcgccagtgccaccgaccagggaaaactccccacaagcaaccaagatgcccactaccccacgacccaggatttagccgctccccctacggcaaccctagccttgagtgatcatttactaatggtgtgtgacggcgttgaaggccatggccagggggatgtggcgagtcagttggcaattcaatccctcaagttgcaattgacaggtttcttccaagggctatttgataccgatgaagtggttcccccggcggtcatcgaacaacagttggcggcctacattcgcattacaaataacttgatcgccgaacgtaacgatcaagaaggacgcacggggggcgatcgcatggccactactctaaccctggccctccaggtaccccaaagacccaaggccgacaaactccaggatagccacagccacgaactctacattgcccaggtgggggacagccgtgcctattggatcactaaagatcaatgcgtttgcttaacggtggatgatgatctgctcagtcgggaagtccaggcgggccgggctatttatcgtcaagggttacagcgtcctgatcacatggccctcacccaagccctagggattaaagggggcgatcgcctccatcctgtgattcgccgcttcgtgtttgctgaagatggggtgttggtggtctgttccgatggcctgagtgaccagcaatttttagagtcccattggcagaccttcgccccggtgattatccagggtcatttgcccccggcggccctgctccagggcttaatcgagaaggcgatcgccaaaaatcctgaagataacattacggcggcgatcgccttctaccgcttcacaacggataccttcacccaggccccggacattgaaacggcccccgccccggaagactttgagccggaatttgtccccccagatctcgccctagacacaacccttgaggcggaactggagtcggaaccagaaacagaaaacagtctatcccagttcaccttaattctggtgagtttagtggcgattcttttgatgttagtcctggcggcctttggcttgaactggctgttaaaccgtgggcctgagccgacgcaaccgggggagccaaatcttgaaacccctacaaacgcagagtag

SEQ ID NO: 210 - protein, 546 aa, Synechococcus sp.
MATSVYQLKTNSTQFANVTQGEDCTLAAIDIGTNSIHMVIVKIQPSLPAFTIVAREKDTVRLGHRDRLTGNLTEAAMDRSLNALRRCQDLATSFQVDSLVAVATSAVREAPNGREFLQRIEAELGLEVDLISGQEEARRIYLGVLSAVDFNQQPHVLIDIGGGSTEISLVESHEARFLSSTKVGAVRLTQDFVNTDPISNREFAALQAYIRGMLERPIEELQEHLFPEEQVQMIGTSGTIETLAAMHAMANLGNVPSPLHGYTFSRQDLSKLIQQMRELNCRERSNLPGMSDKRAEIILAGAIILQEAMDLLQLKKITLCERALREGVIVDWMLSHGLIESRLQYQSSIRERSVMAIAKKYRVDLVASKRTAVFSLSLFDQLQGGLHQWDTEAREMLWAAAILHNCGLYISHAAHHKHSYYLIRNAELLGFNETQLEIVANLARYHRKSKPKKKHENYQNLIHKEHRQMVSELSAIMRLAVALDRRQVGAIAEIQCDFDAKQRLLTLKLIPTHRDDACELELWSLNYNKEIFEEEFAVTVAAHLCP

SEQ ID NO: 211 - DNA, 1641 bp, Synechococcus sp.
atggctacttccgtctatcagcttaaaacgaattccactcaatttgcgaatgtcacccaaggggaggactgtaccctagcagcgattgatatcggcaccaactcaattcacatggtgattgtcaaaattcaacccagcctgcccgcatttacaattgtggcccgggaaaaagatacggtgcgcctcggtcatcgcgatcgcctcacaggaaacctgacggaagccgccatggatcgttctttaaatgccctccgtcgttgtcaggatctagcgacgagttttcaggtggattctttagtagcagtggcaaccagtgccgtgcgagaagcccccaacggtcgagaatttttacaacggattgaagcagaattagggttagaagttgatctaatctccggccaagaagaagcgcgccgtatctacctcggtgttttatcagccgttgactttaaccaacaaccccatgttttgattgatattgggggcggttcgacagaaattagcttggtggaaagccatgaagcacgctttcttagcagcacaaaggtgggagcggtgcggttaacccaggactttgtgaatactgatccgattagtaaccgagaatttgcggccctacaagcttatattcgggggatgttagagcgtcccattgaagaactacaagagcatcttttcccggaagaacaggtacaaatgatcgggacctctggcaccattgaaaccttggcagcaatgcacgcgatggccaatttaggaaatgtgccgagtcccctccatggctatacgttttcgcgtcaggatttgagcaaactgattcaacagatgcgggagcttaattgtcgggagcgctcaaatttaccaggaatgtccgataagcgcgcagaaattattctggcaggggcaatcatcctccaagaagcgatggatctattgcagctgaaaaaaattaccctctgtgaacgggcgttgcgggaaggggtgatcgtcgactggatgctttcccatggtttgattgaaagtcgcctgcaataccaaagttcgattcgggaacggagtgtgatggcgatcgccaaaaaatatcgcgttgatttggtcgccagtaaacgcactgccgtattttccctgagtctctttgatcagctccagggggggctgcaccaatgggacaccgaagcgagggagatgctctgggcggcggcgattctccataactgtggcctttacattagccatgcggctcaccataaacattcctactatctgattcgtaatgcagagctcctcggctttaatgaaacccaattagaaatcgtcgcgaacctcgcccgctaccaccgcaaaagcaagccgaagaaaaaacacgaaaattatcaaaatctcatccacaaagaacaccgacagatggtgagtgagttgagtgcgatcatgcggcttgcggtggcccttgaccgacgccaggtaggggcgatcgccgaaattcagtgtgactttgatgcgaaacaacgcctactcaccctcaagctaatcccaacccatagggatgatgcctgcgaactagagctctggagtttaaactataacaaggagatctttgaagaagaatttgcagtgaccgtggccgcccatctatgcccctaa

SEQ ID NO: 212 - protein, 242 aa, Synechococcus sp.
MKLFVYHTPEATPTDQLPDCAVVIDVLRATTTIATALHAGAEAVQTFADLDELFQFSETWQQTPFLRAGERGGQQVEGCELGNSPRSCTPEMVAGKRLFLTTTNGTRALKRVEQAPTVITAAQVNRQSVVKFLQTEQPDTVWFVGSGWQGDYSLEDTVCAGAIAKSLWNGDSDQLGNDEVIGAISLYQQWQQDLFGLFKLASHGQRLLRLDNEIDIRYCAQSDTLAVLPIQTEPGVLKAYRH

SEQ ID NO: 213 - DNA, 729 bp, Synechococcus sp.
gtgaaactttttgtgtatcacacgcctgaggcgacgccaacggatcaactccccgattgtgctgtggttattgacgtactgcgggccaccacaaccatcgctacggcgctccacgctggagcagaagcagtgcaaacctttgctgacctcgatgaactgtttcaatttagtgaaacttggcagcaaaccccctttctccgggctggggaacggggcgggcaacaggtagaaggctgtgagcttggcaattctccccgcagttgtactccagaaatggtggctgggaagcgcctcttcttaacaaccaccaacggcacgagggccctcaagcgcgttgagcaagcacccacagtgattaccgcagcccaagtgaatcgccagagcgtggtgaagtttctccagacagaacagccagacaccgtttggttcgttggttccggttggcagggggattattccctcgaagataccgtctgtgctggggcgatcgccaagtccctgtggaatggggacagtgaccagttagggaatgacgaagtgattggggcaatttccctttaccaacagtggcagcaagatttatttggcctcttcaagctcgcaagccacggccagcgtctcctgcgcttagacaatgaaatcgatattcgttactgtgcccaaagcgataccctggcggttttaccgatccaaacagagccgggtgtcctcaaagcctatcgccactaa

SEQ ID NO: 214 - protein, 281 aa, Synechococcus sp.
MDQQKLTEVLAIARQIGWGAGDVLQSYYKGDIKNISDKKDGPVTKADLAANHYILEAFQEKLGTEDFAYLSEETYDGNKVEHPWVWIIDPLDGTRDFIDQTGEYAVHICLVHEGRPVIAVVVVPEAEKLYFASKGNGTFVETRDGTVTPIKVSERNQPEDLYLVASRTHRDQRFQDLLDRLPFKDRNYVGSVGCKIAHILEQKSDVYISLSGKSAAKDWDFAAPELILTEAGGKFSYFAGNEVLYNQGDVVKWGGIMASNGPCHAELCQQAIAILAELDRT

SEQ ID NO: 215 - DNA, 846 bp, Synechococcus sp.
atggatcagcaaaagttaacggaagttttggcgatcgcccgacaaatcggttggggtgcaggggatgttctccaaagttattacaaaggagatattaaaaatatttctgataaaaaagatggccctgtcaccaaggcagatttagcagcaaatcactatattctggaagcgtttcaggaaaagttaggcactgaagattttgcctatctcagcgaagaaacctacgacggcaataaagttgaacatccttgggtgtggattattgatcccctcgatggcacccgtgattttattgaccaaacgggagaatatgccgttcacatttgccttgttcatgaaggtcgcccggtcattgcggtagtggtcgtccccgaagcagaaaagctttatttcgcgtcgaaagggaatggcacttttgtggaaactcgtgatggcaccgtcaccccaattaaagtttctgagcgcaatcaaccagaagatttatatttagtcgccagccgtacccaccgggatcaacgcttccaggatttgttagatcgcctaccctttaaagatagaaattatgtggggagtgtcggctgtaaaattgcccatattctcgaacaaaaatccgatgtttatatttctctatcggggaaatctgcagcaaaagattgggattttgcggccccggaactaatcctcacggaagcaggtggaaaatttagttattttgcaggcaatgaagtgctctataaccaaggcgatgtggtgaagtggggcggcattatggcgtctaatgggccgtgtcatgcagaactttgtcagcaggcgatcgccatccttgcagaactagatcgtacatag

SEQ ID NO: 216 - protein, 46 aa, Synechococcus sp.
MIHDDGRSNYSNNRPFQDIFKARFSRRSMLQKSMMLSAAGFIGAIA

SEQ ID NO: 217 - protein, 56 aa, Synechococcus sp.
MIHDDGRSNYSNNRPFQDIFKARFSRRSMLQKSMMLSAAGFIGAIAGNSVLKPSTA

SEQ ID NO: 218 - protein, 47 aa, Synechococcus sp.
MNLNSGVKSLVASMVKPKLKASFKLALLSTLAGLPLGTLIFPPQAIA

SEQ ID NO: 219 - protein, 13 aa, Synechococcus sp.
MVSLAIAPLSLWA

SEQ ID NO: 220 - protein, 27 aa, Synechococcus sp.
MHGNRRQFLTYGGLALGSVLISRGIIA

SEQ ID NO: 221 - protein, 52 aa, Synechococcus sp.
MAYKLLFVCLGNICRSPSAENIMRHLLEQEGLSNKILCDSAGTSSYHIGAAP

SEQ ID NO: 222 - protein, 19 aa, Synechococcus sp.
MAYKLLFVCLGNICRSPSA

SEQ ID NO: 223 - protein, 20 aa, Synechococcus sp.
MSITLPYLRASGSLALTFQA

SEQ ID NO: 224 - protein, 30 aa, Synechococcus sp.
MATSVYQLKTNSTQFANVTQGEDCTLAAID

SEQ ID NO: 225 - protein, 39 aa, Synechococcus sp.
MKLFVYHTPEATPTDQLPDCAVVIDVLRATTTIATALHA

SEQ ID NO: 226 - protein, 48 aa, Synechococcus sp.
MDQQKLTEVLAIARQIGWGAGDVLQSYYKGDIKNISDKKDGPVTKADL

SEQ ID NO: 227 - protein, 30 aa, Synechococcus sp.
AALIEATDYLSCVMVEQNGCMAEYEAKDER

SEQ ID NO: 228 - protein, 23 aa, Synechococcus sp.
AKSDSTSGGATASFAGINLGGDR

SEQ ID NO: 229 - protein, 28 aa, Synechococcus sp.
AKSDSTSGGATASFAGINLGGDRTETNR

SEQ ID NO: 230 - protein, 8 aa, Synechococcus sp.
APVGQALR

SEQ ID NO: 231 - protein, 10 aa, Synechococcus sp.
ENTRSVLDLF

SEQ ID NO: 232 - protein, 22 aa, Synechococcus sp.
GQVTGAQYIVLGQITSYEEGVK

SEQ ID NO: 233 - protein, 13 aa, Synechococcus sp.
GSSEEAYVAVDLR

SEQ ID NO: 234 - protein, 19 aa, Synechococcus sp.
LGGGGRGSSEEAYVAVDLR

SEQ ID NO: 235 - protein, 26 aa, Synechococcus sp.
QNLGAVLSEQELAELGIVRPETGAQR

SEQ ID NO: 236 - protein, 21 aa, Synechococcus sp.
SDSTSGGATASFAGINLGGDR

SEQ ID NO: 237 - protein, 26 aa, Synechococcus sp.
SDSTSGGATASFAGINLGGDRTETNR

SEQ ID NO: 238 - protein, 13 aa, Synechococcus sp.
TETNRAPVGQALR

SEQ ID NO: 239 - protein, 13 aa, Synechococcus sp.
VVDSTTGEVLYAR

SEQ ID NO: 240 - protein, 30 aa, Synechococcus sp.
VVERQNLGAVLSEQELAELGIVRPETGAQR

(19) United States
(12) Patent Application Publication
(10) Pub. No.: US 2015/0093495 A1
(43) Pub. Date: Apr. 2, 2015

(54) Nucleic Acids, Cells, and Methods for Producing Secreted Proteins

(71) Applicant: Pronutria, Inc., Cambridge, MA (US)

(72) Inventors: Gaozhong Shen, Brighton, MA (US); David M. Young, Portsmouth, NH (US); Subhayu Basu, Chestnut Hill, MA (US); Katherine G. Gora, Boston, MA (US); Carine Robichon-Iyer, Arlington, MA (US); Nathaniel W. Silver, Cambridge, MA (US); David Arthur Berry, Brookline, MA (US)

(73) Assignee: Pronutria, Inc., Cambridge, MA (US)

(21) Appl. No.: 14/397,412

(22) PCT Filed: Apr. 29, 2013
(86) PCT No.: PCT/US13/38682

§ 371 Date: Oct. 27, 2014

Related U.S. Application Data
(60) Provisional application No. 61/639,673, filed on Apr. 27, 2012.
(60) Provisional application No. 61/639,691, filed on Apr. 27, 2012.

(51) Int. Cl. A23J 1/00; C12N 9/42; C07K 14/405; C12P 21/00

(52) U.S. Cl. CPC A23J 1/009; C12P 21/00; C12N 9/2448; C07K 14/405; C07K 2319/036
USPC 426/656; 435/257.2; 435/69.8; 435/202; 435/219; 435/69.7; 435/68.1

(57) ABSTRACT

A method for producing a secreted recombinant polypeptide sequence is provided. In some embodiments it comprises providing a recombinant microorganism comprising a recombinant nucleic acid comprising a first nucleic acid sequence encoding the recombinant polypeptide sequence operatively linked to a second nucleic acid sequence encoding a signal peptide; and culturing the recombinant microorganism in a culture medium under conditions sufficient for production and secretion of the recombinant protein by the recombinant microorganism. In some embodiments the coding sequence for the signal peptide is not native to the recombinant microorganism. In some embodiments the recombinant microorganism is photo synthetic. Also provided are recombinant photosynthetic microorganisms, isolated polypeptides comprising a signal peptide comprising an amino acid sequence disclosed herein, and isolated nucleic acids comprising a coding sequence for one of the signal peptides, among other things.

### CROSS REFERENCE TO RELATED APPLICATIONS

This application claims the benefit of U.S. Provisional Application No. 61/639,673, filed Apr. 27, 2012 and U.S. Provisional Application No. 61/639,691, filed Apr. 27, 2012, the entire disclosures of which are hereby incorporated by reference for all purposes.

### INTRODUCTION

The ability of photosynthetic microorganisms, such as cyanobacteria, to use sunlight and CO₂ as energy and carbon sources, respectively, has created much interest in the use of photosynthetic microbes for the sustainable production of biomass, biofuels (e.g., ethanol, butanol, biodiesel, and hydrogen), and bioplastics; furthermore, they can be employed in bioremediation, biofertilization, aquaculture, and the production of biologically active compounds or of high-value products, such as vitamins, nutrients, pharmaceuticals, and proteins of all kinds.

Production of recombinant proteins in photosynthetic microorganisms would be a useful way to manufacture the recombinant proteins of many types for many different purposes. One example is production of nutritive proteins. The agricultural methods required to supply high quality animal protein sources such as casein and whey, eggs, and meat, as well as plant proteins such as soy, require significant energy inputs and have potentially deleterious environmental impacts. Accordingly, it would be useful in certain situations to have alternative sources and methods of supplying proteins for mammalian consumption.

For that purpose of manufacturing recombinant proteins in photosynthetic microorganisms it would be useful to express the recombinant protein in a secreted form so it can be recovered from media that a recombinant photosynthetic microorganism grows in. To this end, the inventors in this disclosure provide methods for producing a secreted recombinant polypeptide sequence. In some embodiments the method comprises providing a recombinant microorganism comprising a recombinant nucleic acid comprising a first nucleic acid sequence encoding the recombinant polypeptide sequence operatively linked to a second nucleic acid sequence encoding a signal peptide; and culturing the recombinant microorganism in a culture medium under conditions sufficient for production and secretion of the recombinant protein by the recombinant microorganism. In some embodiments the coding sequence for the signal peptide is not native to the recombinant microorganism. In some embodiments the recombinant microorganism is photosynthetic. Also provided are recombinant photosynthetic microorganisms, isolated polypeptides comprising a signal peptide comprising an amino acid sequence disclosed herein, and isolated nucleic acids comprising a coding sequence for one of the signal peptides, which can be operatively linked to a nucleic acid sequence encoding a polypeptide sequence of interest, among other things.

### SUMMARY

Disclosed herein is a recombinant microorganism, comprising: one or more recombinant nucleic acid sequences comprising a first nucleic acid sequence encoding a polypeptide sequence operatively linked to a second nucleic acid sequence encoding a signal peptide, wherein the first nucleic acid sequence is heterologous to the microorganism, and wherein the recombinant microorganism secretes increased amounts of the polypeptide relative to an otherwise identical microorganism, cultured under identical conditions, but lacking said at least one or more recombinant nucleic acid sequences.

In some aspects, the recombinant microorganism is a cyanobacterium, wherein the signal peptide is a SEC signal peptide, a Type IV signal peptide, or a Type I signal peptide, and wherein the recombinant microorganism secretes at least 1 mg/L of the polypeptide per 48 hours. In some aspects, the recombinant microorganism is a cyanobacterium, wherein the signal peptide is a SEC signal peptide, a Type IV signal peptide, or a Type I signal peptide, and wherein the recombinant microorganism secretes at least 1, 2, 3, 4, 5, 6, 7, 8, 9, 10, 11, 12, 13, 14, 15, 16, 17, 18, 19, or 20 mg/L of the polypeptide per 48 hours. In some aspects, the recombinant microorganism secretes at least 0.01, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg/L of the polypeptide per 48 hours.

In some aspects, the signal peptide is a SEC signal peptide, a Type IV signal peptide, or a Type I signal peptide. In some aspects, the signal peptide comprises an amino acid sequence selected from SEQ ID NOS: 1-12 or an amino acid sequence shown in Tables 16, 17, 18, and/or 19 or an amino acid sequence at least 90, 91, 92, 93, 94, 95, 96, 97, 98, or 99% identical to a sequence shown in SEQ ID NOS: 1-12 or an amino acid sequence shown in Tables 16, 17, 18, and/or 19. In some aspects, the nucleic acid sequence that encodes a signal peptide is selected from SEQ ID NOS: 13-24 or a nucleotide sequence shown in Tables 16, 17, 18, and/or 19 or a nucleotide sequence at least 90, 91, 92, 93, 94, 95, 96, 97, 98, or 99% identical to a sequence shown in SEQ ID NOS: 13-24 or nucleotide sequence shown in Tables 16, 17, 18, and/or 19. In some aspects, the first nucleic acid sequence encoding a polypeptide sequence is directly linked to the second nucleic acid sequence encoding a signal peptide. In some aspects, the second nucleic acid sequence encoding a signal peptide is located 5′ of the first nucleic acid sequence encoding the polypeptide sequence. In some aspects, the second nucleic acid sequence encoding a signal peptide is located 5′ of the first nucleic acid sequence encoding the polypeptide sequence, and wherein the signal peptide comprises an amino acid sequence selected from SEQ ID NOS: 1-8. In some aspects, the nucleic acid sequence that encodes a signal peptide is selected from SEQ ID NOS: 13-20. In some aspects, the second nucleic acid sequence encoding a signal peptide is located 3′ of the first nucleic acid sequence encoding the polypeptide sequence. In some aspects, the second nucleic acid sequence encoding a signal peptide is located 3′ of the first nucleic acid sequence encoding the polypeptide sequence, wherein the signal peptide comprises an amino acid sequence selected from SEQ ID NOS: 9-12. In some aspects, the nucleic acid sequence that encodes a signal peptide is selected from SEQ ID NOS: 21-24. In some aspects, the second nucleic acid sequence encoding a signal peptide comprises a sequence that is at least 90% or at least 95% identical to a sequence or portion thereof shown in any one of the Tables. Typically the portion thereof is located at one or both ends of a sequence.

In some aspects, the polypeptide sequence is a naturally occurring eukaryotic protein. In some aspects, the polypeptide sequence is a naturally occurring intracellular protein. In some aspects, the polypeptide sequence is a naturally occurring nutritive protein. In some aspects, the polypeptide sequence has a characteristic functional property associated with its native structure, and wherein the polypeptide is capable of exhibiting the characteristic functional property upon expression. In some aspects, the polypeptide sequence is a non-enzymatically active protein. In some aspects, the polypeptide sequence is not naturally folded upon expression.

In some aspects, the at least one recombinant nucleic acid sequence further comprises a third nucleic acid sequence that is an expression control sequence operatively linked to the first nucleic acid sequence and the second nucleic acid sequence. In some aspects, the expression control sequence comprises a promoter. In some aspects, the promoter is an inducible promoter. In some aspects, the promoter is a repressible promoter. In some aspects, the promoter comprises a nucleic acid sequence selected from SEQ ID NOS: 25-42. In some aspects, the recombinant microorganism further comprises a nucleic acid comprising at least one open reading frame that encodes at least one protein selected from SEQ ID NOS: 50-56.

In some aspects, the recombinant nucleic acid is integrated into a chromosome of the recombinant microorganism. In some aspects, the recombinant nucleic acid is integrated into each copy of the chromosome of the recombinant microorganism. In some aspects, the recombinant microorganism comprises a vector comprising the recombinant nucleic acid. In some aspects, the vector is a plasmid. In some aspects, at least one endogenous pilus assembly gene is inactivated in the recombinant microorganism.

In some aspects, said microorganism is a bacterium. In some aspects, said microorganism is a gram-negative bacterium. In some aspects, said microorganism is E. coli. In some aspects, said microorganism is a photosynthetic microorganism. In some aspects, said microorganism is a cyanobacterium. In some aspects, said microorganism is a thermophylic cyanobacterium. In some aspects, said microorganism is a Synechococcus species. In some aspects, the cyanobacterium is a strain selected from Synechococcus sp. PCC 7002, Synechococcus sp. ATCC 29404, Synechocystis sp. PCC 6308, and Synechococcus elongatus sp. PCC 7942-1.

Also disclosed herein is a cell culture comprising a culture media and a microorganism disclosed herein.

Also disclosed herein is a method for producing a polypeptide, comprising: culturing a recombinant microorganism described herein in a culture medium, wherein said recombinant microorganism secretes increased amounts of polypeptide relative to an otherwise identical microorganism, cultured under identical conditions, but lacking said at least one recombinant nucleic acid sequence.

In some aspects, the method further comprises allowing the polypeptide to accumulate in the culture medium. In some aspects, the method further comprises isolating at least a portion of the polypeptide. In some aspects, the method further comprises processing the polypeptide to produce a processed material. In some aspects, the method further comprises recovering the polypeptide from the culture medium during the exponential growth phase. In some aspects, the method further comprises recovering the polypeptide from the culture medium during the stationary phase. In some aspects, the method further comprises recovering the polypeptide from the culture medium at a first time point, continuing the culture under conditions sufficient for production and secretion of the polypeptide by the microorganism, and recovering the polypeptide from the culture medium at a second time point. In some aspects, the method further comprises recovering the polypeptide from the culture medium by a continuous process.

In some aspects, the polypeptide sequence further comprises a tag, and the method further comprises removing the tag from the polypeptide sequence. In some aspects, the polypeptide sequence has a characteristic functional property associated with its native structure, and wherein the polypeptide is capable of exhibiting the characteristic functional property upon expression.

In some aspects, the method further includes separating the signal peptide encoded by the second nucleic acid sequence or a portion thereof from the polypeptide sequence encoded by the first sequence during or after secretion of the polypeptide. In some aspects, the separation separates all but one residue of the signal peptide from the polypeptide sequence.

Also described herein is a composition comprising a polypeptide, wherein said polypeptide is produced by a method disclosed herein. In some aspects, the composition comprises by weight at least 50%, at least 60%, at least 70%, at least 80%, at least 90%, at least 95%, or at least 99% of the polypeptide.

Also disclosed herein is a method for producing a polypeptide, comprising: (i) culturing a recombinant microorganism described herein in a culture medium; and (ii) exposing said recombinant microorganism to light and inorganic carbon, wherein said polypeptide is secreted in an amount greater than that produced by an otherwise identical microorganism, cultured under identical conditions, but lacking said at least one recombinant nucleic acid sequence.

In some aspects, the method further comprises allowing the polypeptide to accumulate in the culture medium. In some aspects, the method further comprises isolating at least a portion of the polypeptide. In some aspects, the method further comprises processing the polypeptide to produce a processed material. In some aspects, the method further comprises recovering the polypeptide from the culture medium during the exponential growth phase. In some aspects, the method further comprises recovering the polypeptide from the culture medium during the stationary phase. In some aspects, the method further comprises recovering the polypeptide from the culture medium at a first time point, continuing the culture under conditions sufficient for production and secretion of the polypeptide by the microorganism, and recovering the polypeptide from the culture medium at a second time point. In some aspects, the method further comprises recovering the polypeptide from the culture medium by a continuous process.

In some aspects, the method further includes separating the signal peptide encoded by the second nucleic acid sequence or a portion thereof from the polypeptide sequence encoded by the first sequence during or after secretion of the polypeptide. In some aspects, the separation separates all but one residue of the signal peptide from the polypeptide sequence.

In some aspects, the polypeptide sequence further comprises a tag, and the method further comprises removing the tag from the polypeptide sequence. In some aspects, the polypeptide sequence has a characteristic functional property associated with its native structure, and wherein the polypeptide is capable of exhibiting the characteristic functional property upon expression.

Also described herein is a composition comprising a polypeptide, wherein said polypeptide is produced by a method disclosed herein. In some aspects, the composition comprises at least 50%, at least 60%, at least 70%, at least 80%, at least 90%, at least 95%, or at least 99% of the polypeptide.

Also disclosed herein is an isolated polypeptide comprising a signal peptide comprising an amino acid sequence selected from SEQ ID NOS: 1-12 or an amino acid sequence shown in Tables 16, 17, 18, and/or 19 or an amino acid sequence at least 90, 91, 92, 93, 94, 95, 96, 97, 98, or 99% identical to a sequence shown in SEQ ID NOS: 1-12 or an amino acid sequence shown in Tables 16, 17, 18, and/or 19.

In some aspects, the polypeptide further comprises a heterologous polypeptide sequence linked to the carboxyl terminus of the signal peptide. In some aspects, the polypeptide further comprises a heterologous polypeptide sequence linked to the carboxyl terminus of the signal peptide, wherein the signal peptide comprises an amino acid sequence selected from SEQ ID NOS: 1-8 or an amino acid sequence at least 90, 91, 92, 93, 94, 95, 96, 97, 98, or 99% identical to a sequence shown in SEQ ID NOS: 1-8. In some aspects, the polypeptide further comprises a heterologous polypeptide sequence linked to the amino terminus of the signal peptide. In some aspects, the polypeptide further comprises a heterologous polypeptide sequence linked to the amino terminus of the signal peptide, wherein the signal peptide comprises an amino acid sequence selected from SEQ ID NOS: 9-12 or an amino acid sequence at least 90, 91, 92, 93, 94, 95, 96, 97, 98, or 99% identical to a sequence shown in SEQ ID NOS: 9-12.

In some aspects, the heterologous polypeptide is a naturally occurring eukaryotic protein. In some aspects, the heterologous polypeptide is a naturally occurring nutritive protein. In some aspects, the heterologous polypeptide is a naturally intracellular protein.

Also disclosed herein is an isolated nucleic acid comprising a first nucleic acid sequence that encodes a signal peptide comprising an amino acid sequence selected from SEQ ID NOS: 1-12 or an amino acid sequence shown in Tables 16, 17, 18, and/or 19 or an amino acid sequence at least 90, 91, 92, 93, 94, 95, 96, 97, 98, or 99% identical to a sequence shown in SEQ ID NOS: 1-12 or an amino acid sequence shown in Tables 16, 17, 18, and/or 19.

In some aspects, the nucleic acid sequence that encodes a signal peptide is selected from SEQ ID NOS: 13-34 or a nucleotide sequence shown in Tables 16, 17, 18, and/or 19 or a nucleotide sequence at least 90, 91, 92, 93, 94, 95, 96, 97, 98, or 99% identical to a sequence shown in SEQ ID NOS: 13-34 or a nucleotide sequence shown in Tables 16, 17, 18, and/or 19.

In some aspects, the nucleic acid sequence further comprises a second nucleic acid sequence encoding a polypeptide sequence operatively linked to the first nucleic acid sequence. In some aspects, the first nucleic acid sequence encoding a signal peptide is located 5′ of the second nucleic acid sequence encoding the polypeptide sequence, wherein the signal peptide comprises an amino acid sequence selected from SEQ ID NOS: 1-8 or an amino acid sequence at least 90, 91, 92, 93, 94, 95, 96, 97, 98, or 99% identical to a sequence shown in SEQ ID NOS: 1-8. In some aspects, the nucleic acid sequence that encodes a signal peptide is selected from SEQ ID NOS: 13-20. In some aspects, the first nucleic acid sequence encoding a signal peptide is located 3′ of the second nucleic acid sequence encoding the polypeptide sequence, wherein the signal peptide comprises an amino acid sequence selected from SEQ ID NOS: 9-12 or an amino acid sequence at least 90, 91, 92, 93, 94, 95, 96, 97, 98, or 99% identical to a sequence shown in SEQ ID NOS: 9-12. In some aspects, the nucleic acid sequence that encodes a signal peptide is selected from SEQ ID NOS: 21-24. In some aspects, the polypeptide is a naturally occurring eukaryotic protein. In some aspects, the polypeptide is a naturally occurring intracellular protein. In some aspects, the polypeptide is a naturally occurring nutritive protein.

In some aspects, the nucleic acid sequence further comprises a third nucleic acid sequence that is an expression control sequence operatively linked to the first nucleic acid sequence that encodes a signal peptide and the second nucleic acid sequence that encodes a polypeptide sequence. In some aspects, the expression control sequence comprises a promoter. In some aspects, the promoter is an inducible promoter. In some aspects, the promoter is a repressible promoter. In some aspects, the promoter comprises a nucleic acid sequence selected from SEQ ID NOS: 25-42. In some aspects, further comprising at least one open reading frame that encodes at least one protein selected from SEQ ID NOS: 50-56.

Also disclosed herein is a vector comprising a nucleic acid disclosed herein. In some aspects, the vector is a plasmid.

### BRIEF DESCRIPTION OF THE DRAWINGS

FIG. 1 shows the structures of four types of bacterial N-terminal signal peptides

FIG. 2 shows an example of assignment of a signal peptide in a secreted bacterial protein using the Signal 4.0 program. In this case the secreted protein is SP1.

FIG. 3 shows a map of the SG2 operon.

FIG. 4 shows a map of the SG8 operon.

FIG. 5 shows expression of recombinant YFP using different promoters.

FIG. 6 shows expression of recombinant YFP in engineered Synechocossus sp. ATCC 29404 strains.

FIG. 7A illustrates the general structure of a secretory protein overexpression cassette comprising the Pcpc* promoter, an N-terminal secretion signal peptide, yfp reporter gene, and the selection marker aadA.

FIG. 7B illustrates the general structure of a secretory protein overexpression cassette comprising the Pcpc* promoter, a C-terminal secretion signal peptide, yfp reporter gene, and the selection marker aadA.

FIG. 8 shows the strategy used to replace the SYNPCC7002-A2804 and SYNPCC7002-A2803 genes with a recombinant gene encoding YFP.

FIG. 9 shows Type IV Secretion system components in PCC 7002 Blasted against the E. coli Type IV secretion system.

FIG. 10 shows OD₇₃₀ₙₘ of different strains over the course of the six day experiment.

FIG. 11 shows the concentration of lichenase in lysate and supernatant samples over time.

FIG. 12 shows the concentration of lichenase/μL/OD₇₃₀ₙₘ in lysates and supernatants and the calculated secretion rate (ng/ul/hr). Left is wt; left-middle is pES163; right-middle is pES168; and right is pES171.

FIG. 13 shows the concentration of total protein in the supernatant under different growth conditions. Front is 0 μM cumate; middle is 25 μM cumate; and rear is 75 μM cumate.

### DETAILED DESCRIPTION

Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art. Certain references and other documents cited herein are expressly incorporated herein by reference. Additionally, all Genbank or other sequence database records cited herein are hereby incorporated herein by reference. In case of conflict, the present specification, including definitions, will control. The materials, methods, and examples are illustrative only and not intended to be limiting.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 3d ed., Cold Spring Harbor Laboratory Press, Cold Spring Harbor, N.Y. (2001); Ausubel et al., Current Protocols in Molecular Biology, Greene Publishing Associates (1992, and Supplements to 2002); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999). Many molecular biology and genetic techniques applicable to cyanobacteria are described in Heidorn et al., “Synthetic Biology in Cyanobacteria: Engineering and Analyzing Novel Functions,” Methods in Enzymology, Vol. 497, Ch. 24 (2011), which is hereby incorporated herein by reference.

This disclosure refers to sequence database entries (e.g., Genbank records) for certain amino acid and nucleic acid sequences that are published on the internet, as well as other information on the internet. The skilled artisan understands that information on the internet, including sequence database entries, is updated from time to time and that, for example, the reference number used to refer to a particular sequence can change. Where reference is made to a public database of sequence information or other information on the internet, it is understood that such changes can occur and particular embodiments of information on the internet can come and go. Because the skilled artisan can find equivalent information by searching on the internet, a reference to an internet web page address or a sequence database entry evidences the availability and public dissemination of the information in question.

Before the present proteins, compositions, methods, and other embodiments are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

The term “comprising” as used herein is synonymous with “including” or “containing”, and is inclusive or open-ended and does not exclude additional, unrecited members, elements or method steps.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe).

As used herein, the term “isolated” refers to a substance or entity that has been (1) separated from at least some of the components with which it was associated when initially produced (whether in nature or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

The term “peptide” as used herein refers to a short polypeptide, e.g., one that typically contains less than about 50 amino acids and more typically less than about 30 amino acids. The term as used herein encompasses analogs and mimetics that mimic structural and thus biological function.

The term “polypeptide” encompasses both naturally-occurring and non-naturally occurring proteins, and fragments, mutants, derivatives and analogs thereof. A polypeptide may be monomeric or polymeric. Further, a polypeptide may comprise a number of different domains each of which has one or more distinct activities. For the avoidance of doubt, a “polypeptide” may be any length greater two amino acids.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from a cell in which it was synthesized.

The term “polypeptide fragment” as used herein refers to a polypeptide that has a deletion, e.g., an amino-terminal and/or carboxy-terminal deletion compared to a full-length polypeptide, such as a naturally occurring protein. In an embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, or at least 12, 14, 16 or 18 amino acids long, or at least 20 amino acids long, or at least 25, 30, 35, 40 or 45, amino acids, or at least 50 or 60 amino acids long, or at least 70 amino acids long.

The term “fusion protein” refers to a polypeptide comprising a polypeptide or fragment coupled to heterologous amino acid sequences. Fusion proteins are useful because they can be constructed to contain two or more desired functional elements that can be from two or more different proteins. A fusion protein comprises at least 10 contiguous amino acids from a polypeptide of interest, or at least 20 or 30 amino acids, or at least 40, 50 or 60 amino acids, or at least 75, 100 or 125 amino acids. The heterologous polypeptide included within the fusion protein is usually at least 6 amino acids in length, or at least 8 amino acids in length, or at least 15, 20, or 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, a protein has “homology” or is “homologous” to a second protein if the nucleic acid sequence that encodes the protein has a similar sequence to the nucleic acid sequence that encodes the second protein. Alternatively, a protein has homology to a second protein if the two proteins have similar amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89.

The following six groups each contain amino acids that are conservative substitutions for one another: 1) Serine, Threonine; 2) Aspartic Acid, Glutamic Acid; 3) Asparagine, Glutamine; 4) Arginine, Lysine; 5) Isoleucine, Leucine, Methionine, Alanine, Valine, and 6) Phenylalanine, Tyrosine, Tryptophan.

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

An exemplary algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Exemplary parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, or at least about 20 residues, or at least about 24 residues, or at least about 28 residues, or more than about 35 residues. When searching a database containing sequences from a large number of different organisms, it may be useful to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990). For example, percent sequence identity between amino acid sequences can be determined using FASTA with its default parameters (a word size of 2 and the PAM250 scoring matrix), as provided in GCG Version 6.1, herein incorporated by reference.

In some embodiments, polymeric molecules (e.g., a polypeptide sequence or nucleic acid sequence) are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% similar. The term “homologous” necessarily refers to a comparison between at least two sequences (nucleotides sequences or amino acid sequences). In some embodiments, two nucleotide sequences are considered to be homologous if the polypeptides they encode are at least about 50% identical, at least about 60% identical, at least about 70% identical, at least about 80% identical, or at least about 90% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for nucleotide sequences to be considered homologous. In some embodiments of nucleotide sequences less than 60 nucleotides in length, homology is determined by the ability to encode a stretch of at least 4-5 uniquely specified amino acids. In some embodiments, two protein sequences are considered to be homologous if the proteins are at least about 50% identical, at least about 60% identical, at least about 70% identical, at least about 80% identical, or at least about 90% identical for at least one stretch of at least about 20 amino acids.

As used herein, a “modified derivative” refers to polypeptides or fragments thereof that are substantially homologous in primary structural sequence to a reference polypeptide sequence but which include, e.g., in vivo or in vitro chemical and biochemical modifications or which incorporate amino acids that are not found in the reference polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands that bind to labeled antiligands (e.g., antibodies), fluorophores, chemiluminescent agents, enzymes, and antiligands that can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002).

As used herein, “polypeptide mutant” or “mutein” refers to a polypeptide whose sequence contains an insertion, duplication, deletion, rearrangement or substitution of one or more amino acids compared to the amino acid sequence of a reference protein or polypeptide, such as a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the reference protein, and/or truncations of the amino acid sequence at either or both the amino or carboxy termini. A mutein may have the same or a different biological activity compared to the reference protein.

In some embodiments, a mutein has, for example, at least 85% overall sequence homology to its counterpart reference protein. In some embodiments, a mutein has at least 90% overall sequence homology to the wild-type protein. In other embodiments, a mutein exhibits at least 95% sequence identity, or 98%, or 99%, or 99.5% or 99.9% overall sequence identity.

As used herein, a “polypeptide tag for affinity purification” is any polypeptide that has a binding partner that can be used to isolate or purify a second protein or polypeptide sequence of interest fused to the first “tag” polypeptide. Several examples are well known in the art and include a His-6 tag, a FLAG epitope, a c-myc epitope, a Strep-TAGII, a biotin tag, a glutathione 5-transferase (GST), a chitin binding protein (CBP), a maltose binding protein (MBP), or a metal affinity tag.

As used herein, “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism is recombinant, for example, if it is synthesized from an mRNA synthesized from a recombinant gene present in the cell.

The term “polynucleotide”, “nucleic acid molecule”, “nucleic acid”, or “nucleic acid sequence” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

A “synthetic” RNA, DNA or a mixed polymer is one created outside of a cell, for example one synthesized chemically.

The term “nucleic acid fragment” as used herein refers to a nucleic acid sequence that has a deletion, e.g., a 5′-terminal or 3′-terminal deletion compared to a full-length reference nucleotide sequence. In an embodiment, the nucleic acid fragment is a contiguous sequence in which the nucleotide sequence of the fragment is identical to the corresponding positions in the naturally-occurring sequence. In some embodiments, fragments are at least 10, 15, 20, or 25 nucleotides long, or at least 20, 30, 40, 50, 60, 70, 80, 90, 100, 110, 120, 130, 140, or 150 nucleotides long. In some embodiments a fragment of a nucleic acid sequence is a fragment of an open reading frame sequence. In some embodiments such a fragment encodes a polypeptide fragment (as defined herein) of the protein encoded by the open reading frame nucleotide sequence.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32, and even more typically at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, or at least about 90%, or at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tm) for the specific DNA hybrid under a particular set of conditions. “Stringent washing” is performed at temperatures about 5° C. lower than the Tm for the specific DNA hybrid under a particular set of conditions. The Tm is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

As used herein, an “expression control sequence” refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences and fusion partner sequences.

As used herein, “operatively linked” or “operably linked” expression control sequences refers to a linkage in which the expression control sequence is contiguous with the gene of interest to control the gene of interest, as well as expression control sequences that act in trans or at a distance to control the gene of interest.

As used herein, a “vector” is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

The term “recombinant host cell” (or simply “recombinant cell” or “host cell”), as used herein, is intended to refer to a cell into which a recombinant nucleic acid such as a recombinant vector has been introduced. In some instances the word “cell” is replaced by a name specifying a type of cell. For example, a “recombinant microorganism” is a recombinant host cell that is a microorganism host cell. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “recombinant host cell,” “recombinant cell,” and “host cell”, as used herein. A recombinant host cell may be an isolated cell or cell line grown in culture or may be a cell which resides in a living tissue or organism.

As used herein, the term “heterotrophic” refers to an organism that cannot fix carbon and uses organic carbon for growth.

As used herein, the term “autotrophic” refers to an organism that produces complex organic compounds (such as carbohydrates, fats, and proteins) from simple inorganic molecules using energy from light (by photosynthesis) or inorganic chemical reactions (chemosynthesis).

A. Secreted Proteins and Nucleic Acids Encoding them

The inventors have identified and isolated secreted proteins from cyanobacteria. The newly identified secreted proteins and the genes that encode them are listed herein. For example, Table A lists the strain a protein was isolated from and a note regarding what is currently known about the natural function of the protein.

TABLE A

| Secreted | Gene Encoding | | |
|---|---|---|---|
| Protein | Secreted Protein | Origin Strain | Note |
| SP1 | SG1 | Synechococcus sp. PCC | Assembly extra- |
| SEQ ID NO: 57 | (SYNPCC7002_2435) | 7002 | cellular matrix |
| | SEQ ID NO: 66 | | |
| SP2 | SG2 | Synechococcus sp. PCC | secretion system |
| SEQ ID NO: 58 | (SYNPCC7002_A2594) | 7002 | |
| | SEQ ID NO: 67 | | |
| SP3 | SG3 | Synechococcus sp. PCC | Pili biosynthesis, |
| SEQ ID NO: 59 | (SYNPCC7002_A2335) | 7002 | cell mobility |
| | SEQ ID NO: 68 | | |
| SP4 | SG4 | Synechococcus elongates | Type IV secretion |
| SEQ ID NO: 60 | (SYNPCC7942_0049) | sp. PCC 7942-1 | system |
| | SEQ ID NO: 69 | | |
| SP5 | SG5 | Synechococcus elongates | Secreted outer |
| SEQ ID NO: 61 | (SYNPCC7942_0048) | sp. PCC 7942-1 | membrane protein |
| | SEQ ID NO: 70 | | |
| SP6 | SG6 | Synechocystis sp. PCC | PilT domain- |
| SEQ ID NO: 62 | SEQ ID NO: 71 | 6308 | containing protein |
| SP7 | SG7 | Synechocystis sp. PCC | PilM-like, type II |
| SEQ ID NO: 63 | SEQ ID NO: 72 | 6308 | secretion component |
| SP8 | SG8 | Synechococcus sp. ATCC | Secreted outer |
| SEQ ID NO: 64 | SEQ ID NO: 73 | 29404 | membrane protein |
| SP9 | SG9 | Synechococcus sp. ATCC | CsgG-like protein |
| SEQ ID NO: 65 | SEQ ID NO: 74 | 29404 | |

As described in the examples, the secreted proteins were identified in some instances based on their accumulation in growth media in which their strain of origin was grown. On that basis it is believed that the secreted proteins have many uses, including as indicators that can be monitored to measure the rate of generation of secreted proteins by a host microorganism cultured under a particular set of conditions. Production of the protein can be measured using any one or more of many different methods, such as SDS-PAGE and/or optionally use of an antibody that specifically binds to the secreted protein.

The nucleotide sequences that encode the secreted proteins are also useful. For example, the nucleotide sequences can be used to make the secreted proteins. The nucleotide sequences can also be used to create recombinant microorganisms that make the secreted proteins. In some embodiments the recombinant microorganism is not the same as the microorganism that the secreted protein was isolated from.

B. Signal Peptides and Nucleic Acids Encoding them

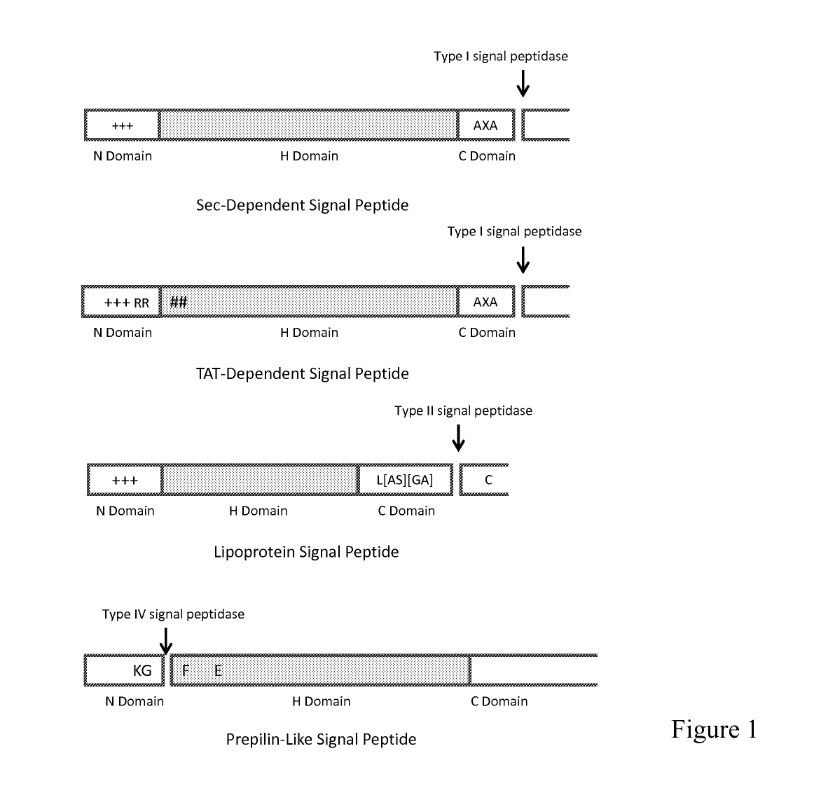

Nearly all secreted bacterial proteins are synthesized as preproteins that contain N-terminal sequences known as signal peptides. These signal peptides serve as address labels which influence the final destination of the protein and the mechanisms by which they are transported. Most signal peptides can be placed into one of four groups (FIG. 1) based on their translocation mechanism (e.g. Sec- or Tat-mediated) and the type of signal peptidase used to cleave the signal peptide from the preprotein.

In bacteria, most secretory proteins cross the cytoplasmic membrane via one of two pathways depending on whether they are folded or remain unfolded prior to translocation. In most cases proteins are transported across the membrane in an unfolded state by the Sec-pathway. Protein export through the Sec-pathway occurs post-translationally and requires the preprotein to be maintained in an unfolded conformation prior to insertion into the translocation pore which is composed of the SecY, -G, and -E proteins. In many cases, the protein is kept in the unfolded state by a chaperone called SecB however, as described below, in some cases analogous chaperones such as CsaA or general chaperones such as DnaK, GroESL, etc also function in the pathway. Sec-dependent signal peptides contain an AXA motif in their C-domain that acts as a signal for type I signal peptidase cleavage (FIG. 1).

The Twin-arginine or Tat pathway is responsible for exporting a small subset of secreted proteins that must be folded in the cytoplasm prior to export. Tat signal peptides tend to be slightly longer than Sec-pathway signals and they contain a conserved and distinctive RRX## where R is the amino acid arginine, X is any amino acid and ## are hydrophobic amino acids (FIG. 1). The twin arginine motif serves to direct these preproteins to the Tat-translocation machinery which is encoded by the tatABC. Like the Sec-secretion signals, Tat-pathway signal peptides also contain AXA target sequences in their C-domain to direct cleavage by a type I signal peptidase.

The third type of common N-terminal signal is the lipoprotein signal peptide (FIG. 1). Although proteins carrying this type of signal are transported via the Sec translocase, their peptide signals tend to be shorter than normal Sec-signals and they contain a distinct sequence motif in the C-domain known as the lipo box (L[AS][GA]C) at the −3 to +1 position. The cysteine at the +1 position is lipid modified following translocation whereupon the signal sequence is cleaved by a type II signal peptidase.

The fourth type of signal peptide is a specialized signal known as a type IV or prepilin signal peptide (FIG. 1). These signal peptides are distinguished from others by their type IV peptidase cleavage domain being localized between the N- and H-domain rather than in the C-domain like other signal peptides.

As described in the Examples, the inventors have identified eight different N-terminal signal peptides from five of the secreted proteins listed in Table 1, and two additional N-terminal signal peptides. The signal peptides and the naturally occurring nucleic acid sequences that encode them are listed in Table B. The identification and use of other signal peptides are also described in the Examples.

TABLE B

| N-Terminal | | | |
|---|---|---|---|
| Signal Peptide | Naturally-Occurring | | |
| and Sequence | Nucleotide Sequence | | |
| Identification | Encoding the Signal | | |
| Number | Peptide | Strain of Origin | Gene |
| NSP1 | NSG1 | Synechococcus sp. | SG1 |
| (SEQ ID NO: 1) | (SEQ ID NO: 13) | PCC 7002 | (SYNPCC7002_2435) |
| NSP2 | NSG2 | Synechococcus sp. | SG2 |
| (SEQ ID NO: 2) | (SEQ ID NO: 14) | PCC 7002 | SYNPCC7002_A2594 |
| NSP3 | NSG3 | Synechococcus sp. | SG3 |
| (SEQ ID NO: 3) | (SEQ ID NO: 15) | PCC 7002 | SYNPCC7002_A2335 |
| NSP4 | NSG4 | Synechococcus sp. | SG4 |
| (SEQ ID NO: 4) | (SEQ ID NO: 16) | PCC 7002 | SYNPCC7942_0049 |
| NSP5 | NSG5 | Synechococcus sp. | SYNPCC7002_A2803 |
| (SEQ ID NO: 5) | (SEQ ID NO: 17) | PCC 7002 | |
| NSP6 | NSG6 | Synechococcus sp. | SYNPCC7002_A1602 |
| (SEQ ID NO: 6) | (SEQ ID NO: 18) | PCC 7002 | |
| NSP7 | NSG7 | Synechococcus | SG8 |
| (SEQ ID NO: 7) | (SEQ ID NO: 19) | sp.ATCC 29404 | |
| NSP8 | NSG8 | Synechococcus | SG8 |
| (SEQ ID NO: 8) | (SEQ ID NO: 20) | sp.ATCC 29404 | |

NSP 5 and NSP 6 are derived from Synechococcus sp. PCC 7002 homologues of SP6 and SP7.

Identification of the signal peptides and the nucleic acids encoding them provides tools to create recombinant nucleic acid sequences useful to express recombinant proteins in photosynthetic microorganisms.

In some embodiments a C-terminal signal peptide is used instead. Examples of suitable C-terminal signal peptides include those listed in Table C.

TABLE C

| C-Terminal | Naturally- | |
|---|---|---|
| Signal Peptide | Occurring Nucleo- | |
| and Sequence | tide Sequence | |
| Identification | Encoding the | Strain of |
| Number | Signal Peptide | Origin |
| SYNPCC7002_ | (SEQ ID NO: 21) | Synechococcus |
| A1178 | | sp. |
| (SEQ ID NO: 9) | | PCC 7002 |
| SYNPCC7002_1 | (SEQ ID NO: 22) | Synechococcus |
| 634 | | sp. |
| (SEQ ID NO: 10) | | PCC 7002 |
| SYNPCC7002_ | (SEQ ID NO: 23) | Synechococcus |
| A2605 | | sp. |
| (SEQ ID NO: 11) | | PCC 7002 |
| SYNPCC7002_ | (SEQ ID NO: 24) | Synechococcus |
| A2813 | | sp. |
| (SEQ ID NO: 12) | | PCC 7002 |

The signal peptides can be attached to a polypeptide sequence different than the protein the signal peptide is derived from, to create a recombinant polypeptide sequence. Accordingly, this disclosure provides a polypeptide comprising a signal peptide comprising an amino acid sequence selected from SEQ ID NOS: 1-12 or the amino acid sequences shown in Tables 16, 17, 18, and/or 19, a mutein of an amino acid sequence selected from SEQ ID NOS: 1-12 or the amino acid sequences shown in Tables 16, 17, 18, and/or 19, and a derivative of an amino acid sequence selected from SEQ ID NOS: 1-12 or the amino acid sequences shown in Tables 16, 17, 18, and/or 19. In some embodiments the polypeptide further comprises a heterologous polypeptide sequence attached to the carboxyl terminus of the signal peptide, wherein the signal peptide comprises an amino acid sequence selected from SEQ ID NOS: 1-8, a mutein of an amino acid sequence selected from SEQ ID NOS: 1-8, and a derivative of an amino acid sequence selected from SEQ ID NOS: 1-8. In some embodiments the polypeptide further comprises a heterologous polypeptide sequence attached to the amino terminus of the signal peptide, wherein the signal peptide comprises an amino acid sequence selected from SEQ ID NOS: 9-12, a mutein of an amino acid sequence selected from SEQ ID NOS: 9-12, and a derivative of an amino acid sequence selected from SEQ ID NOS: 9-12.

In some embodiments of the polypeptide, the heterologous polypeptide sequence attached to the carboxyl terminus of the signal peptide is a naturally occurring eukaryotic protein, or a mutein or derivative thereof. In some embodiments of the polypeptide, the heterologous polypeptide sequence attached to the carboxyl terminus of the signal peptide is a naturally occurring intracellular protein, or a mutein or derivative thereof. In some embodiments of the polypeptide, the heterologous polypeptide sequence attached to the carboxyl terminus of the signal peptide is a nutritive protein, or a mutein or derivative thereof.

In some embodiments the recombinant polypeptide is isolated. In some embodiments the recombinant polypeptide is present in a cell that synthesizes the recombinant polypeptide or in culture media that a cell is cultured in.

### C. Recombinant Nucleic Acids

This disclosure provides nucleic acids encoding signal peptides active in photosynthetic microorganisms. The nucleic acids can be used to create nucleic acid constructs that encode one of the signal peptides fused to a nucleic acid sequence encoding polypeptide sequence different than the polypeptide sequence that the signal peptide is derived from.

For example, in some embodiments a nucleic acid is provided that comprises a first nucleic acid sequence that encodes a signal peptide comprising an amino acid sequence selected from SEQ ID NOS: 1-12 or the amino acid sequences shown in Tables 16, 17, 18, and/or 19, a mutein of an amino acid sequence selected from SEQ ID NOS: 1-12 or the amino acid sequences shown in Tables 16, 17, 18, and/or 19, and a derivative of an amino acid sequence selected from SEQ ID NOS: 1-12 or the amino acid sequences shown in Tables 16, 17, 18, and/or 19. In some embodiments of the nucleic acid the nucleic acid sequence that encodes a signal peptide is selected from SEQ ID NOS: 13-24 or the nucleotide sequences shown in Tables 16, 17, 18, and/or 19, the naturally occurring sequences that encode those signal peptides. In some embodiments the nucleic acid further comprises a second nucleic acid sequence encoding a recombinant polypeptide sequence operatively linked to the first nucleic acid sequence. In this context “operatively linked” means that the first nucleic acid sequence that encodes a signal peptide and the second nucleic acid sequence encoding a recombinant polypeptide sequence are part of a contiguous nucleic acid sequence with a structure such that following transcription and translation of the contiguous nucleic acid sequence the resulting polypeptide sequence comprises the signal peptide encoded by the first nucleic acid sequence and the recombinant polypeptide sequence encoded by the second nucleic acid sequence.

In some embodiments the signal peptide is an N-terminal signal peptide. Examples include SEQ ID NOS: 1-8. Accordingly, in some embodiments of the nucleic acid the first nucleic acid sequence encoding a signal peptide is located upstream of the second nucleic acid sequence encoding the recombinant polypeptide sequence. In some embodiments the signal peptide comprises an amino acid sequence selected from SEQ ID NOS: 1-8, a mutein of an amino acid sequence selected from SEQ ID NOS: 1-8, and a derivative of an amino acid sequence selected from SEQ ID NOS: 1-8. In some embodiments the nucleic acid sequence that encodes a signal peptide is selected from SEQ ID NOS: 13-20.

In some embodiments the signal peptide is a C-terminal signal peptide. Examples include SEQ ID NOS: 9-12. Accordingly, in some embodiments of the nucleic acid the first nucleic acid sequence encoding a signal peptide is located downstream of the second nucleic acid sequence encoding the recombinant polypeptide sequence. In some embodiments the signal peptide comprises an amino acid sequence selected from SEQ ID NOS: 9-12, a mutein of an amino acid sequence selected from SEQ ID NOS: 9-12, and a derivative of an amino acid sequence selected from SEQ ID NOS: 9-12. In some embodiments the nucleic acid sequence that encodes a signal peptide is selected from SEQ ID NOS: 21-24.

In some embodiments the nucleic acid further comprises a third nucleic acid sequence that is an expression control sequence operatively linked to the first nucleic acid sequence that encodes a signal peptide and the second nucleic acid sequence that encodes a heterologous polypeptide sequence. In this context “operatively linked” means that the expression control sequence directs expression of the first and second nucleic acid sequences. In some embodiments the expression control sequence comprises a promoter. In some embodiments the promoter is an inducible promoter. In some embodiments the promoter is a repressible promoter. In some embodiments the promoter is constitutive. Various types of suitable promoters are disclosed herein. In some embodiments the promoter comprises a nucleic acid sequence selected from SEQ ID NOS: 25-42 and derivatives thereof.

In some embodiments of the nucleic acid the recombinant polypeptide is a naturally occurring eukaryotic protein, or a mutein or derivative thereof. In some embodiments of the nucleic acid the heterologous polypeptide is a naturally occurring intracellular protein, or a mutein or derivative thereof. By expressing the naturally occurring intracellular protein fused to a signal peptide, the intracellular protein can be secreted by a recombinant microorganism comprising the nucleic acid sequence. In some embodiments of the nucleic acid the heterologous polypeptide is a naturally occurring nutritive protein, or a mutein or derivative thereof.

In some embodiments the nucleic acid further comprises an intervening nucleic acid sequence between the nucleic acid sequence encoding the signal peptide and the nucleic acid sequence encoding the recombinant polypeptide sequence that is selected from a naturally occurring eukaryotic protein, or a mutein or derivative thereof; a naturally occurring intracellular protein, or a mutein or derivative thereof; and a naturally occurring intracellular protein, or a mutein or derivative thereof. Transcription and translation of the nucleic acid produces a polypeptide sequence comprising the signal peptide, the polypeptide sequence encoded by the intervening sequence, and the recombinant polypeptide sequence that is selected from a naturally occurring eukaryotic protein, or a mutein or derivative thereof; a naturally occurring intracellular protein, or a mutein or derivative thereof; and a naturally occurring intracellular protein, or a mutein or derivative thereof. The polypeptide sequence encoded by the intervening sequence can be any sequence, such as a tag, such as a poly-His tag. In some embodiments the intervening sequence comprises a number of amino acids selected from 1 to 3 amino acids, from 2 to 5 amino acids, from 5 to 10 amino acids, from 20 to 50 amino acids, from 50 to 100 amino acids, and over 100 amino acids.

In some embodiments of the nucleic acid the nucleic acid is isolated. In some embodiments it is present in a recombinant microorganism.

Also provided are vectors, including expression vectors, which comprise at least one of the nucleic acid molecules disclosed herein. The vectors can thus be used to express at least one recombinant protein in a recombinant microbial host cell. In some embodiments the isolated nucleic acid (such as a vector) further comprises a nucleic acid sequence that encodes at least one protein selected from SEQ ID NOS: 50-56.

Suitable vectors for expression of nucleic acids in microorganisms are well known to those of skill in the art. Suitable vectors for use in cyanobacteria are described, for example, in Heidorn et al., “Synthetic Biology in Cyanobacteria: Engineering and Analyzing Novel Functions,” Methods in Enzymology, Vol. 497, Ch. 24 (2011). Exemplary replicative vectors that can be used for engineering cyanobacteria as disclosed herein include pPMQAK1, pSL1211, pFC1, pSB2A, pSCR119/202, pSUN119/202, pRL2697, pRL25C, pRL1050, pSG111M, and pPBH201.

Other vectors such as pJB161 which are capable of receiving nucleic acid sequences disclosed herein may also be used. Vectors such as pJB161 comprise sequences which are homologous with sequences present in plasmids endogenous to certain photosynthetic microorganisms (e.g., plasmids pAQ1, pAQ3, and pAQ4 of certain Synechococcus species). Examples of such vectors and how to use them is known in the art and provided, for example, in Xu et al., “Expression of Genes in Cyanobacteria: Adaptation of Endogenous Plasmids as Platforms for High-Level Gene Expression in Synechococcus sp. PCC 7002,” Chapter 21 in Robert Carpentier (ed.), “Photosynthesis Research Protocols,” Methods in Molecular Biology, Vol. 684, 2011, which is hereby incorporated herein by reference. Recombination between pJB161 and the endogenous plasmids in vivo yield engineered microbes expressing the genes of interest from their endogenous plasmids. Alternatively, vectors can be engineered to recombine with the host cell chromosome, or the vector can be engineered to replicate and express genes of interest independent of the host cell chromosome or any of the host cell's endogenous plasmids.

A further example of a vector suitable for recombinant protein production is the pET system (Novagen®). This system has been extensively characterized for use in E. coli and other microorganisms. In this system, target genes are cloned in pET plasmids under control of strong bacteriophage T7 transcription and (optionally) translation signals; expression is induced by providing a source of T7 RNA polymerase in the host cell. T7 RNA polymerase is so selective and active that, when fully induced, almost all of the microorganism's resources are converted to target gene expression; the desired product can comprise more than 50% of the total cell protein a few hours after induction. It is also possible to attenuate the expression level simply by lowering the concentration of inducer. Decreasing the expression level may enhance the soluble yield of some target proteins. In some embodiments this system also allows for maintenance of target genes in a transcriptionally silent un-induced state.

In some embodiments of using this system, target genes are cloned using hosts that do not contain the T7 RNA polymerase gene, thus alleviating potential problems related to plasmid instability due to the production of proteins potentially toxic to the host cell. Once established in a non-expression host, target protein expression may be initiated either by infecting the host with λCE6, a phage that carries the T7 RNA polymerase gene under the control of the λ pL and pI promoters, or by transferring the plasmid into an expression host containing a chromosomal copy of the T7 RNA polymerase gene under lacUV5 control. In the second case, expression is induced by the addition of IPTG or lactose to the bacterial culture or using an autoinduction medium. Other plasmids systems that are controlled by the lac operator, but do not require the T7 RNA polymerase gene and rely upon E. coli's native RNA polymerase include the pTrc plasmid suite (Invitrogen) or pQE plamid suite (QIAGEN).

In other embodiments it is possible to clone directly into expression hosts. Two types of T7 promoters and several hosts that differ in their stringency of suppressing basal expression levels are available, providing great flexibility and the ability to optimize the expression of a wide variety of target genes.

### D. Promoters

Promoters useful for expressing the recombinant genes described herein include both constitutive and inducible/repressible promoters. Examples of inducible/repressible promoters include nickel-inducible promoters (e.g., PnrsA, PnrsB; see, e.g., Lopez-Mauy et al., Cell (2002) v. 43: 247-256) and urea repressible promoters such as PnirA (described in, e.g., Qi et al., Applied and Environmental Microbiology (2005) v. 71: 5678-5684). Additional examples of inducible/repressible promoters include PnirA (promoter that drives expression of the nirA gene, induced by nitrate and repressed by urea) and Psuf (promoter that drives expression of the sufB gene, induced by iron stress).

Examples of constitutive promoters include Pcpc (promoter that drives expression of the cpc operon), Prbc (promoter that drives expression of rubisco), PpsbAII (promoter that drives expression of the D1 protein of photosystem II reaction center), Pcro (lambda phage promoter that drives expression of cro). In other embodiments, a PaphI1 and/or a lacIq-Ptrc promoter can used to control expression. Where multiple recombinant genes are expressed in an engineered microorganism, the different genes can be controlled by different promoters or by identical promoters in separate operons, or the expression of two or more genes may be controlled by a single promoter as part of an operon.

Further non-limiting examples of inducible promoters include, but are not limited to, those induced by expression of an exogenous protein (e.g., T7 RNA polymerase, SP6 RNA polymerase), by the presence of a small molecule (e.g., IPTG, galactose, tetracycline, steroid hormone, abscisic acid), by absence or low concentration of small molecules (e.g., CO₂, iron, nitrogen), by metals or metal ions (e.g., copper, zinc, cadmium, nickel), and by environmental factors (e.g., heat, cold, stress, light, darkness), and by growth phase. In some embodiments, the inducible promoter is tightly regulated such that in the absence of induction, substantially no transcription is initiated through the promoter. In some embodiments, induction of the promoter does not substantially alter transcription through other promoters. Also, generally speaking, the compound or condition that induces an inducible promoter is not naturally present in the organism or environment where expression is sought.

In some embodiments, the inducible promoter is induced by limitation of CO₂ supply to a cyanobacteria culture. By way of non-limiting example, the inducible promoter may be the promoter sequence of Synechocystis PCC 6803 that are up-regulated under the CO₂-limitation conditions, such as the crop genes, ntp genes, ndh genes, sbt genes, chp genes, and rbc genes, or a variant or fragment thereof.

In some embodiments, the inducible promoter is induced by iron starvation or by entering the stationary growth phase. In some embodiments, the inducible promoter may be variant sequences of the promoter sequence of cyanobacterial genes that are up-regulated under Fe-starvation conditions such as isiA, or when the culture enters the stationary growth phase, such as isiA, phrA, sigC, sigB, and sigH genes, or a variant or fragment thereof.

In some embodiments, the inducible promoter is induced by a metal or metal ion. By way of non-limiting example, the inducible promoter may be induced by copper, zinc, cadmium, mercury, nickel, gold, silver, cobalt, and bismuth or ions thereof. In some embodiments, the inducible promoter is induced by nickel or a nickel ion. In some embodiments, the inducible promoter is induced by a nickel ion, such as Ni²⁺. In another exemplary embodiment, the inducible promoter is the nickel inducible promoter from Synechocystis PCC 6803. In another embodiment, the inducible promoter may be induced by copper or a copper ion. In yet another embodiment, the inducible promoter may be induced by zinc or a zinc ion. In still another embodiment, the inducible promoter may be induced by cadmium or a cadmium ion. In yet still another embodiment, the inducible promoter may be induced by mercury or a mercury ion. In an alternative embodiment, the inducible promoter may be induced by gold or a gold ion. In another alternative embodiment, the inducible promoter may be induced by silver or a silver ion. In yet another alternative embodiment, the inducible promoter may be induced by cobalt or a cobalt ion. In still another alternative embodiment, the inducible promoter may be induced by bismuth or a bismuth ion.

In some embodiments, the promoter is induced by exposing a cell comprising the inducible promoter to a metal or metal ion. The cell may be exposed to the metal or metal ion by adding the metal to the microbial growth media. In certain embodiments, the metal or metal ion added to the microbial growth media may be efficiently recovered from the media. In other embodiments, the metal or metal ion remaining in the media after recovery does not substantially impede downstream processing of the media or of the bacterial gene products.

Further non-limiting examples of constitutive promoters include constitutive promoters from Gram-negative bacteria or a bacteriophage propagating in a Gram-negative bacterium. For instance, promoters for genes encoding highly expressed Gram-negative gene products may be used, such as the promoter for Lpp, OmpA, rRNA, and ribosomal proteins. Alternatively, regulatable promoters may be used in a strain that lacks the regulatory protein for that promoter. For instance P_{lac}, P_{tac}, and P_{trc}, may be used as constitutive promoters in strains that lack Lacl. Similarly, P22 P_{R} and P_{L} may be used in strains that lack the lambda C2 repressor protein, and lambda P_{R} and P_{L} may be used in strains that lack the lambda C1 repressor protein. In one embodiment, the constitutive promoter is from a bacteriophage. In another embodiment, the constitutive promoter is from a Salmonella bacteriophage. In yet another embodiment, the constitutive promoter is from a cyanophage. In some embodiments, the constitutive promoter is a Synechocystis promoter. For instance, the constitutive promoter may be the PpsbAll promoter or its variant sequences, the Prbc promoter or its variant sequences, the P_{cpc} promoter or its variant sequences, and the PrnpB promoter or its variant sequences.

In some embodiments the promoter comprises a sequence selected from SEQ ID NO: 25-42, variants of SEQ ID NO: 25-42, and derivatives of SEQ ID NO: 25-42.

### E. Host Cells

Also provided are host cells transformed with the nucleic acid molecules or vectors disclosed herein, and descendants thereof. In some embodiments the host cells are of a microorganism. In some embodiments the host cells are photosynthetic. In some embodiments, the host cells carry the nucleic acid sequences on vectors, which may but need not be freely replicating vectors, such as plasmids. In other embodiments, the nucleic acids have been integrated into the chromosome of the host cells and/or into an endogenous plasmid of the host cells. The transformed host cells find use, e.g., in the production of recombinant proteins.

“Microorganisms” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A variety of host microorganisms can be transformed with a nucleic acid sequence disclosed herein and can in some embodiments produce a recombinant protein encoded by the nucleic acid sequence. Suitable host microorganisms include both autotrophic and heterotrophic microbes. In some applications the autotrophic microorganism allows for a reduction in the fossil fuel and/or electricity inputs required to make a recombinant protein encoded by a recombinant nucleic acid sequence introduced into the host microorganism. This, in turn, in some applications reduces the cost and/or the environmental impact of producing the recombinant protein and/or reduces the cost and/or the environmental impact in comparison to the cost and/or environmental impact of manufacturing alternative proteins.

Photosynthetic microorganisms that can be transformed with the nucleic acid molecules or vectors disclosed herein, and descendants thereof, include eukaryotic algae, as well as prokaryotic cyanobacteria, green-sulfur bacteria, green non-sulfur bacteria, purple sulfur bacteria, and purple non-sulfur bacteria.

Algae and cyanobacteria include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chiysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chiysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichiysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodennatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protodenna, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spennatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium.

Additional cyanobacteria include members of the genus Chamaesiphon, Chroococcus, Cyanobacterium, Cyanobium, Cyanothece, Dactylococcopsis, Gloeobacter, Gloeocapsa, Gloeothece, Microcystis, Prochlorococcus, Prochloron, Synechococcus, Synechocystis, Cyanocystis, Dermocarpella, Stanieria, Xenococcus, Chroococcidiopsis, Myxosarcina, Arthrospira, Borzia, Crinalium, Geitlerinemia, Leptolyngbya, Limnothrix, Lyngbya, Microcoleus, Oscillatoria, Planktothrix, Prochlorothrix, Pseudanabaena, Spirulina, Starria, Symploca, Trichodesmium, Tychonema, Anabaena, Anabaenopsis, Aphanizomenon, Cyanospira, Cylindrospermopsis, Cylindrospennum, Nodularia, Nostoc, Scylonema, Calothrix, Rivularia, Tolypothrix, Chlorogloeopsis, Fischerella, Geitieria, Iyengariella, Nostochopsis, Stigonema and Thermosynechococcus.

Green non-sulfur bacteria include but are not limited to the following genera: Chloroflexus, Chloronema, Oscillochloris, Heliothrix, Herpetosiphon, Roseiflexus, and Thermomicrobium.

Green sulfur bacteria include but are not limited to the following genera: Chlorobium, Clathrochloris, and Prosthecochloris.

Purple sulfur bacteria include but are not limited to the following genera: Allochromatium, Chromatium, Halochromatium, Isochromatium, Marichromatium, Rhodovulum, Thermochromatium, Thiocapsa, Thiorhodococcus, and Thiocystis.

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

In some embodiments a non-photosynthetic microorganism is transformed with the nucleic acid molecules or vectors disclosed herein. Such microorganisms include Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis. In some embodiments those organisms are engineered to fix carbon dioxide while in other embodiments they are not.

### F. Methods of Making Secreted Polypeptides

One or more of the recombinant nucleic acids disclosed herein can be introduced into a host microorganism and the host microorganism can be used to produce a recombinant secreted polypeptide sequence. Accordingly, this disclosure provides a method for producing a secreted recombinant polypeptide sequence. In some embodiments the method comprises providing a recombinant photosynthetic microorganism comprising a recombinant nucleic acid comprising a first nucleic acid sequence encoding the recombinant polypeptide sequence operatively linked to a second nucleic acid sequence encoding a signal peptide; and culturing the recombinant photosynthetic microorganism in a culture medium under conditions sufficient for production and secretion of the recombinant protein by the recombinant photosynthetic microorganism. In some embodiments the coding sequence for the signal peptide is not native to the recombinant photosynthetic microorganism. In some embodiments of the method, the signal peptide comprises an amino acid sequence selected from SEQ ID NOS: 1-12 or the amino acid sequences shown in Tables 16, 17, 18, and/or 19, a mutein of an amino acid sequence selected from SEQ ID NOS: 1-12 or the amino acid sequences shown in Tables 16, 17, 18, and/or 19, and a derivative of an amino acid sequence selected from SEQ ID NOS: 1-12 or the amino acid sequences shown in Tables 16, 17, 18, and/or 19.

This disclosure also provides an alternative method for producing a secreted recombinant polypeptide sequence. In some embodiments the alternative method comprises providing a recombinant microorganism comprising a recombinant nucleic acid comprising a first nucleic acid sequence encoding the recombinant polypeptide sequence operatively linked to a second nucleic acid sequence encoding a signal peptide; and culturing the recombinant microorganism in a culture medium under conditions sufficient for production and secretion of the recombinant protein by the recombinant microorganism. In some embodiments of the alternative method the signal peptide comprises an amino acid sequence selected from SEQ ID NOS: 1-12 or the amino acid sequences shown in Tables 16, 17, 18, and/or 19, a mutein of an amino acid sequence selected from SEQ ID NOS: 1-12 or the amino acid sequences shown in Tables 16, 17, 18, and/or 19, and a derivative of an amino acid sequence selected from SEQ ID NOS: 1-12 or the amino acid sequences shown in Tables 16, 17, 18, and/or 19. In some embodiments of the methods the nucleic acid sequence that encodes a signal peptide is selected from SEQ ID NOS: 13-24 or the nucleotide sequences shown in Tables 16, 17, 18, and/or 19.

In some embodiments of the methods, the second nucleic acid sequence encoding a signal peptide is located upstream of the first nucleic acid sequence encoding the recombinant polypeptide sequence, wherein the signal peptide comprises an amino acid sequence selected from SEQ ID NOS: 1-8, a mutein of an amino acid sequence selected from SEQ ID NOS: 1-8, and a derivative of an amino acid sequence selected from SEQ ID NOS: 1-8. In some embodiments of the methods, the nucleic acid sequence that encodes a signal peptide is selected from SEQ ID NOS: 13-20.

In some embodiments of the methods, the second nucleic acid sequence encoding a signal peptide is located downstream of the first nucleic acid sequence encoding the recombinant polypeptide sequence, wherein the signal peptide comprises an amino acid sequence selected from SEQ ID NOS: 9-12, a mutein of an amino acid sequence selected from SEQ ID NOS: 9-12, and a derivative of an amino acid sequence selected from SEQ ID NOS: 9-12. In some embodiments of the methods, the nucleic acid sequence that encodes a signal peptide is selected from SEQ ID NOS: 21-24.

In some embodiments of the methods, the recombinant polypeptide sequence is a naturally occurring eukaryotic protein, or a mutein or derivative thereof. In some embodiments of the methods, the recombinant polypeptide sequence is a naturally occurring nutritive protein, or a mutein or derivative thereof. In some embodiments of the methods the recombinant polypeptide sequence is a naturally occurring intracellular protein, or a mutein or derivative thereof.

In some embodiments of the methods, the recombinant nucleic acid, further comprises third nucleic acid sequence that is an expression control sequence operatively linked to the first nucleic acid sequence encoding the recombinant polypeptide sequence and the second nucleic acid sequence encoding a signal peptide. In some embodiments, the expression control sequence comprises a promoter. In some embodiments the promoter is an inducible promoter. In some embodiments the promoter is a repressible promoter. In some embodiments the promoter comprises a nucleic acid sequence selected from SEQ ID NOS: 25-41 and derivatives thereof.

In some embodiments of the methods, the recombinant microorganism further comprises a nucleic acid comprising at least one open reading frame that encodes at least one protein selected from SEQ ID NOS: 50-56.

In some embodiments of the methods, the nucleic acid is integrated into a chromosome of the recombinant microorganism. In some embodiments of the methods, the nucleic acid is integrated into each copy of the chromosome of the recombinant microorganism. In some embodiments of the methods, the recombinant microorganism comprises a vector comprising the recombinant nucleic acid. In some embodiments the vector is a plasmid.

In some embodiments of the methods, at least one endogenous pilus assembly gene is inactivated in the recombinant microorganism.

In some embodiments of the methods, the recombinant microorganism is thermophylic. In some embodiments of the methods, the recombinant microorganism is a cyanobacterium. In some embodiments of the methods, the cyanobacterium is a strain selected from Synechococcus sp. PCC 7002, Synechococcus sp. ATCC 29404, Synechocystis sp. PCC 6308, and Synechococcus elongatus sp. PCC 7942-1.

In some embodiments the methods further comprise recovering the secreted recombinant protein from the culture medium. In some embodiments the secreted recombinant protein is recovered from the culture medium during the exponential growth phase. In some embodiments the secreted recombinant protein is recovered from the culture medium during the stationary phase. In some embodiments the secreted recombinant protein is recovered from the culture medium at a first time point, the culture is continued under conditions sufficient for production and secretion of the recombinant protein by the microorganism, and the recombinant protein is recovered from the culture medium at a second time point. In some embodiments the secreted recombinant protein is recovered from the culture medium by a continuous process.

Skilled artisans are aware of many suitable methods available for culturing recombinant cells to produce (and optionally secrete) a recombinant nutritive protein as disclosed herein, as well as for purification and/or isolation of expressed recombinant proteins. The methods chosen for protein purification depend on many variables, including the properties of the protein of interest. Culture conditions can also have an effect on solubility and localization of a given target protein. Many approaches can be used to purify target proteins expressed in recombinant microbial cells as disclosed herein, including without limitation ion exchange and gel filtration.

In some embodiments a peptide fusion tag is added to the recombinant protein making possible a variety of affinity purification methods that take advantage of the peptide fusion tag. In some embodiments, the use of an affinity method enables the purification of the target protein to near homogeneity in one step. Purification may include cleavage of part or all of the fusion tag with enterokinase, factor Xa, thrombin, or HRV 3C proteases, for example. In some embodiments, before purification or activity measurements of an expressed target protein, preliminary analysis of expression levels, cellular localization, and solubility of the target protein is performed.

While Escherichia coli is widely regarded as a robust host for heterologous protein expression, it is also widely known that over-expression of many proteins in this host is prone to aggregation in the form of insoluble inclusion bodies. One of the most commonly used methods for either rescuing inclusion body formation, or to improve the titer of the protein itself is to include an amino-terminal maltose-binding protein (MBP) [Austin B P, Nallamsetty S, Waugh D S. Hexahistidine-tagged maltose-binding protein as a fusion partner for the production of soluble recombinant proteins in Escherichia coli. Methods Mol. Biol. 2009; 498:157-72], or small ubiquitin-related modifier (SUMO) [Saitoh H, Uwada J, Azusa K. Strategies for the expression of SUMO-modified target proteins in Escherichia coli. Methods Mol. Biol. 2009; 497:211-21; Malakhov M P, Mattern M R, Malakhova O A, Drinker M, Weeks S D, Butt T R. SUMO fusions and SUMO-specific protease for efficient expression and purification of proteins. J Struct Funct Genomics. 2004; 5(1-2):75-86; Panavas T, Sanders C, Butt T R. SUMO fusion technology for enhanced protein production in prokaryotic and eukaryotic expression systems. Methods Mol. Biol. 2009; 497:303-17] fusion to the protein of interest. These two proteins are expressed extremely well, and in the soluble form, in Escherichia coli such that the protein of interest is also effectively produced in the soluble form. The protein of interest can be cleaved by designing a site specific protease recognition sequence (such as the tobacco etch virus (TEV) protease) in-between the protein of interest and the fusion protein [1].

### G. Recombinant Polypeptides

The recombinant polypeptide produced by a recombinant host cell can be any type of protein. In some embodiments it is a naturally occurring protein. In some embodiments it is a variant and/or a derivative of a naturally occurring protein. In some embodiments it is a protein that is designed without reference to any naturally occurring protein. The recombinant polypeptide can be a protein that naturally occurs as an intracellular protein or as an extracellular protein.

In some embodiments the recombinant protein is itself the product of interest. In other words, the recombinant microorganism is used, among other things, to produce the protein and the protein is then recovered from the cell culture. In other embodiments the recombinant protein is an enzyme and the enzyme is involved in a pathway that synthesizes the product of interest. In other words, the recombinant microorganism is used, among other things, to produce the protein which then acts on a substrate to catalyze formation of a reaction product that is itself a product of interest or an intermediate in production of a product of interest. In some such embodiments the product of interest is a protein or a peptide. In some embodiments the product of interest is a fatty acid (such as for example a free fatty acid). In some embodiments the product of interest is a biofuel. In some embodiments the product of interest is a hydrocarbon. In some embodiments the product of interest is a plastic. In some embodiments the product of interest is a wax. In some embodiments the product of interest is a solvent. In some embodiments the product of interest is an oil. The product of interest is in some embodiments formed in the growth media comprising the microorganism, while in other embodiments the recombinant enzyme is itself recovered from the growth media comprising the microorganism and then used to catalyze production of the product of interest.

A “biofuel” refers to any fuel that derives from a biological source. Biofuel can refer to one or more hydrocarbons, one or more alcohols, one or more fatty esters or a mixture thereof. A “hydrocarbon” refers generally to a chemical compound that consists of the elements carbon (C), hydrogen (H) and optionally oxygen (O). There are three types of hydrocarbons, aromatic hydrocarbons, saturated hydrocarbons and unsaturated hydrocarbons such as alkenes, alkynes, and dienes.

In some embodiments the product of interest is selected from alcohols such as ethanol, propanol, isopropanol, butanol, fatty alcohols; esters such as fatty acid esters, wax esters; hydrocarbons and alkanes such as propane, octane, diesel, JP8; polymers such as terephthalate, 1,3-propanediol, 1,4-butanediol, polyols, PHA, PHB, acrylate, adipic acid, .epsilon.-caprolactone, isoprene, caprolactam, rubber; commodity chemicals such as lactate, DHA, 3-hydroxypropionate, .gamma.-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, HPA, lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-ADCA/cephalosporin, erythromycin, polyketides, statins, paclitaxel, docetaxel, terpenes, peptides, steroids, omega fatty acids and other such suitable products of interest. Such products are useful in the context of fuels, biofuels, industrial and specialty chemicals, additives, as intermediates used to make additional products, such as nutritional supplements, neutraceuticals, polymers, paraffin replacements, personal care products and pharmaceuticals. These compounds can also be used as feedstock for subsequent reactions for example transesterification, hydrogenation, catalytic cracking via either hydrogenation, pyrolisis, or both or epoxidations reactions to make other products.

Alkanes, also known as paraffins, are chemical compounds that consist only of the elements carbon (C) and hydrogen (H) (i.e., hydrocarbons), wherein these atoms are linked together exclusively by single bonds (i.e., they are saturated compounds) without any cyclic structure. n-Alkanes are linear, i.e., unbranched, alkanes. Together, acyl-ACP reductase (AAR) and alkanal decarboxylative monooxygenase (ADM) enzymes function to synthesize n-alkanes from acyl-ACP molecules. In some embodiments the recombinant protein is an AAR or ADM enzyme. Exemplary full-length nucleic acid sequences for genes encoding AAR are presented as SEQ ID NOs: 1, 5, and 13 of U.S. Pat. No. 7,955,820, and the corresponding amino acid sequences are presented as SEQ ID NOs: 2, 6, and 10, respectively. Exemplary full-length nucleic acid sequences for genes encoding ADM are presented as SEQ ID NOs: 3, 7, 14 of U.S. Pat. No. 7,955,820, and the corresponding amino acid sequences are presented as SEQ ID NOs: 4, 8, and 12, respectively. Those nucleic acid and amino acid sequences of U.S. Pat. No. 7,955,820 are hereby incorporated herein by reference. Additional nucleic acids that can be used include any of the genes encoding the AAR and ADM enzymes in Table 1 and Table 2, respectively, of U.S. Pat. No. 7,955,820, which tables are hereby incorporated herein by reference.

In some embodiments the enzyme is a component of the mevalonate pathway, selected from (a) an enzyme capable of combining two molecules of acetyl-coenzyme A to form acetoacetyl-CoA, such as acetyl-CoA thiolase; (b) an enzyme capable of condensing acetoacetyl-CoA with another molecule of acetyl-CoA to form 3-hydroxy-3-methylglutaryl-CoA (HMG-CoA), such as HMG-CoA synthase; (c) an enzyme capable of converting HMG-CoA to mevalonate, such as HMG-CoA reductase; (d) an enzyme capable of phosphorylating mevalonate to form mevalonate 5-phosphate, such as mevalonate kinase; (e) an enzyme capable of adding a second phosphate group to mevalonate 5-phosphate to form mevalonate 5-pyrophosphate, such as phosphomevalonate kinase; (f) an enzyme capable of converting mevalonate 5-pyrophosphate into IPP, such as mevalonate pyrophosphate decarboxylase; and (g) an enzyme capable of converting IPP to DMAPP, such as IPP isomerase.

In some embodiments the enzyme is a member of the DXP pathway, selected from (a) an enzyme capable of condensing pyruvate with D-glyceraldehyde 3-phosphate to make 1-deoxy-D-xylulose-5-phosphate, such as 1-deoxy-D-xylulose-5-phosphate synthase; (b) an enzyme capable of converting 1-deoxy-D-xylulose-5-phosphate to 2C-methyl-D-erythritol-4-phosphate, such as 1-deoxy-D-xylulose-5-phosphate reductoisomerase; (c) an enzyme capable of converting 2C-methyl-D-erythritol-4-phosphate to 4-diphosphocytidyl-2C-methyl-D-erythritol, such as 4-diphosphocytidyl-2C-methyl-D-erythritol synthase; (d) an enzyme capable of converting 4-diphosphocytidyl-2C-methyl-D-erythritol to 4-diphosphocytidyl-2C-methyl-D-erythritol-2-phosphate, such as 4-diphosphocytidyl-2C-methyl-D-erythritol kinase; (e) an enzyme capable of converting 4-diphosphocytidyl-2C-methyl-D-erythritol-2-phosphate to 2C-methyl-D-erythritol 2,4-cyclodiphosphate, such as 2C-methyl-D-erythritol 2,4-cyclodiphosphate synthase; (f) an enzyme capable of converting 2C-methyl-D-erythritol 2,4-cyclodiphosphate is converted to 1-hydroxy-2-methyl-2-(E)-butenyl-4-diphosphate, such as 1-hydroxy-2-methyl-2-(E)-butenyl-4-diphosphate synthase; and (g) an enzyme capable of converting 1-hydroxy-2-methyl-2-(E)-butenyl-4-diphosphate into either IPP or its isomer, DMAPP, such as isopentyl/dimethylallyl diphosphate synthase.

In some embodiments the recombinant polypeptide sequence is a nutritive protein. A “nutritive protein” is a protein that occurs naturally in an edible species. In its broadest sense, an “edible species” encompasses any species known to be eaten without deleterious effect by at least one type of mammal A deleterious effect includes a poisonous effect and a toxic effect. In some embodiments an edible species is a species known to be eaten by humans without deleterious effect. Some edible species are an infrequent but known component of the diet of only a small group of a type of mammal in a limited geographic location while others are a dietary staple throughout much of the world. In other embodiments an edible species is one not known to be previously eaten by any mammal, but that is demonstrated to be edible upon testing. Edible species include but are not limited to Gossypium turneri, Pleurotus cornucopias, Glycine max, Oryza sativa, Thunnus obesus, Abies bracteata, Acomys ignitus, Lathyrus aphaca, Bos gaurus, Raphicerus melanotic, Phoca groenlandica, Acipenser sinensis, Viverra tangalunga, Pleurotus sajor-caju, Fagopyrum tataricum, Pinus strobus, Ipomoea nil, Taxus cuspidata, Ipomoea wrightii, Mya arenaria, Actinidia deliciosa, Gazella granti, Populus tremula, Prunus domestica, Larus argentatus, Vicia villosa, Sargocentron punctatissimum, Silene latifolia, Lagenodelphis hosei, Spisula solidissima, Crossarchus obscurus, Phaseolus angularis, Lathyrus vestitus, Oncorhynchus gorbuscha, Alligator mississippiensis, Pinus halepensis, Larus canus, Brassica napus, Silene cucubalus, Phoca fasciata, Gazella bennettii, Pinus taeda, Taxus canadensis, Zamia furfuracea, Pinus yunnanensis, Pinus wallichiana, Asparagus officinalis, Capsicum baccatum, Pinus longaeva, Taxus baccata, Pinus sibirica, Citrus sinensis, Sargocentron xantherythrum, Bison bison, Gazella thomsonii, Vicia sativa, Branta canadensis, Apium graveolens, Acer campestre, Coriandrum sativum, Silene conica, Lactuca sativa, Capsicum chinense, Abies veitchii, Capra hircus, Gazella spekei, Oncorhynchus keta, Ipomoea obscura, Cucumis melo var. conomon, Phoca hispida, Vulpes vulpes, Ipomoea quamoclit, Solanum habrochaites, Populus sp., Pinus rigida, Quercus lyrata, Phaseolus coccineus, Larus ridibundus, Sargocentron spiniferum, Thunnus thynnus, Vulpes lagopus, Bos gaurus frontalis, Acer opalus, Acer palmatum, Quercus ilex, Pinus mugo, Grus antigone, Pinus uncinata, Prunus mume, Oncorhynchus tschawytscha, Gazella subgutturosa, Vulpes zerda, Pinus coulteri, Gossypium barbadense, Acer pseudoplatanus, Oncorhynchus nerka, Sus barbatus, Fagopyrum esculentum subsp. Ancestrale, Cynara cardunculus, Phaseolus aureus, Populus nigra, Gossypium schwendimanii, Solanum chacoense, Quercus rubra, Cucumis sativus, Equus burchelli, Oncorhynchus kisutch, Pinus radiata, Phoca vitulina richardsi, Grus nigricollis, Abies grandis, Oncorhynchus masou, Spinacia oleracea, Solanum chilense, Addax nasomaculatus, Ipomoea batatas, Equus grevyi, Abies sachalinensis, Pinus pinea, Hipposideros commersoni, Crocus nudiflorus, Citrus maxima, Acipenser transmontanus, Gossypium gossypioides, Viverra zibetha, Quercus cerris, Anser indicus, Pinus balfouriana, Silene otites, Oncorhynchus sp., Viverra megaspila, Bos mutus grunniens, Pinus elliottii, Equus hemionus kulan, Capra ibex ibex, Allium sativum, Raphanus sativus, Pinus echinata, Prunus serotina, Sargocentron diadema, Silene gallica, Brassica oleracea, Daucus carota, Oncorhynchus mykiss, Brassica oleracea var. alboglabra, Gossypium hirsutum, Abies alba, Citrus reticulata, Cichorium intybus, Bos sauveli, Lama glama, Zea mays, Acorus gramineus, Vulpes macrotis, Ovis amnion darwini, Raphicerus sharpei, Pinus contorta, Bos indicus, Capra sibirica, Pinus ponderosa, Prunus dulcis, Solanum sogarandinum, Ipomoea aquatica, Lagenorhynchus albirostris, Ovis canadensis, Prunus avium, Gazella dama, Thunnus alalunga, Silene pratensis, Pinus cembra, Crocus sativus, Citrullus lanatus, Gazella rufifrons, Brassica tournefortii, Capra falconeri, Bubalus mindorensis, Pinus palustris, Prunus laurocerasus, Grus vipio, Ipomoea purpurea, Pinus leiophylla, Lagenorhynchus obscurus, Raphicerus campestris, Brassica rapa subsp. Pekinensis, Acmella radicans, Ipomoea triloba, Pinus patula, Cucumis melo, Pinus virginiana, Solanum lycopersicum, Pinus dens flora, Pinus engelmannii, Quercus robur, Ipomoea setosa, Pleurotus djamor, Hipposideros diadema, Ovis aries, Sargocentron microstoma, Brassica oleracea var. italica, Capra cylindricornis, Populus kitakamiensis, Allium textile, Vicia faba, Fagopyrum esculentum, Bison priscus, Quercus suber, Lagophylla ramosissima, Acrantophis madagascariensis, Acipenser baerii, Capsicum annuum, Triticum aestivum, Xenopus laevis, Phoca sibirica, Acipenser naccarii, Actinidia chinensis, Ovis dalli, Solanum tuberosum, Bubalus carabanensis, Citrus jambhiri, Bison bonasus, Equus asinus, Bubalus depressicornis, Pleurotus eryngii, Solanum demissum, Ovis vignei, Zea mays subsp. Parviglumis, Lathyrus tingitanus, Welwitschia mirabilis, Grus rubicunda, Ipomoea coccinea, Allium cepa, Gazella soemmerringii, Brassica rapa, Lama vicugna, Solanum peruvianum, Xenopus borealis, Capra caucasica, Thunnus albacares, Equus zebra, Gallus gallus, Solanum bulbocastanum, Hipposideros terasensis, Lagenorhynchus acutus, Hippopotamus amphibius, Pinus koraiensis, Acer monspessulanum, Populus deltoides, Populus trichocarpa, Acipenser guldenstadti, Pinus thunbergii, Brassica oleracea var. capitata, Abyssocottus korotneffi, Gazella cuvieri, Abies homolepis, Abies holophylla, Gazella gazella, Pinus parviflora, Brassica oleracea var. acephala, Cucurbita pepo, Pinus armandii, Abies mariesii, Thunnus thynnus orientalis, Citrus unshiu, Solanum cheesmanii, Lagenorhynchus obliquidens, Acer platanoides, Citrus limon, Acrantophis dumerili, Solanum commersonii, Gossypium arboreum, Prunus persica, Pleurotus ostreatus, Abies firma, Gazella leptoceros, Salmo salar, Homarus americanus, Abies magnifica, Bos javanicus, Phoca largha, Sus cebifrons, Solanum melongena, Phoca vitulina, Pinus sylvestris, Zamia floridana, Vulpes corsac, Allium porrum, Phoca caspica, Vulpes chaeta, Taxus chinensis, Brassica oleracea var. botrytis, Anser anser anser, Phaseolus lunatus, Brassica campestris, Acer saccharum, Pinus pumila, Solanum pennellii, Pinus edulis, Ipomoea cordatotriloba, Populus alba, Oncorhynchus clarki, Quercus petraea, Sus verrucosus, Equus caballus przewalskii, Populus euphratica, Xenopus tropicalis, Taxus brevifolia, Lama guanicoe, Pinus banksiana, Solanum nigrum, Sus celebensis, Brassica juncea, Lagenorhynchus cruciger, Populus tremuloides, Pinus pungens, Bubalus quarlesi, Quercus gamelliflora, Ovis orientalis musimon, Bubalus bubalis, Pinus luchuensis, Sus philippensis, Phaseolus vulgaris, Salmo trutta, Acipenser persicus, Solanum brevidens, Pinus resinosa, Hippotragus niger, Capra nubiana, Asparagus scaber, Ipomoea platensis, Sus scrofa, Capra aegagrus, Lathyrus sativus, Sargocentron tiere, Hippoglossus hippoglossus, Acorus americanus, Equus caballus, Bos taurus, Barbarea vulgaris, Lama guanicoe pacos, Pinus pinaster, Octopus vulgaris, Solanum crispum, Hippotragus equinus, Equus burchellii antiquorum, Crossarchus alexandri, Ipomoea alba, Triticum monococcum, Populus jackii, Lagenorhynchus australis, Gazella dorcas, Quercus coccifera, Anser caerulescens, Acorus calamus, Pinus roxburghii, Pinus tabuliformis, Zamia fischeri, Grus carunculatus, Acomys cahirinus, Cucumis melo var. reticulatus, Gallus lafayettei, Pisum sativum, Pinus attenuata, Pinus clausa, Gazella saudiya, Capra ibex, Ipomoea trifida, Zea luxurians, Pinus krempfii, Acomys wilsoni, Petroselinum crispum, Quercus palustris, Triticum timopheevi, Meleagris gallopavo, Brassica oleracea, Brassica oleracea, Beta vulgaris, Solanum lycopersicum, Phaseolus vulgaris, Xiphias gladius, Morone saxatilis, Micropterus salmoides, Placopecten magellanicus, Sprattus sprattus, Clupea harengus, Engraulis encrasicolus, Cucurbita maxima, Agaricus bisporus, Musa acuminata x balbisiana, Malus domestica, Meleagris gallopavo, Anas platyrhynchos, Vaccinium macrocarpum, Rubus idaeus x strigosus, Vaccinium angustifolium, Fragaria ananassa, Rubus fruticosus, Cucumis melo, Ananas comosus, Cucurbita pepo, Cucurbita moschata, Sus scrofa domesticus, Ocimum basilicum, Rosmarinus officinalis, Foeniculum vulgare, Rheum rhabarbarum, Carica papaya, Mangifera indica, Actinidia deliciosa, Prunus armeniaca, Prunus avium, Cocos nucifera, Olea europaea, Pyrus communis, Ficus carica, Passiflora edulis, Oryza sativa subsp. Japonica, Oryza sativa subsp. Indica, Coturnix coturnix, Saccharomyces cerevisiae.

In some embodiments the nutritive protein is an abundant protein in food. In some embodiments the abundant protein in food is selected from chicken egg proteins such as ovalbumin, ovotransferrin, and ovomucuoid; meat proteins such as myosin, actin, tropomyosin, collagen, and troponin; cereal proteins such as casein, alpha1 casein, alpha2 casein, beta casein, kappa casein, beta-lactoglobulin, alpha-lactalbumin, glycinin, beta-conglycinin, glutelin, prolamine, gliadin, glutenin, albumin, globulin; chicken muscle proteins such as albumin, enolase, creatine kinase, phosphoglycerate mutase, triosephosphate isomerase, apolipoprotein, ovotransferrin, phosphoglucomutase, phosphoglycerate kinase, glycerol-3-phosphate dehydrogenase, glyceraldehyde 3-phosphate dehydrogenase, hemoglobin, cofilin, glycogen phosphorylase, fructose-1,6-bisphosphatase, actin, myosin, tropomyosin a-chain, casein kinase, glycogen phosphorylase, fructose-1,6-bisphosphatase, aldolase, tubulin, vimentin, endoplasmin, lactate dehydrogenase, destrin, transthyretin, fructose bisphosphate aldolase, carbonic anhydrase, aldehyde dehydrogenase, annexin, adenosyl homocysteinase; pork muscle proteins such as actin, myosin, enolase, titin, cofilin, phosphoglycerate kinase, enolase, pyruvate dehydrogenase, glycogen phosphorylase, triosephosphate isomerase, myokinase; and fish proteins such as parvalbumin, pyruvate dehydrogenase, desmin, and triosephosphate isomerase.

In some embodiments the recombinant polypeptide sequence is a nutritive protein that is not naturally occurring. In some embodiments the recombinant polypeptide sequence comprises a first polypeptide sequence comprising a fragment of a naturally-occurring nutritive protein. In some embodiments the recombinant polypeptide sequence further comprises a second polypeptide sequence. In some embodiments the second polypeptide sequence consists of from 3 to 10, 5 to 20, 10 to 30, 20 to 50, 25 to 75, 50 to 100 or 100 to 200 amino acids. In some embodiments the second polypeptide sequence is not derived from a naturally-occurring nutritive protein. In some embodiments the second polypeptide sequence is selected from a tag for affinity purification, a protein domain linker, and a protease recognition site. In some embodiments the tag for affinity purification is a polyhistidine-tag. In some embodiments the protein domain linker comprises at least one copy of the sequence GGSG. In some embodiments the protease is selected from pepsin, trypsin, and chymotrypsin. In some embodiments the recombinant polypeptide sequence further comprises a third polypeptide sequence comprising a fragment of at least 50 amino acids of a naturally-occurring nutritive protein. In some embodiments the first and third polypeptide sequences are the same. In some embodiments the first and third polypeptide sequences are different. In some embodiments the first and third polypeptide sequences are derived from the same naturally-occurring nutritive protein. In some embodiments the order of the first and third polypeptide sequences in the isolated recombinant nutritive protein is the same as the order of the first and third polypeptide sequences in the naturally-occurring nutritive protein. In some embodiments the order of the first and third polypeptide sequences in the isolated recombinant nutritive protein is different than the order of the first and third polypeptide sequences in the naturally-occurring nutritive protein. In some embodiments the first and third polypeptide sequences are derived from different naturally-occurring nutritive proteins. In some embodiments the second polypeptide sequence is flanked by the first and third polypeptide sequences.

In some embodiments the recombinant polypeptide sequence comprises at least 50 amino acids that are at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.5% homologous to at least one naturally occurring nutritive protein amino acid sequence or to at least one fragment of at least 50 amino acids of at least one naturally occurring nutritive protein amino acid sequence.

In some embodiments the polypeptide sequence can be linked (operably, directly, or via a linker) to a second polypeptide sequence. In some aspects, the second polypeptide sequence is an enzyme. In some aspects, the enzyme is glucoamylase.

In some embodiments the polypeptide sequence can be a food or feed enzyme such as a starch and/or sugar processing enzyme, a dairy enzyme, a bakery enzyme, a brewing enzyme, or a fruit processing enzyme. In some embodiments the recombinant polypeptide sequence can be an industrial enzyme such as a bioethanol enzyme, a detergent, a paper/pulp processing enzyme, a wastewater treatment enzyme, a leath processing enzyme, or a textile enzyme.

In some embodiments the polypeptide sequence can be a food processing enzyme such as an amylase or a protease. In some embodiments the polypeptide sequence can be a baby food enzyme such as trypsin. In some embodiments the polypeptide sequence can be a brewing industry enzyme such as a barley enzyme, amylase, glucanase, protease, betaglucanase, arabinoxylanase, amyloglucosidase, pullulanase, protease, or acetolactatedecarboxylase (ALDC). In some embodiments the polypeptide sequence can be a fruit juice enzyme such as a cellulase or pectinase. In some embodiments the polypeptide sequence can be a dairy enzyme such as rennin, lipase, or lactase. In some embodiments the polypeptide sequence can be a meat tenderizer enzyme such as papain. In some embodiments the polypeptide sequence can be a starch enzyme such as amylase, amyloglucosidase, glucoamylase, or glucose isomease. In some embodiments the polypeptide sequence can be a paper enzyme such as amylase, xylanase, cellulase, or ligninase. In some embodiments the polypeptide sequence can be a biofuel enzyme such as a cellulase or ligninase. In some embodiments the polypeptide sequence can be biological detergent such as protease, amylase, lipase, or cellulase. In some embodiments the polypeptide sequence can be a contact lens cleaner enzyme such as a protease. In some embodiments the polypeptide sequence can be a rubber enzyme such as catalase. In some embodiments the polypeptide sequence can be photograph enzyme such as protease. In some embodiments the polypeptide sequence can be a molecular biology enzyme such as a restriction enzyme, DNA ligase, or a polymerase.

### Computer Implementation

In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

Described herein is a computer-implemented method for identifying one or more candidate signal peptides, comprising: obtaining a data set comprising amino acid sequence data for one or more candidate signal peptides, wherein each candidate signal peptides comprises at least the first 40 amino acids of an amino acid sequence selected from a plurality of protein sequences from a microorganism proteome; and identifying, by a computer processor, one or more candidate signal peptides using an interpretation function.

In some aspects, at least 50% of identified candidate signal peptides are capable of directing secretion of a lichenase polypeptide having an activity greater than 0.5 μg lichenase/mL/OD730 from a recombinant microorganism, wherein the recombinant microorganism comprises one or more recombinant nucleic acid sequences comprising a first nucleic acid sequence encoding the lichenase polypeptide sequence operatively linked to a second nucleic acid sequence encoding the candidate signal peptide.

In some aspects, at least 50, 51, 52, 53, 54, 55, 56, 57, 58, 59, 60, 61, 62, 63, or 64% of identified candidate signal peptides are capable of directing secretion of lichenase polypeptide having an activity greater than 0.5 μg lichenase/mL/OD730 from the recombinant microorganism. In some aspects, at least 50, 51, or 52% of identified candidate signal peptides are capable of directing secretion of lichenase polypeptide having an activity greater than 0.75 μg lichenase/mL/OD730 from the recombinant microorganism. In some aspects, at least 37% of identified candidate signal peptides are capable of directing secretion of lichenase polypeptide having an activity greater than 1.0 μg lichenase/mL/OD730 from the recombinant microorganism. In some aspects, at least 23% of identified candidate signal peptides are capable of directing secretion of lichenase polypeptide having an activity greater than 1.25 μg lichenase/mL/OD730 from the recombinant microorganism. In some aspects, the data set comprises amino acid sequence data for the whole microorganism proteome.

### EXAMPLES

### Example 1

### Identification of Cyanobacterial Secreted Proteins

We hypothesized that the signal peptides of secreted proteins from cyanobacteria are well suited for use in constructing secretion systems for engineering microorganisms such as photosynthetic microorganisms such as cyanobacteria. As such, we performed a study to identify proteins secreted at high levels from a variety of host cyanobacteria strains and to identify the signal peptides of those proteins, and the nucleic acid sequences that encode the secreted proteins and the signal peptides.

Isolation of Naturally Secreted Extracellular Proteins from Liquid Cultures.

For isolation of extracellular proteins, liquid cultures of different cyanobacterial strains were grown to late-exponential growth phase. After pelleting cells through high-speed centrifugation, the supernatants were collected and further purified using a Millipore 0.22 μm filter unit. Following purification, extracellular protein samples were concentrated using either TCA precipitation or 3 kDa cut-off membrane filters.

Purification of the Most Abundant Protein Bands for Gene Identification.

Strains Synechococcus sp. PCC 7002; Synechococcus sp. ATCC 29404; Synechocystis sp. PCC 6308; and Synechococcus elongatus sp. PCC 7942-1 were cultured and extracellular proteins were isolated from the culture medium using SDS-PAGE (data not shown).

Gene Identification Through LC-MS Fingerprinting and N-Terminal Sequencing.

To identify the putative genes for these newly identified naturally secreted proteins, liquid chromatography-mass spectrometry (LC-MS) analysis and N-terminal sequencing was used to identify the genes of the secreted proteins through Finger-printing analysisdone. The genomic sequences of Synechococcus sp. PCC 7002 and Synechococcus elongatus sp. PCC 7942-1 are available in the GenBank, and we determined the genomic sequences of Synechococcus sp. ATCC 29404 and Synechocystis sp. PCC 6308, so LC-MS and sequencing data was used to identify genes of Synechococcus sp. PCC 7002, Synechococcus sp. ATCC 29404, Synechocystis sp. PCC 6308, and Synechococcus elongatus sp. PCC 7942-1 that encode the secreted proteins. That allowed determination of the full amino acid sequence of each secreted protein and the nucleic acid sequence of the gene encoding each secreted protein.

Genes for secreted proteins were verified by protein sequence analysis (high fingerprinting coverage) and secretion signal peptide predictions. Nine secreted proteins were identified (SP1-SP9; SEQ ID NOS: 57-65) and are listed in Table 1. The genes that encode those proteins (SG1-SG9; SEQ ID NOS: 66-74) are also listed in Table 1.

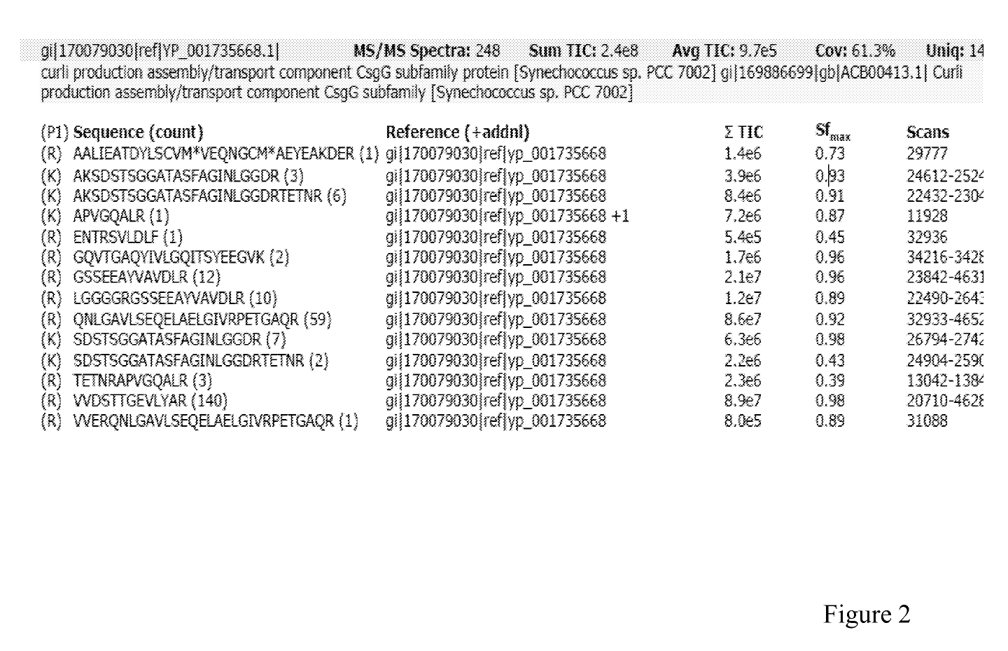

Exemplary results for the SP1 protein (SEQ ID NO: 57) (encoded by SYNPCC7002_A2435; SG1; SEQ ID NO: 66) are presented in FIG. 2. The Signal 4.0 program calculates a high probability that the N-terminal portion of this protein is a secretion signal sequence. Using the same method, the secretion leaders have also been analyzed and identified for other newly identified secreted proteins. The sequences and secretion cleavage sites of the identified secreted proteins provide putative secretion leader sequences that can be used to design recombinant expressed proteins and nucleic acids that encode them.

This approach was used to identify eight new N-terminal signal peptides (SEQ ID NOS: 1-8), which are listed in Table 2. The N-terminal signal peptides are encoded by SEQ ID NOS: 13-20.

Identification of a Potentially New Secretion System in Synechococcus Sp. PCC 7002.

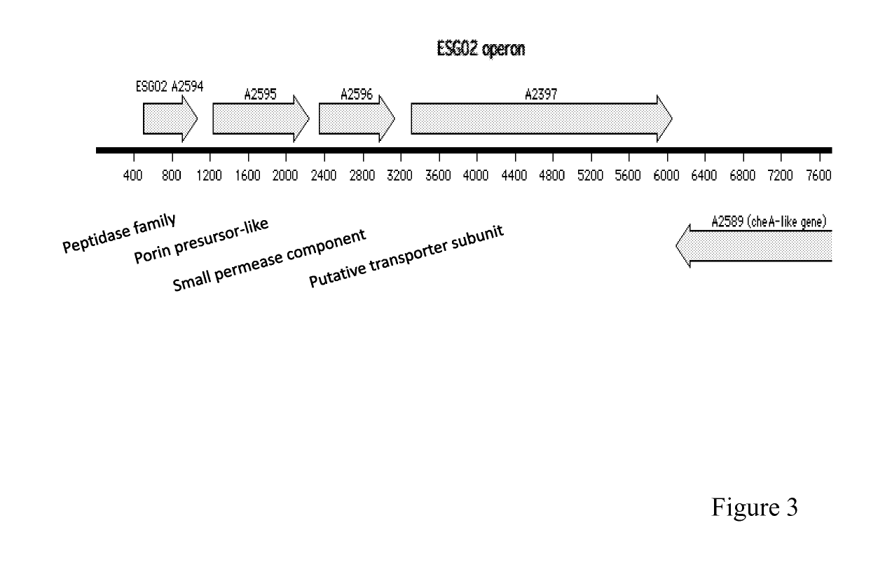

Based on the bioinformatics analysis, similarity has been shown in comparison of the SP1 (SEQ ID NO: 57) and SP2 (SEQ ID NO: 58) proteins. Both appear to be involved in the production of extracellular fibers, which suggests their involvement in secretion functions. Interestingly, the SP2 gene appears to be part of an operon containing four genes (FIG. 3). The genes in this operon are: SYNPCC7002_A2594 (SP2) (SEQ ID NO: 67), SYNPCC7002_A2595 (SEQ ID NO: 43), SYNPCC7002_A2596 (SEQ ID NO: 44), and SYNPCC7002_A2597 (SEQ ID NO: 45), which encode the protein sequences of SEQ ID NOS: 58, 50, 51, and 52, respectively. The possible functions of proteins encoded by the operon have been assessed by Blast analysis using Cyanobase (http://genome.kazusa.or.jp/cyanobase/) and NCBI Blast (http://blast.ncbi.nlm.nih.gov/Blast.cgi).

The second gene in the putative SYNPCC7002_A2594 operon, A2595, encodes a hypothetical protein that exhibites some similarity to proteins with functions in porin-like transporting, ATP-binding protease or chaperone. The third gene, A2596, encodes a 267 aa hypothetical protein with some similarity to proteins functioning as small permease components. The fourth gene, A2597, encodes a hypothetical protein with high similarity to putative ABC-type transporter proteins. Thus, it seems as if A2596 and 2597 encode transporter core components. Based on the functional similarity between SG2 and SG1 and the gene organization of the SYNPCC7002_A2594 operon (A2594-A2595-A2596-A2597), it is possible that functions of the SYNPCC7002_A2594 operon are associated with SG1 secretion, and secretion leader processing (cleavage after secretion) and possible assembly of the secreted SG1 protein.

Identification of a Potentially New Secretion System in Synechococcus Sp. ATCC 29404.

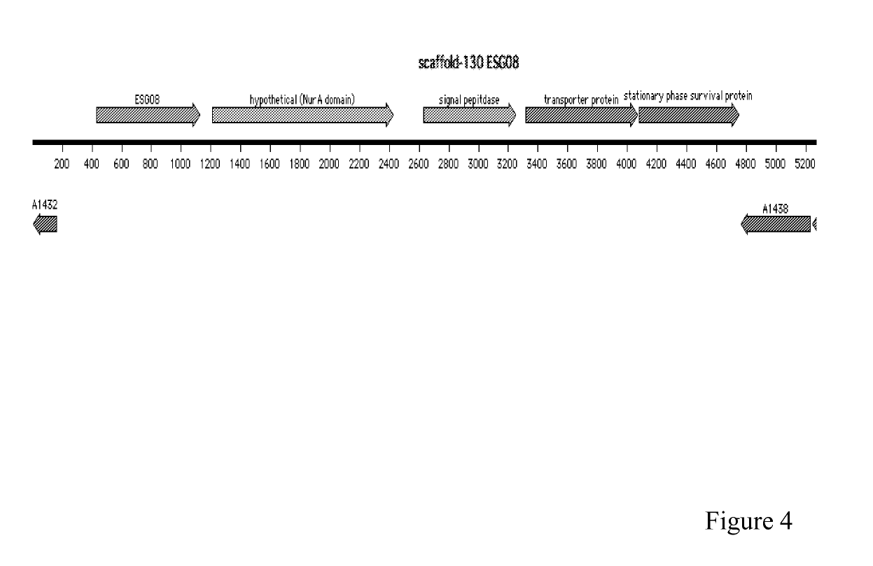

Identification of SG8 (SEQ ID NO: 73) and its surrounding sequences also led to the identification of a putative operon on Contig-130 of the sequenced Synechococcus sp. ATCC 29404 genome. The sequences of the SG8 operon genes are presented in SEQ ID NOS: 73, 46-49 (Table 10). The possible functions of the gene products have been determined by Blast analysis using Cyanobase (http://genome.kazusa.or.jp/cyanobase/) and NCBI Blast (http://blast.ncbi.nlm.nih.gov/Blast.cgi). In the operon, the SG8 gene encodes the secreted protein SP8 that was identified in the extracellular protein fraction. The second gene located downstream of SG8 encodes a hypothetical protein with high similarity with proteins such as the type II secretory pathway component PulF-like proteins. The third gene encodes a signal peptidase, which may assume function in processing the secretion leader. The fourth and the fifth genes encode proteins containing domains with similarities to proteins with transporter or chaperon functions. Based on this analysis, it's possible that the SG8 operon encodes components of the novel Type-II protein secretion system in cyanobacteria, which most likely plays roles in assisting secretion of the SG8 protein. FIG. 4.

Based on the similarities of the protein components of the putative Type IV SG2 secretion system and the putative Type II SG8 secretion system with the orthologs from heterotrophs (Koster, M., Bitter, W., and Tommassen, J. (2000) Protein secretion mechanisms in gram-native bacteria. Int. Med. Microbiol. 290: 325-331; Pallen, M. J., Chaudhuri, R. R., and Henderson, I. R. (2003) Genomic analysis of secretion systems. Curr. Opin. Micriobiol. 6: 519-527; Henderson, I. R., Navarro-Garcia, F., Desvaux, M., Fernandez, R. C., and Ala'Aldeen, D. (2004) Type V protein secretion pathway: the autotransporter story. Microbiol. Mol. Biol. Rev. 68: 692-744.), it is reasonable to assume that similar secretion systems exist in heterotrophic organisms, such as E. coli. Thus, gene expression plasmids comprising sequences encoding the signal peptides of SP1 and SP8 can be used to secrete a heterologous protein in a heterotroph. However, efficiency of the heterologous protein secretion could be lower compared to that in cyanobacteria. As demonstrated below, we have successfully secreted recombinant proteins in Synechococcus sp. PCC 7002 and Synechococcus sp. ATCC 29404 using the secretion leaders disclosed herein.

### Example 2

### Expression of Recombinant Proteins

Cyanobacteria Strains

The strains used in this example were Synechococcus sp. PCC 7002 and Synechococcus strain ATCC 29404 (PCC 73109).

Recombinant Plasmids

The recombinant plasmids used in this study were constructed from the pAQ1 plasmid of Synechococcus sp. PCC 7002 and the pContig41 plasmid of Synechococcus sp. ATCC 29404 (SEQ ID NO: 75). The sequence of the 4809 by pAQ1 plasmid has been determined and can be found in the database (Akiyama et al., 1998) (http://g.kazusa.or.jp/cgi-bin/gbrowse/SYNPCC7002/?name=pAQ1). Based on the annotations of the sequenced Synechococcus sp. ATCC 29404 genome, pContig41 contains two plasmid partition genes and several genes with high homology to genes located on plasmids in the Synechococcus sp. PCC 7002 genome. Therefore, the 12002 by of pContig41 is likely a plasmid. Gene expression constructs were generated for integration of expression cassettes into an intergenic region on the pContig41 plasmid.

Gene expression cassettes are designed with promoters selected from cyanobacteria and also from heterotrophic organisms. For integration of the gene expression cassettes into the plasmid of pAQ1, two flanking regions with pAQ1 DNA sequences were cloned for insertion of the gene expression cassettes. Specifically, gene expression platforms have been constructed using various promoters identified in cyanobacteria screens, including Pcpc (SEQ ID NO: 25), Pcpc* (SEQ ID NO: 26), Psuf (SEQ ID NO: 27), Prbc (SEQ ID NO: 28), Pnir (SEQ ID NO: 31), Ppsa (SEQ ID NO: 29), and PpsbAII (SEQ ID NO: 30). In order to design recombinant expression cassettes comprising the promoters, positioned to function in coordination with the transcription initiation site and other regulatory elements, three considerations have been used to select sequences of the promoter: 1) intragenic region upstream of the first gene in an operon; and 2) size of between 200-500 bp.

To construct gene expression vectors with different promoters, an expression cassette was first constructed by cloning the Pcpc promoter operatively linked to the reporter gene yfp (Accession number AA048597.1). The aadA gene confers spectinomycin resistance to allow selection of the transformants and was placed downstream of yfp. The vectors also include a gene that confers resistance to ampicillin (Anp^{r}). Additional constructs containing different promoters have also been generated using Pcpc (SEQ ID NO: 25), Pcpc* (SEQ ID NO: 26), Psuf (SEQ ID NO: 27), Prbc (SEQ ID NO: 28), Pnir (SEQ ID NO: 31), Ppsa (SEQ ID NO: 29), and PpsbAII (SEQ ID NO: 30). Digestion of the Pcpc construct with Eco RI and Nco I allows the replacement of the Pcpc promoter with a different promoter. The resulting expression vectors have been used to transform cells of Synechococcus sp. PCC 7002. Segregations of the transformants was achieved by re-streaking and screening colonies on A⁺ media containing spectinomycin. Full segregations of the engineered strain with yfp overexpression controlled by different promoters was confirmed by PCR analysis.

Use of the Plasmids

Recombinant plasmids were introduced into cyanobacterial hosts to evaluate expression of recombinant YFP. Fluorescence emission from YFP was used to compare the expression levels of the reporter gene yfp in strains with different promoters. Yfp expression was analyzed by measuring the fluorescence emission from YFP proteins, fluorescence emission amplitude was measured at emission at 527 nm with excitation at 480 nm. Liquid cultures of different strains, including a wild-type strain control, were grown to late exponential phase. Cell density and fluorescence emission were measured in microplates using the BioTek Multi-Mode Microplate Reader and cell density was adjusted to OD730=0.4. Cell density is monitored with measuring OD at 730 nm. The density of each culture was normalized using the optical density at 730 nm.

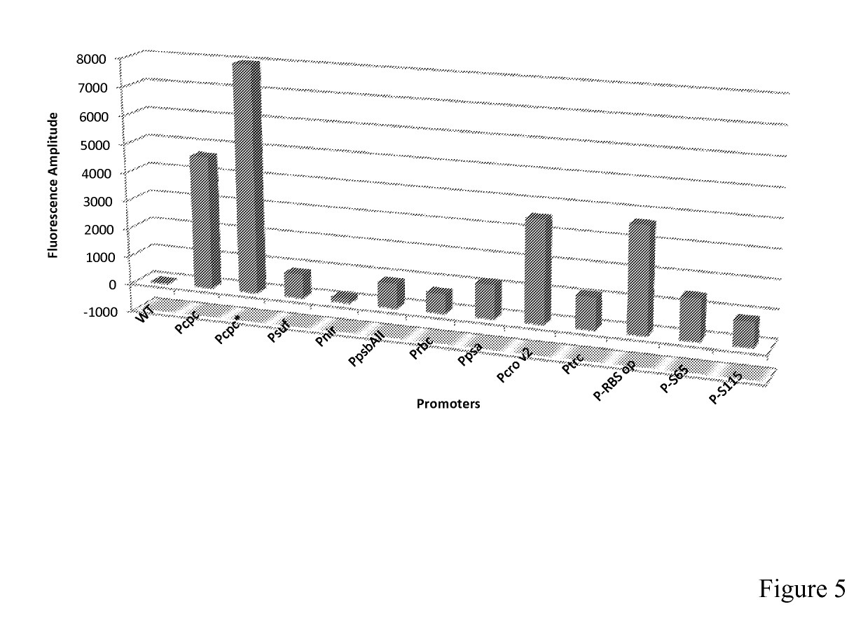

Results of these experiments are presented in FIG. 5. YFP overproduction in these engineered strains was also analyzed by SDS-PAGE and Western blot assays using a polyclonal anti-YFP antibody [Invitrogen]. The promoter of the cpcBACEF (Pcpc* (SEQ ID NO: 26), encoding the major components of the light-harvesting phycobilisome) from the high-temperature tolerant Thermosynechococcus elongates BP-1 proved to be the best promoter in our constructs for gene expression in Synechococcus sp. PCC 7002. Recombinant plasmids comprising the Pcpc* promoter have been introduced successfully into other cyanobacteria, including Synechococcus elongatus PCC 7942 and Synechocossus sp. ATCC 29404. (Data not shown.)

The results presented in FIG. 5 include experiments analyzing a modified Pcpc* promoter. In P-RBS-op the ribosome-binding site was modified from “AGGAGA” to “GGAG” and the spacing between the RBS and the start codon was reduced to 9 bp; and 2) In P-S65 65 nucleotides between the transcription starting site and the ribosome binding site were deleted, and in P-S115 115 nucleotides between the transcription starting site and the ribosome binding site were deleted. Based on the results of the gene expression level comparison of those strains with promoter modifications, changes in the sequences of the Pcpc* promoter lead to the reduction of the promoter strength.

In addition to constructs using cyanobacterial promoters, we have also constructed expression platforms using promoters from heterotrophs, such as the Ptrc (SEQ ID NOS: 34 and 35) and Pcro promoters (SEQ ID NOS: 34 and 35). Expression experiments demonstrated those promoters worked well, but that they were not as strong as the Pcpc* (FIG. 5).

Expression vectors for protein overexpression in Synechocossus sp. ATCC 29404 (PCC 73109), were constructed using the Pcpc* promoter, the reporter gene yfp, the aadA gene conferring spectinomycin resistance for screening the transformants DNA fragments from the intergenic region were cloned and inserted into sites flanking the gene expression cassette. The new construct was used to transform cells of Synechocossus sp. ATCC 29404. Four different transformants were segregated for comparison.

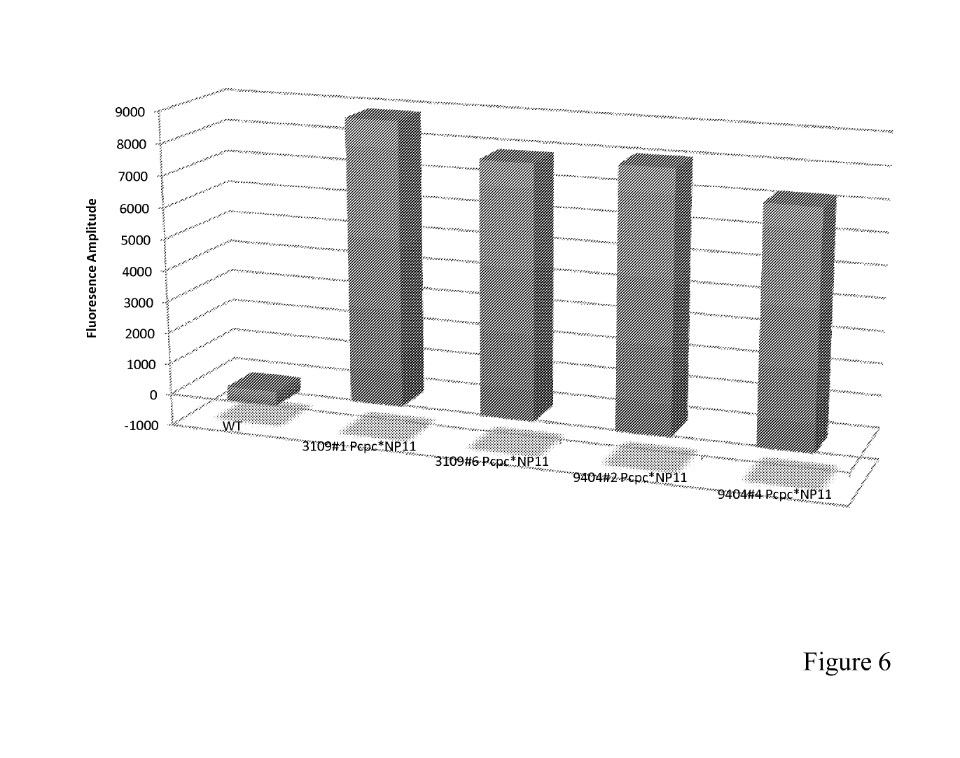

Expression levels of the yfp reporter gene in Synechococcus ATCC 29404 were measured in same fashion as described above for the Synechococcus sp. PCC 7002 experiments. As demonstrated in FIG. 6, the YFP protein was successfully overexpressed in all four engineered Synechocossus sp. ATCC 29404 strains. These results for protein overproduction in the newly sequenced organism Synechocossus sp. ATCC 29404 demonstrate that the platform for expression recombinant proteins has been successfully established in Synechocossus sp. ATCC 29404.

### Example 3

### Expression of Recombinant Proteins with N-Terminal Secretion Leaders

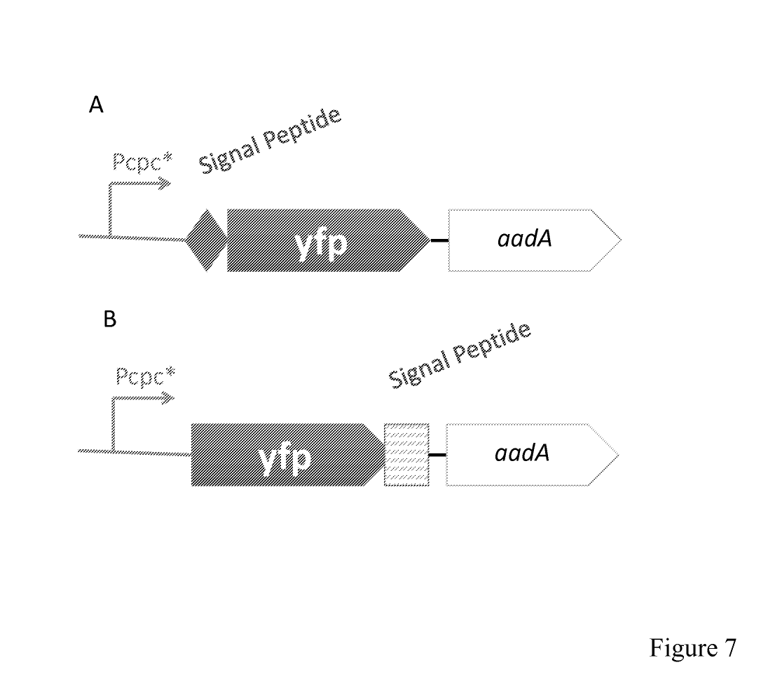

Secretory protein overexpression and secretion platforms have been constructed for two marine cyanobacterium strains, Synechococcus sp. PCC 7002 and Synechocossus sp. ATCC 29404. FIG. 7A illustrates the general structure of the secretory protein overexpression cassette, comprising the Pcpc* promoter, an N-terminal secretion signal peptide, yfp reporter gene, and the selection marker aadA. To facilitate strain-specific integration of the expression cassette, DNA flanking fragments from either the Synechococcus sp. PCC 7002 genome or the Synechocossus sp. ATCC 29404 genome were designed and inserted so that they flanked the cassette.

Protein expression and secretion directed by the N-terminal secretion leader sequences was investigated in Synechococcus sp. PCC 7002. Constructs as described in the preceding paragraph, each comprising a different secretion leader sequence, were transformed into Synechococcus sp. PCC 7002. Segregation of the transformants was performed by repeated restreaking of colonies on spectinomycin plates. Expression of secreted YFP was measured for each engineered strain. Specifically, liquid cultures of the different engineered strains were grown to late exponential growth phase. After pelleting cells by centrifugation, the supernatants were further purified using a Millipore Stervex GP 0.22 μm filter unit. The extracellular proteins isolated from different engineered strains were concentrated for protein analysis by SDS-PAGE electrophoresis and confirmed by immunodetection through Western blotting analysis. YFP protein has been detected in the supernatant of engineered strains containing the newly identified secretion leaders from the SP1, SP3, SP4 and SP8 genes. With application of the SP3 and SP4 secretion leaders, proteins detected in the supernatant from cells of the engineered strains can be respectively measured as 1.2 mg/L and 0.8 mg/L. Also, the recombinant strains have been engineered using the secretion leader SP1 and SP8, and YFP was detected following purification and protein analysis of the extracellular proteins from the cultures.

### Example 4

### Expression of Recombinant Proteins with C-Terminal Secretion Signal Peptides

To examine protein secretion using alternative C-terminal signal peptides, potential C-terminal signal peptides are selected from four genes that encode S-layer proteins in Synechococcus sp. PCC 7002 (Sara, M. and Sleyter, U. B. (2000) S-layer proteins. J. Bacteriol. 182: 859-868; and Smarda, J., Smajs, D., Komrska, J., and Krzyzanek, V. (2002) S-layers on cell walls of cyanobacteria. Micron 33: 257-277.): SYNPCC_{—}7002_A1178 (SEQ ID NO: 9), SYNPCC_{—}7002_A1634 (SEQ ID NO: 10), SYNPCC_{—}7002 A2605 (SEQ ID NO: 11), and SYNPCC_{—}7002 A2813 (SEQ ID NO: 12). Following the strategy as outlined in FIG. 7B, gene expression constructs are generated through in frame fusion of nucleic acid sequences encoding the C-terminal signal peptides (SEQ ID NOS: 21-24) at the C-terminal end of the yfp gene. Those constructs are used to transform cells of Synechococcus sp. PCC 7002. Segregations of the transformants is achieved through restreaking and screening colonies on A+ media plates with addition of spectinomycin. Full segregations of the engineered strains are confirmed by PCR analysis.

Expression of the C-terminal tagged YFP proteins in the engineered strains is detected using Western blot analysis. Using the same method described above for checking secretion in the engineered strains with the N-terminal signal peptides, secretion efficiency is examined through analysis of the extracellular proteins isolated from the culture media for the engineered strains: C-A1178, C-A1634, C-A2605 and C-A2813, each containing a recombinant plasmid comprising a nucleic acid sequence encoding the secretion leader of one of SEQ ID NOS: 9-12).

### Example 5

### Expression of Recombinant Secreted Proteins in a Host Comprising a Deleted Type IV Pilus Assembly Protein Gene

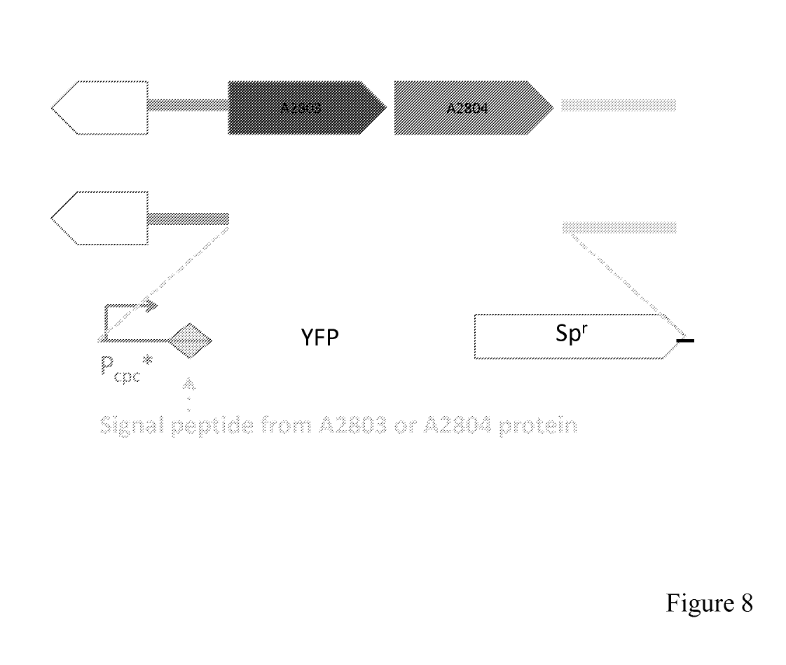

To optimize a system for high level secretion of heterologous proteins, we engineered host strains to minimize their levels of naturally secreted proteins to enhance to purity and overall expression of recombinant proteins of interest. An example of such proteins are those for pilus assembly (Bhaya, D., Bianco, N. R., Bryant, D. A., and Grossman, A. (2000) Type IV pilus biogenesis and motility in the cyanobacterium Synechococystis sp. PCC 6803). Our results show that YFP protein can be secreted with use of the N-terminal signal peptide from the SP1, SP3, SP4, and SP8 proteins and the C-terminal secretion leaders of certain S-layer proteins, especially SYN7002-A1178. The SG3 and SG4 genes are predicted to have function in pilus assembly. To increase the YFP secretion with the secretion leaders (LA2335 and LA2804) encoded by SG3 and SG4 by minimizing the competition from natural secretion of the pilus assembly proteins (SYNPCC7002-A2804 and SYNPCC7002-A2803), strains comprising secretory protein expression platforms have been constructed by integration of the gene expression cassette with deletion of the SYNPCC7002-A2804 and SYNPCC7002-A2803 genes, as illustrated in FIG. 8.

Two DNA fragments, one lying upstream of A2804 and the other lying downstream of A2803 were cloned to flank the secretory protein expression cassette as illustrated in FIG. 8. Three constructs carrying the expression cassettes with L2804, L2803 and L2335 secretion leaders were generated. Those constructs were used to transform cells of Synechococcus sp. PCC 7002. Incorporation of those expression cassettes by double crossover into the chromosome led to deletion of the A2804 and A2803 genes. Colonies were selected and segregations of the transformants was achieved on A+ media plates containing spectinomycin. Full segregations of the engineered strain with YFP overexpression controlled by different promoters have been confirmed by PCR analysis.

### Example 6

### Expression of Recombinant Secreted Proteins

The following protocol was used to characterize engineered protein expression in strains (L2335, L2803 and L2803) with deletion of the original genes encoding the naturally secreted protein(s):

(1) Engineered strains grown in liquid cultures to the late exponential growth phase at about OD730=1.5.

(2) Cells harvested in sterile centrifuge tubes through low speed centrifugation.

(3) Cells re-suspended into new growth media with addition of protease inhibitor 1 mM protease inhibitor PMSF (or 0.1 mM protease cocktail) to the final OD730=1.

(4) The liquid cultures were grown at normal growth conditions for about 15-18 hours.

(5) For isolation of the extracellular proteins, cells were pelleted through high-speed centrifugation. The supernatant was further purified using the Millipore 0.22 μm filter units. The extracellular proteins were concentrated using 10 kDa cut-off membrane filter systems.

Using the methods outlined above, extracellular proteins from different engineered strains have been purified and analyzed by protein analysis. Protein oproduction has been characterized in three genetically engineered strains: L2335, L2803 and L2804. YFP protein was successfully overexpressed and detected in the supernatant using the newly identified secretion signal peptides from SP3 and SP4, respectively, measured as 1.2 mg/L and 0.8 mg/L.

### Example 7

### Engineering Strains for Secretion of Recombinant Proteins

Optimization of Signal Peptides

For most bacteria, approximately 90% of all secreted proteins are translocated across the inner membrane via a Sec-dependent system. Proteins secreted via the Sec system are initially synthesized with N-terminal hydrophobic signal peptides (SP) consisting of a positively charged N domain followed by a longer, hydrophobic H domain and a C domain consisting of three amino acids which form either type I or type II signal peptidase recognition sites. Signal peptides play an important role in the translocation process including interacting with SecA, the signal recognition protein and the signal peptidase. The general structure of signal peptides is well conserved across most living cells. Previous work in both bacteria and yeast has demonstrated that non-native signal peptides fused to heterologous proteins can facilitate their secretion and, in many cases, heterologous signal peptides can be found which result in higher levels of secretion than signal peptides from the host organism. To date, there is still no way to predict which signal peptide/target protein pairs are optimum. Therefore, identification of particularly useful signal peptides for secretion of a recombinant protein of interest in a host strain is performed in some embodiments herein by testing different signal peptide-protein of interest pairs to identify those that work best (Brockmeier et al, 2006. Systematic Screening of All Signal Peptides from Bacillus subtilis: A Powerful Strategy in Optimizing Heterologous Protein Secretion in Gram-positive Bacteria. J. Molecular Biology 362:393-402; Degering et al, 2010. Optimization of Protease Secretion in Bacillus subtilis and Bacillus licheniformis by Screening of Homologous and Heterologous Signal Peptides. App Environ Micro, (76)1-9:6370-6376).

As an example of such an approach, the cyanobacterium Synechococcus sp. ATCC 29404 is used as a host strain for expression and secretion of recombinant proteins. A library of nucleic acids encoding signal peptides is generated by searching predicted open reading frames (ORF) from the genome sequence of a cyanobacterial strain Synechococcus elongatus PCC 7942, which is closely related to Synechococcus sp. ATCC 29404, to identify sequences that encode signal peptides at the N-terminus of proteins encoded by the Synechococcus elongatus PCC 7942 genome. In some embodiments, generating the signal peptide library from a non-identical but closely related strain reduces the probability of recombination occurring between an engineered allele and a native gene in the genome of a recombinant host. Even so, in an alternative approach the signal peptide library is generated using the host strain's own genome sequence. To design the SP library, the predicted protein products of the Synechococcus elongatus PCC 7942 genome were analyzed using the signal peptide identification program SignalP 4.0 (Petersen et al. 2011) to identify SPs with D-scores ≧0.6. This analysis identified 362 putative signal peptides in Synechococcus elongatus PCC 7942 ranging in size from 16- to 60-amino acids.

PCR is used to amplify the Synechococcus elongatus PCC 7942 DNA sequences encoding the signal peptides ranging in size from 19- to 38-amino acids. PCR primer pairs are designed such that the forward primer contains a 5′-tail with an NcoI restriction site while the reverse primer has an NdeI site engineered into it. PCR reactions are carried out under standard conditions using Phusion® High-Fidelity PCR Kit (New England Biolabs). PCR products are purified and digested with NcoI and NdeI and ligated in plasmid pAQ1-cpc*-yfp which is digested with NcoI and NdeI generating gene fusions in which the signal peptide coding sequence is inserted in frame with a yfp reporter gene. Expression of the fusion protein is driven by the upstream cpc* promoter which is cloned from the DNA upstream of the cpc operon from Thermosynechococcus elongatus strain BP-1.

Constructs containing the signal peptide/yfp fusions are transformed into Synechococcus sp. ATCC 29404 as described in above. Following segregation, expression cultures of each strain are grown in A+ medium as described above and total YFP expression (i.e intracellular+extracellular) and secreted YFP expression is analyzed and compared for each strain to identify those with a high level of secretion. Although YFP, an easily detectable target protein, is used in this example, the strategy can be used for any target protein. Proteins that are not detectible by a screenable phenotype are detected and measured using high-throughput protein analysis techniques such as Microfluidics LabChip® Technology (Caliper Life Sciences).

This approach can be done using signal peptides from any bacteria whether they are closely related to the host strain (e.g. Synechococcus sp. PCC 7002) or from much more distant group such as E. coli.

Overexpression of SecA and Putative Secretion Chaperones

In most organisms, the Sec-mediated pathway is responsible for a majority of protein secretion and SecA is the motor that drives the translocation of proteins by the pathway. The Sec secretion system transports unfolded proteins out of the cell which is in contrast to systems such as the Tat (Twin Arginine Transport) system which acts on folded proteins. In many Gram-negative bacteria, SecB plays a role in Sec-mediated secretion by binding precursor proteins with signal peptides as they come off of the ribosome and inhibiting their folding. SecB then “hands off” the unfolded precursor to SecA which starts the translocation process. Overexpression of SecA and SecB have been shown to increase secretion in other bacteria (Leloup. et al., 1999. Differential Dependence of Levansucrase and α-Amylase Secretion on SecA (Div) during the Exponential Phase of Growth of Bacillus subtilis. J. Bact. 181(6):1820-1826). Although cyanobacteria such as Synechococcus and Synechocystis possess SecA homologs, the members of these genera lack SecB. In this way, the cyanobacteria strains are more similar to Gram-positive bacteria like Bacillus subtilis which also lacks SecB than to other Gram-negative bacteria. Interestingly, some sequenced cyanobacteria genomes such as those of Synechococcus elongatus PCC 7942 and Synechococcus elongatus PCC6301 encode homologs of the B. subtilis putative secretion chaperone, CsaA. Over-expression of the B. subtilis CsaA in E. coli secB mutants was shown to stimulate protein export (Muller, et al., 2000. Chaperone-like activities of the CsaA protein of Bacillus subtilis. Microbiology 146:77-88). In addition, the B. subtilis CsaA was shown to specifically interact with the SecA homologs from both E. coli and B. subtilis in a manner similar to SecB (Muller, et al., 2000b. Interaction of Bacillus subtilis CsaA with SecA and the precursor proteins. Biochem. J. 348:367-373). Together these data imply that CsaA homologs function in an analogous fashion to SecB with regard to protein secretion. As such, overexpression of a heterologous CsaA in a cyanobacterial production host is used to improve protein secretion.

Accordingly, the SecB and CsaA homolog pairs from divergent strains are expressed in a cyanobacterial protein production host strain to facilitate protein secretion. For example, using strain Synechococcus sp. ATCC 29404 as the production host, SecA and CsaA from Synechococcus elongatus PCC 7942 are overexpressed by cloning the genes plus promoters disclosed herein into integration vectors such as those described above.

Over-Expression of Cytoplasmic Chaperones

In some instances heterologous proteins form insoluble aggregates in the cytoplasm when overexpressed. Once formed the proteins in these aggregates become unavailable for secretion and may inhibit translation and secretion of other proteins. In addition to dedicated secretion chaperones like SecB and CsaA, bacteria encode a variety of additional chaperones which, when expressed at high enough levels can minimize the aggregation of heterologous proteins and maintain those that are expressed in translocation-competent forms. Therefore, the expression and secretion of heterologous proteins can be improved by over-expression of these other chaperones (Nishihara et al., 1998. Chaperone Coexpression Plasmids: Differential and Synergistic Roles of DnaK-DnaJ-GroE and GroEL-GroES in Assisting Folding of an Allergen of Japanese Cedar Pollen, Cryj2, in Escherichia coli. Appl. Environ. Microbiol. 64: 1694-1699.).

Accordingly, in some embodiments intracellular protein chaperones are overexpressed in a cyanobacterial protein production host strain. For example, using strain Synechococcus sp. ATCC 29404 as the production host, DnaK, DnaJ, GroES, and GroEL homologs from Synechococcus elongatus PCC 7942 are overexpressed by cloning the genes for those chaperones plus promoters (such as those disclosed herein) into integration vectors such as those described above.

PCR Mutagenesis of secA

SecA plays a central role in protein translocation both as an energy source and as part of the “proofreading” system that helps ensure that only those proteins that are meant to be secreted are targeted out of the cytoplasm (Karamyshev et al., 2005. Selective SecA Association with Signal Sequences in Ribosome-bound Nascent Chains. J. Biol. Chem. 280(45):37930-37940). As such, SecA can inhibit or reduce the efficiency with which heterologous proteins are transported out of the cell. By mutagenizing a non-native SecA, and overexpressing it in a host strain the efficiency of secretion for heterologous proteins can be increased. To do so, the secA homologue from Synechococcus elongatus PCC 7942 is cloned by PCR amplification under mutagenic conditions (Cadwell et al., 1994. Mutagenic PCR. In, PCR Methods and Applications. Cold Spring Harbor Laboratories) using primers containing restriction sites that allow cloning of the mutagenized population of secA into an expression vector such as PAQ1-cpc*-yfp or similar cyanobacterial vector. In order to identify secA variants that improve secretion of heterologous target proteins, host strains containing mutagenized SecA plus secretion reporter constructs such as signal peptide::yfp fusions are then grown in high throughput assays to identify strains in which increased secreted Yfp is present in the culture supernatants.

Sequences Referenced in This Example
Synechococcus elongatus PCC 7942 secA
(Synpcc7942_0289)
ATGCTGAATTTGCTGCTGGGCGATCCCAACGTCCGCAAAGTCAAAAAGTACAAACC
CCTCGTCACTGAAATCAATCTGTTGGAAGAGGACATTGAGCCACTGTCCGACAAGG
ATTTAATTGCCAAAACGGCTGAGTTTCGCCAGAAGCTCGACAAGGTTTCCCACTCGC
CAGCTGCAGAGAAGGAATTGCTGGCGGAGTTGCTGCCCGAAGCCTTTGCGGTCATG
CGCGAAGCCAGTAAACGAGTGCTGGGGCTGCGCCACTTTGATGTGCAGATGATCGG
CGGCATGATTCTGCACGACGGTCAGATTGCCGAGATGAAGACGGGTGAAGGGAAAA
CCCTCGTCGCTACGCTGCCGTCCTATCTCAATGCACTGTCGGGTAAAGGTGCGCACG
TCGTCACCGTCAACGACTACTTGGCTCGCCGCGACGCGGAATGGATGGGACAAGTC
CACCGCTTCCTAGGCTTGAGTGTTGGCCTAATCCAGCAGGGAATGTCGCCGGAAGAG
CGTCGCCGCAACTACAACTGCGACATTACCTACGCTACCAACAGCGAACTGGGCTTT
GATTACCTGCGCGACAACATGGCCGCAGTGATTGAAGAGGTAGTCCAGCGTCCCTTC
AACTACGCCGTGATCGACGAGGTGGACTCGATTCTGATCGACGAAGCCCGGACACC
CTTGATCATTTCCGGTCAGGTCGATCGCCCGAGCGAAAAATACATGCGGGCATCGGA
AGTCGCGGCGCTCTTGCAGCGATCGACGAATACGGACAGTGAAGAAGAGCCGGATG
GCGATTACGAAGTTGACGAAAAAGGCCGTAATGTCCTGCTGACGGATCAAGGCTTT
ATCAACGCTGAGCAATTGTTAGGTGTCAGCGATCTGTTTGACTCCAATGACCCTTGG
GCTCACTACATCTTTAATGCGATTAAGGCCAAGGAGCTGTTCATTAAAGATGTGAAC
TACATCGTGCGCGGTGGCGAGATTGTCATCGTCGATGAGTTCACAGGGCGCGTGATG
CCTGGGCGTCGCTGGAGTGATGGTCTGCATCAGGCCGTGGAGTCGAAGGAAGGCGT
TGAGATTCAACCCGAAACCCAAACCCTTGCTTCGATTACTTACCAAAACTTCTTCCT
GCTCTACCCCAAACTGTCGGGCATGACCGGTACGGCGAAGACAGAAGAGTTGGAGT
TTGAGAAGACTTACAAGCTAGAAGTAACCGTTGTTCCGACCAACCGAGTCAGCCGTC
GTCGGGATCAGCCTGATGTCGTCTACAAAACTGAGATCGGCAAGTGGCGTGCGATC
GCAGCGGACTGTGCTGAACTGCACGCGGAAGGTCGTCCTGTTCTGGTCGGTACTACC
AGTGTTGAGAAGTCGGAGTTCCTGTCACAACTGCTGAATGAGCAGGGCATCCCCCAC
AACCTGCTCAACGCCAAACCCGAAAACGTAGAACGCGAGGCGGAAATCGTTGCACA
GGCAGGCCGTCGGGGTGCCGTCACGATTTCGACCAACATGGCAGGTCGCGGGACCG
ACATCATCTTGGGCGGTAATGCGGACTACATGGCGCGGCTGAAGCTGCGCGAGTATT
GGATGCCGCAACTGGTCAGCTTCGAAGAGGATGGCTTTGGCATTGCTGGGGTTGCTG
GTTTAGAGGGCGGTCGCCCGGCAGCGCAAGGTTTTGGGTCGCCCAACGGCCAGAAG
CCACGCAAGACTTGGAAAGCGTCGTCGGATATTTTCCCAGCAGAACTGAGTACTGA
GGCCGAAAAGCTGCTGAAAGCAGCGGTAGACCTCGGGGTGAAAACCTACGGCGGTA
ACAGCCTCTCGGAGCTGGTAGCGGAAGACAAGATCGCTACGGCGGCTGAGAAGGCG
CCGACGGATGATCCGGTGATTCAAAAACTGCGGGAAGCCTACCAGCAAGTCCGCAA
AGAATACGAAGCAGTCACGAAGCAGGAGCAAGCCGAGGTCGTTGAACTGGGCGGC
CTGCATGTGATTGGTACGGAACGCCACGAGTCACGCCGAGTGGATAACCAGTTGCG
CGGTCGTGCCGGTCGGCAAGGGGACCCAGGATCCACGCGTTTCTTCCTGAGCTTGGA
AGATAACCTGCTGCGGATTTTTGGTGGCGATCGCGTGGCCAAACTGATGAATGCCTT
CCGCGTCGAAGAAGATATGCCGATCGAGTCGGGCATGCTGACGCGATCGCTCGAGG
GTGCTCAGAAGAAGGTCGAGACCTACTACTACGACATCCGCAAGCAGGTGTTTGAG
TACGACGAGGTGATGAACAACCAGCGTCGTGCCATCTATGCAGAACGCCGCCGTGT
TCTCGAAGGACGAGAGCTAAAAGAACAAGTGATTCAGTACGGCGAACGGACGATGG
ATGAAATCGTCGATGCCCACATCAATGTGGATTTGCCGTCGGAAGAGTGGGATCTGG
AAAAGCTGGTCAATAAGGTCAAGCAGTTCGTCTATCTGCTTGAAGACCTAGAGGCC
AAGCAACTGGAAGACCTGTCTCCTGAGGCGATCAAGATCTTCCTGCACGAGCAATTG
CGGATTGCCTACGACCTCAAAGAAGCCCAGATCGATCAAATCCAGCCAGGCTTGAT
GCGGCAGGCCGAACGCTACTTCATCCTTCAGCAGATCGACACGCTCTGGCGTGAGC
ATTTGCAGGCGATGGAAGCCTTGCGCGAATCCGTCGGTCTGCGGGGCTATGGGCAA
AAAGATCCACTGCTGGAGTATAAGAGTGAGGGCTACGAGCTGTTCCTCGAGATGAT
GACGGCGATTCGCCGCAACGTGATCTACTCGATGTTCATGTTCGATCCGCAGCCTCA
AGCCCGTCCACAAGCTGAGGTGGTTTAG
Synechococcus elongatus PCC 7942 SecA (YP_399308.1)
MLNLLLGDPNVRKVKKYKPLVTEINLLEEDIEPLSDKDLIAKTAEFRQKLDKVSHSPAAE
KELLAELLPEAFAVMREASKRVLGLRHFDVQMIGGMILHDGQIAEMKTGEGKTLVATL
PSYLNALSGKGAHVVTVNDYLARRDAEWMGQVHRFLGLSVGLIQQGMSPEERRRNYN
CDITYATNSELGFDYLRDNMAAVIEEVVQRPFNYAVIDEVDSILIDEARTPLIISGQVDRP
SEKYMRASEVAALLQRSTNTDSEEEPDGDYEVDEKGRNVLLTDQGFINAEQLLGVSDLF
DSNDPWAHYIFNAIKAKELFIKDVNYIVRGGEIVIVDEFTGRVMPGRRWSDGLHQAVES
KEGVEIQPETQTLASITYQNFFLLYPKLSGMTGTAKTEELEFEKTYKLEVTVVPTNRVSR
RRDQPDVVYKTEIGKWRAIAADCAELHAEGRPVLVGTTSVEKSEFLSQLLNEQGIPHNL
LNAKPENVEREAEIVAQAGRRGAVTISTNMAGRGTDIILGGNADYMARLKLREYWMPQ
LVSFEEDGFGIAGVAGLEGGRPAAQGFGSPNGQKPRKTWKASSDIFPAELSTEAEKLLK
AAVDLGVKTYGGNSLSELVAEDKIATAAEKAPTDDPVIQKLREAYQQVRKEYEAVTKQ
EQAEVVELGGLHVIGTERHESRRVDNQLRGRAGRQGDPGSTRFFLSLEDNLLRIFGGDR
VAKLMNAFRVEEDMPIESGMLTRSLEGAQKKVETYYYDIRKQVFEYDEVMNNQRRAIY
AERRRVLEGRELKEQVIQYGERTMDEIVDAHINVDLPSEEWDLEKLVNKVKQFVYLLE
DLEAKQLEDLSPEAIKIFLHEQLRIAYDLKEAQIDQIQPGLMRQAERYFILQQIDTLWREH
LQAMEALRESVGLRGYGQKDPLLEYKSEGYELFLEMMTAIRRNVIYSMFMFDPQPQAR
PQAEVV
Synechococcus elongatus PCC 7942 csaA
(Synpcc7942_0179)
TTGGAGGTGCATCCCATAGAAACTATTACCTTCGACAAGTTTCTGAAGGTTGAGCTT
CGTGTCGGCAAGATTGTTGATGCAACTGAGTTTGTGGGTGCGCGGAGGCCAGCCTAC
ATCCTGCATATCGACTTCGGTGAAGAGATTGGTGTCAAGAAATCAAGTGCGCAGATC
ACCGCACTCTACAAGCCGGAAGAACTGATCGGTGGGCTTGTCGTAGCAGTGGTCAA
CTTTCCATGTAAGCAAATCGGTCTGCTTATGTCTGATTGCCTTGTCACGGGATTCCAG
AGCGAGAACAGAGAAGTAGCGCTCTGCATCCTTGACAAGTCCGTTCTGCTGGGCTCA
AAATTGCTTTAA
Synechococcus elongatus PCC 7942 CsaA
(YP_399198.1)
MEVHPIETITFDKFLKVELRVGKIVDATEFVGARRPAYILHIDFGEEIGVKKSSAQITALY
KPEELIGGLVVAVVNFPCKQIGLLMSDCLVTGFQSENREVALCILDKSVLLGSKLL
Synechococcus elongatus PCC 7942 dnaK
(Synpcc7942_2073)
ATGGCCAAAGTTGTCGGAATCGACCTCGGAACCACCAACTCTTGCGTGGCTGTCATG
GAGGGCGGCAAGCCCACTGTGATCGCTAATGCGGAAGGTTTTCGCACCACTCCTTCA
GTCGTTGCTTTTGCGAAAAACCAAGACCGCCTCGTGGGTCAAATCGCCAAACGCCA
GGCGGTGATGAACCCCGAGAACACCTTCTACTCGGTTAAGCGCTTCATCGGCCGTCG
TCCGGATGAAGTCACGAACGAACTGACCGAAGTGGCCTACAAAGTCGATACTTCGG
GCAATGCCGTCAAGCTGGATAGCTCCAATGCTGGCAAGCAGTTCGCTCCTGAAGAA
ATTTCGGCGCAGGTGCTGCGCAAACTGGCCGAAGACGCCAGCAAATACCTGGGTGA
AACCGTCACCCAAGCCGTGATCACGGTTCCGGCCTACTTCAATGACTCCCAGCGCCA
AGCGACCAAAGACGCTGGCAAAATCGCCGGCCTAGAAGTGCTGCGGATCATCAACG
AGCCGACGGCAGCCGCGCTGGCCTACGGTCTTGATAAGAAGAGCAACGAACGCATC
CTTGTCTTTGACTTGGGCGGCGGTACTTTCGACGTCTCGGTCTTGGAAGTGGGCGAC
GGCGTTTTTGAAGTGCTGGCGACCTCGGGTGATACCCACCTCGGTGGCGACGACTTC
GACAAAAAAATCGTTGACTTCCTGGCTGGTGAATTCCAGAAGAACGAAGGCATCGA
TCTGCGCAAAGACAAGCAGGCTCTGCAGCGTCTGACGGAAGCCGCTGAGAAAGCCA
AAATCGAGCTGTCCAGCGCCACTCAAACTGAAATCAACCTGCCCTTCATCACGGCAA
CCCAAGACGGGCCGAAGCACCTCGACCTGACCTTAACCCGCGCCAAGTTTGAAGAA
TTGGCTTCGGATCTGATCGATCGCTGCCGGATTCCGGTGGAGCAAGCGATCAAAGAT
GCCAAGTTGGCCCTGAGCGAAATTGACGAAATCGTCTTGGTCGGTGGTTCGACCCGG
ATTCCTGCGGTGCAGGCGATCGTCAAGCAAATGACGGGCAAAGAGCCCAACCAAAG
TGTCAACCCCGATGAGGTGGTGGCGATCGGTGCGGCGATTCAAGGTGGCGTCTTGGC
TGGGGAAGTCAAAGACATCCTGCTGCTCGACGTGACGCCACTATCCTTGGGGGTAG
AAACCCTTGGTGGCGTGATGACTAAGTTGATCCCACGCAACACCACTATCCCCACCA
AGAAGTCGGAAACCTTCTCGACGGCGGCGGACGGTCAAACCAACGTCGAAATCCAC
GTGCTCCAAGGCGAGCGCGAAATGGCCAGCGACAACAAGAGCTTGGGAACCTTCCG
GCTGGATGGCATTCCGCCGGCTCCCCGTGGCGTGCCCCAAATCGAAGTGATCTTCGA
CATCGACGCTAACGGCATCCTCAATGTCACGGCCAAAGACAAAGGGTCGGGCAAAG
AGCAGTCGATCAGCATCACCGGCGCTTCGACCTTGTCTGACAACGAAGTCGATCGCA
TGGTCAAAGACGCCGAAGCGAATGCAGCAGCGGACAAAGAACGGCGCGAACGTAT
CGACCTGAAGAACCAAGCCGACACGCTGGTCTATCAGTCTGAGAAACAACTCAGCG
AGCTGGGTGACAAGATCTCGGCTGATGAGAAAAGCAAAGTCGAAGGCTTTATCCAA
GAGCTGAAAGATGCCTTGGCTGCCGAAGACTACGACAAGATCAAGTCGATCATCGA
GCAACTGCAGCAAGCTCTCTACGCCGCTGGCAGCAGCGTCTACCAGCAGGCTAGCG
CTGAAGCTTCGGCCAACGCCCAAGCCGGTCCTTCCTCGTCCTCGAGCAGCAGCTCTG
GCGATGATGATGTGATTGACGCAGAGTTCTCTGAGTCGAAGTAA
Synechococcus elongatus PCC 7942 DnaK
(YP_401090.1)
MGKVIGIDLGTTNSCVAVLEGGKPIIVTNREGDRTTPSIVAVGRKGDRIVGRMAKRQAV
TNAENTVYSIKRFIGRRWEDTEAERSRVTYTCVPGKDDTVNVTIRDRVCTPQEISAMVL
QKLRQDAETFLGEPVTQAVITVPAYFTDAQRQATKDAGAIAGLEVLRIVNEPTAAALSY
GLDKLHENSRILVFDLGGSTLDVSILQLGDSVFEVKATAGNNHLGGDDFDAVIVDWLAD
NFLKAESIDLRQDKMAIQRLREASEQAKIDLSTLPTTTINLPFIATATVDGAPEPKHIEVEL
QREQFEVLASNLVQATIEPIQQALKDSNLTIDQIDRILLVGGSSRIPAIQQAVQKFFGGKTP
DLTINPDEAIALGAAIQAGVLGGEVKDVLLLDVIPLSLGLETLGGVFTKIIERNTTIPTSRT
QVFTTATDGQVMVEVHVLQGERALVKDNKSLGRFQLTGIPPAPRGVPQIELAFDIDADG
ILNVSARDRGTGRAQGIRITSTGGLTSDEIEAMRRDAELYQEADQINLQMIELRTQFENL
RYSFESTLQNNRELLTAEQQEPLEASLNALASGLESVSNEAELNQLRQQLEALKQQLYAI
GAAAYRQDGSVTTIPVQPTFADLIGDNDNGSNETVAIERNDDDATVTADYEAIE
Synechococcus elongatus PCC 7942 dnaJ
(Synpcc7942_2074)
ATGGCTGCTGACTACTACCAACTGCTTGGCGTTGCTCGCGACGCAGACAAGGACGA
AATTAAACGTGCTTATCGGCGTTTGGCTCGCAAGTACCATCCAGATGTGAACAAGGA
GCCAGGCGCTGAAGACAAGTTCAAAGAAATCAACCGCGCCTACGAGGTGCTGTCGG
AGCCTGAAACCCGCGCTCGCTACGACCAATTTGGGGAAGCGGGTGTCTCTGGTGCC
GGAGCCGCTGGTTTCCAAGATTTTGGCGACATGGGTGGATTCGCTGACATCTTTGAA
ACCTTCTTCAGCGGGTTTGGAGGCATGGGCGGGCAACAAGCCTCCGCTCGCCGGCG
CGGACCCACTCGGGGTGAAGACCTACGGCTGGATTTGAAACTGGATTTCCGAGATG
CCATCTTTGGTGGCGAGAAAGAAATTCGGGTCACCCATGAAGAAACTTGCGGCACC
TGTCAGGGGAGTGGGGCTAAGGCCGGAACCCGGCCGCAAACTTGTACGACCTGTGG
TGGTGCAGGCCAAGTCCGACGAGCAACCCGGACGCCCTTCGGCAGCTTTACCCAAG
TTTCAGTCTGTCCCACCTGCGAGGGCAGCGGGCAGATGATCGTTGATAAGTGCGATG
ACTGTGGCGGAGCAGGGCGTCTACGGCGGCCGAAGAAACTGAAGATCAATATTCCA
GCTGGGGTGGATAGCGGTACGCGGCTGCGAGTAGCCAATGAAGGCGATGCGGGGCT
GCGCGGTGGGCCGCCGGGCGACCTTTACGTCTATTTGTTCGTCAGTGAGGACACCCA
GTTCCGGCGGGAAGGCATCAATCTCTTCTCCACCGTGACCATCAGCTACCTGCAAGC
CATTTTGGGCTGCAGCCTAGAAGTTGCGACTGTAGACGGCCCCACCGAGCTGATCAT
TCCGCCCGGAACACAACCCAATGCCGTACTGACGGTGGAGGGCAAGGGCGTGCCAC
GACTGGGGAATCCGGTCGCTCGGGGCAATCTTTTGGTCACAATTAAGGTGGAAATTC
CCACCAAAATTAGCGCTGAAGAACGCGAACTGTTGGAAAAAGTGGTGCAAATTCGC
GGCGATCGCGCTGGAAAAGGAGGGATTGAAGGCTTCTTCAAAGGAGTCTTTGGCGG
ATGA
Synechococcus elongatus PCC 7942 DnaJ
(YP_400830.1)
MAILEQGNITIHTDNIFPIIKKSLYSEHEIFLRELISNAVDAIQKLKMVSYAGELEGEIGDPQ
ITLSIDRDRKQLKIADNGIGMTADEIKRYINQVAFSSAEDFIEKYKGGADQPIIGHFGLGF
YSAFIVADRVEIETLSYQKGATPVHWTCDGSPSFELSEGSRTERGTTIILNLSEEELEYLEP
ARIRQLVKTYCDFMPVPIALEGEVLNKQ
Synechococcus elongatus PCC 7942 groES
(Synpcc7942_2314)
ATGGCAGCTGTATCTCTGAGTGTTTCGACCGTGACGCCCCTGGGCGATCGCGTTTTT
GTGAAAGTCGCTGAAGCCGAAGAAAAAACTGCTGGCGGCATCATCCTGCCCGATAA
CGCTAAAGAGAAGCCCCAAGTCGGCGAAATCGTGGCAGTTGGCCCTGGCAAACGCA
ACGACGACGGCAGCCGCCAAGCGCCTGAAGTCAAAATCGGCGACAAAGTTCTCTAC
TCCAAGTACGCCGGTACTGACATCAAACTCGGCAACGACGACTACGTGTTGCTGTCC
GAGAAAGACATCTTGGCCGTTGTTGCCTAG
Synechococcus elongatus PCC 7942 GroES
(YP_401331.1)
MAAVSLSVSTVTPLGDRVFVKVAEAEEKTAGGIILPDNAKEKPQVGEIVAVGPGKRNDD
GSRQAPEVKIGDKVLYSKYAGTDIKLGNDDYVLLSEKDILAVVA
Synechococcus elongatus PCC 7942 groEL
(Synpcc7942_2313)
ATGGCTAAACGGATCATTTACAACGAAAACGCCCGTCGCGCCCTTGAAAAAGGCAT
CGACATTCTGGCGGAAGCCGTTGCAGTCACCCTCGGCCCCAAAGGTCGCAACGTTGT
TCTTGAGAAAAAGTTCGGCGCACCGCAAATCATCAATGACGGTGTGACGATCGCCA
AAGAAATCGAACTGGAAGACCACATCGAAAACACCGGTGTGGCGCTGATTCGTCAA
GCCGCTTCCAAAACCAACGACGCAGCCGGTGACGGCACCACCACCGCAACCGTCTT
GGCGCACGCTGTGGTCAAAGAAGGTCTGCGTAACGTGGCTGCTGGCGCTAACGCCA
TTTTGCTGAAGCGCGGGATCGACAAAGCCACCAACTTCTTGGTTGAGCAAATCAAGT
CCCACGCTCGTCCGGTCGAAGACTCCAAGTCGATCGCCCAAGTCGGTGCAATCTCGG
CTGGCAACGACTTTGAAGTCGGCCAAATGATCGCCGATGCTATGGACAAAGTCGGC
AAAGAAGGCGTCATCTCGCTGGAAGAAGGCAAATCGATGACCACCGAACTGGAGGT
CACCGAAGGGATGCGTTTCGACAAGGGCTACATCTCGCCCTACTTTGCCACCGACAC
CGAGCGGATGGAAGCCGTCTTTGACGAGCCCTTCATCTTGATCACCGACAAGAAAAT
CGGTTTGGTTCAAGACTTGGTGCCCGTGCTGGAGCAAGTGGCTCGCGCTGGCCGTCC
GCTGGTGATCATCGCCGAGGACATCGAGAAAGAAGCCCTCGCCACCTTGGTCGTCA
ACCGTCTGCGTGGCGTGCTCAACGTTGCTGCAGTCAAAGCGCCTGGTTTCGGCGATC
GCCGCAAAGCCATGCTGGAAGACATTGCTGTCCTGACTGGTGGTCAACTGATCACTG
AAGACGCAGGTCTGAAGCTGGATACCACCAAGCTTGATCAGCTGGGTAAAGCCCGC
CGGATCACGATCACCAAAGACAACACCACGATCGTGGCTGAAGGCAACGAAGCGGC
TGTGAAGGCCCGCGTTGACCAAATCCGTCGCCAAATCGAAGAAACTGAGTCGTCCT
ACGACAAAGAGAAGCTGCAAGAGCGCTTGGCTAAGCTCTCCGGTGGCGTTGCAGTC
GTCAAAGTTGGCGCGGCAACCGAAACTGAAATGAAAGACCGCAAACTGCGTCTGGA
AGATGCGATCAACGCCACCAAAGCGGCGGTTGAAGAAGGCATCGTCCCTGGTGGCG
GCACCACCTTGGCGCACCTCGCTCCTCAGCTGGAAGAGTGGGCAACCGCTAACCTCA
GCGGTGAAGAGCTGACCGGCGCTCAAATCGTGGCTCGTGCCTTGACGGCTCCGCTG
AAGCGGATTGCTGAAAACGCTGGCCTCAACGGTGCTGTGATCTCCGAGCGCGTCAA
AGAACTGCCCTTCGACGAAGGCTACGACGCCTCCAACAACCAGTTCGTGAATATGTT
CACGGCTGGCATCGTTGACCCGGCCAAAGTGACTCGTAGTGCCCTGCAAAACGCAG
CTTCGATCGCAGCCATGGTGCTGACGACCGAGTGCATTGTGGTCGACAAACCGGAA
CCGAAAGAAAAAGCCCCGGCTGGTGCTGGCGGCGGCATGGGCGACTTCGACTACTA
A
Synechococcus elongatus PCC 7942 GroEL
(YP_401330.1)
MAKRIIYNENARRALEKGIDILAEAVAVTLGPKGRNVVLEKKFGAPQIINDGVTIAKEIEL
EDHIENTGVALIRQAASKTNDAAGDGTTTATVLAHAVVKEGLRNVAAGANAILLKRGI
DKATNFLVEQIKSHARPVEDSKSIAQVGAISAGNDFEVGQMIADAMDKVGKEGVISLEE
GKSMTTELEVTEGMRFDKGYISPYFATDTERMEAVFDEPFILITDKKIGLVQDLVPVLEQ
VARAGRPLVIIAEDIEKEALATLVVNRLRGVLNVAAVKAPGFGDRRKAMLEDIAVLTGG
QLITEDAGLKLDTTKLDQLGKARRITITKDNTTIVAEGNEAAVKARVDQIRRQIEETESSY
DKEKLQERLAKLSGGVAVVKVGAATETEMKDRKLRLEDAINATKAAVEEGIVPGGGTT
LAHLAPQLEEWATANLSGEELTGAQIVARALTAPLKRIAENAGLNGAVISERVKELPFDE
GYDASNNQFVNMFTAGIVDPAKVTRSALQNAASIAAMVLTTECIVVDKPEPKEKAPAG
AGGGMGDFDY

### Example 8

### Type I Pathway Leader Identification and Use

Many gram negative bacteria employ Type I secretion systems to export proteins outside of the cell. Type I systems consist of three components: 1) an ABC transporter localized to the inner membrane, 2) a membrane fusion protein (MFP) that spans the periplasmic space, and 3) outer membrane protein (OMP). The Type I secretion apparatus forms a continuous proteinaceous conduit that allows proteins to move from the cytoplasm to the external milieu bypassing the inner and outer membranes and the periplasm. ATP hydrolysis by the ABC transporter drives protein secretion. Unlike N-terminal “sec” tags, Type I secretion signal, so called RTX repeats, are located at the C-terminal of the secreted protein and are not cleaved during secretion. The alpha hemolysin system of E. coli was the first characterized and prototypical type I secretion system. The majority of components are encoded in a single hlyABD operon where HlyA is the secreted protein, HlyB is the ABC transporter, and HlyD is the MFP. The OMP, TolC, is encoded elsewhere in the genome. Like many Type I secreted effector proteins, HlyA is a pore forming toxin secreted by pathogenic E. coli to lyse and kill eukaryotic host cells. Other Type I secreted effectors include metalloendopeptidases, lipases, S-layer proteins, and bacteriocins (Omori 2003). These diverse proteins all contain characteristic RTX repeats that target them for export through the Type I secretion apparatus.

The cyanobacterium PCC 7002 genome encodes a putative Type I secretion system. Like E. coli, the ABC transporter and MPF are present in a single predicted operon consisting of SYNPCC7002_G0068, SYNPCC7002_G0069, and SYNPCC7002_G0070 (Microbes Online). SYNPCC7002_G0069, and SYNPCC7002_G0070 encode hlyB and hlyD homologs, respectively. SYNPCC7002_G0068 encodes a SurA homolog, a parulin-like peptidyl-prolyl cis-trans isomerase. A tolC homolog, SYNPCC7002_A0585 is encoded elsewhere in the genome. The genetic locus adjacent to the “Type I secretion operon”, SYNPCC7002_G0067, encodes a phosphatase protein with putative C terminal RTX repeats suggesting that SYNPCC7002_G0067 is secreted by a Type I mechanism. Our homology searches showed that SYNPCC7002_G0067 is the only RTX containing protein in PCC 7002. Both SYNPCC7002_G0067 and the “Type I secretion operon” mRNA are up-regulated by phosphate limitation and SYNPCC7002_G0067 is found in PCC 7002 supernatant upon phosphate limitation (Ludwig and Byrant., 2011 Transcription profiling of the model cyanobacterium Synechococcus sp. Strain PCC 7002 by Next-Gen (SOLiD) Sequencing of cDNA. Front Microbiol. 2:41.). SYNPCC7002_G0067 is a phosphatase that is secreted into the external milieu by a Type I system in response to phosphate limitation.

To identify putative C-terminal Type I secretion signals, we performed a computational screen for native cyanobacterial proteins secreted by Type I systems. We began with a list of known Type I secreted proteins (Delepelaire et al, 2004. Type I secretion in gram-negative bacteria. Biochim Biophysics Acta. November 11; 1694(1-3):149-61) and Blasted them against the following genomes: Synechococcus sp. PCC 7002, Synechococcus sp. PCC6803, Anabaena sp. PCC7120, and Synechococcus elongatus PCC 7942. We identified putative Type I secreted proteins based on homology of known Type I secreted proteins and chose the terminal 300 base pairs as a putative Type I secretion leader sequence. See Table 16.

To test the activity of the putative Type I secretion leader sequences, we devised a strategy to engineer a series genetic constructs to introduce a reporter gene fused to the putative Type I secretion signal into PCC 7002. The genetic constructs consisted of an E. coli plasmid backbone, a promoter system, a tag, a reporter gene, the putative Type I secretion leader, an antibiotic resistance cassette, and two PCC 7002 targeting sequences. The E. coli plasmid backbone facilitates the cloning and propagation of the genetic constructs in conventional E. coli hosts. The FLAG tag allows immunological detection of the fusion protein. The promoter system controls the expression of the reporter gene. We employed Pcpc, a high level constitutive promoter from Synechococcus sp. PCC6803 cpcB gene operon. We employed Pcro/cum, an inducible promoter consisting of the Pcro promoter from lambda phase with the cumate operator at the +1 position and the cumate repressor from Pseudomonas putida F1 divergently expressed from the Pkan promoter. The Pcro/cum system is inducible with the addition of cumate. We employed a truncated version of licB from Clostridium thermocellum. LicB (can be labeled NP280 in the Tables and Figures) encodes lichenase (beta-1,3-1,4-glucanase). Lichenase releases glucose when it cleaves its natural substrate, lichenan. The glucose released from the enzymatic reaction can be measured by a standard Dinitrosalicylic acid assay to measure the activity of lichenase and infer its concentration from this measurement. We employed spectinomycin as the antibiotic resistance cassette. We employed two 500 base pair sequences to target the expression cassette to a specific locus on pAQ3 in PCC 7002. A summary of the constructs is provided in Table D.

TABLE D

| | | | | Leader |
|---|---|---|---|---|
| | | | | sequence |
| | | | Gene of | in Table |
| Construct # | Insertion site | Promoter | interest | 16 |
| C0 | pAQ3 | pcpc | YFP | None |
| C1 | pAQ3 | pcpc | NP280 | F4 |
| C2 | pAQ3 | pcpc | NP280 | F5 |
| C3 | pAQ3 | pcpc | NP280 | F6 |
| C4 | pAQ3 | pcpc | NP280 | F7 |
| C5 | pAQ3 | pcpc | NP280 | F8 |
| C6 | pAQ3 | pcpc | NP280 | F9 |
| C7 | pAQ3 | pcpc | NP280 | F10 |
| C8 | pAQ3 | pcpc | NP280 | F11 |
| C9 | pAQ3 | pcpc | NP280 | F12 |
| C10 | pAQ3 | pcpc | NP280 | F13 |
| C11 | pAQ3 | pcpc | NP280 | F14 |
| C12 | pAQ3 | pcpc | NP280 | F15 |
| C13 | pAQ3 | pcpc | NP280 | F16 |
| C14 | pAQ3 | pcpc | NP280 | F17 |
| C27 | pAQ3 | pcpc | NP280 | F18 |
| C28 | pAQ3 | pcpc | NP280 | F19 |
| C15 | pAQ3 | pcro-cumO | NP280 | F4 |
| C16 | pAQ3 | pcro-cumO | NP280 | F5 |
| C17 | pAQ3 | pcro-cumO | NP280 | F8 |
| C18 | pAQ3 | pcro-cumO | NP280 | F18 |
| C19 | pAQ3 | pcro-cumO | NP280 | F9 |
| C20 | pAQ3 | pcro-cumO | NP280 | F10 |
| C21 | pAQ3 | pcro-cumO | NP280 | F11 |
| C22 | pAQ3 | pcro-cumO | NP280 | F21 |
| C23 | pAQ3 | pcro-cumO | NP280 | F12 |
| C24 | pAQ3 | pcro-cumO | NP280 | F13 |
| C25 | pAQ3 | pcro-cumO | NP280 | F14 |
| C26 | pAQ3 | pcro-cumO | NP280 | F15 |
| C29 | pAQ3 | pcro-cumO | NP280 | F16 |
| C30 | pAQ3 | pcro-cumO | NP280 | F19 |
| C31 | pAQ3 | pcro-cumO | NP280 | none |
| G | pAQ3 | pcro-cumO | NP280 | none |
| A1 | pAQ3 | pcpc | NP280 | F1 |
| A2 | pAQ3 | pcpc | NP280 | F2 |
| A3 | pAQ3 | pcpc | NP280 | F3 |
| A4 | pAQ3 | pcpc | YFP | none |
| A9 | pAQ3 | pcro-cumO | NP280 | F1 |
| A10 | pAQ3 | pcro-cumO | NP280 | F2 |
| A11 | pAQ3 | pcro-cumO | NP280 | F3 |

PCC 7002 was transformed with genetic constructs using natural competence. Transformants were selected on solid A+ agar plates with spectinomycin selection. Transformations were passed on spectinomycin selection plates to isolate fully segregated strains when possible. Engineered strains were grown in A+ media (A+) and A+ media without phosphate (P−) in 96 DWB, 35 C, 800 RMP, 5% CO2, spectinomycin. Expression from the Pcro/cum promoter was induced with 50 μM cumate. Lichenase activity was assayed in filtered supernatants and cell lysates using Dinitrosalicylic acid assay. Lichenase fusion protein concentrations were calculated based on assumptions on the specific activity of lichenase. Lichenase fusion protein concentrations were also measured using silver staining of SDS-PAGE gels and western blotting against the FLAG tag.

We were able to identify multiple Type 1 secretion signals that allow for the export of a heterologous protein in PCC 7002. The results showed that 1392, 1337, sll1951, all2654, all0364, alr1403, and G0067_F1 were the best secretion signals. As shown in Table E, many of the engineered strains showed significant levels of lichenase activity in the supernatant. Genetic constructs with the Pcpc promoter resulted in higher levels of lichenase activity in the supernatant. Growth on P− generally gave increased lichenase activity in the supernatant consistent with the up-regulation of the type I secretion system by phosphate limitation. Lichenase activity increased with the time of cultivation and the strongest signals were detected at 48 hrs growth post induction. Most of the engineered strains showed significant lichenase activity in the cell lysates (Table F), positive control for fusion protein expression, the ability of the C-terminal signal to direct secretion determines how much lichenase activity we can measure in the supernatant.

TABLE E

Lichenase (ug/ml) in Supernatants

| # | A+ | A+ | P− | P− |
|---|---|---|---|---|
| C0 | 0.054653 | 0.058556 | 0.0855706 | 0.077879 |
| C1 | 0.417702 | 0.274239 | 0.3480511 | 0.739368 |
| C2 | −0.00586 | 0.204947 | 0.3249759 | 0.122106 |
| C3 | 0.117113 | 0.147367 | 0.2307521 | 1.160491 |
| C4 | 0.929093 | 0.495777 | 2.1623398 | 0.334591 |
| C5 | 0.217634 | 0.31718 | 0.9268544 | 1.136454 |
| C6 | 0.471378 | 0.365977 | 0.2365209 | 0.178833 |
| C7 | 0.393303 | 0.82662 | 0.4172768 | 0.352858 |
| C8 | 1.407303 | 0.878345 | 1.5873824 | 0.836476 |
| C9 | 0.387448 | 0.409894 | 0.1836402 | 0.15095 |
| C10 | 0.903719 | 0.489921 | 1.1085717 | 0.687449 |
| C11 | 0.4421 | 0.63924 | 0.2749796 | 0.371126 |
| C12 | 0.443076 | 1.199428 | 1.0210782 | 0.927816 |
| C13 | 0.645095 | 0.663638 | 0.6163005 | 1.036462 |
| C14 | 0.146714 | 0.256556 | 0.2980548 | 0.171141 |
| C27 | 0.469426 | 0.388423 | 0.3701649 | 0.131721 |
| C28 | 0.084907 | 0.055628 | 0.0528807 | 0.036536 |
| C15 | 0.039038 | 0.077099 | 0.0673027 | 0.120183 |
| C16 | 0.046845 | 0.045869 | 0.0201908 | 0.03269 |
| C17 | 0.049773 | 0.174693 | 0.0692256 | 0.082686 |
| C18 | 0.099546 | 0.058556 | 0.1932549 | 0.203831 |
| C19 | 0.131752 | 0.083931 | 0.6018785 | 0.303824 |
| C20 | 0.155174 | 0.041965 | 0.1115302 | 0.110569 |
| C21 | 0.107353 | 0.758304 | 0.1115302 | 0.600917 |
| C22 | 0.081003 | 0.202995 | 0.088455 | 0.093262 |
| C23 | 0.083931 | 0.059532 | 0.1211449 | 0.091339 |
| C24 | 0.082955 | 0.065388 | 0.2153687 | 0.09807 |
| C25 | 0.128824 | 0.113209 | 0.0874935 | 0.222099 |
| C26 | 0.063436 | 0.025374 | 0.0509578 | 0.114415 |
| C29 | 0.109305 | 0.075147 | 0.0990311 | 0.157681 |
| C30 | 0.086859 | 0.076123 | 0.0874935 | 0.054804 |
| C31 | 0.078075 | 0.051725 | 0.0923009 | 0.09807 |
| G | 0.027491 | 0.017299 | 0.1377625 | 0.138126 |
| A1 | 1.162343 | 1.015952 | 1.2864431 | 0.86532 |
| A2 | 0.711459 | 0.887128 | 0.3653575 | 0.499963 |
| A3 | 1.114522 | 0.47333 | 0.6143776 | 0.729754 |
| A4 | 0.048797 | 0.002928 | 0.0499963 | 0.073072 |

TABLE F

| # | A+ | A+ | P− | P− |
|---|---|---|---|---|
| C0 | 0.059082 | 0.087729 | 0.0863104 | 0.01233 |
| C1 | 1.675794 | 1.613131 | 1.5007432 | 1.33693 |
| C2 | 1.903172 | 1.75278 | 1.4021028 | 1.416194 |
| C3 | 1.595227 | 1.362478 | 1.2136292 | 0.77327 |
| C4 | 1.913914 | 2.049983 | 1.4760831 | 1.530688 |
| C5 | 1.835138 | 1.598808 | 1.4161943 | 1.421479 |
| C6 | 1.550467 | 1.632825 | 1.5465406 | 1.613475 |
| C7 | 1.623873 | 1.643567 | 1.3193154 | 1.486652 |
| C8 | 1.691907 | 1.632825 | 1.5659164 | 1.52188 |
| C9 | 1.716973 | 1.571952 | 1.6469424 | 1.535972 |
| C10 | 1.623873 | 1.568371 | 1.6258052 | 1.530688 |
| C11 | 1.60955 | 1.652519 | 1.698024 | 1.592338 |
| C12 | 1.548677 | 1.641777 | 1.4426159 | 1.477845 |
| C13 | 1.659681 | 1.756361 | 1.520119 | 1.491936 |
| C14 | 1.595227 | 1.471691 | 0.6947715 | 0.799678 |
| C27 | 1.586275 | 1.598808 | 1.5113119 | 1.602907 |
| C28 | 0.008952 | 0.005371 | 0.0317058 | 0.03699 |
| C15 | 0.008952 | 1.206715 | 0.6112182 | 1.305224 |
| C16 | 0.956062 | 1.742038 | 1.0357242 | 0.81026 |
| C17 | 0.956062 | 1.111825 | 0.2923983 | 0.972313 |
| C18 | 1.042 | 0.961433 | 1.3668741 | 1.507789 |
| C19 | 1.808282 | 1.65789 | 1.5183576 | 1.407387 |
| C20 | 1.688327 | 0.04655 | 1.4091486 | 0.521385 |
| C21 | 1.666842 | 1.754571 | 1.3457369 | 1.546541 |
| C22 | 1.693698 | 1.79754 | 1.3756813 | 1.493697 |
| C23 | 1.724134 | 1.699069 | 1.451423 | 1.474322 |
| C24 | 1.627454 | 1.453787 | 1.3880114 | 1.48489 |
| C25 | 1.699069 | 1.605969 | 1.4338087 | 1.127319 |
| C26 | 0.510258 | 0.571131 | 0.1162548 | 0.137392 |
| C29 | 1.722344 | 1.666842 | 1.3844885 | 1.379204 |
| C30 | 0.00179 | 0.014323 | 0.3029669 | 0.035229 |
| C31 | 0.241701 | 0.18978 | 0.0457973 | 0.035229 |
| G | 0.085106 | 0.063472 | 0.068354 | 0.049618 |
| A1 | 1.554048 | 1.546887 | 1.5394948 | 1.537733 |
| A2 | 1.695488 | 1.665052 | 1.6363738 | 1.583531 |
| A3 | 1.571952 | 1.709811 | 1.7015469 | 1.490175 |
| A4 | 0.032227 | 0.035808 | 0.0299444 | 0.038752 |

We also characterized the secretion signal for SYNPCC7002_G0067. We designed 3 C-terminal fragments F1, F2, F3 292, 202, and 101 amino acids, respectively. We designed genetic constructs with Pcpc-FLAG-lichenase-F targeted to pAQ3. The longest secretion signal, F1 resulted in the most lichenase activity in the supernatant but all three secretion signals gave lichenase activity in the supernatant (Table G). In addition to performing the lichenase activity assay, we analyzed the supernatants and lysates with anti-FLAG western blots. Lysate and supernatant samples contained a major FLAG tagged protein. However the size of the protein is ˜30 kDA while the expected size of the F1, F2, and F3 lichenase fusion proteins is 63, 53, and 43 kDA respectively. The 30 kDA fragment is consistent with a truncated FLAG-lichenase protein fragment suggesting the fusion protein is subject to cleave. It is unclear if the truncated protein is being secreted or a small fraction of the full length protein is secreted and cleaved during the secretion process or in the supernatant.

TABLE G

| | | Lichenase (ug/ml) | | | |
|---|---|---|---|---|---|
| | | A+ | P− | A+ | P− |
| # | Plasmid | Lysate | Lysate | Super | Super |
| A9 | Flag-NP280::G0067-F1 | 2.154 | 1.086 | 1.272 | 0.231 |
| A10 | Flag-NP280::G0067-F2 | 2.059 | 1.096 | 1.271 | 0.246 |
| A11 | Flag-NP280::G0067-F3 | 1.998 | 1.101 | 1.141 | 0.236 |

Thus, we have identified native C-terminal leader sequences, constructed protein expression cassettes including constitutive and inducible promoters and the C-terminal leader targeted to a specific genetic locus, and demonstrated secretion of heterologous protein(s).

To increase secretion of heterologous protein by Type I in PCC 7002 the native SYNPCC7002_G0067 and/or the Type I secretion homologs SYNPCC7002_A2175 and SYNPCC7002_A2531 can be deleted. The expression of the Type I secretion operon can be up-regulated by increasing the strength of the native promoter, expressing the operon from a plasmid using the native promoter or a stronger promoter. The operon can be refactored to tune the ratio of protein for optimal secretion. Protein secretion can be made phosphate-independent by not using the native promoter. sphR, a trxn factor controlling the response to P limitation, can be overexpressed to up-regulate the expression of the Type I secretion operon under media replete conditions.

### Example 9

### Type IV Pathway Leader Identification and Use

Many gram negative bacteria possess Type IV pili (subsequently referred to simply as pili), long filamentous structures on the surface of the cell. Pili have been implicated in diverse cellular functions including twitching motility (Craig and Li 2008. Type IV pili; paradoxes in form and function. Curr Opin Struct Biol. 2008 Aor; 18(2)267-77). Pili consist of homopolymers of pilin proteins. Pilins are approximately 20 kDA in size and are characterized by a conserved N-terminal signal sequence and a structurally conserved N-terminal alpha helical domain (Giltner et al, 2012. Type IV pilin proteins:versatile molecular modules. Microbiol. Mol Biol Rev. 2012 December; 76(4):740-72)). The conserved signal sequence directs the insertion of the so-called prepilin into the cytoplasmic membrane by the Sec pathway. The signal sequence is then cleaved and the N-terminal amine is methylated by a prepilin peptidase (PilD) to produce a mature pilin (Giltner et al, 2012. Type IV pilin proteins:versatile molecular modules. Microbiol. Mol Biol Rev. 2012 December; 76(4):740-72). Although the precise mechanism is unknown, the cleaved pilin subunits are organized into a filament through a Type IV secretion system. The prototypical Type IV secretion system can be divided into four functional parts: 1) The major pilin (PiIA) that is polymerized into a filament. 2) The ATPases (PilB and PilT) that polymerize pilin subunits onto the growing filament. 2) The inner membrane platform (PilC, PilM, PilN, PilO, and PilP) that spans the inner membrane. 3) The porin (PilQ) that allows the growing filament to pass through the outer membrane (Korotkov et al, 2012. The type II secretion system: biogenesis, molecular architecture and mechanism. Nat Rev Microbiol. 2012 Apr. 2; 10(5):336-51). Pilin subunits are assembled in a helical manner are held together by hydrophobic interactions of N-terminal alpha helical domain (Giltner et al, 2012. Type IV pilin proteins:versatile molecular modules. Microbiol. Mol Biol Rev. 2012 December; 76(4):740-72).

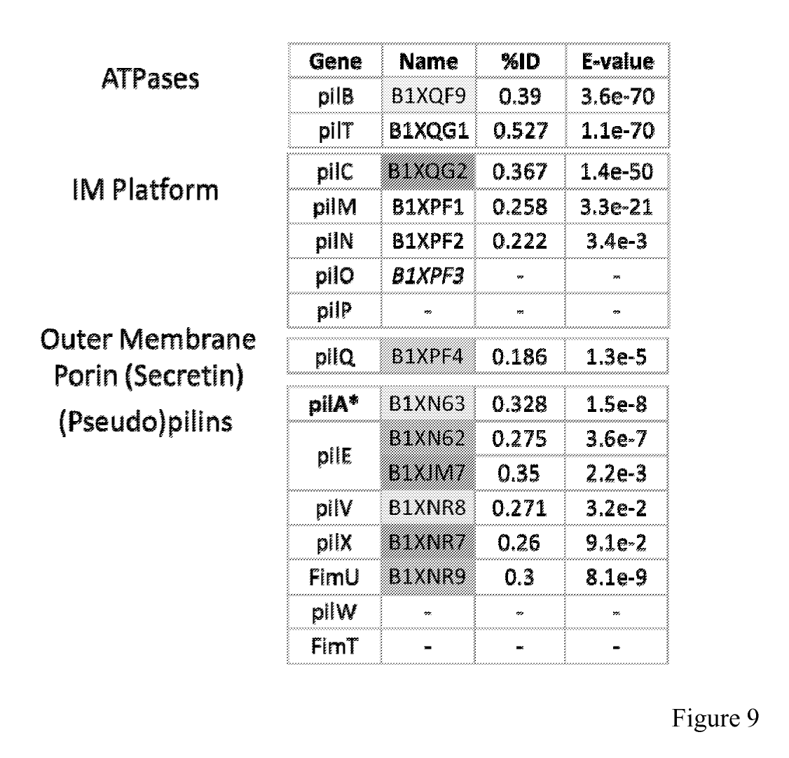

Many bacterial genomes contain homologs of the Type IV secretion system and pilins. In fact twitching, a form of flagella-independent motility has been documented in Synechocystis sp. PCC 6803 (Bhaya et al, 2000. Type IV pilus biogenesis and motility in the cyanobacterium Synechocystis sp. Strain PCC7803. Mol. Microbiol. 2000 August; 37(4); 213-6). Twitching is movement across a solid surface by extension, tethering, and retraction of Type IV pili (Mattick, 2002. Type IV pili and twitching motility. Annu Rev Microbiol. 2002; 56:289-314) A previous study discovered that PiIA (Sll1694) is also a major secreted protein in the freshwater cyanobacterium Synechocystis sp. PCC 6803 (Sergeyenko and Los, 2000. Identification of secreted proteins of the cyanobacterium Synechocystis sp. Strain PCC 6803. FEMS Microbiol Lett. 200 December 15: 193(2):213-6). A subsequent study showed that an N-terminal region of Sll1694 could direct the secretion of a reporter protein (Sergeyenko and Los, 2003. Cyanobacterial leader peptides for protein secretion. FEMS Microbiol Lett. 2003, Jan. 28; 218(2):351-7). The saltwater cyanobacterium PCC 7002 contains a homolog of the Type IV secretion system as well as 6 pilin homologs (A2804, A2803, A2335, A1603, A1602, and A1604). FIG. 9.

We screened for the secretion of five pilin homologs of PCC 7002 (A2804, A2803, A2335, A1602, and A1604). We engineered a series of genetic constructs to introduce a tagged version of each pilin homolog a specific genetic locus. The genetic constructs consist of an E. coli plasmid backbone, a promoter system, a pilin gene, a tag, an antibiotic resistance cassette, and two PCC 7002 targeting sequences. The E. coli plasmid backbone facilitates the cloning and propagation of the genetic constructs in conventional E. coli hosts. The promoter system controls the expression of the reporter gene. We employed Pcpc, a high level constitutive promoter from Synechococcus sp. PCC6803 cpcB gene operon. The tag is a FLAG tag that allows immunological detection of the fusion protein. We employed spectinomycin as the antibiotic resistance cassette. We employed two 500 base pair sequences to target the expression cassette to a specific locus on pAQ3 in PCC 7002.

PCC 7002 was transformed with genetic constructs using natural competence. Transformants were selected on solid A+ agar plates with spectinomycin selection. Transformations were passed on spectinomycin selection plates to isolate fully segregated strains when possible. Engineered strains were grown in PB1.1 media in a 96 DWB, 35 C, 800 RMP, 2% CO2, 70 μmol/m²/sec illumination, spectinomycin selection (100 ug/mL). Cultures were sampled at 24 hours (day 1), 48 hours (day 2), and 5 day time points. Samples were normalized to OD and collected by centrifugation at 15,000×g for 5 minutes. Supernatants were filtered through a 0.2 micron filter to remove any possible contaminating cells. Supernatant samples were assayed with an anti-FLAG dot-blot.

We detected significant quantities of tagged pilin in the supernatant for every construct tested. Tagged pilin protein accumulated over time. Table H presents the results of this experiment as ug/mL, and Table I presents the results as ug/mL/OD. A1602 and A2804 were secreted at the highest levels (approximately 6 mg/L/OD and 12 mg/L/OD respectively). A1604, A2335, and A2803 were detected a lower levels but above background levels. We performed anti-Rubisco dot blots on the supernatants and did not detect excess cell lysis in these strains indicating the pilin proteins were selectively secreted into the external milieu as opposed to released upon cell lysis.

TABLE H

| | Lichenase (ug/ml) | | |
|---|---|---|---|
| Group | Day 1 | Day 2 | Day 5 |
| A1602-FLAG | 7.454743 | 11.49144 | 55.4652 |
| A1604-FLAG | 0.542286 | 2.24238 | 18.1533 |
| A2335-FLAG | 0.030282 | 0.662218 | 6.379954 |
| A2803-FLAG | 0.803657 | 6.16245 | 10.66163 |
| A2804-FLAG | 15.46414 | 33.79599 | 108.0636 |
| A1602-C222- | 0.007156 | 0.006675 | 0.054764 |
| FLAG | | | |
| A1604-C222- | 0.057394 | 0.220838 | 2.036783 |
| FLAG | | | |
| A2335-C222- | 0.054766 | 0.057278 | 0.762666 |
| FLAG | | | |
| A2803-C222- | 0.019326 | 0.029827 | 0.285915 |
| FLAG | | | |
| A2804-C222- | 0.033304 | 0.20221 | 0.079488 |
| FLAG | | | |
| 7002 wt | 0.011367 | 0.004896 | 0.002171 |
| pES672 | 0.015253 | 0.007241 | |

TABLE I

| | Lichenase (ug/ml/OD) | | |
|---|---|---|---|
| Group | Day 1 | Day 2 | Day 5 |
| A1602-FLAG | 5.812665 | 5.277353 | 8.156648 |
| A1604-FLAG | 0.422835 | 0.930448 | 2.825417 |
| A2335-FLAG | 0.020813 | 0.205897 | 0.786435 |
| A2803-FLAG | 0.541183 | 2.144393 | 1.504286 |
| A2804-FLAG | 11.0065 | 12.78335 | 12.88389 |
| A1602-C222- | 0.005371 | 0.003085 | 0.010844 |
| FLAG | | | |
| A1604-C222- | 0.046567 | 0.105728 | 0.385207 |
| FLAG | | | |
| A2335-C222- | 0.035795 | 0.01827 | 0.088553 |
| FLAG | | | |
| A2803-C222- | 0.013491 | 0.011261 | 0.041969 |
| FLAG | | | |
| A2804-C222- | 0.027411 | 0.09745 | 0.012372 |
| FLAG | | | |
| 7002 wt | 0.008811 | 0.002046 | 0.000283 |
| pES672 | 0.008415 | 0.001923 | |

We evaluated the five pilin homologs of PCC 7002 (A2804, A2803, A2335, A1602, and A1604) for the ability to direct the secretion of another heterologous protein. We engineered genetic constructs identical to the previous section with the addition of a 65 amino acid fragment of myosin from Bos taurus (NPa) fused to the C-terminal of the pilin before the C-terminal FLAG tag. The sequence of NPa is listed in Table J below. We detected traces of A1604-NPa and A2804-NPa in the supernatant demonstrating these pilins can direct the secretion of heterologous protein.

We evaluated the ability for A2804 and A1604 to direct the secretion of seven additional heterologous proteins. We engineered genetic constructs to introduce each combination of A2084 and A1604 with a C-terminal fusion to various fragments of serine/threonine-protein kinase MEC1 from fragments Saccharomyces cerevisiae (P38111), identified asNPb, NPc, NPd, NPe, NPf, NPg, and NPh (pES1457, pES1458, pES1428, pES1459, pES1460, pES1461, pES1462, pES1471, pES1472, pES1475, pES1476). See Table J. The promoter was Pcro/cum and the genetic locus was pAQ3.

TABLE J

| | | | | SEQ |
|---|---|---|---|---|
| | | Fragment | | ID |
| NP | DBID | ends | Sequence | NO |
| NPa | Q27991 | 126:190 | DTKKKVDDDLGTIENLEEAKKKLLKDVEVLSQRLEEKALAY | |
| | | | DKLEKTKTRLQQELDDLLVDLDHQ | |
| NPb | P38111 | 1093:1165 | LVLGALLDTSHKFRNLDKDLCEKCAKCISMIGVLDVTKHEF | |
| | | | KRTTYSENEVYDLNDSVQTIKFLIWVINDILV | |
| NPc | P38111 | 1093:1182 | LVLGALLDTSHKFRNLDKDLCEKCAKCISMIGVLDVTKHEF | |
| | | | KRTTYSENEVYDLNDSVQTIKFLIWVINDILVPAFWQSENP | |
| | | | SKQLFVAL | |
| NPd | P38111 | 1093:1162 | LVLGALLDTSHKFRNLDKDLCEKCAKCISMIGVLDVTKHEF | |
| | | | KRTTYSENEVYDLNDSVQTIKFLIWVIND | |
| NPe | P38111 | 1092:1166 | TLVLGALLDTSHKFRNLDKDLCEKCAKCISMIGVLDVTKHE | |
| | | | FKRTTYSENEVYDLNDSVQTIKFLIWVINDILVP | |
| NPf | P38111 | 1093:1168 | LVLGALLDTSHKFRNLDKDLCEKCAKCISMIGVLDVTKHEF | |
| | | | KRTTYSENEVYDLNDSVQTIKFLIWVINDILVPAF | |
| NPg | P38111 | 1091:1164 | ITLVLGALLDTSHKFRNLDKDLCEKCAKCISMIGVLDVTKHE | |
| | | | FKRTTYSENEVYDLNDSVQTIKFLIWVINDIL | |
| NPh | P38111 | 1089:1164 | SDITLVLGALLDTSHKFRNLDKDLCEKCAKCISMIGVLDVTK | |
| | | | HEFKRTTYSENEVYDLNDSVQTIKFLIWVINDIL | |

Engineered strains were grown in PB1.1 media in a 96 DWB, 35 C, 800 RMP, 2% CO2, 70 μmol/m²/sec illumination, spectinomycin selection (100 ug/mL). Cultures were inoculated at OD 0.2 and induced at OD 0.4 with 75 uM cumate. An additional 75 uM cumate was added 12 hrs later. Cells were harvested 48 hrs after the second induction. Induction of fusion protein expression resulted in a growth defect indicative of toxicity. We could detect the secretion in an engineered strain transformed with pES1475. We detected 8.3 mg/L by anti-FLAG dot-blot. We verified presence of A2804 and NPg in the supernatant with mass spec analysis. We could detect a candidate band in supernatant silver stain and we could not detect significant cell lysis indicating that A2804-NPg is specifically secreted into the external milieu.

We performed experiments on pES1475 and pES1475 to search for experimental conditions that result in increased fusion protein secretion. We varied the media (PB 1.1, A+), OD at induction (0.5, 1), cumate level (10 uM or 100 uM). For pES1475, we achieved approximately 6 mg/L/OD fusion protein in the supernatant in A+, induced at OD 0.5 and assayed at 48 hrs post induction. For pES1475, we achieved approximately 3 mg/L/OD fusion protein in the supernatant in cells grown in PB1.1, induced with 10 uM cumate at OD 0.5 and assayed at 48 hrs post induction. These results demonstrate that A2804 is able to direct the secretion of at least three heterologous proteins in PCC 7002.

Thus, we identified secreted pilins in cyanobacteria and demonstrated the use of pilin fusions to secrete heterologous proteins.

### Example 10

### Sec Pathway Leaders and Lichenase Secretion

Different ways of exporting protein into periplasm are by utilizing the “Sec pathway” or “TAT pathway”. This example focused mainly on the “Sec pathway”. The proteins of interest were generally fused with a N-terminal Sec leader which enable them to be recognized by the chaperone protein (secB) to keep in unfolded state after translation and target to peripheral internal membrane protein SecA. The protein then gets exported through a transport sandwich complex comprising of SecY, SecE and SecG through the inner membrane into the periplasm. Under certain conditions and in certain bacteria, the protein can then be secreted to extracellular matrix.

The cyanobacterium PCC 7002 encodes all the machinery related to Sec related translocation. A1259 gene encodes SecA, A1047 gene encodes secY, A1031 gene encodes secE, A2234 gene encodes secG.

Using Sec Leaders from Proteins that are Naturally Secreted by Cyanobacteria

As described above, we identified 8 different Sec leader sequences from proteins that are naturally secreted in different cyanobacteria. In these experiments we have used lichenase as our protein of interest. We integrated the secretion leaders in front of lichenase and observe how it impacts the secretion of lichenase into the extracellular media.

All DNA constructs were constructed using standard cloning procedures. For this study the vectors were as follows: pES163 (pAQ1 integration vector with pcpc*-lichenase), pES168(pAQ1 integration vector with pcpc*-pilus leader from A1602, (MINQPCIVPAEKG)-lichenase), pES171(pAQ1 integration vector with pcpc*-Sec-leader from naturally secreted protein of ECC012 (sec leader derived from SEQ ID No. 64, SP8 with one modification in the second amino acid from Q to E for introducing a restriction site for cloning (MELKKLFVPLLAGMLFLGGTSGAIAEELL)-lichenase), pES186 (pAQ1 integration vector with pcpc*-pilus leader from pilA of Synechocystis PCC 6803 (MASNFKFKLLSQLSKKRAEGG)-lichenase), pES187 (pAQ1 integration vector with pcpc*-negatively charged artificial leader (MEIDGFGGILYTSDEAILGG)-lichenase). All of the constructs were transformed into Synechococcus sp. PCC 7002 using natural transformation method.

10 ml cultures were grown in PB 1.1 media @ 2% CO₂, 70 umol/m²/sec, 200 rpm in 125 ml shake flask starting with OD₇₃₀=0.3 from a starter culture grown for 3 days in 5 ml of A+ media starting from a patch of segregated colony. ECC001:pES186 and ECC001:pES187 didn't grow well and were not used for analysis.

1.175 ml culture was sampled at 18, 41, 65, and 137 hrs, 1 ml culture was centrifuged at 15000×g for 5 mins and the supernatant was filtered using a 0.2 um filter. The pellet was resuspended in 1 ml PB 1.1 media and lyzed using 500 ul glass beads @ 30 Hz for 5 mins in Bead beater. Lyzed samples were centrifuged at 15,000×g for 5 mins and the supernatant was used for lichenase quantification.

The amount of lichenase in the supernatant and lysate was quantified using a Dinitrosalicylic acid assay for detection of lichenase activity. We also looked at the relative level of lichenase in supernatant and lysate using Congo Red assay for detection of lichenase activity. To verify that the cells were secreting lichenase we determined the amount of lysis using rbcL antibody, which looks for rbcl protein (intracellular cytoplasmic protein) using the Dot Blot Analytical Method. Further we also looked at lichenase secretion by running the supernatant samples in a protein gel and using silver stain to look at the protein of interest.

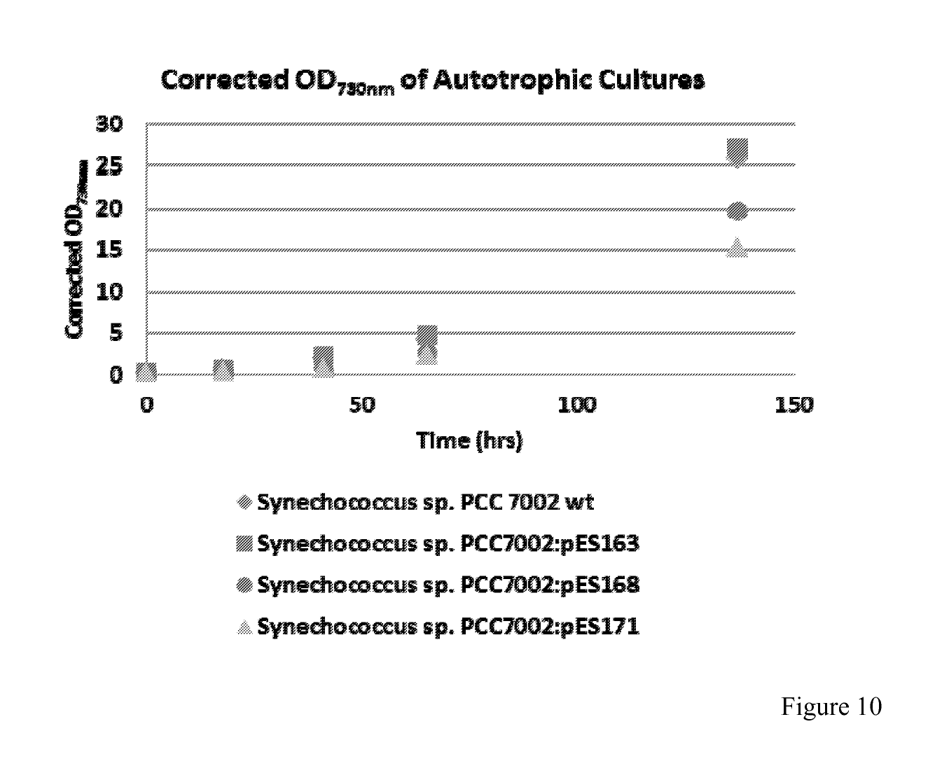

Engineered Synechococcus sp. PCC 7002 strains grew at different rates over the course of the experiment. FIG. 10.

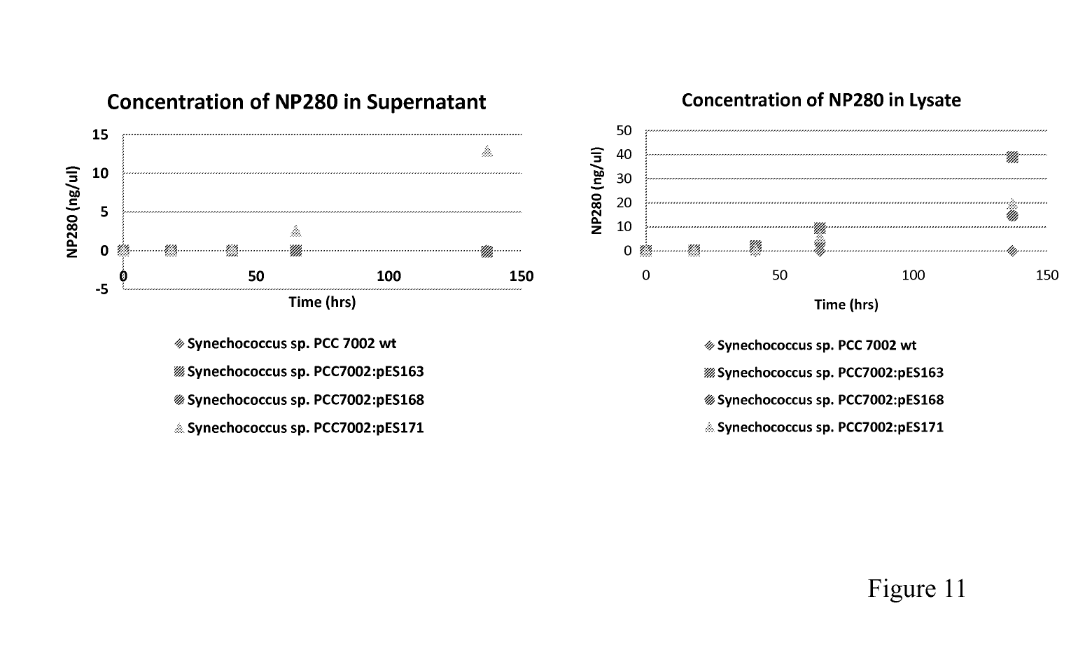

Three cells lines transformed with pES163, pES168 and pES171, respectively, expressed lichenase (NP280). Only the Synechococcus sp. PCC 7002 strain transformed with pES171 exhibited lichenase in the supernatant (FIG. 11).

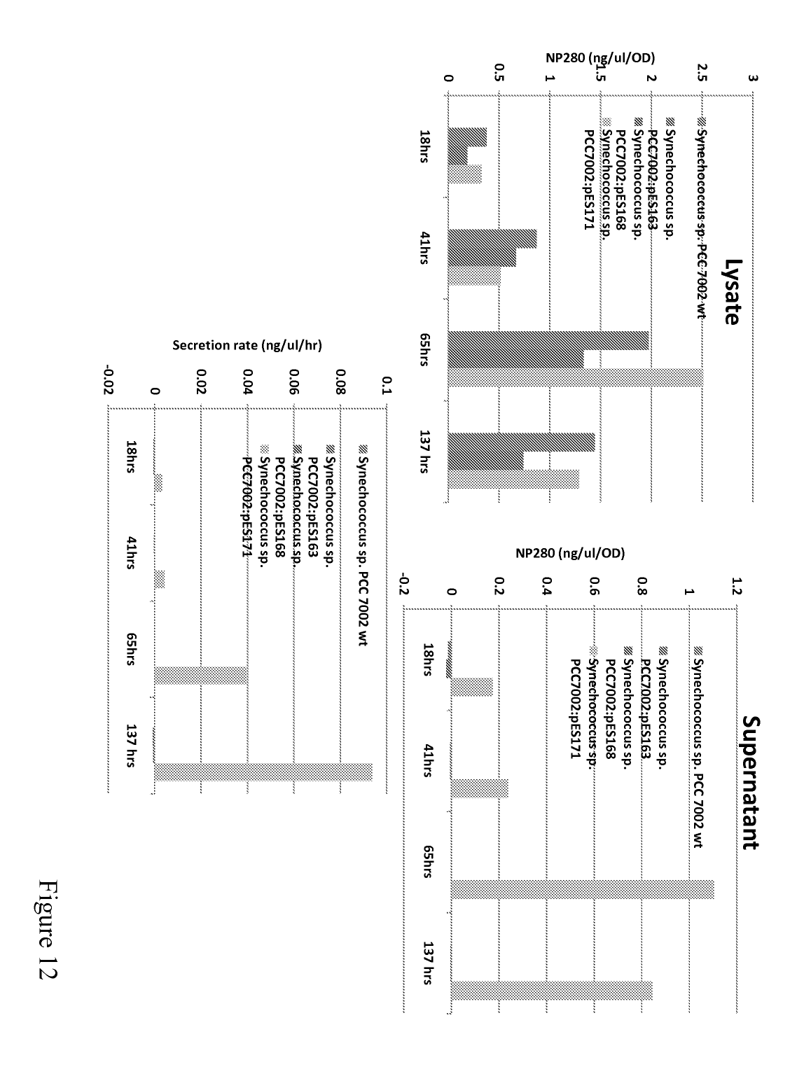

Using an activity assay it was possible to calculate the concentration of lichenase per microliter per OD₇₃₀ₙₘ and thus to calculate the rate of secretion (FIG. 12). Maximum lysate concentration per OD₇₃₀ₙₘ was obtained in the sec-leader2-lichenase (pES171) cells at 65 hrs, at 2.51 ng/uL/OD. Secretion of lichenase reached a secretion rate at 137 hrs of 0.094 ng/uL/hr, and a concentration in the supernatant of 1.10 ng/uL/OD₇₃₀ₙₘ at 65 hrs. Over the course of the experiment the average secretion efficiency of Synechococcus sp. PCC 7002:pES171 was 34.0%.

A parallel qualitative plate activity assay confirmed the presence of active lichenase in lysates and supernatants of PCC 7002. RbcL is an intracellular cytoplasmic protein in Synechococcus sp. PCC 7002, its presence in supernatant would be an indication that cell lysis was occurring and thus a possible source of lichenase detected in the supernatant. An anti-RbcL dot blot was run on supernatant samples to confirm that the presence of lichenase in the supernatant was not the result of cell lysis. With the exception of the outlier 7002:pES163, at 3 days all samples showed less than 1% lysis and lysis of transformed Synechococcus sp. PCC 7002 strains was less than Synechococcus sp. PCC 7002 wild type. The data show that lysis is not a significant contributor to lichenase in the supernatant.

SDS PAGE was run on supernatant samples from 7002 wt, 7002:pES163 and 7002:pES171, OD₇₃₀ₙₘ normalized to 2.0 and silver stained. Lichenase was detected at the appropriate molecular weight in the supernatant sample of 7002:pES171. Neither 7002 wt nor the intracellular expressing strain, 7002:pES163, showed the presence of lichenase.

In this example we successfully detected secretion of a heterologous protein (lichenase) in the phototrophic strain 7002:pES171 using a secretion leader based on SP8 (Seq ID No. 64) derived from Synechococcus sp. ATCC 29404. At 137 hours we observed a titer of 13 mg/L of lichenase in the supernatant. A maximum secretion rate of 0.094 ng/uL/hr was reached at 137 hrs.

### Example 11

### Screening and Using Sec Leaders Identified in Silico from Homologous Proteins Predicted to be Secreted in Cyanobacteria

In-Silico Prediction Model

The 48 sec leaders examined in this study were selected using a combination of 2 measures of predicted efficacy. The first measure was the predicted presence (or lack thereof) of an N-terminal sec signal sequence as identified by a set of in-house developed signal sequence neural networks designed to predict the presence of a sec signal sequence as well as the predicted cleavage site of the leader. The second measure was the sequence homology of the candidate protein to a list of proteins known to be secreted via the sec pathway. These two measures were used in conjunction to assess and rank all known proteins in the proteome of Synechococcus PCC7002.

The neural networks constructed are similar to that used by Nielsen et al (Nielsen et al, 1997. A neural network method for identification of prokaryotic and eukaryotic signal peptides and prediction of these cleavage sites. Int J Neural Syst. 1997 October-December; 8(5-6):581-99) in their SignalP prediction software (Bendtsen et al, 2004. Improved prediction of signal peptides: SignalP 3.0. J Mol. Biol. 2004 Jul. 16; 340(4):783-95). One network was used to assess the S-score, i.e., whether any given position within the first 60 amino acids of a candidate was a member of a sec signal sequence. The second network was used to assess the C-score, i.e., whether any given position within the first 60 amino acids of a candidates sequence was in the P1 position (the final amino acid prior to cleavage) of a sec peptidase cleavage site. For those proteins predicted to contain sec signal sequences, the site with the largest C-score was identified as the most likely cleavage site. The presence of a sec signal sequence was predicted using a discrimination function of both the S- and C-scores at each position. This score accounts for the magnitude of the C-score as well as the shape of the S-score over the N-terminal 60 amino acids and is defined as

$$D = 0.55 * {max}_{i} \sqrt{{C}_{i} * \frac{1}{12} ( {\sum }_{j = 1}^{12} {S}_{i - j} - {\sum }_{j = 0}^{11} {S}_{i + j} )} + 0.45 * 〈 S 〉$$

where i is the amino acid index, Cᵢ is the C-score at position i, Sᵢ is the S-score at position i and [S] is the mean S-score averaged over all indices.

It is a weighted average of the mean S-score and the product of the C-score and derivative of the S-score (averaged over a 12 amino acid window), maximized over all indices. In effect, this score rewards large average S-scores as well as sequences containing positions with simultaneously large C-scores and very negative S-score derivatives (i.e., positions strongly predicted to be part of the very end of a signal sequence). Large D values are indicative of the presence of a sec signal sequence and small values indicate the lack thereof.

Both networks used a 5 fold cross validation strategy with 2 hidden layers, were trained using the gram negative bacteria training dataset provided in the signal 2.0 package, and implemented using the biopython v1.53 toolbox and python v2.6. The S-score network was specifically trained using four pieces of data from each position in each sequence in the training dataset: the amino acid distribution of a window of 40 amino acids that included the 20 residues before and after each position, the amino acid distribution of the first 60 amino acids, the position index, and its identity as a signal sequence, cleavage, or normal residue. The C-score network was trained using similar data but used a 22 amino acid window around each cleavage site that included 20 amino acids N-terminal to the cleavage site and 1 amino acid C-terminal to the cleavage site. Given the disparity between the number of positions in the training set that were members of a signal sequence relative to those that were not, the negative examples were randomly sampled such than an equal number of positive and negative examples were selected for training.

The prediction statistics obtained from the 5 fold cross validation of the trained S and C networks are shown in Table K. Using a D value cutoff of 0.35, the maximal Mathews correlation coefficient (MCC) is very close to 1, indicating a very high degree of correlation between the observed and predicted signal sequences. Similarly, the accuracy, sensitivity, and specificity are all close to 1, which indicates that this network is effective at predicting true positives and true negatives.

TABLE K

| | |
|---|---|
| MCC | 0.84 |
| Accuracy | 0.92 |
| Sensitivity | 0.95 |
| Specificity | 0.89 |

The sequence homology was assessed using a global-global optimal alignment using the FASTA algorithm with the BLOSUM50 substitution matrix, a gap open penalty of 10, and a gap extension penalty of 2 (Pearson 1988).

### Experimental Results

All 48 secretion leaders (Table 18) were fused in N-terminal of lichenase (NP280) and put in downstream of pero-CumO promoter and integrated into pAQ3 plasmid. Flag tag was added to C-terminal of lichenase for detection in Western Blot and DOT-BLOT. One of the leader sequences, leader 10 didn't transform in Synechococcus PCC 7002. The results are summarized below.

All the sequences selected for use in this study were predicted to be sec secretion leaders using the prediction neural network described above. Approximately 64% of the predicted leaders yielded strain activities greater than 0.5 ug Lichenase/mL/OD730, 52% of the leaders yielded activities greater than 0.75 ug Lichenase/mL/OD730, 37% of the leaders yielded activities greater than 1 ug Lichenase/mL/OD730, and 23% of the leaders yielded activities greater than 1.25 ug Lichenase/mL/OD730. Table L.

TABLE L

| | | Average | |
|---|---|---|---|
| | | on | |
| | | triplicates | |
| | | assay | |
| Construct | Leader name | (μg/ml) | Std Dev |
| 1 | L1 | 1.726739 | 0.228139 |
| 2 | L2 | 2.527558 | 0.311013 |
| 3 | L3 | 2.581304 | 0.252593 |
| 4 | L4 | 1.188208 | 0.099042 |
| 5 | L5 | 1.684184 | 0.112257 |
| 6 | L6 | 2.268906 | 0.129492 |
| 7 | L7 | 0.151272 | 0.033177 |
| 8 | L8 | 0.043115 | 0.02438 |
| 9 | L9 | 0.059843 | 0.050764 |
| 11 | L11 | 1.21773 | 0.215532 |
| 12 | L12 | 2.182086 | 0.144285 |
| 13 | L13 | 1.688951 | 0.048235 |
| 14 | L14 | 1.900306 | 0.216161 |
| 15 | L15 | 1.265727 | 0.209958 |
| 16 | L16 | 1.416067 | 0.240349 |
| 17 | L17 | 2.620891 | 0.173872 |
| 18 | L18 | 2.675275 | 0.059384 |
| 19 | L19 | 1.244302 | 0.129536 |
| 20 | L20 | 1.709431 | 0.139605 |
| 21 | L21 | 0.340166 | 0.041554 |
| 22 | L22 | 0.105095 | 0.014782 |
| 23 | L23 | 3.021886 | 0.220369 |
| 24 | L24 | 2.472906 | 0.122287 |
| 25 | L25 | 0.352233 | 0.017862 |
| 26 | L26 | 0.179316 | 0.024029 |
| 27 | L27 | 2.455379 | 0.209371 |
| 28 | L28 | 1.698542 | 0.083767 |
| 29 | L29 | 0.057563 | 0.01025 |
| 30 | L30 | 0.199192 | 0.028373 |
| 31 | L31 | 1.19122 | 0.060153 |
| 32 | L32 | 2.396006 | 0.100785 |
| 33 | L33 | 3.372915 | 0.216646 |
| 34 | L34 | 3.355593 | 0.126665 |
| 35 | L35 | 1.268274 | 0.080773 |
| 36 | L36 | 0.148006 | 0.008027 |
| 37 | L37 | 2.109163 | 0.084408 |
| 38 | L38 | 3.254843 | 0.114229 |
| 39 | L39 | 1.006157 | 0.149531 |
| 40 | L40 | 0.077758 | 0.027898 |
| 41 | L41 | 3.473463 | 0.115754 |
| 42 | L42 | 0.212266 | 0.049036 |
| 43 | L43 | 0.622873 | 0.18702 |
| 44 | L44 | 0.457825 | 0.122579 |
| 45 | L45 | 3.394909 | 0.162431 |
| 46 | L46 | 0.985349 | 0.153765 |
| 47 | L47 | 3.266336 | 0.148609 |
| 48 | L48 | 0.059881 | 0.025127 |
| G no leader | no leader | 0.096049 | 0.01599 |

### Example 12

### Phosphatase Pathway Leader Identification and Use

Phosphate is an essential nutrient for all organisms, present in nucleic acids, phospholipids, and various important solutes such as ATP. Prokaryotes and eukaryotes from various environments (terrestrial, oceanic and freshwater) need phosphate in large amount to maintain their growth and reproduction. A source of phosphate for microbial growth is the inorganic phosphate (Pi), soluble and acquired by active transport. However, the anion Pi often becomes limited in nature and is found in an insoluble form, in complex with organic compounds, and is not easily accessible to cells. Alkaline phosphatases (APases) are able to release free Pi from these organic compounds and thus play an important role in Pi uptake by fulfilling microorganisms phosphate needs for their growth (Plant Physiol. 1988 April; 86(4):1179-84. Identification and Purification of a Derepressible AlkalinePhosphatase from Anacystis nidulans R2Block M A, Grossman A R.; Subcellular localization of marine bacterial alkaline phosphatases—Haiwei Luo et al. PNAS 2009; Appl Environ Microbiol. 2011 August; 77(15): 5178-5183. An Alkaline Phosphatase/Phosphodiesterase, PhoD, Induced by Salt Stress and Secreted Out of the Cells of Aphanothece halophytica, a Halotolerant Cyanobacterium—Hakuto Kageyama et al.).

Three phosphatase gene families (PhoA, PhoX and PhoD) have been reported in Prokaryotes. They are a nonspecific phosphomonoesterases that hydrolyze phosphate ester bonds to free the Pi. They differ in sequence, substrates specificity and metal requirements for their activities, but are generally associated with zinc (Luo 2009 et al. and Kageyana 2011 et al.).

It is well documented that in response to phosphate limitation, microorganisms such as E. coli, Cyanoabacteria (Anacystis nidulans (Synechococcus 6301)) and some eukaryotes (Saccharomyces cerevisiae), increase their production of APases to enhance phosphate uptake (Luo 2009 et al.; Arch. Microbiol. 102, 23-28 (1975)—Phosphate utilization and Alkaline Phosphatase activity in Anacystis nidulans (Synechococcus)—M J A Ihlenfeldt and J Gibson.). Studies carried out in E. coli, and in some Cyanobacteria as well (e.g. Synechococcus sp. WH8102), show that this mechanism is well regulated (ISME J. 2009 July; 3(7):835-49. Microarray analysis of phosphate regulation in the marine cyanobacterium Synechococcus sp. WH8102.Tetu SGBrahamsha et al.).

APases have been reported primarily to be periplasmic in Gram-negative bacteria, but they have also been identified on the cell surface and extracellularly as well. Their role in P cycle and subcellular localization have been documented for marine organisms as Cyanobacteria: between all the autotrophic and heterotrophic marine microorganisms tested, 42% of the APases are cytoplasmic, 30% extracellular, 17% periplasmic, 12% in the outer membrane and 1% in inner membrane (Luo 2009).

Based on APases activity assays, phosphatases are mainly known as periplasmic proteins (Anacystis nidulans (Synechococcus 6301)-1) or as surface exposed and extracellular (e.g. Nostoc commune UTEX 584) (Indian Journal of Fundamental and Applied Life Sciences ISSN: 2231-6345ALKALINE PHOSPHATASE ACTIVITY IN CYANOBACTERIA: PHYSIOLOGICAL AND ECOLOGICAL SIGNIFICANCE V. D. Pandey and Shabina Parveen; Whitton B A, Grainger S L J, Hawley G R W and Simon J W (1991). Cell-bound and extracellular phosphatase activities of cyanobacterial isolates. Microbial Ecology 21 85-98; J Biol. Chem. 1993 Apr. 15; 268(11):7632-5. A protein-tyrosine/serine phosphatase encoded by the genome of the cyanobacterium Nostoc commune UTEX 584. Potts M, et al.). However, questions regarding the mechanisms of secretion (extracellular) or even export (periplasmic) remain unanswered in Cyanobacteria species. Also since more of the studies are based on activity assays, it is not clear if some of the extracellular phosphatases are loosely bound to the cell wall, attached to outer-membrane vesicles, or free in the medium.

Synechococcus PCC7002 encodes 33 putative phosphatases in its genome. Amongst them some were identified with an N-terminal signal peptide with Signal peptide prediction programs (e.g., SYNPCC7002_A0064, SYNPCC7002_A0893, SYNPCC7002_A2155, SYNPCC7002_A2352, SYNPCC7002_A0973), suggesting that they are exported to the periplasm and potentially secreted in the external media. Table 19. The 28 others could be cytoplasmic, anchored in the inner membrane or eventually released in the supernatant if the secretion mechanism does not involve an intermediate step through the periplasm (e.g., Type I secretion system). Transcriptome analysis on PCC7002 grown in various stress conditions report that, under phosphate limitations, transcription for four phosphatases is enhanced for: SYNPCC7002_A2352 up to 72-fold, SYNPCC7002_A0893 up to 145-fold, SYNPCC7002_G0067 up to 61-fold and SYNPCC7002_A0150 up to 35-fold (Synechococcus sp. Strain PCC 7002 Transcriptome: Acclimation to Temperature, Salinity, Oxidative Stress, and Mixotrophic Growth Conditions. Ludwig M, Bryant D A. Front Microbiol. 2012; 3:354).

Identification of the Secreted Phosphatases in Extracellular Environment of PCC7002 Grown Under Normal and Phosphate Limitation Conditions

We determined by mass spectrometric analysis the protein content of two supernatants from PCC7002 grown in standard (A+) and phosphate-limited conditions (P−).

Ten mL of PCC7002 grown in standard A+ medium and P-medium (P− corresponding to A+ medium with low phosphate content (10 uM KH2PO4 instead of 370 uM in A+) during 3 days in standard conditions were harvested by centrifugation at 5000 rcf during 15 min. Supernatants were filtered on 0.22 um membrane and concentrated 10×. Fifteen microliters were loaded on SDS-PAGE and sent for mass spec analysis.

The three proteins most frequently identified in low phosphate medium are the predicated PhoX phosphatase (SYNPCC7002_A0893) with 504 hits, the alkaline phosphatase (PhoA-SYNPCC7002_A2352) with 250 hits, and the Endonuclease/Exonuclease/phosphatase (SYNPCC7002_G0067) with 53 hits. These data demonstrate that phosphatases are the major secreted proteins from PCC7002 under phosphate starvation.

Demonstration of Increased Phosphatase Activity in Extracellular Environment of PCC7002 Under Phosphate Limitation

Using a fluorescent assay, we determined the phosphatase activity in cell lysates and filtered supernatants from PCC7002 grown in standard conditions and under phosphate limitations for 3 days.

From a preculture of PCC 7002 grown in A+ medium, 10 mL of standard media A+ and P− (A+ Low phosphate−A+ medium protocol with 10 uM KH2PO4 instead of 370 uM in A+)+spec100 were inoculated at OD730 0.2 with washed cells. Cells were then grown for 3 days at 35 C in standard conditions of light and CO2. One mL of culture was harvested after 1-2 and 3 days of incubation. Supernatants were harvested after pelleting cells by centrifugation at 5000 rcf during 10 min, filtered on 0.2 um membrane and saved on ice. Cell pellets were resuspended in fresh media and saved on ice. Twenty uL of washed cells and supernatants (in triplicates) were used to perform a Phosphatase activity assay using MUP compound (4-methylumbelliferyl phosphate).

The data demonstrated that PCC7002 supernatants from low phosphate medium have about 200 times more active phosphatases compared to standard conditions. Note that PCC7002 has a phosphatase activity in its supernatants enhanced by about 25 times, when the strain reaches stationary phase (app. OD730 ˜3-5) in standard medium.

We also analyzed the supernatants concentrated 10× by SDS-Page and silver or Coomassie blue stain. Each load is equivalent at 100 uL of supernatants at the time of harvest. OD730 at harvest are mentioned at the bottom of the silver stained gel. The same samples were analysis on SDS-Page stained with Coomassie Blue along with different concentration of BSA (2 to 0.2 ug) for concentration estimation.

The two major proteins detected in phosphate limited conditions have the same molecular mass as the two phosphatases detected by mass spec: SYNPCC7002_A2352 (PhoA—52 kDa) and SYNPCC7002_A0893 (PhoX—67 kDa). See Table 19. PhoX was estimated on Coomassie blue SDS-Page at <0.1 ug/mL after 3 days of growth in low phosphate medium when cells were harvested at OD730 2. Based on the silver stain and the mass spec data, PhoA could be estimated as twice less abundant than PhoX, meaning <0.05 ug/mL.

In A+ medium after 3 days of growth (when PCC7002 cell density was above 5), the 2 proteins identified as being SYNPCC7002_A0893 and SYNPCC7002_A2352 were detectable. This observation corroborates the phosphatase activity assays showing an increase of extracellular phosphatases when cells are getting phosphate deprived and enter in stationary phase.

Demonstration of Increased Secreted Phosphatase from PCC7002 by Overexpressing A2352-Flag

We tested overexpression of A2352 protein fused to a Flag tag at its C-terminal in PCC7002 grown in A+ and P− media.

The gene A2352 was cloned in the vector pES976 under control of the inducible promoter pero-cumR and fused at the 3′ end to the sequence encoding a Flag tag. The final plasmid carrying A2352-flag, named pES1197 (see pES library on Geneious), was transformed in PCC7002. The final strain carrying the expression cassette (pero-cumR-A2352-flag—lox-spec-lox) on pAQ3 plasmid was obtained after selection on A+ medium supplemented with Spectinomycin 100 ug/mL (spec100).

After 3 restreaks on selective medium (agar plate with A+ with spec100), the strain PCC7002 pAQ3-pero-cumR-A2352-Flag was inoculated in 5 mL A+ medium (+spec 100) and incubated for 2 days in standard growth conditions. A preculture of the wild-type strain EA001 was prepared in parallel. Both precultures were washed in P− and then diluted at OD730 0.2 in 10 mL of A+ and P− media (+spec100 when necessary). EA001 pero-cumR-A2352-Flag was then grown for 19 h at 35 C in standard conditions of light and CO2 before being induced with 50 uM cumate. Each culture was harvested after 48, 72 and 120 h of growth. One mL of each culture was harvested by centrifugation at 5000 rcf during 10 min. The supernatants were filtered on 0.2 u membrane, supplemented with inhibitor of proteases (Sigma cat# P2714) and concentrated 10× and analyzed on SDS-Page followed by silver stain detection.

Silver stained gel showed that A23352-Flag was secreted in the supernatant of both media. The secretion rate of A2352-Flag in A+ medium was about 5 to 10 times higher than in P−, possibly due to the higher biomass harvested (OD_{730 ˜7} in A+ and 2 in P−). The concentration of A2352-FLAG secreted per OD in A+ and P− media is likely similar. Western blot with antibodies against the Flag tag confirmed that the protein was highly detected on silver stain is A2352-Flag.

The amount of A2352-Flag secreted in A+ supernatant was estimated using a Coomassie Blue stained gel at 5 ug/mL after 5 days of induction. Thus, overexpression of A2352-Flag from an inducible promoter when cells are grown in A+ medium enhanced A2352-Flag secretion by 100×. During the secretion process, the phosphatase A2352 has its N-terminal signal peptide cleaved (first 47 amino acids).

Method to Improve Growth of PCC7002 and Consequently Improve Secretion of Overexpressed SYNPCC7002_A2352

To improve A2352-Flag secretion by optimizing growth rate of PCC7002, we analyzed A2352-Flag secretion from cells grown in various media known to enhance growth rate of PCC7002. In each media, A2352-Flag was induced with various concentration of cumate (0, and 7 uM). The first media used was PB1.1 containing 10 mL/L of nitrogen, the second media was PB1.1 in which nitrogen was replaced by 10 mM urea at the time of induction of the construct and the third medium was PB1.1 in which 10 mM urea was added every 24 h (urea spike) from the time of induction of the construct. We compared the amount of A2352-Flag secreted in each condition with the standard medium A+ used previously.

In A+ medium, strains reached stationary phase in 4 days at OD ˜9, while when grown in PB1.1, the stationary phase was reached after 7 days at OD ˜30 in PB1.1, 22 in PB1.1+urea and 19 in PB 1.1+urea spikes, indicating PB1.1 is the media that gives the highest biomass. However, the highest biomass is not correlated to the highest rate of secreted protein. In fact, the highest total protein concentration (about 90 ug/mL) was achieved in PB1.1+10 mM urea spikes after 168 h (OD_{730 ˜20}) of induction with 75 uM cumate. The supernatants from the two other PB1.1 media had about 10 to 20 ug/mL secreted proteins. Interestingly, all the cultures grown in PB1.1 secreted more protein when incubated with 75 uM cumate, indicating that the concentration of cumate used to induce A2352-Flag can make a difference in this media.

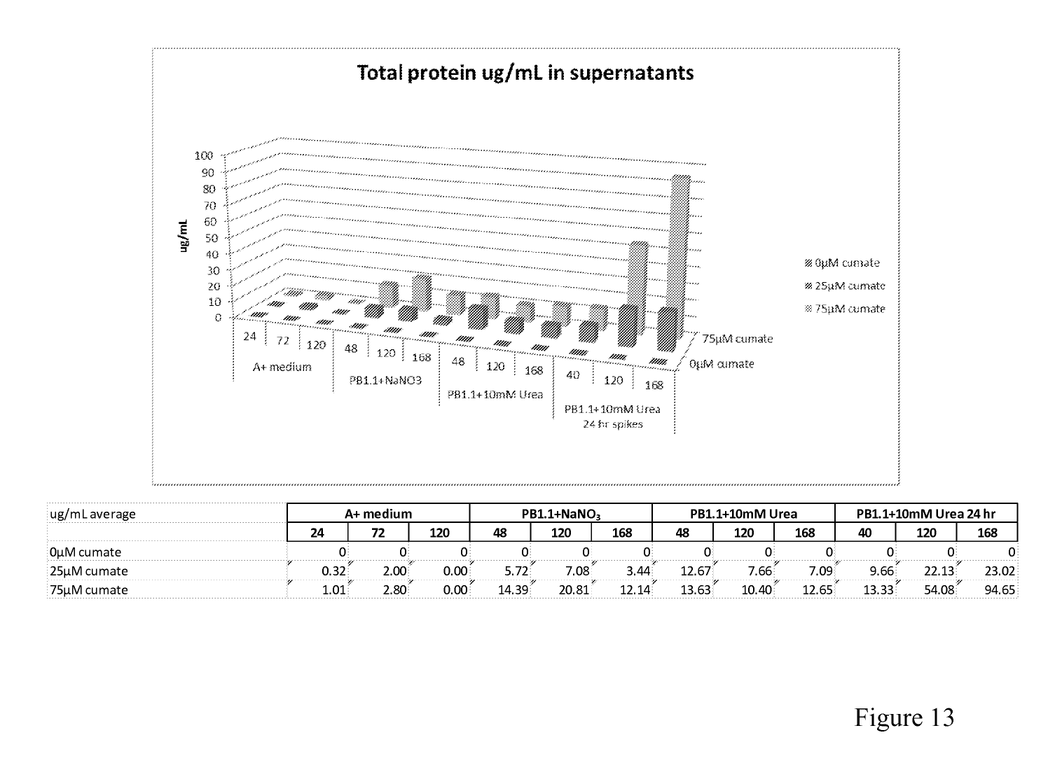

We observed that cells grown in PB1.1+urea spike grew slower but were still green after 8 days of cultures, while the others strains grew faster in PB1.1 (+/−urea) and were yellowish. This observation indicates that PB1.1+urea spikes medium was able to keep the strains healthy even if they were not dividing anymore. More importantly they were still secreting when they reached stationary phase (FIG. 13).

The profile of secreted proteins shows that in PB1.1 many other proteins are released in the supernatants in comparison with A+ medium. Caliper analysis have still estimated A2352-Flag as being 70% of the total amount of protein secreted (Caliper analysis) which gives a concentration of about 60 ug/mL of A2352-Flag secreted from PCC7002 after 8 days of growth in PB1.1+ urea spikes.

Thus, overexpression of A2352-Flag from an inducible promoter enhanced A2352-Flag secretion by 100× in A+ medium and by about 1000× in PB1.1+urea spike.

TABLE 4
NSP1 (SEQ ID NO: 1)
MKTNQLLTSVSRSTALAFLALTLGLGGEKALA
NSP2 (SEQ ID NO: 2)
MKSQNVFSTKSAKLIVGGTIFVSAITAANFTMLSAYA
NSP3 (SEQ ID NO: 3)
MLRLLFLHRKKAAQDFQGFTVIELMIVMIITGILTAIA
NSP4 (SEQ ID NO: 4)
MKNFTFKLLQQLNKKKADKGFTLIELLVVIIIIGILSAIA
NSP5 (SEQ ID NO: 5)
MSSYKAICVWLIHYSKRNNQGFTLIELLVVMIIIGILSA
NSP6 (SEQ ID NO: 6)
MINQPCIVPAEKGFTLIELLTGMLIVGILASISA
NSP7 (SEQ ID NO: 7)
MQLKKLFVPLLAGMLFLGGTSGAIA
NSP8 (SEQ ID NO: 8)
MQLKKLFVPLLAGMLFLGGTSGAIAEELLRTITVTGRGEEAIA

TABLE 5
SYNPCC7002_A1178 (SEQ ID NO: 9)
LESTVAQFTDISGDIYRNEIAQAVNVGFIAGFNDNTFRPTDVLTREQLVSMAIEGLQALPNASLAVPTQVANAPYPD
VAADRWSAAKITWAQANNIVSGYPDGTFQPTQPVTRAELLAVLRRTAEYAKAAQGQPMTLVATNGPIAFSDTAGHWA
NDLAAQMSTYCRVASPLNESGDRFFPDTASQRNYAAAATLRTLQCSVR*
SYNPCC7002_1634 (SEQ ID NO: 10)
LEVLAAPGLVDPLPYLPTFTDVQNHWAKPFIQAIANLGYIHGSAQGQFFPDQPLNRAQFALWIQAIFHPSPRRPRKQ
FFDVPSHLPAAEAIQQGYQGCFFSGFPDHTFQPQQPLRRVHLLVAIAQGLRLPPGDIALTEHYADQEEIPPYAQAAV
ATALQAKIGVLPQEKLMLKPQAIASRAEGLVYCHQALVYGQRLLPLTE*
SYNPCC7002_A2605 (SEQ ID NO: 11)
LELFSQGQVQALNSPYIVTQDVVAVDYRITAGTIIPISYTADKILLTQDEILPVTLTVDANIVNTQGIVLIPQGSEI
QGEFRPSGNGTRFVAQRLELPNGQMYNINAASQVITDTESVRRGTDVGNLLRNAALGTGAAAAIAAITGDRAIATEE
LLIGAGAGILATLIPQFLGLDRVDLLVVETNTDLDLTLANDLILQVNP*
SYNPCC7002_A2813 (SEQ ID NO: 12)
LESLGYLADEAADSTESNGLFNGEYGALAQIAFNLGDRAELGVTYVNSYHDSGAIYDFGGGSAVNGTAWANALGLFG
TEANSYGVQGKFDITDRISLAAYGMYTDAKVSGSSDEFDIWSYGLGVAFNDLGKEGNVLGLFAGAPPYLAEGDLKTP
LQVEGFYKYQLTDGISITPGVIWLKDAAQGVLGEEDAIIGTLRTTFTF*

TABLE 6
NSG1 (SEQ ID NO: 13)
ATGAAAACCAATCAGCTTTTAACATCCGTAAGTCGCTCTACTGCCCTGGCCTTTCTCGCACTCACCCTAGGACTTGG
GGGCGAAAAAGCACTGGCC
NSG2 (SEQ ID NO: 14)
ATGAAATCCCAGAACGTTTTTAGCACCAAATCTGCCAAGCTTATTGTTGGTGGTACGATCTTTGTTTCGGCCATTAC
CGCTGCCAACTTCACAATGCTGTCAGCCTACGCA
NSG3 (SEQ ID NO: 15)
ATGTTGCGTCTTCTCTTTCTCCATCGTAAGAAAGCAGCCCAAGATTTCCAAGGTTTCACCGTGATTGAACTCATGAT
TGTAATGATAATCACGGGCATCTTAACGGCGATCGCC
NSG4 (SEQ ID NO: 16)
ATGAAAAATTTCACTTTTAAGCTTCTGCAACAACTCAACAAGAAGAAAGCTGACAAAGGTTTTACCCTGATTGAACT
GCTCGTTGTAATCATCATCATCGGTATTCTGTCTGCTATCGCC
NSG5 (SEQ ID NO: 17)
ATGTCCAGTTACAAAGCGATTTGTGTTTGGTTAATACACTATAGTAAGAGAAATAATCAAGGATTTACCTTGATTGA
ATTACTCGTCGTTATGATTATCATTGGCATCTTATCAGCA
NSG6 (SEQ ID NO: 18)
ATGATTAATCAACCATGCATTGTTCCCGCTGAAAAAGGCTTTACGCTAATTGAACTCCTTACAGGGATGTTGATTGT
GGGGATTCTAGCTTCAATTTCAGCC
NSG7 (SEQ ID NO: 19)
ATGCAACTGAAAAAACTGTTTGTGCCACTGTTGGCGGGAATGTTGTTCCTGGGGGGAACCTCTGGGGCGATCGCC
NSG8 (SEQ ID NO: 20)
ATGCAACTGAAAAAACTGTTTGTGCCACTGTTGGCGGGAATGTTGTTCCTGGGGGGAACCTCTGGGGCGATCGCCGA
AGAACTATTGCGCACGATCACTGTCACGGGGCGCGGCGAAGAAGCCATTGCC

TABLE 7
SYNPCC7002_A1178 (SEQ ID NO: 21)
CTCGAGTCTACCGTGGCCCAATTTACCGATATTAGTGGGGATATCTACCGCAATGAAATTGCCCAGGCGGTTAACGT
GGGTTTTATCGCCGGGTTTAATGATAACACCTTTCGCCCCACCGATGTGCTCACCCGGGAACAACTCGTCAGTATGG
CCATTGAAGGCCTCCAGGCGCTGCCCAATGCCAGCCTCGCGGTCCCCACCCAAGTTGCCAACGCGCCCTATCCCGAT
GTGGCCGCGGATCGTTGGTCTGCCGCGAAAATTACCTGGGCCCAGGCGAATAACATCGTCAGTGGCTACCCCGATGG
TACCTTTCAACCCACCCAGCCCGTCACCCGCGCCGAACTGTTGGCGGTTCTGCGTCGGACCGCCGAATATGCGAAAG
CCGCGCAGGGTCAGCCCATGACCTTGGTCGCCACCAACGGTCCCATTGCGTTTTCCGATACCGCCGGGCATTGGGCG
AATGATTTGGCCGCGCAAATGAGCACCTATTGTCGCGTTGCCTCCCCCCTCAACGAAAGCGGCGATCGCTTTTTCCC
CGATACCGCCTCTCAACGTAATTACGCCGCGGCCGCGACCTTGCGTACCCTCCAGTGCAGTGTGCGGTAA
SYNPCC7002_1634 (SEQ ID NO: 22)
CTCGAGGTCCTCGCCGCGCCCGGTCTCGTTGATCCCCTGCCCTACTTGCCCACCTTTACCGATGTTCAAAATCACTG
GGCCAAACCCTTTATTCAGGCCATCGCGAACCTCGGCTATATTCATGGTTCCGCGCAAGGGCAGTTTTTCCCCGATC
AACCCTTGAATCGGGCCCAGTTTGCGCTCTGGATTCAAGCCATCTTTCACCCCTCCCCCCGTCGTCCCCGCAAACAA
TTTTTCGATGTGCCCAGCCATCTGCCCGCCGCGGAAGCCATTCAACAGGGTTACCAAGGGTGTTTCTTTAGTGGCTT
TCCCGATCACACCTTTCAGCCCCAACAGCCCCTGCGTCGTGTTCATCTGTTGGTGGCCATTGCGCAAGGTCTGCGTT
TGCCCCCCGGTGATATCGCCTTGACCGAACACTATGCGGATCAGGAAGAAATTCCCCCCTACGCCCAAGCCGCGGTC
GCCACCGCGCTGCAGGCGAAAATTGGTGTTTTGCCCCAAGAAAAACTCATGCTGAAACCCCAGGCCATCGCGTCCCG
GGCCGAAGGTCTCGTGTATTGCCATCAGGCGCTGGTCTACGGTCAACGTCTCCTGCCCCTCACCGAATAA
SYNPCC7002_A2605 (SEQ ID NO: 23)
CTCGAGCTCTTTTCTCAGGGTCAGGTGCAAGCCCTCAATAGTCCCTATATCGTGACCCAAGATGTTGTGGCCGTCGA
TTACCGGATTACCGCGGGGACCATTATCCCCATTTCTTACACCGCCGATAAAATCCTGTTGACCCAGGATGAAATTC
TGCCCGTGACCTTGACCGTCGATGCGAATATCGTTAACACCCAAGGCATTGTGCTCATCCCCCAGGGTTCCGAAATT
CAAGGGGAATTTCGCCCCAGCGGTAATGGGACCCGCTTTGTCGCCCAGCGGCTGGAATTGCCCAACGGTCAAATGTA
CAATATCAACGCCGCGTCCCAAGTTATTACCGATACCGAAAGCGTCCGTCGTGGTACCGATGTTGGTAATCTCCTGC
GTAACGCCGCGCTCGGTACCGGTGCCGCGGCCGCGATTGCCGCGATCACCGGTGATCGTGCCATCGCGACCGAAGAA
TTGCTCATTGGTGCCGGTGCGGGTATTCTCGCCACCCTGATCCCCCAGTTTCTCGGTCTGGATCGCGTGGATCTGTT
GGTCGTTGAAACCAATACCGATTTGGATCTCACCCTGGCCAATGATTTGATTCTCCAAGTCAACCCCTAA
SYNPCC7002_A2813 (SEQ ID NO: 24)
CTCGAGTCCTTGGGCTACCTCGCCGATGAAGCCGCGGATTCTACCGAAAGTAATGGTCTCTTTAACGGCGAATATGG
TGCCCTGGCGCAAATTGCGTTTAATCTCGGGGATCGGGCCGAACTGGGCGTTACCTATGTGAACTCCTACCATGATA
GCGGTGCGATCTATGATTTTGGTGGTGGGAGCGCCGTCAATGGTACCGCCTGGGCGAACGCCCTCGGTCTGTTTGGT
ACCGAAGCCAATTCCTACGGTGTTCAGGGGAAATTTGATATTACCGATCGCATCAGCCTCGCCGCGTATGGCATGTA
CACCGATGCGAAAGTTTCTGGTAGTTCCGATGAATTTGATATTTGGAGTTATGGTCTGGGGGTTGCCTTTAATGATT
TGGGCAAAGAGGGTAACGTGTTGGGTCTCTTTGCGGGTGCCCCCCCCTACCTGGCCGAAGGTGATCTCAAAACCCCC
CTGCAAGTGGAAGGCTTTTATAAATACCAGTTGACCGATGGTATTAGTATCACCCCCGGGGTGATTTGGTTGAAAGA
TGCCGCGCAAGGGGTCCTCGGCGAAGAAGATGCCATTATCGGCACCCTCCGCACCACCTTTACCTTTTAA

TABLE 8
Pcpc (SEQ ID NO: 25)
GTTATAAAATAAACTTAACAAATCTATACCCACCTGTAGAGAAGAGTCCCTGAATATCAAAATGGTGGGATAAAAAG
CTCAAAAAGGAAAGTAGGCTGTGGTTCCCTAGGCAACAGTCTTCCCTACCCCACTGGAAACTAAAAAAACGAGAAAA
GTTCGCACCGAACATCAATTGCATAATTTTAGCCCTAAAACATAAGCTGAACGAAACTGGTTGTCTTCCCTTCCCAA
TCCAGGACAATCTGAGAATCCCCTGCAACATTACTTAACAAAAAAGCAGGAATAAAATTAACAAGATGTAACAGACA
TAAGTCCCATCACCGTTGTATAAAGTTAACTGTGGGATTGCAAAAGCATTCAAGCCTAGGCGCTGAGCTGTTTGAGC
ATCCCGGTGGCCCTTGTCGCTGCCTCCGTGTTTCTCCCTGGATTTATTTAGGTAATATCTCTCATAAATCCCCGGGT
AGTTAACGAAAGTTAATGGAGATCAGTAACAATAACTCTAGGGTCATTACTTTGGACTCCCTCAGTTTATCCGGGGG
AATTGTGTTTAAGAAAATCCCAACTCATAAAGTCAAGTAGGAGATTAATTCC
Pcpc* (SEQ ID NO: 26)
CTGGCCACGAATTTTTGTAATTCCACGATGATCTTTCAACAATCCAGACACAGCCGTTGCCCCCGCCAGCAGAATAA
TGCGGGGATTGACCAAGCGAATCTGCTCTAATAAGTAGGGTATGCAGGCCGCTGCTTCAATGGGTGTTGGGACACGG
TTGCCAGGGGGACGGCACTTCACAATGTTGCAGATATAGGCATCCCGCTCGCTGTCGAGATTGACCGAAGCCAGGAT
TTTATCGAGGAGTTGCCCCGCTTTACCGACAAAGGGGCGGCCACTTTCATCCTCTGCTTGGCCTGGCCCTTCCCCAA
TAATCATCAGCTTGGCAGCAGGATTACCGCGGCTGACCACCACATGGGTACGGGTGGCCGCTAAACCACAGCGTTGA
CACTGCTGACAATGTACCGCAAGGGCCTCCAAGTTGCGGTAGGTGCCGGCGGGAATGGGCACCTCAGCCCGTAGCGG
AATTTGATCGTAGGTGGCAGGATCCAGGGGCGTCGCTGGTGCAGGTTCAGCTTCTGTGGGGCTGTCAAATAAGCTAA
ATTGCAACGGCTCACTCATACAAATCGTAACTTCCTGAGAACAATGTTAAAGAAACTTCACAAAAATTAGGAAAAAC
TTAGGACAAACTAGACCAATTTTATGGCGATCGCTAGAAGCTTAATTTATCTCACAAAAGTATTTTACAAATTAATA
ACTACGGCGAAACAGGTTTCCCACCGCATTGTATAAGAAATACCTGAAGGGTTTAACAACACGGCTGTTGTTTCCCA
GGCCCCTCTGCGGAACAAGCCATCAGCAATCGTTAGGCCTTTCCGGCACGCCAAGAGCGTTGCACGTTTCTTAAAAG
ACACACCAAGGATCAGCTTGGTCGCTCTCGGGTTGCTTGGCACAGCCTTTAGGGAGTTGCTGATTAGCCTCCCTAAA
AATCCTGCCTTATCTCTGTGGGTAGGAATGTCAAGAAGGTCTCACTTCTTTAATAACCTTTAAGGAGAATTGATCC
Psuf (SEQ ID NO: 27)
TTGGCTAGGAAATACTGCTCAAAGGCGGACTTGAGCCAACTGACCCATGGCCCTTGCACGGCCAGAGCTATAGCTGC
TCTTGCCTGTCTTCCGAAGCCGCCACCTACTGCTACTCGTGGCTGGGTCTAGGAGGGGATCCCATCCTGGCAATCCA
GTAGCCGCACTGGTGTTCTCCGTTCACCTGCCAGGTGAGGCGCTCTACCCGGCAGTCGGGCAGGGCGGCGGCAAACA
TCTCCAGCTCGTGGCCGCAGACGCTGGGAAAACGCTGGGCAATTTGGGCAATGGCGCAGTGGTGCTCCACCAATACG
TATTGCTCGGCATACTGCTCAGCGCCTTCTGTTGCCCTGGCCGCAGCCAGGTGGTTGGGGTAGGGGTAGTACTCCGC
CATGTAGCCTTCTGCCTGGCGCAACTGCACCAAGCGCTCCAGCCGCTTGGCCAGGGATCCGCATCCCATCTGGGCTT
GGTACTCTTGGGCTTTGCGCTGCCACTGCTGCTGGAGCAGGGATCCCATCTGTTCGGATCCCAGGGTTTCGGCCAGC
GTATTGAGCAGGCCCAGGGCAAACTCGTCGTAGCTTGTGGGGAATTGCGCTTCCCCTTGGGGGCTAAGCTGATAGAA
GTGCTGGGGGCGGCCCAGGCCACTGGCTTGAGCGACGTGGTGGATCAGCCCCTCCGCCTCTAGATCCTTGAGGTGGC
GGCGAATGGCTTGGGGAGAAATGCCCAAGTGCTCTGCCAGAGTCTGGGCGGTGGCCTGCCCCGCCTTGCGCAAATAG
ACTAGGATGGCCTGCTTGCTGGAAAGGGGCTGCGTGAACCGCCCGGTGATCTTGTGCGCGCTCTCCACCCTCGCCCT
CCTGGTTAGACTGGGTTCACTCGGCTGCCGCTGCCAGCACAGCTACTTTGACAACGGGATCATTGCTACAGTAACCT
GAGGGTTAGTTAAGCAACAACCGTGTTGTTTTAGTTGGCCGTCTGTCTTGACCCTGTCCCTAACCAAGAGCCATCC
Prbc (SEQ ID NO: 28)
GTCATCGCAAATGGCCAGTTTTACCGCCGCCTCTTGATTCTTGAAATTCATGGGCAACACCCCTGGGGCATCCAAGA
GTTCAAGGACGGGGGACAAGCGCACCCAGCGCAGTTGCCGCGTCACCCCCGGACGCGCTGCACTCTCCACCACCCGC
TGCTGCAAGAGGCGATTAATGAGGGCTGATTTGCCCACATTGGGAAACCCTAAAACCACAGCTCGTACGGCTCTTAC
TTGCATGCCCCGCTGTTGACGGCGTTGATTAATGGCCGCTCCCGCCCGTACCGCCGCCTGTTCGAGTCGGCGAATCC
CTTCACCCCGCTGTGCATTGGTGAAAAAAACCGTTTCCCCTTGGGTCTCAAACCATGTCAGCCACTGCTGGCGATCG
CGATCGCTAATCCTATCCATTCCATTGAGCACTAAGAGGCGCTGCTTGCCACTCGCCCACTGACGAATTTGGGGATG
GCAACTGGCAAGGGGAATGCGTGCATCCCGCACCTCAAAGATCACATCCACCTGTTTCAATTGTTCCTTGAGGGCAC
GTTCTGCCTTGGCAATGTGGCCAGGATACCATTGAATGATCGGACTCATAGAAATTCCACCCCCTTTTTTTCAAAAA
CAATAGAGCGATCGCCCCCCATGAGGACCATTAAATTGATTTCAGGCAATCATCAGTGCCTGTTGAATAGAAGGGAG
ACTATAGTTGTACACCTCTAATTAGAAACCTATAAACAACTCTAAAGATTGATAACCCAAACCTATAGAATAATTAA
GAATCTCCAAAGCAACAGTTATTGCATTGGGTATCAACTAAGCTGATCCTAAGCATAAGACCTACGACAATATCACA
GGCTTGCGGATGGTTGCATCATCTCGCTGAGTTTTGCACTGCTCAATTTTGCCCCATTTGCGGGGAATCGTAGCGTT
CACACTACCCATTGCAAAGGTTGCCCGTAGAGATTGCTCTATTCGGCACAGTCACTGTTAAAGAGGAACAACGTCT
Ppsa (SEQ ID NO: 29)
TAGCGCAAGGCGCCCTGGTAGCGAGCAATTTGGCCTTCCAGGCCGTGGTGGAACATGAGCACCACCCGCTCAGGGCC
AGGGGGCAACCGGCGAATGGCCGCCGCCAACTGCTCAATCGACTTGGGGGCCGCCGACCCATACCACTGGGATCCGA
TCACCCGCACCCCACAGGGCAGATCCAGATAGCCCCCCCGCCCACCTGACCAGGGGCGCAGCTCTAAACCCTCCTCG
CCTGCTTCCGGCTCCAGCAGGATCAGGTCGCCGTGATCGGCCAGGTAGCGCAGCCAACTGGTCTTGACACCGTAGGG
GCGGCTGTCGTGGTTGCCCTCAATGGCCAAGACGGGGATCCCGGCCTGCTTCAGCTCCCGCAAGACAATCTGGGCTT
GGTTGAGGACACCGGGCTGAATTTGCCGGTGCTCAAACAGATCCCCGGCAATGAGGACAAAATCCACCGGGTTCTGG
ATGGCATGGCGGCGCACCACATCCCGAAAGGCCAAAAAGAAATCTTTGCTCCGCTCCGGGCTGTTGTAGCGGTCGTA
GCCCAGATGCACATCGGCCAGGTGCAAAAAGGTGCAGGTGGAAGTGGCCATCGCCCGAACCTGCCAGCAAATTGCCC
ACAGTTTAGCAGACAAACCTCAGTGCTCAACTAGATATAGGGATCCCTTCCCGGCCATTTGCGGCTTTGCTCGGGTT
GCCACCCCTAGAGCTAGGCGCCGCCCCGCCGCGCTGCCTGTTGCCCAGTTCCAACCTTTCATGGGCACAGCTTGGGT
TGGTGAAAGTTCGTTACATATATTTACATCTTTATTGGAGAAACGATTGCCAAGCAACCCTAACTCCGATAGGGCAA
GGGATCCCTGGTTTAATTATTGTGAAGCGACGGGGGGGTTACAAAGGCTGACCTATAATGCCAGGTAAATCCCGCCT
TGGGAGAGATCCCCGAGCTTCACGGCTGCAGACAGGCGGGAGCGCTTCGTTCCTTGTCGCAAGAGAGGAGTTCTTG
PpsbAII (SEQ ID NO: 30)
GCAGTCGTCATGATGTTTTGAGTCCAGTGAATTTTTATGTATGTCTAAGGCGTAATGCCTTATGAGCTAATAATAAC
AAAACTTTGCGAATTGTGAAGCACTTCTCAGATCAAACTTGGCGATCGCCCCAACAATCAGCTGTGATCACCTACAG
TCCGGCCTATACCCTCGTTCCCACCTACGAGTGCTTCAATCGCTGCACCTACTGCAACTTCCGGCGCGATCCCGGAA
TGGACGA
Pnir (SEQ ID NO: 31)
GTGGTTTTGTAGCTGGGTCTTTCTGCACTTTACCATCAACTCCATATTCTGCGCCATGACATGGGCAGACAAATTTC
TTCGCTTGGGCTTGCCATGCTACGGTACAGCCTTTGTGGCTACAAGTAGGATTGACAGCAATCAGATTTGCGTCCTT
AGATGTACCCACGACCAACACCGGGCCAATTGGTGAATTTTCGTTGAGTAATTGACCAGTTTTATCTAGTTCAGCAA
CAGTCCCGATCGCTTGCCCCTCTGTAGATGTTGTGGGCTGGGAAGAACAAGCAGCGATCGCTACAGGTAAGCTACTT
GCTATCCAACCCAAACCTACCCAATTGATGAAATCCCGACGTTTCATAGCCACTGAAGTTATGTATTAGTTGTAAAC
AAAAGTCTAGCCTTGTTTTACCAACATTTTTAGCTACTCATTAGTTAAGTGTAATGCAGAAAACGCATATTCTCTAT
TAAACTTACGCATTAATACGAGAATTTTGTAGCTACTTATACTATTTTACCTGAGATCCCGACATAACCTTAGAAGT
ATCGAAATCGTTACATAAACATTCACACAAACCACTTGACAAATTTAGCCAATGTAAAAGACTACAGTTTCTCCCCG
GTTTAGTTCTAGAGTTACCTTCAGTGAAACATCGGCGGCGTGTCAGTCATTGAAGTAGCATAAATCAATTCAAAATA
CCCTGCGGGAAGGCTGCGCCAACAAAATTAAATATTTGGTTTTTCACTATTAGAGCATCGATTCATTAATCAAAAAC
CTTACCCCCCAGCCCCCTTCCCTTGTAGGGAAGTGGGAGCCAAACTCCCCTCTCCGCGTCGGAGCGAAAAGTCTGAG
CGGAGGTTTCCTCCGAACAGAACTTTTAAAGAGAGAGGGGTTGGGGGAGAGGTTCTTTCAAGATTACTAAATTGCTA
TCACTAGACCTCGTAGAACTAGCAAAGACTACGGGTGGATTGATCTTGAGCAAAAAAACTTTATGAGAACCAGCTC
P-cro v1 (SEQ ID NO: 32)
AATTCTCGAGTAACACCGTGCGTGTTGACTATTTTACCTCTGGCGGTGATAATGGTTGCAGGATCCTTTTGCTGGAG
GAAAACC
P-cro v2 (SEQ ID NO: 33)
AATTCTCGAGTAACACCGTGCGTGTTGACTATTTTACCTCTGGCGGTGATAATGGTTGCAGGATCCTTTTAGTGGAG
GTAAACC
P-trc v1 (SEQ ID NO: 34)
AATTCTTGACAATTAATCATCCGGCTCGTATAATGTGTGGAATTGTGAGCGGATAACAATTTCACACAGGAAACAGA
CCC
P-trc v2 (SEQ ID NO: 35)
AATTCTTGACAATTAATCATCCGGCTCGTATAATGTGTGGAATTGTGAGCGGATAACAATTTCATAGTGGAGGTAGA
CCC
P-RBS op (SEQ ID NO: 36)
AATTCGTGCAGGTTCAGCTTCTGTGGGGCTGTCAAATAAGCTAAATTGCAACGGCTCACTCATACAAATCGTAACTT
CCTGAGAACAATGTTAAAGAAACTTCACAAAAATTAGGAAAAACTTAGGACAAACTAGACCAATTTTATGGCGATCG
CTAGAAGCTTAATTTATCTCACAAAAGTATTTTACAAATTAATAACTACGGCGAAACAGGTTTCCCACCGCATTGTA
TAAGAAATACCTGAAGGGTTTAACAACACGGCTGTTGTTTCCCAGGCCCCTCTGCGGAACAAGCCATCAGCAATCGT
TAGGCCTTTCCGGCACGCCAAGAGCGTTGCACGTTTCTTAAAAGACACACCAAGGATCAGCTTGGTCGCTCTCGGGT
TGCTTGGCACAGCCTTTAGGGAGTTGCTGATTAGCCTCCCTAAAAATCCTGCCTTATCTCTGTGGGTAGGAATGTCA
AGAAGGTCTCACTTCTTTAATAACCTTAGTGGAGGTTTGACC
P-S65 (SEQ ID NO: 37)
AATTCGTGCAGGTTCAGCTTCTGTGGGGCTGTCAAATAAGCTAAATTGCAACGGCTCACTCATACAAATCGTAACTT
CCTGAGAACAATGTTAAAGAAACTTCACAAAAATTAGGAAAAACTTAGGACAAACTAGACCAATTTTATGGCGATCG
CTAGAAGCTTAATTTATCTCACAAAAGTATTTTACAAATTAATAACTACGGCGAAACAGGTTTCCCACCGCATTGTA
TAAGAAATACCTGAAGGGTTTAACAACACGGCTGTTGTTTCCCAGGCCCCTCTGCGGAACAAGCCATCAGCAATCGT
TAGGCCTTTCCGGCACGCCTTTAATAACCTTTAAGGAGAATTGATCCATGGCCATCA
P-S115 (SEQ ID NO: 38)
AATTCGTGCAGGTTCAGCTTCTGTGGGGCTGTCAAATAAGCTAAATTGCAACGGCTCACTCATACAAATCGTAACTT
CCTGAGAACAATGTTAAAGAAACTTCACAAAAATTAGGAAAAACTTAGGACAAACTAGACCAATTTTATGGCGATCG
CTAGAAGCTTAATTTATCTCACAAAAGTATTTTACAAATTAATAACTACGGCGAAACAGGTTTCCCACCGCATTGTA
TAAGAAATACCTGAAGGGTTTAACAACACGGCTGTTGTTTCCCAGGCCCCTCTGCGGAACAAGCCATCAGCAATCGT
TAGGCCTTTCCGGCACGCCAAGAGCGTTGCACGTTTCTTAAAAGACACACCAAGGATCAGCTTGGTCGCTTTAATAA
CCT TTAAGGAGAATTGATCCATGGCCATCA
PisiA (SEQ ID NO: 39)
TTGGGCGATCGCCAAAAATCAGCATATATACACCAATTCTAAATAAGATCTTTTACACCGCTACTGCAATCAACCTC
ATCAACAAAATTCCCCTCTAGCATCCCTGGAGGCAAATCCTCACCTGGCCATGGGTTCAACCCTGCTTAACATTTCT
TAATAATTTTAGTTGCTATAAATTCTCATTTATGCCCCTATAATAATTCGGGAGTAAGTGCTAAAGATTCTCAACTG
CTCCATCAGTGGTTTGAGCTTAGTCCTAGGGAAAGATTGGCGATCGCCGTTGTGGTTAAGCCAGAATAGGTCTCGGG
TGGACAGAGAACGCTTTATTCTTTGCCTCCATGGCGGCATCCCACCTAGGTTTCTCGGCACTTATTGCCATAATTTA
TTATTTGTCGTCTCAATTAAGGAGGCAATTCTGTG
PnirA (SEQ ID NO: 40)
CTAAATGCGTAAACTGCATATGCCTTCGCTGAGTGTAATTTACGTTACAAATTTTAACGAAACGGGAACCCTATATT
GATCTCTAC
PnrsRS (SEQ ID NO: 41)
CATCGCCTCTGCCTTTTTTATAACGGTCTGATCTTAGCGGGGGAAGGAGATTTTCACCTGAATTTCATACCCCCTTT
GGCAGACTGGGAAAATCTTGGACAAATTCCCAATTTGAGGTGGTGTG
PpetE (SEQ ID NO: 42)
ATCGCCTTTTTGGGCACGGAGTAGGGCGTTACCCCGGCCCGTTCAACCACAAGTCCCTATAGATACAATCGCCAAGA
AGT

TABLE 9
Gene 1 (SG2): SYNPCC7002_A2594 (SEQ ID NO: 67)
ATGAAATCCCAGAACGTTTTTAGCACCAAATCTGCCAAGCTTATTGTTGGTGGTACGATCTTTGTTTCGGCCATTAC
CGCTGCCAACTTCACAATGCTGTCAGCCTACGCAGTTGATGACACCGCTTCTTTTTCGGGTACGGTCGCTCCAGCTT
GTGCACTCTCCAACGATGATGGTGCAGTAGCATTTGATGCCGGCGACAGAACTTATACAGCCACAGGTAGTGGCGTA
GATGTCACTGAGCTTTCTGAAACTCAGTATGTTGATTTTGAATGTAATACCGACACTGCTACTGTTGCGATCGCTGC
ACCTGTTACTTCAAAACCAATGGCTCCTACAAATGCAAGTGGCTTAGTTGCCACTCATGTTGCTAAATATGCGGTAG
ACGATACTGATACTCTTGTAAATCCAGATCCAACGTCTGGTACGATCATTAATGAGGCTACTGGCGTTGCTGGATTT
TCTCAAGCAGTAAATGCAACTGGCTTATTTAGAGTGGGTGTTGAATCTAAATGGAGCGGAGCTAATGGAATGTTAGC
CGGGGACTATTCTGCTGATATCACTGTAACAGTGACTCCTAACTAA
Gene 2: SYNPCC7002_A2595 (SEQ ID NO: 43)
ATGAATTCTCAAGCCGTTCCATCTCCCAAGTGGTGGTTTCAGATCATCTTCCTCTCTCTGTTTTTGGGGGGACTCCA
AACAAAGCAAGCCTCCGCCCAAACCCCAGGATGCTTTACGACGAATGTCCCCTCTTCCCCTCTCAGCTACGATGTCA
CCAGCACAACCCAAACCGAAAGCTACGCCGTGACATTTCGCTGTACCGATGATGGCACGACCGGAGGAAGCAACCTC
AGCAATGTTGATCTAGATGTGACGCTACTGCCGCTCACTGCACCAACCGCTGGCCCGGCTAATCTGGATCTCGGTTC
TCCGAATGGTGTTACTCATACGATTTCGATTGGTTCGGGTGGTTCCTTTACGAATCTGGTCGATACTCAAACCACCG
TAAATAACAGTGGCTCAACCAATTTAGTCGTGTCAACTGCTGGTGGTAAAGGCGAAAATCTCTTCCTCGATGGTACC
GGAACGATCACGGTGAATATCCAATCCCGCTTTGCACTCCAGGGGAGCACCTCCGAATTTGCCGCTGGCACCTACAC
CACCCAGTTTGAAGTTGATGTAACCCCAGTTGGTGGGGGCACCACTGCTGATGAAACTACCACAATCAGTAGTACGG
TCAGCCCCAGTTGCGTCCTCGATAATGTGATTCGCTTCCGGGAAACAGCCACCCCCTATATCAAAACAGGCAGTGAA
CCCAATGTTTCCCAGCTTCAAGCCAGCGATACAGCGAAGTTTGACTGTAATGCCACCACCGTCGATATCAACTTTAG
TGCAGACAGCGCTACCTACACGCCGCCAACAGGGGGGGCAACCAACCTGACCGCAACCCATCAATTCGCCTATGAAC
TCAATGGCAACGGCTTCAACAATTACAGTGGCCCAGAGCTTATTGAAAACCAAAATACAGATGACAATGGTGATGCA
ACCTTAACGATTCGCTCCACCTGGACGCCGAATAGTGATCAACTGTTCGCTTCAGAATACAACGCCCAAACCACTGT
CACCATTACGGCTAAATAA
Gene 3: SYNPCC7002_A2596 (SEQ ID NO: 44)
ATGGCTTATTCTGTTGTGTCTTGGCGCAAAAACCTTAGCTGGGCGCTCTGTTCTTTGGCTTTACTTTTGCCACTCCC
CCTCAACGCCCAGGTGCAAGTCTCTCCCATGGTGATCAAAACAGAAACCAGCCAGGGGATGGCGAATGGGGTGATCA
GTCTAACAAACCAGGGAACCCAATCCCAGCGGGTACGCCTCTCGGCGGAATCTTTTACCTATACTCGAACTGGTTTT
GCCACCGCAGAGTCCGATCCCTATGACCTCAGTCCTTATTTGATGTTTTCCCCTAGGGAGTTGGTTCTAGAACCCGG
CCAAACGAGACGAGTGCGACTGATTACGCGAATGTTGCCTTCGACGGCAAATGGTGAATATCGGTCGGTGATCTTTG
CTGAACCCCTGCGAGAACGAGATGAAGCGGGGGGCGGTTTGAGTATTCGGGCCCGTGTGGGGGTGACAGTTTACGTG
AAACATGGCCAGGTCAATTTTGCCTTGACTCCCGTTGAGGCGAGCTACGATCCAACGAAACAAGAATTTCAACTTTT
GGTGAGTAACCCCAGCAATGGTACGGTGCAATCAAAAGGCACCTGGACATTGTCACAAAATGATCAGCCTTTGCTCC
AGGCAGATATTGATCAACGTACTGTAATTGCCGGAGGCGATCGCCTTTTCCCCCTAGAGCTGCCCCCAGACCGGACT
AACTTACCAGCGGGAACCTATCAAGTAGCAGGGCAGTTGCAATGGAGCGAATCTGGAGCAGTGACCACAACACCATT
TTCCTTTGATGTCACGGTGCCTGCCGCACGGTAG
Gene 4: SYNPCC7002_A2597 (SEQ ID NO: 45)
GTGGCGCACTCTAACCTGAAAAAGTCTCACATTTTTCCCCGTCGTTTAGAGTATTTACCCTTGACCTTTCGGCTACT
ACTATTCAGCTTTTTCATGCTTTTCCTATTGGGTGCTGAGGTTGTTGATGCCCAACAGGACAGCGAGCCTGCTGATA
ATGGTGCAACGGAAACCACGTCGGAGACTTTCCCTGCATCCTTTGATTTGATTCCAGTGGGGATTAAGCTTGGCGAT
CGCACGGCCAATCCTGGCACCTTGGTTCGGGGTTCAGAAAATGGCATTCAAGCTATTGATTTTTCCAACTGGGCGAT
CGCCTACAATGATGTCCTCAAAGCGCTCCAATTTACGGCAACCCCCCTTGCCGATGGCACCATAGAGTTGCGGTCTC
CGGCGGCAGTCATCAGGCTCGATCCCAGCCTTCTCGATACAGATCCACAATTGGGCTTGGTATTCACCGTCACCCAG
ATCCGCGATCTGCTACAAATTCCGGTGGAGTTTGATATTTCTGAATATGCTATTGTTCTGACCCCTGAGTGGCTGAG
GGCATCAGGTTCTTTGGGATTAACTGGGCGATATTCCCTCCCGGAGCGGCCCATTGTGTTGGAGGGTTTACCCCGCA
TTGAAGCTCCGAATTTGTCTTTTAGTGCCATTGGTCAAGAAGTTCGTGTGACAGGAGGAGGCGATCGCCCCACAGAA
TACGAAGGCGATCTGGTTGGCATCGGGACATTTTTTGGGGGAAGTTGGTACAGCAAGATTGATCAACGCGATTTAAC
CGATCCCCGCAGTTGGCAACTCGAAGAATTTCAATACCTACGCCAAACCCCTAGCACCGACTATGTCATCGGCGATC
AGCGTACCTTTTGGCCAGAGGGCAGTGGTCGCTATACAGGTGTCAGTATCGTGCGCCGCTTTGGCTTTCAACCTCCC
ACCGAATTTACCAATGCCAGCGATGGCTTTAATCCCCAACAACGTCTCAATAGCGATCGCCTAGAGCGTGATATCCG
AGGCCGCGCCGAACCAGGGACCCTCGTTCAACTGGTCAATAAAAATGGCAATTTAATTGTTGGGGAACAACTCGTTG
ATCAGTCTGGCATCTATCGCTTTGAAAATATTCCCAGTGCTTCTACCAATAAAGGCAGAGGCGGCATAGCCGGTAAT
CGCTACGAACTTCGACTTTATCCCAATGGTCAACTGAGTGCCTTCCCAGAAATTCGGGCCGCTGAATTTTCTTCTCT
GCCTGGGCAGCTGAGTAAAGGTACCTCAGCCCTCCTCCTCTCTGCTGGCTTTGAACGGCTTCGACAAGCGGATACTT
TTTTTGGCTCCCTCTCGAATGATCTCCAGGGAGGATTCGCCTACCGTTGGGGCGCCACGGACAATCTCACCCTCGGT
ACGGGTCTTTTTTACGACGGTCAACTTAAAGGTCTAGGGGAATTTTTCTTTCAGCCCGGTCGATTGCCCCTGCGAAT
TACTGGGGCAGCAACCTTTAATAGCGACGAACAACGGGGAGAACAACAATCTGATTTCCGCTACGATCTAAATGTCC
GCTTCAATCCAGGCCAGAGGTTTGATTTTGAGTTTGACAAAGATGAACTGTCTGAGCGCATTCGCACCCGCTGGGAT
GTCAGTGACAAATTTCGTCTTGCCTTCAACAGTAACAGCAGCGATCAAATCGCCCAGGCCACTTGGCGGCTTTTTCC
GGGTTTTAGTACGCGGGTTGGTTGGAGCTTTAACAATAAAGCCCTGGAAGGTGGATTCGACCTCAGTGGTGCCCTTG
GGGATCTTTTAATTCGCAATAGCGTAACCTTTAGTGCCGACCAAAGCCTTGATTGGCGATTGTTTTCCCGCTATCAA
AACCTCACCCTAGACCACCGGCTGCGTGACCGTCAGATTGCAACGGAAGTAGAGTATTTTTTCCGTAATCCTGAAGC
CCTGGTGGATACGGGTCACTCGGTCTTTGCGCGCTACCAAAGTAGCCCCAACGAGGACAACGAGGCCCGGACGAACG
AGCTGCTCGTGGCAGGGTGGCGCTATGAGGCAAATTCGACGGTGGGCGATCGCCTTTCCGACTGGATCGTCGATCTT
GGCTATGGCGTTGGCACCCAGGGAGCAGGATGGCAAATTGCTGTAACCACCAATCAGCTCTTGGGCCTGAATCTAAC
CGCGCGGTACCAAGATATTTCTCTTACGGGTAATGAGTCAAGCTTCAGCCTCCTCATTGGTTCCGATGCAATCCTTT
CACCTAATTTCAGCCTAAAACCCAGTCGCTTTGAACGTTTACGAACAGAGGGTGGCATTGTGGTGATCCCTTTTATC
GATGCCAATCGGAATGGTGTCCAAGATGAAACAGAAACGGCCTATTTGCAAGGGATTGAGGCGGAAACCGCAGACTT
TTTATTCTTGATTAACGAACAGCCCATTAACCGCTTTAGTGAATATGAGCCGGATTTGCGACGGAGAGGAATCTTTG
TGCGACTGCCACCGGATACCTATCGCTTCGATGTAGATCCGGCGGGCTTGCCCCTGGGCTGGCAGACAACGCAGTCG
GCCTTTGCAGTAGAAGTGAGTGCTGGTAGTTACACGCCTATTTATGTGCCCCTTACCCGTGCCTACATTGTCGCGGG
CACGGTGGTCAATGCCCAAGGGAAACCACTGGGTGGGGTAAGGGTCGAAGCAGTCAACCAAAACAACCCCCAGGAGC
GATCATTGTCGGTGACCAATGGCGCAGGTATCTACTATCTAGAATCCGTAGGAACTGGTGTCTATGACCTATTCATC
GATGGCAAACCCGCTAAACCGGGCCAGCTCCGCATTGAGATAGATGCTGAAGAATTTACAGAATTGGATTTGCGCCT
GTAA

TABLE 10
Gene 1: SG8 (SEQ ID NO: 73)
ATGCAACTGAAAAAACTGTTTGTGCCACTGTTGGCGGGAATGTTGTTCCTGGGGGGAACCTCTGGGGCGATCGCCGA
AGAACTATTGCGCACGATCACTGTCACGGGGCGCGGCGAAGAAGCCATTGCCACGAGTCTTTCTGAAGTACGCCTTG
GGGTCGAGGTGCGGGGGGCGACGGCAACCCAAGTCCAGGCAGATATCGCCAAGCGCAGTAACCAAGTGGTGGATTTT
CTCAAGTCCAAAAATGTGGCCAAGCTCACCACCACGGGCATTAACCTCCAGCCGGAATATGACTACAACAATGGCGA
TCGCCGCCTCATCGGTTATCTCGCTACCAATACAGTGAGCTTTGAGGTGCCCACCGCCCAAGCCGGGAGCCTGATGG
ATGAAGCTGTCAAAGCCGGAGCAACCCGCATTGATGGGATTTCTTTCCGAGCCACCGAAGCCGCCCTCACTGAAGCA
GAAAAAACTGCCCTCGCTGAAGCCGCCCAGGATGCGCGCACCCAGGCCCAAACTGTCCTCGGTGCCTTGGGTTTGAG
TCCCCAAGAAATTGTCCAAATCCAGGTCAATGGGGCGACGCCGCCAACCCCCATTTTTAAAACCATGGATACGGCAC
GAATCGCCCTTGAAAGTGCAGCACCTTCTCCGGTAGAAGGGGGTGAACAGACGGTGAATGCTTCCGTAACCCTGACG
ATCCGTTACTAA
Gene 2: (SEQ ID NO: 46)
ATGTTAGATCTAATCAAACTTGCGGGACAACTGCCAGACATGGGGGCGCACCTCCAGGAACAGGCTGTCACGGGACG
AGAACGAATCGAGCGGGGAATTTCTCTGCTCCGGGAAGCCCAGGCGGATTTCCAGACCCTCCAGGCCCACCAAAATA
CCTGGGGCGATCGCCTCATTTTTAACCATGGCATTCCCCTCGAACCCCTGGAGACTCGCGTTCCCATTTCGCCCCCT
TCCCAAGCCCACACCGTTTTTGCCACGGATGGCTCCCAAATTGCTCCGTCTCACCATGAAATTGCCTATTGTTATTT
GATTAATATTGGTCGAGTGATGCTCCACTACGGCCAAAGCTTGCACCCATTGCTGGATCATCTGCCGGAGATTTTCT
ATCGCAGCGAAGATCTGTACACCTCCCGCAAATGGGGCATCCGCACCGATGAATGGCTCGGTTATCGCCGCACCGCC
TCCGAAGCTGAAGTGCTCGCTGAGATGGCCTGTAAATGGGTGTTACCCCCCGGTGCCCACGGTCATATTCCCAATGT
GGCGATGGTGGATGGCTCTCTGGTCTATTGGTTTTTAGAAAATTTGCCCGCCGAAGCCCGCCAACAAATTCTCGAAC
CCCTCCTAGGGGCCTGGCAACAACTCCGAGAAACCCGTATTCCGCTGATTGGCTACATTAGTTCCACCCGCAGTGTA
GAGGCGGTTCATTTCCTGCGGCTCCAGGCTTGCCCCCACGACAAACCCGATTGTCAAAGCCATTGCCTCGACGGCGA
AACCAAGGAACGTAAAGCAGAATTTCGCGAAACTCTTCCCTGCCAAACCATTGAACCGTTGCGGGATAGCACTCTTT
TTGAGCAACTGTTGCAACCGGGCGATCGCAGTGGGCTTTGGCTCAGTCAGGCACGCATTTTAAATCATTATCCAGAA
GCGGATCAGGTTTGTTTTTGTTATCTCCATGTGGGGACGGAGGTGGCGCGGATCGAGATGCCCCGCTGGGTCGCGGC
AGATCCTCAACTCCTCGATCAAACCCTAGGCATTGTCCTCGGCCAAGTGCAAAAGGGGTTTGGGTATCCCGTGGCGA
TCGCCGAAGCCCATAATCAAGCTGTGATCCGGGGTGGCGATCGCGCCCGATTTTTTGCGCTCCTCGAACAACAACTC
CTCAAAGCAGGGTTAACCAACGTAGGTATCTCTTACAAAGAAACCCGCAAACGGGGTTCCGTGGCTTAA
Gene 3: (SEQ ID NO: 47)
ATGCCCGAAATGCCCGAAAACTCTCAATTTCCCGTTGAACCGCCCCAGAAACCCAGTGGCACGGAGCAACAGCATGA
AGAAAATCCCTGGGTAGAGACCATCAAGACCCTTGTGACCGCTGGTATTTTGGCCATTGGGATCCGCACTTTCGTCG
CCGAGGCCCGCTACATTCCCTCCGAGTCGATGCTGCCGACCCTAGAAGTGAACGATCGCCTAATCATTGAAAAAATC
AGCTATCACTTCAAAAATCCCCAACGGGGAGATGTGGTGGTCTTTAACCCGACAGAAATTCTCCAGCAGCAAAACTA
TCGGGATGCTTTTATTAAGCGGGTGATCGGGATTCCCGGGGATACCGTACAAGTCAGCGGCGGCACCGTTTTTATCA
ATGGGGAAGCCCTCGAAGAAGACTATATCAACGAAGCCCCAGAATATGACTACGGCCCCGTGACGATTCCAGAAGAT
CACTACCTCGTCCTTGGCGATAACCGCAACAATAGCTATGATTCCCACTATTGGGGTTTTGTCCCCCGTGAAAAGCT
TGTGGGGAAAGCCTTTATTCGTTTTTGGCCCTTTAATCGCGTGGGCATCCTCAACGAAGAGCCGCAATTTGCCGACG
AAGAACCGATTACACCCTAG
Gene 4: (SEQ ID NO: 48)
ATGAGTGAACCATCCCCTTTGCTGCAAGCATCAGGTCTCCATAAAAGTTTCGGTGGCATCCGTGCGGTGCAAAATGC
TTCGATTACGGTGCCCCGCGGACAGATTACGGGGTTGATTGGCCCCAATGGGGCGGGCAAAACGACGTTGTTTAATT
TGCTCTCGAATTTTATTACGCCGGATCGGGGGACAGTTATTTTTAACGGCCAGGAAGTGCAGCATTTACCGTCTCAC
CAGATTGCGGCACGGGGTTTTGTGCGTACCTTCCAAGTGGCACGGGTGTTATCGCGGTTATCGGTACTAGACAATAT
GTTGCTGGCGGCCCAACAGCAAACGGGGGAAAACTTCCTGCGGGTGTGGCAACAGGGGAAAATTCGTCGCCAAGAAA
AGGCAAATCGGGAAAAGGCGATCGCCATCTTAGAATCCGTCGGTCTAGGGAAAAAAGCCCAGGATTACGCTGGTGCC
CTGTCGGGGGGACAACGCAAACTCCTGGAAATGGCCAGGGCTTTGATGAGCGATCCCCAGTTAATTTTGTTGGATGA
GCCTGCGGCGGGCGTGAATCCCACTTTGATCAACCAAATTTGTGAACACATTGTCCGCTGGAACCAGCAGGGAATTT
CTTTTTTGATCATTGAGCACAATATGGATGTGATCATGTCCTTGTGTAACCACATCTGGGTACTGGCAGAGGGGAGC
AATTTGGCGGACGGAACCCCCGAAGATATCCAGTGTAATGAACAGGTTTTAGAGGCTTATTTGGGATCGTAA
Gene 5: (SEQ ID NO: 49)
ATGCGCGTTTTATTAACAAATGACGACGGGATTGATGCCCCTGGGATTGCAACCTTACAAAAGGCGATCTCCCCCCA
TGCGAGAGAAGTAGTGACGGTGGCCCCCCAAACACAGATGTCGGAATGTGGCCATCGGTTTACGGTTTATGCTCCCA
TTCCGGTGGAGCAACGGACGAAAAATGCCTATGCGGTGGCAGGTACGCCAGCAGATTGTACACGCTTGGGTCTCACG
CAGTTTGCGGCAGATGTTGATTGGGTGCTGTCGGGGGTAAATGCAGGGGGAAACCTCGGCGTGGATATTTACACTTC
AGGAACGGTGGCGGCGGTGCGGGAAGCGACAATCCTCGGTAAGCGGGCGATCGCCTTTTCCCATTTCATCCAGCGGC
CTTTAGAGATTGACTGGGATCTTGTCACCCACTGGACGGGGAAACTTTTGGCGCAATTATTGACCCAGGAACTACCG
GAAAAGCATTTTTGGAATGTGAATTTTCCCCATTTAACGGGAGACTCTGACCCGGAAATTATTTTCTGTGAGCGCAG
CACCGACCCGATGCAAGTGCGCTATGAAGCACGGGATCAACAGTTCCATTATGTCGGTTCCTACCCTGAGCGCCCCC
GGGCCGCTGGTACCGATGTGGATGTCTGTTTTTCAGGGAATATTGCCGTAACCCAAATTTCGATCTAG

TABLE 11
SG2 operon Gene1 DNA translation (i.e., SP2) (SEQ ID NO: 58)
MKSQNVFSTKSAKLIVGGTIFVSAITAANFTMLSAYAVDDTASFSGTVAPACALSNDDGAVAFDAGDRTYTATGSGV
DVTELSETQYVDFECNTDTATVAIAAPVTSKPMAPTNASGLVATHVAKYAVDDTDTLVNPDPTSGTIINEATGVAGF
SQAVNATGLFRVGVESKWSGANGMLAGDYSADITVTVTPN*
SG2 operon Gene2 DNA translation (SEQ ID NO: 50)
MNSQAVPSPKWWFQIIFLSLFLGGLQTKQASAQTPGCFTTNVPSSPLSYDVTSTTQTESYAVTFRCTDDGTTGGSNL
SNVDLDVTLLPLTAPTAGPANLDLGSPNGVTHTISIGSGGSFTNLVDTQTTVNNSGSTNLVVSTAGGKGENLFLDGT
GTITVNIQSRFALQGSTSEFAAGTYTTQFEVDVTPVGGGTTADETTTISSTVSPSCVLDNVIRFRETATPYIKTGSE
PNVSQLQASDTAKFDCNATTVDINFSADSATYTPPTGGATNLTATHQFAYELNGNGFNNYSGPELIENQNTDDNGDA
TLTIRSTWTPNSDQLFASEYNAQTTVTITAK*
SG2 operon Gene3 DNA translation (SEQ ID NO: 51)
MAYSVVSWRKNLSWALCSLALLLPLPLNAQVQVSPMVIKTETSQGMANGVISLTNQGTQSQRVRLSAESFTYTRTGF
ATAESDPYDLSPYLMFSPRELVLEPGQTRRVRLITRMLPSTANGEYRSVIFAEPLRERDEAGGGLSIRARVGVTVYV
KHGQVNFALTPVEASYDPTKQEFQLLVSNPSNGTVQSKGTWTLSQNDQPLLQADIDQRTVIAGGDRLFPLELPPDRT
NLPAGTYQVAGQLQWSESGAVTTTPFSFDVTVPAAR*
SG2 operon Gene4 DNA translation (SEQ ID NO: 52)
VAHSNLKKSHIFPRRLEYLPLTFRLLLFSFFMLFLLGAEVVDAQQDSEPADNGATETTSETFPASFDLIPVGIKLGD
RTANPGTLVRGSENGIQAIDFSNWAIAYNDVLKALQFTATPLADGTIELRSPAAVIRLDPSLLDTDPQLGLVFTVTQ
IRDLLQIPVEFDISEYAIVLTPEWLRASGSLGLTGRYSLPERPIVLEGLPRIEAPNLSFSAIGQEVRVTGGGDRPTE
YEGDLVGIGTFFGGSWYSKIDQRDLTDPRSWQLEEFQYLRQTPSTDYVIGDQRTFWPEGSGRYTGVSIVRRFGFQPP
TEFTNASDGFNPQQRLNSDRLERDIRGRAEPGTLVQLVNKNGNLIVGEQLVDQSGIYRFENIPSASTNKGRGGIAGN
RYELRLYPNGQLSAFPEIRAAEFSSLPGQLSKGTSALLLSAGFERLRQADTFFGSLSNDLQGGFAYRWGATDNLTLG
TGLFYDGQLKGLGEFFFQPGRLPLRITGAATFNSDEQRGEQQSDFRYDLNVRFNPGQRFDFEFDKDELSERIRTRWD
VSDKFRLAFNSNSSDQIAQATWRLFPGFSTRVGWSFNNKALEGGFDLSGALGDLLIRNSVTFSADQSLDWRLFSRYQ
NLTLDHRLRDRQIATEVEYFFRNPEALVDTGHSVFARYQSSPNEDNEARTNELLVAGWRYEANSTVGDRLSDWIVDL
GYGVGTQGAGWQIAVTTNQLLGLNLTARYQDISLTGNESSFSLLIGSDAILSPNFSLKPSRFERLRTEGGIVVIPFI
DANRNGVQDETETAYLQGIEAETADFLFLINEQPINRFSEYEPDLRRRGIFVRLPPDTYRFDVDPAGLPLGWQTTQS
AFAVEVSAGSYTPIYVPLTRAYIVAGTVVNAQGKPLGGVRVEAVNQNNPQERSLSVTNGAGIYYLESVGTGVYDLFI
DGKPAKPGQLRIEIDAEEFTELDLRL*

TABLE 12
SG8 operon Genet DNA translation (i.e., SP8) (SEQ ID NO: 64)
MQLKKLFVPLLAGMLFLGGTSGAIAEELLRTITVTGRGEEAIATSLSEVRLGVEVRGATATQVQADIAKRSNQVVDF
LKSKNVAKLTTTGINLQPEYDYNNGDRRLIGYLATNTVSFEVPTAQAGSLMDEAVKAGATRIDGISFRATEAALTEA
EKTALAEAAQDARTQAQTVLGALGLSPQEIVQIQVNGATPPTPIFKTMDTARIALESAAPSPVEGGEQTVNASVTLT
IRY*
SG8 operon Genet DNA translation (SEQ ID NO: 53)
MLDLIKLAGQLPDMGAHLQEQAVTGRERIERGISLLREAQADFQTLQAHQNTWGDRLIFNHGIPLEPLETRVPISPP
SQAHTVFATDGSQIAPSHHEIAYCYLINIGRVMLHYGQSLHPLLDHLPEIFYRSEDLYTSRKWGIRTDEWLGYRRTA
SEAEVLAEMACKWVLPPGAHGHIPNVAMVDGSLVYWFLENLPAEARQQILEPLLGAWQQLRETRIPLIGYISSTRSV
EAVHFLRLQACPHDKPDCQSHCLDGETKERKAEFRETLPCQTIEPLRDSTLFEQLLQPGDRSGLWLSQARILNHYPE
ADQVCFCYLHVGTEVARIEMPRWVAADPQLLDQTLGIVLGQVQKGFGYPVAIAEAHNQAVIRGGDRARFFALLEQQL
LKAGLTNVGISYKETRKRGSVA*
SG8 operon Gene3 DNA translation (SEQ ID NO: 54)
MPEMPENSQFPVEPPQKPSGTEQQHEENPWVETIKTLVTAGILAIGIRTFVAEARYIPSESMLPTLEVNDRLIIEKI
SYHFKNPQRGDVVVFNPTEILQQQNYRDAFIKRVIGIPGDTVQVSGGTVFINGEALEEDYINEAPEYDYGPVTIPED
HYLVLGDNRNNSYDSHYWGFVPREKLVGKAFIRFWPFNRVGILNEEPQFADEEPITP*
SG8 operon Gene4 DNA translation (SEQ ID NO: 55)
MSEPSPLLQASGLHKSFGGIRAVQNASITVPRGQITGLIGPNGAGKTTLFNLLSNFITPDRGTVIFNGQEVQHLPSH
QIAARGFVRTFQVARVLSRLSVLDNMLLAAQQQTGENFLRVWQQGKIRRQEKANREKAIAILESVGLGKKAQDYAGA
LSGGQRKLLEMARALMSDPQLILLDEPAAGVNPTLINQICEHIVRWNQQGISFLIIEHNMDVIMSLCNHIWVLAEGS
NLADGTPEDIQCNEQVLEAYLGS*
SG8 operon Gene5 DNA translation (SEQ ID NO: 56)
MRVLLTNDDGIDAPGIATLQKAISPHAREVVTVAPQTQMSECGHRFTVYAPIPVEQRTKNAYAVAGTPADCTRLGLT
QFAADVDWVLSGVNAGGNLGVDIYTSGTVAAVREATILGKRAIAFSHFIQRPLEIDWDLVTHWTGKLLAQLLTQELP
EKHFWNVNFPHLTGDSDPEIIFCERSTDPMQVRYEARDQQFHYVGSYPERPRAAGTDVDVCFSGNIAVTQISI*

TABLE 13
SP1 (SEQ ID NO: 57)
MKTNQLLTSVSRSTALAFLALTLGLGGEKALAQWQPTISVPEFKNETNGSYWWWNSSTSQELADALSNELTATGNFR
VVERQNLGAVLSEQELAELGIVRPETGAQRGQVTGAQYIVLGQITSYEEGVKEESTGFGLSGIRIGGVRLGGGGRGS
SEEAYVAVDLRVVDSTTGEVLYARTVEGKAKSDSTSGGATASFAGINLGGDRTETNRAPVGQALRAALIEATDYLSC
VMVEQNGCMAEYEAKDERRRENTRSVLDLF*
SP2 (SEQ ID NO: 58)
MKSQNVFSTKSAKLIVGGTIFVSAITAANFTMLSAYAVDDTASFSGTVAPACALSNDDGAVAFDAGDRTYTATGSGV
DVTELSETQYVDFECNTDTATVAIAAPVTSKPMAPTNASGLVATHVAKYAVDDTDTLVNPDPTSGTIINEATGVAGF
SQAVNATGLFRVGVESKWSGANGMLAGDYSADITVTVTPN*
SP3 (SEQ ID NO: 59)
MLRLLFLHRKKAAQDFQGFTVIELMIVMIITGILTAIALPAFLNQVDKSRYAKARLQMRCMLQELKVYRLNHGSYPP
DQNRNVPYYPGSECFKVHTGYVRDRPDINRNNNTDIPFHSVYDYERWDYNSGCYIAVTFFGKNGLRRFTQAAINEIS
TTGFHFYDGTDDDLVLVVDITDSPCD*
SP4 (SEQ ID NO: 60)
MSESLRLRYLQYLAQRKDEQGEEEKGFTLVELLVVIIIVGILAAVALPNLLAQTDKAYASEGKSAVGAALRTLSAAT
LDPNYVTNASCTQLGIGSSAGNFDLTCGNASQVTAAGSGKAANINVTGTIGTDGKFTVIATKGSATL*
SP5 (SEQ ID NO: 61)
MSDSLRLRYLQYLAQRKDEQGEEEKGFTLVELLVVIIIVGILAAVALPNLLDQTDKAYASEGKSAVGAALRTLSAAT
LDPNYVTNASCTQLGIGSSAGNFNITCGNASQVTAAGSGKAANINVTGTIGTDGKFTVIATKGSATL*
SP6 (SEQ ID NO: 62)
MSESLRLRYLQYLAQRKDEQGEEEKGFTLVELLVVIIIVGILAAVALPNLLAQTDKAYASEGKSAVGAALRTLSAAT
LDPNYVTNASCTQLGIGSSAGNFDLTCGNASQVTAAGSGKAANINVTGTIGTDGKFTVIATKGSATL*
SP7 (SEQ ID NO: 63)
MALEYMIEDLMEQLVEMGGSDMHIQAGAPVYFRVSGKLEPINEEVLTPQESQKLIFSMLNNSQRKELEQNWELDCSY
GVKGLARFRINVYKERGCYAACLRALSSKIPNFEQLGLPNIVREMAERPRGLILVTGQTGSGKTTTLAAILDLINRT
RAEHILTIEDPIEYVFPNVRSLFHQRQRGEDTKSFSNALRAALREDPDIVLVGELRDLETIALAITAAETGHLVFGT
LHTNSAAGTIDRMLDVFPANQQAQIRAMLSNSLLAVFAQNLVKKKSPKPGEFGRALVQEIMVITPAIANLIREGKAA
QIYSAIQTGAKLGMQTMEQGLATLVVSGVISLEEGLAKSGKPDELQRLIGGMTPQVAAKRR*
SP8 (SEQ ID NO: 64)
MQLKKLFVPLLAGMLFLGGTSGAIAEELLRTITVTGRGEEAIATSLSEVRLGVEVRGATATQVQADIAKRSNQVVDF
LKSKNVAKLTTTGINLQPEYDYNNGDRRLIGYLATNTVSFEVPTAQAGSLMDEAVKAGATRIDGISFRATEAALTEA
EKTALAEAAQDARTQAQTVLGALGLSPQEIVQIQVNGATPPTPIFKTMDTARIALESAAPSPVEGGEQTVNASVTLT
IRY*
SP9 (SEQ ID NO: 65)
MKTNQLLTSVSRSTALAFLALTLGLGGEKALAQWQPTISVPEFKNETNGSYWWWNSSTSQELADALSNELTATGNFR
VVERQNLGAVLSEQELAELGIVRPETGAQRGQVTGAQYIVLGQITSYEEGVKEESTGFGLSGIRIGGVRLGGGGRGS
SEEAYVAVDLRVVDSTTGEVLYARTIEGQAKSDSTSGGATASFAGINLGGDRTETNRAPVGQALRAALIEATDYLSC
VMVEQNGCMAEYEAKDERRRENTQSVLDLF*

TABLE 14
SG1 (SEQ ID NO: 66)
ATGAAAACCAATCAGCTTTTAACATCCGTAAGTCGCTCTACTGCCCTGGCCTTTCTCGCACTCACCCTAGGACTTGG
GGGCGAAAAAGCACTGGCCCAGTGGCAACCGACTATTTCTGTCCCAGAATTTAAAAACGAAACCAATGGCAGCTATT
GGTGGTGGAACAGCAGCACCTCCCAAGAACTGGCCGATGCCCTCAGCAATGAGCTTACTGCCACTGGCAACTTCCGC
GTTGTTGAACGGCAAAACCTAGGGGCCGTCCTGTCAGAACAGGAATTAGCTGAATTGGGAATTGTTCGCCCAGAAAC
GGGAGCCCAACGGGGCCAAGTCACAGGGGCGCAATACATCGTGCTCGGTCAGATCACCTCCTACGAAGAAGGGGTCA
AGGAAGAATCGACTGGCTTTGGGCTCAGTGGTATTCGGATCGGTGGCGTCCGGCTCGGCGGTGGTGGCCGTGGCTCT
AGTGAAGAAGCCTACGTTGCCGTGGATCTACGGGTTGTTGACTCAACCACTGGGGAAGTGCTCTATGCGCGTACCGT
TGAAGGAAAGGCAAAGTCTGATTCGACTTCCGGAGGTGCAACGGCTAGTTTTGCTGGGATTAATCTTGGTGGCGATC
GCACCGAAACAAATCGCGCTCCCGTTGGCCAAGCGCTCCGGGCGGCCTTGATTGAAGCCACTGATTATCTCAGTTGT
GTGATGGTCGAACAAAATGGCTGCATGGCTGAATATGAAGCGAAGGACGAGCGCCGTCGGGAAAATACCCGGAGTGT
CCTTGATCTTTTCTAG
SG2 (SEQ ID NO: 67)
ATGAAATCCCAGAACGTTTTTAGCACCAAATCTGCCAAGCTTATTGTTGGTGGTACGATCTTTGTTTCGGCCATTAC
CGCTGCCAACTTCACAATGCTGTCAGCCTACGCAGTTGATGACACCGCTTCTTTTTCGGGTACGGTCGCTCCAGCTT
GTGCACTCTCCAACGATGATGGTGCAGTAGCATTTGATGCCGGCGACAGAACTTATACAGCCACAGGTAGTGGCGTA
GATGTCACTGAGCTTTCTGAAACTCAGTATGTTGATTTTGAATGTAATACCGACACTGCTACTGTTGCGATCGCTGC
ACCTGTTACTTCAAAACCAATGGCTCCTACAAATGCAAGTGGCTTAGTTGCCACTCATGTTGCTAAATATGCGGTAG
ACGATACTGATACTCTTGTAAATCCAGATCCAACGTCTGGTACGATCATTAATGAGGCTACTGGCGTTGCTGGATTT
TCTCAAGCAGTAAATGCAACTGGCTTATTTAGAGTGGGTGTTGAATCTAAATGGAGCGGAGCTAATGGAATGTTAGC
CGGGGACTATTCTGCTGATATCACTGTAACAGTGACTCCTAACTAA
SG3 (SEQ ID NO: 68)
ATGTTGCGTCTTCTCTTTCTCCATCGTAAGAAAGCAGCCCAAGATTTCCAAGGTTTCACCGTGATTGAACTCATGAT
TGTAATGATAATCACGGGCATCTTAACGGCGATCGCCTTGCCTGCCTTTTTAAATCAAGTGGACAAGTCCCGATATG
CTAAAGCGCGGCTGCAAATGCGCTGTATGCTTCAAGAGCTCAAAGTTTATCGCCTGAATCACGGCAGTTACCCCCCG
GATCAAAATCGAAATGTTCCTTACTATCCTGGGTCTGAGTGTTTTAAGGTACATACAGGGTATGTTAGGGATAGACC
GGATATCAATCGAAATAATAATACAGATATTCCATTTCATTCTGTCTATGATTATGAACGCTGGGATTACAATTCTG
GCTGTTATATTGCGGTGACATTTTTTGGCAAAAATGGTCTGAGAAGATTTACTCAAGCTGCCATTAATGAAATATCC
ACCACTGGATTTCATTTCTATGATGGAACTGATGATGATTTGGTCTTGGTTGTGGATATTACTGATAGTCCCTGTGA
TTAA
SG4 (SEQ ID NO: 69)
ATGTCTGAATCGCTCCGTCTACGTTATCTGCAATATCTTGCCCAGCGTAAAGACGAACAAGGTGAAGAAGAAAAAGG
TTTCACCCTTGTCGAGTTGCTGGTCGTTATCATCATCGTTGGCATCTTGGCAGCAGTTGCATTACCGAACCTGTTGG
CTCAAACAGATAAAGCCTACGCCTCTGAAGGTAAATCAGCAGTCGGTGCTGCTCTTCGTACCCTTAGTGCGGCGACA
CTAGACCCTAACTACGTCACCAATGCGTCTTGTACACAGCTTGGTATTGGTAGCAGTGCAGGTAACTTTGACCTAAC
TTGTGGCAATGCTAGCCAAGTAACGGCTGCGGGAAGTGGTAAAGCAGCGAATATTAACGTGACTGGCACAATCGGGA
CAGACGGTAAGTTTACCGTTATTGCAACCAAAGGCAGCGCAACTCTTTAA
SG5 (SEQ ID NO: 70)
ATGTCTGACTCCCTCCGTCTTCGTTATCTACAATATCTTGCCCAGCGTAAAGACGAACAAGGTGAAGAAGAAAAAGG
TTTTACCCTTGTCGAGTTGCTGGTCGTTATCATTATCGTTGGCATCTTGGCAGCAGTTGCATTACCGAACCTGTTGG
ATCAAACAGATAAAGCCTATGCCTCTGAAGGCAAATCAGCAGTCGGTGCTGCTCTTCGTACCCTTAGTGCGGCGACA
CTAGATCCTAACTACGTCACCAATGCGTCTTGTACACAGCTTGGTATTGGTAGCAGTGCAGGTAACTTTAACATAAC
TTGCGGCAATGCTAGCCAAGTAACGGCTGCTGGAAGTGGTAAAGCAGCGAATATTAACGTGACTGGCACAATCGGGA
CAGACGGTAAATTTACCGTTATTGCAACCAAAGGCAGCGCAACTCTTTAA
SG6 (SEQ ID NO: 71)
ATGTCTGAATCGCTCCGTCTACGTTATCTGCAATATCTTGCCCAGCGTAAAGACGAACAAGGTGAAGAAGAAAAAGG
TTTCACCCTTGTCGAGTTGCTGGTCGTTATCATCATCGTTGGCATCTTGGCAGCAGTTGCATTACCGAACCTGTTGG
CTCAAACAGATAAAGCCTACGCCTCTGAAGGTAAATCAGCAGTCGGTGCTGCTCTTCGTACCCTTAGTGCGGCGACA
CTAGACCCTAACTACGTCACCAATGCGTCTTGTACACAGCTTGGTATTGGTAGCAGTGCAGGTAACTTTGACCTAAC
TTGTGGCAATGCTAGCCAAGTAACGGCTGCGGGAAGTGGTAAAGCAGCGAATATTAACGTGACTGGCACAATCGGGA
CAGACGGTAAGTTTACCGTTATTGCAACCAAAGGCAGCGCAACTCTTTAA
SG7 (SEQ ID NO: 72)
ATGGCTTTGGAATACATGATCGAAGACCTCATGGAGCAGTTGGTGGAAATGGGCGGCTCCGATATGCACATTCAAGC
GGGGGCACCGGTTTATTTCCGGGTGAGCGGCAAATTAGAACCGATTAACGAGGAAGTTTTAACTCCCCAGGAAAGCC
AAAAGTTAATCTTCAGCATGCTGAACAATTCCCAACGGAAAGAACTAGAACAAAATTGGGAATTGGACTGTTCCTAT
GGCGTGAAAGGTTTAGCTCGTTTCCGGATTAACGTTTACAAAGAACGGGGTTGTTATGCCGCCTGTTTACGGGCCCT
TTCTTCTAAAATTCCCAACTTTGAACAATTGGGACTGCCCAACATTGTGCGGGAAATGGCGGAACGCCCCCGGGGAC
TAATTCTAGTGACGGGACAAACTGGCTCCGGTAAAACCACCACTTTGGCAGCAATTTTAGACTTAATTAACCGCACC
AGGGCCGAACATATTCTCACCATCGAAGATCCGATCGAGTATGTGTTTCCCAACGTGCGCAGTCTTTTTCACCAGCG
GCAACGGGGGGAAGATACGAAAAGTTTCTCCAATGCTCTGCGGGCAGCGTTACGGGAAGATCCGGACATTGTACTGG
TGGGAGAATTGCGGGATTTGGAAACCATTGCCCTTGCCATCACTGCGGCAGAAACCGGACACTTGGTTTTTGGCACT
CTCCACACCAACTCAGCAGCGGGCACCATTGACCGGATGTTGGATGTGTTTCCGGCTAACCAACAGGCCCAAATTAG
AGCCATGTTATCCAACTCTTTACTAGCGGTATTTGCCCAAAACTTAGTCAAGAAAAAGTCCCCCAAACCCGGGGAGT
TTGGCCGGGCCCTAGTGCAGGAAATTATGGTCATTACCCCGGCGATCGCCAACCTAATTCGGGAAGGCAAAGCGGCC
CAGATTTATTCCGCCATTCAAACCGGAGCAAAACTAGGTATGCAGACCATGGAACAGGGCCTGGCCACGTTGGTGGT
GTCGGGGGTAATTTCCCTGGAAGAAGGTTTAGCTAAGAGTGGTAAGCCGGACGAGCTACAGCGCTTAATCGGTGGCA
TGACCCCCCAGGTTGCCGCTAAACGTCGTTAG
SG8 (SEQ ID NO: 73)
ATGCGCGTTTTATTAACAAATGACGACGGGATTGATGCCCCTGGGATTGCAACCTTACAAAAGGCGATCTCCCCCCA
TGCGAGAGAAGTAGTGACGGTGGCCCCCCAAACACAGATGTCGGAATGTGGCCATCGGTTTACGGTTTATGCTCCCA
TTCCGGTGGAGCAACGGACGAAAAATGCCTATGCGGTGGCAGGTACGCCAGCAGATTGTACACGCTTGGGTCTCACG
CAGTTTGCGGCAGATGTTGATTGGGTGCTGTCGGGGGTAAATGCAGGGGGAAACCTCGGCGTGGATATTTACACTTC
AGGAACGGTGGCGGCGGTGCGGGAAGCGACAATCCTCGGTAAGCGGGCGATCGCCTTTTCCCATTTCATCCAGCGGC
CTTTAGAGATTGACTGGGATCTTGTCACCCACTGGACGGGGAAACTTTTGGCGCAATTATTGACCCAGGAACTACCG
GAAAAGCATTTTTGGAATGTGAATTTTCCCCATTTAACGGGAGACTCTGACCCGGAAATTATTTTCTGTGAGCGCAG
CACCGACCCGATGCAAGTGCGCTATGAAGCACGGGATCAACAGTTCCATTATGTCGGTTCCTACCCTGAGCGCCCCC
GGGCCGCTGGTACCGATGTGGATGTCTGTTTTTCAGGGAATATTGCCGTAACCCAAATTTCGATCTAG
SG9 (SEQ ID NO: 74)
ATGAAAACCAATCAGCTTTTAACATCCGTAAGTCGCTCTACTGCCCTGGCCTTTCTCGCACTTACCCTAGGACTTGG
GGGCGAAAAAGCACTGGCCCAGTGGCAACCGACTATTTCTGTCCCAGAATTTAAAAACGAAACCAATGGCAGCTATT
GGTGGTGGAACAGCAGCACCTCCCAAGAACTAGCCGATGCCCTCAGCAATGAGCTTACTGCCACTGGCAACTTCCGC
GTCGTTGAACGGCAAAACCTAGGGGCCGTCCTGTCAGAACAGGAATTAGCTGAATTGGGAATTGTTCGCCCAGAAAC
GGGAGCCCAACGGGGCCAAGTCACAGGGGCGCAATACATCGTGCTCGGTCAGATCACCTCCTACGAAGAAGGGGTCA
AGGAAGAATCGACTGGCTTTGGGCTCAGTGGTATTCGGATCGGTGGCGTCCGGCTCGGCGGTGGTGGCCGTGGCTCT
AGTGAAGAAGCCTACGTTGCCGTGGATCTACGGGTTGTTGACTCTACCACTGGGGAAGTTCTCTATGCGCGTACCAT
TGAAGGACAGGCAAAATCTGATTCGACTTCCGGAGGTGCAACAGCGAGTTTTGCTGGCATTAATCTTGGTGGCGATC
GCACCGAAACAAATCGCGCTCCCGTTGGCCAAGCGCTCCGGGCGGCCTTGATTGAAGCCACTGATTATCTCAGTTGT
GTGATGGTCGAACAAAATGGCTGCATGGCCGAATATGAAGCGAAGGACGAGCGCCGCCGGGAAAATACCCAGAGTGT
CCTTGATCTTTTCTAGACCGTTGA

TABLE 15
pContig41 (SEQ ID NO: 75)
CCAGCCGCTTGCCAGCCGTTATCAGTAAAGTTAATCTCTGTGCCAGCTCCCAGATCAATTAAAGGGACAAAAGCAAA
TTCGTCAGGGTTATCAAAATTAAAACCGATGATAGCAATATCACCTGCGGACAAAATCGTAGCCGTCACGTTAAATT
CTCCCTTTGAATTTGAATTAAGTTAAATCGGGTAAATCGGAAATGTTTTGTTAATTTGAGAAAAGCCATGATTAATG
AAAAATCCTCATGCATCATTGCATTTGTTAATTTGCAAGTCTGAGGGAATCGCCTCACAATACAAGGACTTCAAAGT
TGCATGCCGATTCTTCTAATCACTCATCTGCCAAAATGTTCATCGAAGTTAGGAGGTAGTTTTAGGTATCCTAAATT
CGACGATTATATTTGGGGTCAATATGATCTATGCTGATGAAACTAATAACATCGAGCTCTCAAAAAATGCCGCTAAT
AGAAGCGGTTCGAAATAGTGACGCTATCTTTCGTACCATCAACATTGTTCAAATTGACACCTAAATTGAAATTTTGC
CTTAAAGAAAGCTACAGATTTTGAGCTGAGTTAGGTTTAGTTGTCATACACTTTAAAAACTTATCCAACAAAATAAT
CTCCTAGTTTCCTAAAGAAACAGTAAAGCCTGCCTTAAATCTTGGAGAAAATACGCGGAAATCCCAGGAAAATACTT
AGGTTTTCTCAGCGACTGCTTATAAGGTAAAGATAAAGAGACAGGATGTCTTGCTTCTATATTTAGTAAGGTCAAGC
AAAAGTATGACGAGAACACAATAGGTTACAATTTTACTGGATGGCAAAGTATATTTTGGTTTGTGAGAAATAATTTA
GTTTTCTCTAAATGTATAGTCAATCACATTAGATTACTGTGGAATTTTATCCATCACCATAATTCATTATCAATCCC
CCCCTTTAGTATTATCTTAAATAAAGTTTTACTTTTAATTTTTTAATCCCACAGCATTTTTATGGTGATTAGGGTGC
AATTTGGGGTTTGCATGACTTATAGCTAATTCAGGGATATTGCCAAAAGTCTATTCTGTTGACTCCAAACAAAAGTT
TGTTCAGTCTCGATGGAAGTAATTTAAACTTGTGCGATCACCTTCAAAAGCTAAATTTTTCCCGAAGCATGACGATC
AACAAAATAGACTGTATTTCTCAGAAATGGTTTTACAGTGGAGAGTGTCCAACTTTTCAAAGATTACTCTATGATTA
CCGTTCCTGGGGGAACTATCAAACCCCATTGGTTTGTCCGTCGTGATCTCGATGGTTTTTTTGGCCTCGCACTGAAT
AATTTTGTCCAAATCCTAGTTATTGTTAGTCTGACTCAAGGGGTGCTGCAATTTCCGGTCGAACTGCTTTATGGCCG
CATTTTGCCAGGTAGTGCCCTTAGTTTAATCGTCGGCAATGCTTATTACAGTTGGCTCGCCTATAAGCAGGGTTGCG
CAGAACAGCGGGACGATATTGCTGCCCTTCCCTATGGCATCAATACCATCAGTTTATTTGCTTATATTTTTCTCGTG
ATGTTGCCTGTCCGTCTCCAGGCGATCGCCACCGTCGCCGACATCCTGAAAAAAACAAAAATTCTGCTGATTAACTT
TCCAACCCTTTCTTTATCAACCTCAATCCCCTTAAAAATCATGATTCAAGAGATTGCCACTAGCATTCGTACTACAG
TCTTTCTTTGGTTACTTACTGCTTTGATTTACCCTTTGCTGATTTTCATGATCGGTCAGGGCCTTTTTCCAATCCAA
GCCAACGGCAGTCTAATTGTGAATAACCAAGGGCAAGTCATTGGCTCTAGCCTCATCGGTCAAGCCTTTAATAGCGA
AGACTATTTCTGGGGTCGCCCTAGCGCAGTCAATTACAGTATCGGCGAAGATGCCGCCCCTACCGGACTATCCGGAG
CGACGAACCTAGCTCCCAGTAATCCTGACCTGTTGGCATTGGTTAAGGAAAGAGCCGAAATCCTGCGGGCTGCCGAT
CTTGAGCCGACTGCTGACCTGCTCTACAGTTCTGGCTCTGGCCTCGACCCCCATATTTCTCCTACTAGTGCGATCGC
TCAAATCGACCGGATTGCCGCCGCTCGTAATCTTTCCCCTGATGACTTAAAAATCTTAATTGAACAGAATACCGAAG
GACGATTCTTAGGAATTTTTGGTGAACCTGCTGTCAACACAGTCACCCTTAATTTGGCCCTTGATCAGCTCTAAAGC
TTTGAAAACAGGAACTTTCTAGCCTGCATATTCTCTGACTATCCTCTTGCTCTTTTAGATGTACAACCCTACTGCGA
TCGCCGCTACTAACTACCAAATACTCCCCCGTCGAGGCAAACACAAAATTTTTATTGGCATGGCCCCTGGAGTTGGT
AAAACATACCGAATGCTCGAAGAAGGACATGCCCTTAAGCAAGATGGTGTTGATGTGGCTATTGGCTTATTAGAAGC
CCATGGGCGGGAGGAAACAACCCTGAAAGCTGAAGGCTTAGAGCTAATTCCTCGTAAACAAGTTTATTGTCGAAATG
TTTTATTGCAAGAAATGGATACCGAGGAAATCTTAAGGCGATCGCCCCAGTTAGTCCTGATAGATGAACTAGCCCAC
ACGAATATACCCGGCTCTAAACAAGAAAAACGTTATCAAGATGTTGAAAAAATTCTAAATGCCGGAATCGATGTCTA
TTCAACGGTTAATATTCAGCATCTAGAAAGCTTAAATGATCTCGTATATCGCATTTCTGGGGTTGTAGTAAGAGAAC
GTATTCCCGACCGAATCATTGATGATGCTGATGAAATTGTTGTAGTTGATGTAACCCCAGAAACATTACAAGAACGA
CTTCAGGAAGGAAAAATTTATGCGCCTGAAAAAATTGATCAAGCTCTACAAAACTTTTTTCGTCGCAGTAATTTAAT
TGCTTTAAGAGAATTAGCGCTACGTGAAATTGCCGATAACATTGAAGAAGAATCCATTTCTGAAACTAAAAATCTCC
ATTACAATATTCGAGAACGGGTTTTAGTATGTATTTCAACTTACCCAAATTCTATTCAGCTTTTACGCCGGGGCGCT
CGAATTGCCAATCATATGAATGGACAATTGTTTGTGTTATTTGTAGCACCTGCGGGTAAATTTTTATCAAAATCAGA
AGCATTACATATCGAGACATGCAAACGATTGTGTCAAGAATTTACAGGGGAATTCTTAAGGATAGAATCACCGAATA
TTGTTACCACAATCGTCAAAATTGCAACCCAAGAACGTATTACTCAAATTGTTTTAGGGGAAACTCGTCGCTCCCGA
TTTCAATTATTATTCAAAGGCTCTATTGTACAGCAGCTAATGAGAGCATTACCCCAAGTTGATATACATATCATTGC
CAATAATTAAAATTCAACTAACTGGACAAGAGAACGCACTTAAACAGCAAGCATCGATTATAAAAAGTATCGTCTCA
ATCCTCTATTCAGAAGATATCCTGATGAAAGCAAAGCCGTAAAGATTGCTCGAAAATTCAAGCATCTCCCCCCAAAG
GCGGCGTTTTTTTATGTTTTTATGAGAGTCCAAGATAAGGGACAAGATAAATTTGAATTCCGGGGACGGACGGGGGC
GATCGCATAACCTCTACTGCTTCTAATGATTTTGATGATCAAAAAAATGAAGAAGATCAATAACCATGACCTTGCCT
CCAGGATCCGTTCTCCAATTTTTTTAACCTGCGGAATGTAGGAATTAGGGCAGCGCGAAAGTACGGGTATGTAAATT
ATTTTGTGCAAGCGGTCAAAATCAATAAATCAGTAACGGTGTTTATTTAGCAATGGACCCGTTTTGGTCGCTAAGGC
TAAAACCATTGATATATAAACTTTTCAAGCATGAGTATAATAACTCATTAAAAGTTCGACTTTGACAAACCGTATTC
TTCAGCAAATATATAACTGTCCCATTTATGATGCTTAGAGCAATAACCCAAAAAGTCTAGAAGTCTTATAAATAGTG
GGTTTGGCAAGAATAATTGTCCCATTTAAACATAGAAGTGGGACAGTATTCTGACTTTCTAGGAACTCAAAAGTTGA
CCTCCAGAATTGCTTAGTGAGCAAAACGGATCCATTGCTAAATAAACACCAAATAGTGATGACCTTCAAGAATTGGC
TAAGGGTTGCGACCTACTCATCTTGCCAACGACCCCGGATGTAGTCAGTCTAGAACCGATGCTGGCGATCGCCAATG
ATGTGGGAGATGCAAAGTATCGAGCATTACTCACAATTGTGCCCCCATACCCTAGCAAGGAAGGGGAAACGATGCGT
AATGAGTTAATCGCAAACGGTATCCCTACTTTTCAAAGCATGATTCGCCGTAGCGCTGCTTTTCAAAAAGCGGCTTT
AGCTGGTAAACCAGTAAACCAGATGTCTGGTAGAGATAGAATCCCTTGGAATGACTTTGAGGCACTGGGTAAAGAAA
TTATGGAGGTACTAAGAAAATGAGCGGCAAATTTGGAGACATCATCGGCAGAGCAAAACAAACCAGTAAACCAGATA
ACCAGATATCTGACCAACAAAATAATCAGCAATCTGCTCAGTCCACCGAGACCGAAAAAATGGTGAATCTTTGTGCG
AAGGTTCCCAAGTCTCTCAGGCAGCATTGGGCGGCAGAGGCTAAACGAAACGGCATCACCATGACAGAAGTAATCAT
TGATGCTCTTAACCAGAAGTTTGGCAAACCATAAAACCAGATTTAATGCTATGGTCGCTTTTCTTTTGACGAAATGA
CCCCATAAAACCCCAGATTTTTCGTTGGGATTGTAGTTCAAAGAGCTGGGTGAATCTCCTTTTATTCAGGCAGGATG
CAGTCGAAGCGGCTCTATCCCGCCGTTTCCTTTTAAACCAGTGGGAAAAGGCATCGGTGGGCAACCTCACCACAGTC
ATCAGGGCGATGCAGGATTTAGAGGAAATTATCGATGATGTCCCGGCGGCGATCGCCTCTTGCTAGAAGCTAAATGA
CCGGGTTAGAGGCTGTTTCATTTTGACTCTGCTGTCCGAAATGGAGGTAAAGGGGTTGAAGTAATAGGCGTTTGCTT
AAGTTCAGTCATTGATAGGCTTGAGCTAATTTTTCTGTAACCTCTGAAAATAGCGTGGCTCGATAAAACCGCTTACT
TGTCGATTGACAAGTAAAAAAATAGCACCTATCTTGGAATCAAGACATGAATCGAAATCGACATCCCAATAAAGAAA
TCGAAGCGGCGCTGGAGTACGCAGAAGCTAACGGCTGGAGAGTGGAAATAGGCGGCGGGCATTGTTGGGGGAGACTC
CTATGCCCCGAAAACAAGGGCTGTCGCAATCGTCTGTTTTGCGCTAATTCGATTTGGTCTACGCCGAAAAATCCTCA
AGGTCATGCCAGAAATATTCGTAAATGGGTTGATGGCTGCGATGAGAATAGGGAATGAATAAAGGACTAAAACCATG
AAATACTATAACTTTGAGCTATCTTTCAGGCTCCCTGGTGTCAATACAAACCCTGAGCAATACTTAGACGCACTATT
TGAGGCTGGCTGCGACGATGCAATGATCGGTATGGGCCGTAATGGGTTTATAGGAGCTGACTTTAGTCGGGAATCTG
AATCTTTAGAGTTAGCTGTCGAGTCCGCAATTAAAGATATCGAGAGCGCTATTCCCGGAGCTGTACTGATTGAGGCT
GGGCCAGATTTAGTCGGGGCTTCAGATATTGCTGCTATTCTCGGATGTAGTCGCCAGAATATTAGACAGCACCTGAC
AATGGCTGATGAAAACGGCCCTATTCCTGTATACCAAGGTAAACGCGACCTGTGGCATCTTGCAGAAGTTCTGATCT
GGTTACGAGATGCTAAAGGGTTAAAAATTGAACCTGAACTAATAGAAGTCGCGGCTTACGTGATGACATTCAACTCG
AATTGCCAGTCTAAAAAAACCAAAAAAGTAGCGGCCTTGGCTTAATCATCTATGGTCAACTACAAGCCTTTTTTACG
AATCCGATTCACGCCTGAGAGCGATTTATAAAGAAGGCTCCTCGGCGTAATAATTTTTCTTATGGTCGCCGCCCGCT
TTGGTTGAACTTACCCTCAAAAGTTTCAACACATTCAAAAATCCTCTCAATTTTCCGACCTAAGGAAATCAGTCTCC
GATAGCGGCGATTTTGCCTCCGTCAGAAAAAAACCTCCAGAGGTGCGTCAGGTGTGGGGAAATTTTTTCTTTATCAC
CAGGAGGTAATTGATTATCATTGCTCAGCTCTTTTTGGTCATCTGCTGGAGATTTGGTGATACTCTACTGGCTTTTT
GCTGGTCTTTTGTCTCTTGTTTGCTGGATATTCTCAATAAATCTGGATAATCTAGCAGTTATTGCAAATACAATCTT
TCTTGAGTTGTTCAGGCTTTAGCAAAGCACCATCTAATCCCCACCAGATAACTAGCATTTCACCATCAAGACTAATT
ACAAACAATTGTACTGATTAATCTTGCGTCCATTATTTAATTTATTCGTCCGTCCGCCACTTAAAGGAGAGTGATTA
AAAACGGGGGAATCAAGCAGCATTAGGCACTCCAGTTTCCTTCAGTGGCGGAGAATACCATCGGTCAACCACTTCTT
TCCAGTCTTGATTTAGGACTTTAGTTCTCACCTGTAGTAATAGATGAGCACCCTTCGGAGTCCATCGCATTTGCTGC
TTCTTCTCCATTCGTTTAGCCACGACTTGATTGACTGTAGATTCCACAAATCCCGTTGAGATGCGCTCCCCACAGCG
GTAACGTTCTCCATAGTTGGGAATAAAATGGCCATTGTTCTGAATATACGTGTAGAACTCAGCAATAGCCCGCCGCA
TCTTTTTGAGTTTCGGATAATTACTTTCGAGAAAGTCAGCATCCTCCTCCAGCCATTCCAGCTCTTGTAATGCCCTG
AATACATTCCCATGCCAGAGATACCATTTGACACTCTTTAGCCTCTCTACCATAGACTTGCCATTGTCTAAATCATA
GTGCTTCAACCCCCTGAGATATTGACTGAGCTGAGTGATCCGCATGGTGACATGAAACCAGTCCAGAAGATATTCCG
CTTGAGGATTGAGAAATCGCTGCAAGTCTCGGACTGTATCGCCTCCATCAGAAAAGAAGGTAACCTGCTAATTCATT
TGCATTCCTTGGGATTTTAGGAGTTCAAATAACCGTCGCTTTGGTTTGGTGTCATAGGTCTGGACAAAACCAAAGCT
TTTGCCAGGTCCTTCATCTGGAATACTCTTGCCCACAATCAGTTCAAAATGGCGCTGTTGTTTACTCCCTTTAGAAC
AGTGAGCATGGATATAGGCACCATCAATTCCTACATTGAGGGGAAGCTCAGGTCGAGGGAGTTGCTCCCATTGGCGA
GGGCTACCTTCTACATACATAATTTGTTCTTCTCCCAATTCATCATCGAGCTTTTGCCCCATTTGATGCAGGTGATA
TCTCACGGTAGAAGGATTAAGGGTGCCGTTCAATGGCAACACCTCATCAAGTAACTGGACACTCAGACCATAGGAAA
CCAGGGAGGCAAATTTTGTCTGCAGGTAAAGGTATTCAGGAGACTGCCGTTCAGTCAGGAGTAAGGCTAATGGGCTA
AAGCTTTTTGTTGCTTGGGACTGGCAGGGACAATGAAAAAATCGAGGACTAGTTAACGTCAGCTTCCCAAATACAGA
ACGATAAATTAGCTTATGTTTCCCTTTGCATTTACGCGGTTGACCACAGTCTGGACAAAACGCCTGTTGCTCTACAT
ATTGACTGACCTGTGACGAAACCATTTCTTGTTGGATCCCTTGCAGAATCTGTTTTGATTCGGCCAAAGTCAAGCCT
AGATTAGTAGGAGTCAATGGGCCTCGCTCCAATTGAGCGACATCCTCAATGATTTTTATGCTGCCATCATCGGCTTC
AATCCTTAAGCTGATTTTGATATTCATAAGCCTAGAAAATAATGTCCTCTCGAAAAGCTGGGTCGTCACTCAAGGGA
TTTTGCTGTAAGCGATGATTGAGTTTTGGTAACTCAAGGTTGTCTAGTAGCAGCCAATAACTCAGTTGCCGAAGTTC
ACTCAGATGTGGCTTAAGCTCATCATCTCCTTGGAAAAGAATTTCAGCCATGCGGTGGAGTACCATTCCTGTAGAGA
ATTCCTGTCTAAGTTCCTGGAAAGCCCAAGCTCTTCCACAATCTTCAATGGGGCCATTTCGTTTACCCTTCAATACA
AATCGGGTAAGTTTTTTCGGGCTCAAGTTCAGTTATCTTTTCAACTCGCAGTTGTACCTGCCAGTTATCGTAATAGT
TGTACTCGTAGCGAAACCGTTCTCGCACCCGCAAACCCAGACTTAAGAGAGTCACAGAATGGGGATCATCGCTGAAG
CATTCCCCACCAATATGATGGATGCCGTAGGAGTTGCCGTGGATGACGAAGCGATGTAAATATGTGTCGCTCCAGCC
AAAAGCGATCTGTAGAATAAAATGTAGTTGAGCAATCGTTGTGTCGCCTCTCACTAGGAACCGACGCCAGATCATTG
GGCTGACACCGACAATAACAGCTTTGATCTGAAATACATGTGAGCCTGAGCCACTATCCATACCACTACATCAACGA
ATAGACTCAATAGTCTTATAAAGTGTACTTTGCAAGACTGTGCAAAACAGTGTTTTATTTAATGTCTCATTATGGCT
TTATCATCGTGTCTAAGACGTTATAAAACATTACGGTGAGTCATCATGGCGATGGTTGGCTACGCGCGAGTCAGTTC
TGTTGGGCAGAGTCTGGAAGTTCAAATCGAGAAGCTCAAGCATTGCGACAAGATTTTCAAAGAAAAGTGCAGTGGTG
TCTCTTACAAACGCCCTAGGCTTAAAGCCTGTCTTGAATATGTCAGAGAAGGCGACACATTAGTTGTGACTCGTTTA
GATCGCCTGGCTCGCTCAACCCTGCACCTGTGCGAAATTGCGGAACAATTGGAATCTAAGCAGGTGAATCTTCAAGT
GCTTGAACAGAGTATTGATACTGGAGATGCGACCGGACGGCTTTTATTCAATATGTTGGGGGCGATCTCCCAATTTG
AAACGGAAATACGTGCGGAACGTCAAATGGATGGCATCCAAAATGCGAAAGCTCGTGGGGTTCAGTTTGGCCGTAAA
AAGCAGCTCACGCCAGGTCAATGCCAAGAACTACGTAAAAGACGCTCACAAGGGGTATTAATCAAAACGTTGATGGA
AGTCTAAGGCAACTATCTATCGCTATTTGAAGGAAGCGGAAACGGTAAAAAGTTGATGAGAATTTAGATCCCTGCGA
TCCACTCTGAGTATACTTTCCCCCGTTTTTAATCACTCTCGATCTCCCACAGTATTCAAACCGCTCCTGGGCTGCCA
TGAGACTCTCTTCTAGGCGGTCTTTTGTGACATATTCGTCCAAGGGCAGCAGAATCCCTAGGTCTATGGTTGGTGAT
TCCTGAAGCTGGGTCTGGATATGGGATAGGACTCCCAGAATTTTGTACAGAGCCTTGATGTATTTCAGGTCATTGTT
GCGCACCGCGGAACCCTGAAACTTCTTGGCACCGGCACCCAGGAGATAGCAGGCATCGCCATGGGATACCCAAGCAT
TCTCCACATGGGTATACCCCATCATCCACCCAGAGGAGCGTGGGTAGTCTGGGGGCACTGCCGCGCAGTAGGGGGAC
ATCACAAAACATTGGGTAGAAGTACCCTCTAGGCAATAGGCCACTTTTGTTGCACTACTACCGGGGTCAATCACAAT
TTTGAGTTTAGACATAATTTATTCCTCCTACTGATGAAGAGAAATTGCCCTGAACAGCACGGATAGAAGTGATTTAT
TTGTTACACAACTTCATAATGGTTCGTCTGTAAAAGCTGTGATTGTCTTGGGCTGGTCTTGGTTCGGGGGGGCGAAT
TTTTGCTGAGAATTTTCTCTTATTGATGCTTAGGCTGAAAAGTAAACTTGTATTACTGTTTACTCTGATGGTTAATT
GCTGGTTATCCGCTGGGGGTTGGGTGGTATTTTGCTAACATCTGGATAGGTCAATGCAAGTTCTATTCGCAATAGTT
GTTGAATGATCCCGGTTTCTTGAGAATTATCAGCAAACAACAGGCAATGAGCCACCTAGCCATGGGCAAAGCGCCAT
CAACTTCTGACTGTCTAGCCACGGAATCAGAAGCACGCTCTGCCGTTTATCCTCTAGTCAAACCAAGGAAATTTCCC
CACACCTTATACACCTCCGGAGGTTTTTTCTGGCGGTGGCAAAATCGCTGCTATCGGAAGCAGATTTCCTTGGGTCG
GAAATTTGAGGAAATTTTCCGAGATTTTGGCGATCGCCCATACAACTTTGAAAAAAGTGAGTAATCAAAACAGATAT
TTTGTTCGTTTCAGTCGTAATGCCTATTGAAATAGGAGGCCCGTATTCTATATCCTTGCCATGGGCATCGGGTCGTT
CACCAGCGAGGCCATCTAATGTCCTATCAGCCGCCCTTCACCATTACCCCCCAAATCATTAACCAGATCAGTATTAT
CTCTCAGTAGATCGGGGCACTCGACCATTCGCCTCTTACCTCCTCCCCAGCGAAAGCCTGATCAAATAGCACCCCAT
TCATTTGCTTTATGCTGGATATCATTAATACAACCCTGCGCCAAAACCTGACATCCTCCAGTCCACTATCAACAACA
AAAAATAACCCAGGCGATCAAGTAAATGATCAAGCCGATCAAGTAAGCGATCAAGTAAAACAACTCCTTGCCATTAT
GGATGACCAATTTTGGAGCACTAATGCTCTAATGGAATCTTTTTCTCTCACCCGTAAACCCACCTTCCGCAAAAACT
ATCTCTATCCCGCGATCCAGGCTGGGCTAGTGGTGATGAAATATCCGGATAATCCCCGTCATCCCCAGCAAAAGTAC
AAAAAGGTAGATGGGTAAGTTTGTCGCTATGCCAGGAATAGCATCACCTCCAACGAAATTACAAGCATCAATCGAAC
TCCTGGCGCTAGACGCAGCGTATCTCTTTGCCCCTGGTGCAAGATGCGAGTGGTTGAAAAGTGTACCCAGTTGTAAG
GATGGATCGCTAACAGTGGGACGTTTCAGTCCCGTAGTCGGGATTTAGTGGTTGGAAAGTTGAGTTCCCAGGAGCAT
TAAAATTTAACATTCATGAATCGTAATTGTATTTTATCCGCTACTACCCATACTCTAACTATGACTCAACAACAGAC
AGCCTGTTCTTCAGTAATAACATCAGAACAAGTTCTTGAAACGCTGAGGAACTATCCCAATCTCTTTGAAAAATTTA
GTATTAAAACTCTGGCATTATTTGGGTCAACTGCTCATAATCAAGCCACAGCGACCAGTGACTTAGACTTTGTTGTT
GAATTTCAAAATGAAACAACCCTTAGCTTGTACATGGACTTAAAGTTTTTCCTGGAAGAATTATTTAATAAACCGGT
TGACTTAGCAACCAAAAAGTCTTTAAAAGAAATCATTCGTGAACAAGTATTGAATGAGGCTAAATATGTCTAGGAGC
CTTAAGCTTTACCTCAATGATATTCTAACAAGCATTGACAAAATCCAGGAATATAGTGAGGGTCTGGAAAAAGAAGC
ATTCTTAAAGCACTCACTTATCTTTGATGCAGTGACCCTTAACCTACAAATCATTGGAGAAGCCAGCAAGAAAATCC
CAGAACAAATCCGAAATCAGTATCCACATATTCCATGGCGAAACATCATTGGCCTGAGAAACATTATTGCCCACACA
TACTTTTACTTGGACGAAGACATTCTTTGGCACACTATCCAGCATGAACTGGAACCACTACAAAAGTGCATCCAGGA
ACTCTGGGATAAAGAAGCATAACTAACCACTAAGTACAGGACAAAAATTGTATGGGGTGAAACCCTGACCCAAGTAG
AGGTTGGGTAGAAACGGTAAAAAAGCGACGTAATAAGTCAAAACCATAGCGAAACAAACTCCGCGCT

TABLE 16
Type I Signal Sequences

| | | | | SEQ | | SEQ |
|---|---|---|---|---|---|---|
| Species of | | Leader | | ID | | ID |
| Origin | Gene | Name | DNA Seq. | NO | Prot. Seq. | NO |
| Synechococcus | SYNPC | F1 | CTCAACTTTTTAGCAA | | LNFLASGGDGYPFP | |
| sp. | C7002_ | | GTGGTGGTGATGGCTA | | TGDSVNRVDLTDL | |
| PCC 7002 | G0067 | | TCCATTTCCCACAGGT | | DGDGQDDNQLTGD | |
| | | | GACTCAGTCAACCGAG | | ATFAADGTEQDAL | |
| | | | TTGATCTCACTGACCT | | AEYLLDNFSTPETA | |
| | | | TGATGGCGATGGTCAA | | FAQEDVGRTLDERI | |
| | | | GACGACAATCAGCTA | | QNLNFRDDSVLGES | |
| | | | ACCGGGGATGCCACCT | | TNGQVIFRAINLISSI | |
| | | | TCGCAGCAGATGGAA | | FQRVLGPFSNNVNL | |
| | | | CTGAACAGGATGCTCT | | GNILFSDEEGVEDS | |
| | | | AGCTGAGTATTTACTA | | FDIFNTNLRQRILG | |
| | | | GATAACTTCAGTACTC | | NRNNTTSLNNLDN | |
| | | | CAGAAACAGCATTTGC | | QMWGRNNSDDVM | |
| | | | TCAAGAGGATGTAGG | | NALGGDDSGYGQS | |
| | | | CCGTACTCTGGATGAA | | DDDILRGDRGNGIL | |
| | | | CGTATCCAAAACCTCA | | NGGIGDDILTGGKG | |
| | | | ACTTCCGTGATGATAG | | LGTFVLNSGGAGV | |
| | | | TGTACTTGGTGAATCT | | NTITDFELGIDRIVL | |
| | | | ACTAAGGCCAGGTTA | | GNLSVNEVQLADT | |
| | | | TCTTTAGAGCGATAAA | | SINTMMSASPSDLL | |
| | | | TTTAATTTCCTCTATTT | | GIFTGVQLSGFESE | |
| | | | TTCAAAGAGTTTTGGG | | VFA | |
| | | | GCCATTTAGCAACAAT | | | |
| | | | GTGAATCTGGGTAACA | | | |
| | | | TCCTCTTCAGTGATGA | | | |
| | | | AGAAGGTGTTGAAGA | | | |
| | | | TAGCTTTGATATCTTC | | | |
| | | | AACACAAATTTACGGC | | | |
| | | | AGCGTATCCTGGGGAA | | | |
| | | | TCGCAACAACACCACT | | | |
| | | | TCCTTAAATAACCTGG | | | |
| | | | ATAACCAGATGTGGG | | | |
| | | | GCCGCAATAACTCGGA | | | |
| | | | TGATGTGATGAACGCC | | | |
| | | | TTGGGCGGTGACGATT | | | |
| | | | CCGGGTACGGCCAGA | | | |
| | | | GTGATGATGACATCCT | | | |
| | | | GCGCGGCGATCGCGG | | | |
| | | | CAACGGCATCCTGAAT | | | |
| | | | GGTGGCATAGGTGATG | | | |
| | | | ACATTCTCACGGGTGG | | | |
| | | | CAAGGGTCTAGGAAC | | | |
| | | | CTTTGTCCTCAACTCC | | | |
| | | | GGCGGGGCAGGCGTT | | | |
| | | | AATACCATCACTGACT | | | |
| | | | TTGAACTCGGCATTGA | | | |
| | | | CCGTATTGTCTTAGGC | | | |
| | | | AACTTAAGCGTTAACG | | | |
| | | | AGGTTCAGTTGGCTGA | | | |
| | | | CACATCTATTAACACT | | | |
| | | | ATGATGTCGGCTAGTC | | | |
| | | | CCAGTGATCTACTAGG | | | |
| | | | CATCTTTACCGGTGTA | | | |
| | | | CAGCTCAGTGGTTTTG | | | |
| | | | AAAGCGAGGTTTTTGC | | | |
| | | | ATAA | | | |
| Synechococcus | SYNPC | F2 | GATGATGACATCCTGC | | DDDILRGDRGNGIL | |
| sp. | C7002_ | | GCGGCGATCGCGGCA | | NGGIGDDILTGGKG | |
| PCC 7002 | G0067 | | ACGGCATCCTGAATGG | | LGTFVLNSGGAGV | |
| | | | TGGCATAGGTGATGAC | | NTITDFELGIDRIVL | |
| | | | ATTCTCACGGGTGGCA | | GNLSVNEVQLADT | |
| | | | AGGGTCTAGGAACCTT | | SINTMMSASPSDLL | |
| | | | TGTCCTCAACTCCGGC | | GIFTGVQLSGFESE | |
| | | | GGGGCAGGCGTTAAT | | VFA | |
| | | | ACCATCACTGACTTTG | | | |
| | | | AACTCGGCATTGACCG | | | |
| | | | TATTGTCTTAGGCAAC | | | |
| | | | TTAAGCGTTAACGAGG | | | |
| | | | TTCAGTTGGCTGACAC | | | |
| | | | ATCTATTAACACTATG | | | |
| | | | ATGTCGGCTAGTCCCA | | | |
| | | | GTGATCTACTAGGCAT | | | |
| | | | CTTTACCGGTGTACAG | | | |
| | | | CTCAGTGGTTTTGAAA | | | |
| | | | GCGAGGTTTTTGCATA | | | |
| | | | A | | | |
| Synechococcus | SYNPC | F3 | GTACTTGGTGAATCTA | | VLGESTNGQVIFRA | |
| sp. | C7002_ | | CTAATGGCCAGGTTAT | | INLISSIFQRVLGPFS | |
| PCC 7002 | G0067 | | CTTTAGAGCGATAAAT | | NNVNLGNILFSDEE | |
| | | | TTAATTTCCTCTATTTT | | GVEDSFDIFNTNLR | |
| | | | TCAAAGAGTTTTGGGG | | QRILGNRNNTTSLN | |
| | | | CCATTTAGCAACAATG | | NLDNQMWGRNNS | |
| | | | TGAATCTGGGTAACAT | | DDVMNALGGDDS | |
| | | | CCTCTTCAGTGATGAA | | GYGQSDDDILRGD | |
| | | | GAAGGTGTTGAAGAT | | RGNGILNGGIGDDI | |
| | | | AGCTTTGATATCTTCA | | LTGGKGLGTFVLNS | |
| | | | ACACAAATTTACGGCA | | GGAGVNTITDFELG | |
| | | | GCGTATCCTGGGGAAT | | IDRIVLGNLSVNEV | |
| | | | CGCAACAACACCACTT | | QLADTSINTMMSAS | |
| | | | CCTTAAATAACCTGGA | | PSDLLGIFTGVQLS | |
| | | | TAACCAGATGTGGGGC | | GFESEVFA | |
| | | | CGCAATAACTCGGATG | | | |
| | | | ATGTGATGAACGCCTT | | | |
| | | | GGGCGGTGACGATTCC | | | |
| | | | GGGTACGGCCAGAGT | | | |
| | | | GATGATGACATCCTGC | | | |
| | | | GCGGCGATCGCGGCA | | | |
| | | | ACGGCATCCTGAATGG | | | |
| | | | TGGCATAGGTGATGAC | | | |
| | | | ATTCTCACGGGTGGCA | | | |
| | | | AGGGTCTAGGAACCTT | | | |
| | | | TGTCCTCAACTCCGGC | | | |
| | | | GGGGCAGGCGTTAAT | | | |
| | | | ACCATCACTGACTTTG | | | |
| | | | AACTCGGCATTGACCG | | | |
| | | | TATTGTCTTAGGCAAC | | | |
| | | | TTAAGCGTTAACGAGG | | | |
| | | | TTCAGTTGGCTGACAC | | | |
| | | | ATCTATTAACACTATG | | | |
| | | | ATGTCGGCTAGTCCCA | | | |
| | | | GTGATCTACTAGGCAT | | | |
| | | | CTTTACCGGTGTACAG | | | |
| | | | CTCAGTGGTTTTGAAA | | | |
| | | | GCGAGGTTTTTGCA | | | |
| Synechococcus | SYNPC | F18 | GCAACAGGGATACAG | | ATGIQLIDQLPDGL | |
| sp. | C7002_ | | TTAATCGATCAGTTGC | | YYVSGTGTDWTCP | |
| PCC 7002 | A2175 | | CTGATGGTCTTTATTA | | LVSFTAPGPPTPED | |
| | | | TGTTTCGGGCACAGGC | | LRDIECSYNGTLTP | |
| | | | ACAGATTGGACTTGTC | | GATAPTLTITVYVQ | |
| | | | CGCTTGTGAGCTTTAC | | DTAPSTLENFVTVF | |
| | | | CGCCCCAGGCCCGCCA | | GDQPDPNDDNNTD | |
| | | | ACCCCCGAAGATCTGA | | LDRTTITDGVANAP | |
| | | | GGGATATTGAATGCAG | | DLILVKRITAVISEN | |
| | | | TTACAACGGAACCCTT | | NTTNYTVYRDDTS | |
| | | | ACCCCAGGAGCCACG | | SDSTAANDNAPFW | |
| | | | GCACCAACCTTGACGA | | PGYSAGNQSNTFTV | |
| | | | TTACGGTGTATGTCCA | | GELGLEAKPNDTV | |
| | | | AGACACCGCCCCCAGT | | EYTIYFLNQGNAPA | |
| | | | ACTCTCGAAAATTTTG | | SNIKICDRLSQYLD | |
| | | | TTACAGTCTTTGGCGA | | YSPDAYGSSMGIKL | |
| | | | TCAACCCGATCCCAAT | | NFNNSETNLTGVA | |
| | | | GACGACAATAATACG | | DVDAGQFFGPDLTP | |
| | | | GATTTAGACCGGACAA | | SGCIRPDNLQPMTA | |
| | | | CGATTACTGATGGTGT | | ADNPNGTLRVELA | |
| | | | TGCTAACGCTCCTGAT | | NVDPATSPATPANS | |
| | | | TTAATTCTTGTAAAAC | | YGYIRFRARVK | |
| | | | GTATTACTGCGGTTAT | | | |
| | | | CAGCGAAAACAATAC | | | |
| | | | TACAAATTACACTGTG | | | |
| | | | TATAGGGATGATACGA | | | |
| | | | GTAGTGACAGTACCGC | | | |
| | | | AGCTAATGATAATGCG | | | |
| | | | CCGTTTTGGCCTGGTT | | | |
| | | | ATAGTGCGGGCAATCA | | | |
| | | | AAGTAACACCTTCACA | | | |
| | | | GTGGGAGAGTTAGGC | | | |
| | | | CTTGAGGCTAAACCGA | | | |
| | | | ATGATACAGTTGAATA | | | |
| | | | CACTATTTATTTCCTC | | | |
| | | | AATCAAGGCAATGCCC | | | |
| | | | CGGCCAGCAATATCAA | | | |
| | | | AATTTGCGATCGCCTA | | | |
| | | | TCCCAATACTTAGATT | | | |
| | | | ATTCGCCAGATGCTTA | | | |
| | | | CGGTTCATCTATGGGT | | | |
| | | | ATTAAACTGAACTTTA | | | |
| | | | ACAACAGTGAAACCA | | | |
| | | | ATTTAACGGGCGTTGC | | | |
| | | | TGATGTTGATGCGGGA | | | |
| | | | CAATTTTTCGGCCCTG | | | |
| | | | ACCTTACCCCTAGCGG | | | |
| | | | ATGTATTCGTCCAGAC | | | |
| | | | AACCTACAACCCATGA | | | |
| | | | CCGCCGCCGATAATCC | | | |
| | | | AAATGGAACCCTGAG | | | |
| | | | AGTTGAGCTAGCTAAT | | | |
| | | | GTTGACCCCGCGACTA | | | |
| | | | GCCCTGCGACTCCAGC | | | |
| | | | TAATTCCTATGGCTAT | | | |
| | | | ATCCGCTTCCGGGCTC | | | |
| | | | GTGTGAAATAA | | | |
| Synechococcus | SYNPC | F8 | GCTGGTCGCAACGTTA | | AGRNVSATNNVNA | |
| sp. | C7002_ | | GCGCAACAAACAATG | | GNNINAANNVEAG | |
| PCC 7002 | A2531 | | TCAATGCTGGCAATAA | | QDVNAVRNVSAGN | |
| | | | CATCAATGCCGCCAAC | | NVNVGNNANVGN | |
| | | | AATGTTGAAGCGGGTC | | NLQVGQDAFINRN | |
| | | | AAGATGTCAATGCTGT | | AVVGGVLDVTGNA | |
| | | | CCGTAACGTCAGCGCT | | QFDSNVNVTGETTL | |
| | | | GGTAACAATGTCAATG | | NGLTTTNGINNTGA | |
| | | | TTGGCAATAACGCTAA | | INTDTLNAAGAVDI | |
| | | | CGTTGGGAATAATCTG | | QGLTTTNGIDNTGA | |
| | | | CAAGTCGGTCAAGAC | | ITTDTLDVAGTLEV | |
| | | | GCCTTCATTAACAGAA | | DGTTTLNGPTTINN | |
| | | | ACGCGGTCGTGGGAG | | DLTVQNNTTLGDA | |
| | | | GCGTTCTAGACGTTAC | | AGDTLDVNAGNVF | |
| | | | CGGAAACGCACAATTC | | FNNLPSSSSTDLLVI | |
| | | | GATAGTAATGTTAATG | | ESDGRVGVNNNIID | |
| | | | TTACTGGCGAAACAAC | | DLRSGIAATIAMDN | |
| | | | TCTCAACGGTTTAACC | | AEAELRPGHRFAIG | |
| | | | ACAACCAATGGCATCA | | IGLGVYEDETAIGT | |
| | | | ACAACACCGGAGCTAT | | SGKFLFTDPNSTGT | |
| | | | CAATACTGATACTCTA | | AVTFKASAGFGLTT | |
| | | | AATGCAGCCGGTGCTG | | DSFAAGAGLGLSF | |
| | | | TGGATATTCAGGGTTT | | | |
| | | | AACGACAACTAATGG | | | |
| | | | CATCGACAATACCGGT | | | |
| | | | GCGATTACAACTGATA | | | |
| | | | CTCTCGACGTGGCAGG | | | |
| | | | CACCCTGGAAGTAGAT | | | |
| | | | GGTACAACTACTCTCA | | | |
| | | | ATGGTCCCACGACTAT | | | |
| | | | CAATAATGATCTAACT | | | |
| | | | GTTCAAAACAATACAA | | | |
| | | | CACTTGGCGATGCTGC | | | |
| | | | CGGTGATACTCTAGAC | | | |
| | | | GTCAATGCTGGCAATG | | | |
| | | | TTTTCTTCAATAACCTT | | | |
| | | | CCCAGCAGCAGCTCCA | | | |
| | | | CTGACCTCCTCGTTAT | | | |
| | | | CGAAAGTGACGGTCG | | | |
| | | | AGTCGGTGTAAACAAC | | | |
| | | | AATATCATTGATGATC | | | |
| | | | TCAGATCTGGTATTGC | | | |
| | | | TGCCACCATTGCGATG | | | |
| | | | GATAACGCAGAAGCT | | | |
| | | | GAACTTCGTCCTGGTC | | | |
| | | | ATCGCTTTGCCATCGG | | | |
| | | | TATTGGTCTCGGGGTC | | | |
| | | | TACGAAGACGAAACT | | | |
| | | | GCAATTGGTACTTCTG | | | |
| | | | GTAAGTTCCTCTTTAC | | | |
| | | | CGATCCCAACAGCACT | | | |
| | | | GGAACCGCTGTTACTT | | | |
| | | | TCAAAGCAAGTGCTGG | | | |
| | | | TTTCGGTCTTACTACC | | | |
| | | | GATAGCTTCGCTGCCG | | | |
| | | | GTGCAGGTCTCGGCCT | | | |
| | | | AAGCTTCTAA | | | |
| Anabaena | a110364 | F14 | ATCGTTACGGAAAACG | | MVTENANEGIDTV | |
| sp. PCC | | | CTAACGAAGGTATAG | | QSSVTYTLGANVE | |
| 7120 | | | ACACAGTTCAGTCATC | | NLTLTGTGAINGTG | |
| | | | TGTTACTTATACTCTG | | NSLNNTITGNSGNN | |
| | | | GGCGCGAATGTAGAA | | TLNGDAGNDFLIAG | |
| | | | AATTTGACTCTGACTG | | NGNDILNGGTGND | |
| | | | GTACGGGTGCAATCAA | | TMLGGGGNDTYIV | |
| | | | CGGTACAGGTAACAGT | | DSIGDYVLENANQ | |
| | | | CTCAACAATACGATCA | | GTDLVQSSISYTLG | |
| | | | CTGGCAACAGTGGCA | | NSLENLTLTGTSAI | |
| | | | ATAATACCCTCAATGG | | NGTGNRLNNVITG | |
| | | | CGATGCTGGTAATGAT | | NSGNNTLNGGDGN | |
| | | | TTCCTGATTGCTGGCA | | DTLNGSAGVDTLL | |
| | | | ATGGTAATGACATTCT | | GGNGNDILVGGTG | |
| | | | CAATGGTGGTACAGGC | | NDTLTGGVGRDRF | |
| | | | AATGATACGATGCTTG | | TFNSRSEGIDRITDF | |
| | | | GTGGCGGAGGTAACG | | NVVDDTIVVSAAG | |
| | | | ACACCTACATTGTTGA | | FGGGLVVGAAIASS | |
| | | | TAGTATAGGCGACTAC | | QFLLGSAATTASHR | |
| | | | GTTTTGGAAAATGCCA | | FLYDRNNGALFFD | |
| | | | ACCAAGGTACAGACTT | | QDGTGAIAKVQFA | |
| | | | AGTTCAGTCATCTATC | | TLNTGLSLTNADIL | |
| | | | AGCTATACATTAGGCA | | VVA | |
| | | | ATAGTTTAGAGAATTT | | | |
| | | | GACTCTCACAGGTACA | | | |
| | | | TCTGCAATCAATGGTA | | | |
| | | | CAGGTAACCGTCTTAA | | | |
| | | | CAACGTCATTACAGGT | | | |
| | | | AACAGTGGCAACAAT | | | |
| | | | ACCCTAAATGGTGGAG | | | |
| | | | ATGGCAATGATACTCT | | | |
| | | | TAATGGTAGTGCAGGT | | | |
| | | | GTTGATACTCTCCTTG | | | |
| | | | GTGGTAACGGTAATGA | | | |
| | | | CATCCTCGTTGGTGGT | | | |
| | | | ACTGGTAACGATACAC | | | |
| | | | TAACAGGGGGTGTAG | | | |
| | | | GACGCGATCGCTTTAC | | | |
| | | | ATTCAATTCTCGTAGT | | | |
| | | | GAAGGTATCGACAGA | | | |
| | | | ATTACCGATTTTAACG | | | |
| | | | TGGTTGATGACACTAT | | | |
| | | | TGTTGTCTCTGCGGCT | | | |
| | | | GGCTTTGGTGGCGGGT | | | |
| | | | TGGTTGTAGGTGCGGC | | | |
| | | | GATCGCATCTAGTCAG | | | |
| | | | TTTTTACTAGGTTCAG | | | |
| | | | CCGCCACTACTGCTAG | | | |
| | | | CCACCGATTCCTCTAC | | | |
| | | | GACCGAAACAACGGC | | | |
| | | | GCTCTCTTCTTTGATC | | | |
| | | | AGGATGGCACGGGTG | | | |
| | | | CGATCGCTAAAGTTCA | | | |
| | | | ATTTGCTACCCTCAAT | | | |
| | | | ACTGGACTGTCCTTGA | | | |
| | | | CCAATGCAGATATTCT | | | |
| | | | CGTTGTTGCTTAG | | | |
| Anabaena | a112654 | F12 | TTGCGAGTCTTTGATG | | MRVFDAEGNELAK | |
| sp. PCC | | | CAGAAGGTAATGAAC | | TDFDDFQAAPDEVF | |
| 7120 | | | TGGCGAAGACCGATTT | | SAFNDPYLEFTAET | |
| | | | TGATGACTTTCAAGCC | | TGTYYVGISQIGND | |
| | | | GCACCGGATGAGGTGT | | YYDPNVVGSGSGW | |
| | | | TCTCAGCCTTTAATGA | | LFADFGIENGEYTV | |
| | | | CCCTTACTTAGAGTTC | | SFNLTPEQPTNPVG | |
| | | | ACCGCTGAAACAACTG | | TSGDDTLIGTDEEE | |
| | | | GTACTTACTATGTTGG | | SLFGNGGNDILYAR | |
| | | | CATCAGTCAGATTGGT | | GGDDKLFGGAGDD | |
| | | | AATGACTATTATGATC | | LLDGGEGNDALFG | |
| | | | CGAATGTGGTTGGTAG | | GAGTDTLLGGAGN | |
| | | | TGGTTCTGGTTGGCTA | | DYLTGGTGDNLLD | |
| | | | TTCGCTGATTTCGGAA | | GGDGNDLLYGNGG | |
| | | | TTGAAAATGGTGAGTA | | QDTLLGGAGDDIIY | |
| | | | CACAGTTAGTTTTAAT | | SGSGDDLINGGLGN | |
| | | | CTGACTCCAGAACAAC | | DIIFLNGGQDTIVV | |
| | | | CCACTAACCCCGTTGG | | AQGAGIDTINNFQV | |
| | | | GACTTCAGGTGATGAT | | SLGQKVGLSGGITF | |
| | | | ACCCTGATTGGGACTG | | EQLTFSQSGLDTLI | |
| | | | ACGAGGAAGAGAGCC | | QVGDEALAVLKFV | |
| | | | TGTTTGGTAATGGTGG | | QSSSLSSAAFTVV | |
| | | | TAATGACATACTCTAT | | | |
| | | | GCTAGAGGCGGTGAT | | | |
| | | | GACAAGCTATTTGGCG | | | |
| | | | GTGCTGGTGACGACCT | | | |
| | | | CTTAGATGGTGGCGAG | | | |
| | | | GGTAATGACGCGTTGT | | | |
| | | | TTGGTGGTGCTGGTAC | | | |
| | | | AGATACCTTGCTTGGT | | | |
| | | | GGTGCTGGTAATGATT | | | |
| | | | ACTTAACTGGTGGTAC | | | |
| | | | TGGCGACAATCTATTA | | | |
| | | | GATGGGGGTGACGGT | | | |
| | | | AATGATCTCCTCTATG | | | |
| | | | GTAATGGTGGTCAAGA | | | |
| | | | TACTTTACTGGGCGGT | | | |
| | | | GCTGGTGATGACATTA | | | |
| | | | TCTACAGTGGCTCTGG | | | |
| | | | TGATGACTTGATTAAT | | | |
| | | | GGTGGTCTTGGTAATG | | | |
| | | | ACATCATCTTCTTGAA | | | |
| | | | TGGTGGTCAAGATACT | | | |
| | | | ATAGTTGTGGCTCAAG | | | |
| | | | GTGCGGGTATTGACAC | | | |
| | | | TATCAACAATTTCCAA | | | |
| | | | GTCAGTTTGGGTCAAA | | | |
| | | | AGGTTGGTTTGAGTGG | | | |
| | | | TGGTATCACTTTTGAG | | | |
| | | | CAACTAACTTTCAGTC | | | |
| | | | AAAGTGGTTTGGATAC | | | |
| | | | GCTGATTCAGGTCGGT | | | |
| | | | GATGAGGCTCTGGCTG | | | |
| | | | TGTTGAAGTTTGTTCA | | | |
| | | | ATCTAGTAGTCTGAGT | | | |
| | | | TCTGCGGCGTTTACTG | | | |
| | | | TTGTTTAA | | | |
| Anabaena | a112655 | F21 | ACCAGAGCGTCATTAG | | TRASLGEFVIFNED | |
| sp. PCC | | | GTGAGTTTGTTATCTT | | GTPAVTWEGIAGFP | |
| 7120 | | | TAATGAAGATGGTACA | | EPDGTGGGFFVTLT | |
| | | | CCTGCTGTCACCTGGG | | EPTASLSLKVFDDG | |
| | | | AAGGTATTGCTGGCTT | | ANEGIESLTFNLVD | |
| | | | CCCTGAACCAGATGGC | | GEQYQVSPDAGSIA | |
| | | | ACTGGTGGCGGTTTCT | | LTISDTPTNPVGDA | |
| | | | TCGTCACTTTAACCGA | | GDNILVGDGNNNS | |
| | | | ACCGACAGCATCCCTC | | LFGNAGNDRIFGGL | |
| | | | AGCCTGAAGGTGTTTG | | GNDYLFGGADDDL | |
| | | | ATGATGGTGCTAATGA | | LNGGDGNDALFGG | |
| | | | AGGTATTGAAAGCTTA | | AGNNTLLGGAGND | |
| | | | ACCTTCAATTTGGTGG | | YLTGGAGNNLLDG | |
| | | | ATGGAGAACAGTATC | | GDGNDILYGGNGN | |
| | | | AAGTCAGCCCTGATGC | | NTLLGGAGNDIIYS | |
| | | | TGGTAGTATTGCTCTG | | GSGDDLINGGLGN | |
| | | | ACTATCAGTGATACCC | | DTIFLNGGQDTVVV | |
| | | | CAACCAATCCTGTTGG | | AQGAGIDTINNFQV | |
| | | | TGATGCTGGTGACAAC | | SLGQKVGLSGGLTF | |
| | | | ATCCTAGTTGGTGATG | | EQLTLTQSGLDTLV | |
| | | | GCAATAACAACAGTTT | | KVGDETLAVLKFV | |
| | | | GTTTGGTAATGCTGGC | | QSSDLSSSAFTTV | |
| | | | AATGACCGCATCTTTG | | | |
| | | | GTGGTCTGGGTAATGA | | | |
| | | | CTACCTGTTTGGCGGT | | | |
| | | | GCTGACGACGACCTCT | | | |
| | | | TAAATGGTGGCGACG | | | |
| | | | GTAACGACGCGCTGTT | | | |
| | | | TGGTGGTGCTGGTAAT | | | |
| | | | AACACCCTATTAGGTG | | | |
| | | | GTGCTGGTAATGACTA | | | |
| | | | CTTAACTGGTGGTGCT | | | |
| | | | GGCAATAACCTCTTAG | | | |
| | | | ATGGAGGTGACGGTA | | | |
| | | | ACGATATCCTCTATGG | | | |
| | | | TGGTAATGGTAATAAT | | | |
| | | | ACTTTACTAGGTGGTG | | | |
| | | | CTGGTAATGACATCAT | | | |
| | | | CTACAGTGGCTCTGGT | | | |
| | | | GATGACCTGATTAACG | | | |
| | | | GTGGTCTTGGTAATGA | | | |
| | | | CACCATTTTCTTGAAT | | | |
| | | | GGTGGACAAGATACT | | | |
| | | | GTGGTTGTGGCTCAAG | | | |
| | | | GTGCAGGTATTGACAC | | | |
| | | | TATCAACAATTTCCAA | | | |
| | | | GTCAGTTTGGGTCAAA | | | |
| | | | AGGTTGGTTTGAGTGG | | | |
| | | | TGGACTTACCTTTGAG | | | |
| | | | CAATTGACTTTGACTC | | | |
| | | | AAAGCGGTTTGGATAC | | | |
| | | | GTTGGTGAAAGTTGGT | | | |
| | | | GATGAAACTCTGGCTG | | | |
| | | | TGTTGAAGTTTGTTCA | | | |
| | | | ATCTAGTGATTTGAGT | | | |
| | | | TCTTCAGCTTTTACAA | | | |
| | | | CGGTCTAA | | | |
| Anabaena | a112793 | F11 | ACGGCGAATCCTGATA | | TANPDSNIYPVKVN | |
| sp. PCC | | | GCAATATCTATCCAGT | | RGDRTIEVEGFQGV | |
| 7120 | | | TAAAGTCAACCGTGGC | | GRGSNPSLEVRETF | |
| | | | GATCGCACTATTGAGG | | DELIFTGEGLVAKN | |
| | | | TAGAAGGGTTTCAGGG | | LLLTQTGDDLVVSF | |
| | | | AGTAGGACGGGGAAG | | EGVDDTQVILKDFA | |
| | | | CAATCCCTCGCTGGAA | | LENLDNLPIPGGQH | |
| | | | GTGCGGGAAACCTTTG | | GQIGNIMFDGDETL | |
| | | | ATGAACTCATATTTAC | | QDSFDVFDADSTQ | |
| | | | AGGAGAGGGTTTAGTT | | NRIWNRNTVTFLN | |
| | | | GCCAAAAACTTGCTCC | | DLDNHVRGFDNSD | |
| | | | TTACCCAAACTGGTGA | | DVINGQGGNDIIGG | |
| | | | TGATTTAGTTGTCAGT | | LSGDDILRGGEGND | |
| | | | TTTGAAGGGGTTGATG | | ILYAGTGTDILVGG | |
| | | | ATACCCAAGTGATTCT | | LGNDTLYLGSDRHI | |
| | | | CAAGGACTTTGCTTTA | | DTVIYRQGDGSDVI | |
| | | | GAAAACCTGGATAACT | | HQFQRGAGGDLLQ | |
| | | | TGCCGATTCCTGGTGG | | FEGIEAIDVVVHGR | |
| | | | TCAGCATGGTCAGATT | | NTYFHLGDGVTGN | |
| | | | GGTAACATCATGTTTG | | TGFGSGELLAELRG | |
| | | | ATGGTGATGAAACCCT | | VGGFTSDNIGLNLA | |
| | | | GCAAGATAGTTTTGAT | | SGNTAQFLFA | |
| | | | GTCTTTGACGCAGACT | | | |
| | | | CCACGCAAAACAGAA | | | |
| | | | TTTGGAATCGCAACAC | | | |
| | | | CGTCACCTTCCTGAAT | | | |
| | | | GATTTAGATAATCATG | | | |
| | | | TACGTGGCTTTGACAA | | | |
| | | | CTCCGATGATGTCATC | | | |
| | | | AACGGTCAAGGTGGT | | | |
| | | | AATGACATTATTGGGG | | | |
| | | | GTTTGAGTGGCGATGA | | | |
| | | | TATTTTGCGCGGTGGT | | | |
| | | | GAAGGTAATGATATCC | | | |
| | | | TTTATGCTGGAACAGG | | | |
| | | | TACTGATATTCTCGTA | | | |
| | | | GGTGGGCTAGGAAAC | | | |
| | | | GATACCCTGTATTTGG | | | |
| | | | GAAGTGATCGCCACAT | | | |
| | | | TGATACAGTAATATAT | | | |
| | | | CGTCAAGGTGATGGCA | | | |
| | | | GTGATGTGATCCATCA | | | |
| | | | GTTCCAGCGTGGTGCA | | | |
| | | | GGCGGAGATTTATTGC | | | |
| | | | AATTTGAAGGTATCGA | | | |
| | | | GGCGATCGATGTAGTG | | | |
| | | | GTGCATGGCCGCAATA | | | |
| | | | CCTATTTCCATTTAGG | | | |
| | | | TGACGGGGTGACTGG | | | |
| | | | AAATACAGGATTTGGT | | | |
| | | | TCAGGTGAGTTATTAG | | | |
| | | | CCGAGTTACGCGGTGT | | | |
| | | | CGGGGGATTTACCTCA | | | |
| | | | GATAACATCGGGTTAA | | | |
| | | | ATCTGGCATCTGGCAA | | | |
| | | | TACTGCACAGTTCTTG | | | |
| | | | TTTGCATAA | | | |
| Anabaena | a117128 | F13 | TCCCTTTCTGGTACAT | | SLSGTSSADVLNGF | |
| sp. PCC | | | CTAGTGCAGATGTTCT | | GGDDYIEGLAGND | |
| 7120 | | | CAACGGCTTTGGTGGT | | TIDGGIGRFDRLFG | |
| | | | GATGATTATATAGAAG | | GDGDDAITDPDGIL | |
| | | | GTTTAGCTGGGAATGA | | GAHGGLGNDTINV | |
| | | | CACAATAGATGGTGG | | TFAANWDNDSNPN | |
| | | | GATTGGAAGATTTGAT | | NSPRSDGKITGGYG | |
| | | | CGGTTGTTTGGCGGTG | | DDNITVTMNNSKFF | |
| | | | ATGGAGATGATGCAAT | | INMKGDEPVNNAQ | |
| | | | TACCGATCCAGATGGA | | GGNDVITLLGSYQN | |
| | | | ATCTTAGGAGCGCATG | | AIVDLGGGDDTFIG | |
| | | | GTGGTTTAGGCAACGA | | GNGSDNVSGGAGN | |
| | | | TACAATCAACGTTACT | | DTIFGFGGNDNLTG | |
| | | | TTTGCTGCCAACTGGG | | NDGDDILVGGSGN | |
| | | | ATAATGATAGTAATCC | | DRLTGGSGKDIFSF | |
| | | | CAACAACTCCCCACGT | | SSLADGIDTITDFSV | |
| | | | TCTGATGGCAAGATTA | | ADDKIRVNAAGFG | |
| | | | CTGGAGGCTACGGCG | | SGLVAGNLDASQF | |
| | | | ACGATAACATTACAGT | | VLGSSAQDGSDRFI | |
| | | | AACGATGAATAATAG | | YNQATGALLFDVD | |
| | | | CAAGTTCTTCATCAAC | | GIGANTAVQIATLS | |
| | | | ATGAAGGGTGATGAG | | NKIAINSTSIVIV | |
| | | | CCAGTTAATAACGCTC | | | |
| | | | AAGGCGGTAATGATGT | | | |
| | | | AATTACACTATTAGGA | | | |
| | | | AGCTACCAAAATGCA | | | |
| | | | ATTGTTGACCTGGGAG | | | |
| | | | GTGGCGACGATACTTT | | | |
| | | | TATAGGTGGCAATGGC | | | |
| | | | AGTGATAATGTCTCTG | | | |
| | | | GTGGTGCTGGCAACGA | | | |
| | | | TACCATTTTTGGTTTC | | | |
| | | | GGAGGTAATGACAAC | | | |
| | | | TTAACTGGCAATGACG | | | |
| | | | GTGATGATATTCTCGT | | | |
| | | | CGGTGGTAGCGGTAAC | | | |
| | | | GATCGCTTAACTGGTG | | | |
| | | | GTAGTGGGAAAGATA | | | |
| | | | TTTTTAGCTTCTCTTCT | | | |
| | | | CTTGCTGATGGCATTG | | | |
| | | | ACACCATTACAGACTT | | | |
| | | | TAGCGTTGCTGATGAC | | | |
| | | | AAAATTCGTGTCAATG | | | |
| | | | CTGCTGGGTTCGGTAG | | | |
| | | | TGGGCTTGTAGCTGGT | | | |
| | | | AATCTGGACGCATCAC | | | |
| | | | AATTTGTCTTGGGTTC | | | |
| | | | ATCTGCACAAGATGGA | | | |
| | | | AGCGATCGCTTTATCT | | | |
| | | | ACAATCAAGCAACTG | | | |
| | | | GCGCTCTGTTGTTTGA | | | |
| | | | TGTTGACGGTATAGGG | | | |
| | | | GCGAATACTGCCGTTC | | | |
| | | | AAATTGCCACTCTGTC | | | |
| | | | AAATAAAATTGCGATT | | | |
| | | | AACTCTACAAGTATTG | | | |
| | | | TAATTGTCTAA | | | |
| Anabaena | alr0791 | F15 | AACAATGCCGTCAATC | | NNAVNRLEGGDGN | |
| sp. PCC | | | GCTTAGAAGGCGGTG | | DWLIGKDGNDILIG | |
| 7120 | | | ACGGCAATGACTGGTT | | GNGNDRLNGETGE | |
| | | | AATCGGTAAAGATGGT | | DTLEGGLGNDVYEI | |
| | | | AACGATATCCTGATTG | | DSVGDVIIEAADAG | |
| | | | GCGGTAATGGTAATGA | | IDTVISSVDWTLGV | |
| | | | CCGACTCAATGGCGAG | | NLENLTLVGNQAT | |
| | | | ACTGGTGAGGATACAT | | LGIGNDLDNRITGN | |
| | | | TAGAAGGTGGTTTAGG | | NADNVLFGEAGND | |
| | | | TAACGACGTTTATGAA | | ILNGGAGNDELFGS | |
| | | | ATTGATAGTGTAGGCG | | DGNDILNGGAGND | |
| | | | ACGTAATTATTGAAGC | | ELFGSDGNDILNGG | |
| | | | CGCAGATGCAGGAAT | | AGNDELFGGAGND | |
| | | | AGATACAGTCATCTCA | | ILNGGTGADSFSFG | |
| | | | TCGGTAGATTGGACTT | | NPGNPFNNSDFGID | |
| | | | TAGGGGTGAATCTGGA | | TVADFAVGVDDIK | |
| | | | AAACTTGACTTTGGTG | | LDKVSFSALTSVVG | |
| | | | GGTAATCAAGCCACAT | | NGFSVGGEFASVSN | |
| | | | TAGGCATAGGCAATG | | DTLAAISNGLIVYS | |
| | | | ATCTGGATAACCGCAT | | LGSGRLFYNQNGS | |
| | | | TACTGGTAATAATGCT | | ADGLGSGAHFATL | |
| | | | GATAATGTCTTGTTTG | | SGAPTLTANNFVIF | |
| | | | GTGAAGCTGGTAATGA | | | |
| | | | CATCCTGAATGGTGGT | | | |
| | | | GCTGGTAACGATGAGT | | | |
| | | | TGTTTGGTAGTGATGG | | | |
| | | | TAATGACATCCTGAAT | | | |
| | | | GGCGGTGCTGGCAAC | | | |
| | | | GATGAGTTGTTTGGTA | | | |
| | | | GTGATGGTAATGACAT | | | |
| | | | CCTGAATGGTGGTGCT | | | |
| | | | GGCAACGATGAGTTGT | | | |
| | | | TTGGTGGTGCTGGTAA | | | |
| | | | TGACATCCTGAATGGT | | | |
| | | | GGTACTGGTGCTGATT | | | |
| | | | CCTTCAGTTTTGGTAA | | | |
| | | | TCCGGGTAATCCCTTC | | | |
| | | | AACAATAGTGATTTTG | | | |
| | | | GTATAGATACTGTTGC | | | |
| | | | TGATTTTGCAGTTGGT | | | |
| | | | GTGGATGACATTAAGT | | | |
| | | | TAGATAAGGTCAGCTT | | | |
| | | | CTCCGCTCTAACTAGT | | | |
| | | | GTGGTTGGCAATGGTT | | | |
| | | | TTAGTGTAGGTGGTGA | | | |
| | | | GTTTGCCAGTGTCAGT | | | |
| | | | AACGATACATTGGCGG | | | |
| | | | CAATTAGCAATGGGTT | | | |
| | | | GATTGTTTACAGTTTA | | | |
| | | | GGTAGTGGTCGCTTGT | | | |
| | | | TCTATAACCAAAATGG | | | |
| | | | TAGTGCTGATGGTTTG | | | |
| | | | GGTTCTGGCGCTCACT | | | |
| | | | TTGCTACACTCTCCGG | | | |
| | | | CGCTCCCACTCTCACT | | | |
| | | | GCTAATAATTTCGTGA | | | |
| | | | TTTTTTAG | | | |
| Anabaena | alr1403 | F16 | GACACCGTTGTTTATG | | DTVVYDGNYADY | |
| sp. PCC | | | ACGGTAATTATGCAGA | | GISFLSNGDLQVID | |
| 7120 | | | TTATGGTATCTCTTTCC | | KNLTNGNDGTDTIR | |
| | | | TGAGCAATGGTGATTT | | GVEVINFRQGGSYG | |
| | | | GCAAGTCATTGACAAG | | VVTGTTGNDVLTA | |
| | | | AACCTCACCAATGGAA | | SNMWSFVFGGGGN | |
| | | | ATGACGGTACTGACAC | | DIITGGTGNDTLDG | |
| | | | CATCAGGGGTGTAGA | | STGNDTLIGGAGND | |
| | | | AGTCATCAACTTTAGA | | TLIGGAGVDTAVY | |
| | | | CAAGGCGGAAGTTAT | | AGNYADYGISFLSN | |
| | | | GGAGTGGTCACAGGT | | GDLQVIDKNLTNG | |
| | | | ACTACAGGTAATGATG | | NDGTDILKGVEVIN | |
| | | | TATTGACCGCATCAAA | | FTQGGSYGVVTGT | |
| | | | TATGTGGTCATTCGTC | | TGNNVLTASNMWS | |
| | | | TTCGGTGGTGGCGGTA | | FVFGGNGNDTITGG | |
| | | | ACGACATTATTACTGG | | TGNDTLVGGLGAD | |
| | | | TGGGACTGGCAACGAT | | TLTGGLGADKFVF | |
| | | | ACCTTGGATGGTAGTA | | NSLSEGIDVIKDFS | |
| | | | CTGGCAATGATACGTT | | WQQGDKIQILGSSF | |
| | | | GATTGGTGGCGCTGGC | | GATSTSQFSFDQNT | |
| | | | AATGATACGTTGATTG | | GGLFFNAQQFATLE | |
| | | | GTGGTGCTGGTGTTGA | | NKPAGFLTNADIQI | |
| | | | TACTGCCGTTTATGCG | | V | |
| | | | GGAAATTATGCAGATT | | | |
| | | | ATGGTATCTCTTTCCT | | | |
| | | | GAGCAATGGTGATTTG | | | |
| | | | CAAGTCATTGACAAGA | | | |
| | | | ACCTCACCAATGGAAA | | | |
| | | | TGACGGTACTGACATC | | | |
| | | | CTCAAGGGTGTAGAA | | | |
| | | | GTCATCAACTTTACAC | | | |
| | | | AAGGCGGAAGTTATG | | | |
| | | | GAGTGGTCACAGGTAC | | | |
| | | | TACTGGTAATAATGTA | | | |
| | | | TTGACCGCATCAAATA | | | |
| | | | TGTGGTCATTTGTCTT | | | |
| | | | CGGTGGTAATGGTAAC | | | |
| | | | GACACTATTACTGGCG | | | |
| | | | GCACTGGCAATGATAC | | | |
| | | | TTTAGTCGGAGGGCTT | | | |
| | | | GGTGCTGATACCCTCA | | | |
| | | | CAGGTGGACTTGGGGC | | | |
| | | | TGATAAATTTGTCTTT | | | |
| | | | AACTCTCTTTCTGAAG | | | |
| | | | GAATTGATGTGATCAA | | | |
| | | | AGACTTTTCTTGGCAA | | | |
| | | | CAAGGAGATAAGATT | | | |
| | | | CAAATTCTCGGCTCTA | | | |
| | | | GTTTTGGTGCAACTTC | | | |
| | | | CACTAGTCAGTTCAGC | | | |
| | | | TTTGACCAGAATACAG | | | |
| | | | GTGGTTTATTCTTTAA | | | |
| | | | CGCCCAGCAATTTGCC | | | |
| | | | ACTCTTGAGAACAAAC | | | |
| | | | CTGCTGGTTTCTTGAC | | | |
| | | | AAATGCTGACATCCAA | | | |
| | | | ATTGTTTAG | | | |
| Anabaena | alr4238 | F19 | AATGATTTCGGTGTCA | | NDFGVTGTTTNPD | |
| sp. PCC | | | CGGGAACTACCACCA | | GTISIRVSPLAERLA | |
| 7120 | | | ATCCTGATGGGACAAT | | LLELPDNLPVTQPL | |
| | | | TAGCATTAGAGTTTCC | | DIQFGSSGSDNITAE | |
| | | | CCACTAGCTGAAAGAC | | PGQILFTGDGADTV | |
| | | | TGGCTCTCTTGGAACT | | DSPGNNTISTGNGD | |
| | | | CCCCGATAATTTACCA | | DTVFVGSDASVSTG | |
| | | | GTCACACAACCATTAG | | NGNDQIFIGVESPA | |
| | | | ATATTCAGTTCGGCTC | | SNTTANGGNGDDEI | |
| | | | CTCTGGTAGTGATAAT | | TVIEAGGSNNLFGA | |
| | | | ATTACGGCGGAACCTG | | AGNDTLQVIEGSRQ | |
| | | | GTCAAATATTATTCAC | | FAFGGSGNDTLTSN | |
| | | | AGGTGATGGTGCCGAT | | GSYNRLNGGSGDD | |
| | | | ACGGTAGATTCTCCTG | | KLFSSVNDSLFGGD | |
| | | | GGAATAATACTATCTC | | GDDVLFAGQAGSN | |
| | | | CACGGGCAACGGTGA | | RLTGGAGADQFWI | |
| | | | TGATACGGTATTTGTG | | ANGSLPTSKNTVTD | |
| | | | GGCAGTGATGCTTCTG | | FAVGVDKIGLGGIG | |
| | | | TCTCTACTGGTAATGG | | VTQFSALSLVQQG | |
| | | | TAACGATCAAATCTTC | | ADTLVKLGATELV | |
| | | | ATCGGTGTCGAGAGTC | | ALQGITSTSLSVTDF | |
| | | | CAGCCAGCAATACCAC | | VFAVSLVG | |
| | | | AGCTAATGGTGGTAAT | | | |
| | | | GGTGACGACGAAATC | | | |
| | | | ACCGTGATTGAAGCAG | | | |
| | | | GTGGAAGTAATAACCT | | | |
| | | | TTTTGGCGCAGCAGGT | | | |
| | | | AATGATACTCTGCAAG | | | |
| | | | TCATTGAAGGTTCTCG | | | |
| | | | TCAATTTGCCTTTGGT | | | |
| | | | GGTTCTGGTAACGACA | | | |
| | | | CCCTCACAAGTAACGG | | | |
| | | | TAGCTATAACCGTCTC | | | |
| | | | AATGGTGGTTCAGGAG | | | |
| | | | ATGACAAATTATTCTC | | | |
| | | | CAGTGTGAATGACTCT | | | |
| | | | TTGTTCGGTGGTGATG | | | |
| | | | GTGATGATGTGCTATT | | | |
| | | | TGCAGGTCAAGCTGGT | | | |
| | | | AGTAACCGCCTCACTG | | | |
| | | | GTGGCGCTGGTGCTGA | | | |
| | | | CCAGTTTTGGATTGCT | | | |
| | | | AATGGTAGTTTACCAA | | | |
| | | | CTAGCAAGAATACTGT | | | |
| | | | GACTGATTTTGCAGTC | | | |
| | | | GGTGTTGACAAAATCG | | | |
| | | | GACTGGGTGGAATTGG | | | |
| | | | TGTGACACAATTTAGC | | | |
| | | | GCTTTGAGTCTGGTAC | | | |
| | | | AGCAAGGCGCTGATA | | | |
| | | | CTTTGGTGAAACTAGG | | | |
| | | | GGCGACTGAGTTAGTT | | | |
| | | | GCATTACAAGGAATTA | | | |
| | | | CTTCAACTAGTCTGAG | | | |
| | | | TGTGACTGACTTTGTT | | | |
| | | | TTTGCTGTAAGTTTGG | | | |
| | | | TGGGTTAG | | | |
| Anabaena | alr7304 | F10 | AGTTGGACATTAGATG | | SWTLDDNLENLTL | |
| sp. PCC | | | ATAATTTAGAAAATCT | | TGSNAINGTGNALR | |
| 7120 | | | CACTCTCACAGGCAGC | | NTITGNSADNILSG | |
| | | | AATGCTATTAATGGGA | | GDNDDTLRGNAGN | |
| | | | CTGGTAATGCGCTGAG | | DILNGGAGNDSLD | |
| | | | AAATACCATCACAGGT | | GGLGDDVMTGGAS | |
| | | | AACAGTGCTGATAATA | | NDTYFVDSSNDTIIE | |
| | | | TCCTGTCTGGTGGTGA | | EADGGTDTVRASIT | |
| | | | TAACGATGACACTCTC | | LTLGDHLENLILIG | |
| | | | AGAGGAAATGCTGGC | | NSPIDGTGNALRNN | |
| | | | AACGATATTCTCAATG | | ITGNVANNILSGGA | |
| | | | GAGGTGCTGGTAACG | | DNDTIISGDGDDTL | |
| | | | ATTCCTTAGATGGTGG | | YGDSGNDTLTGGN | |
| | | | ACTTGGTGACGATGTA | | GNDILVGGMGSDR | |
| | | | ATGACAGGTGGCGCTA | | LTGGNGKDTFAFS | |
| | | | GTAATGATACTTATTT | | APITDGIDTITDFNP | |
| | | | CGTTGATAGCAGCAAT | | LDDLLRVDAAGFG | |
| | | | GACACCATCATAGAA | | GGLVAGTLLASQF | |
| | | | GAAGCTGATGGGGGA | | VLGTAAKTTSDRFI | |
| | | | ACTGATACTGTTCGTG | | YNQSTGALFFDVD | |
| | | | CCAGTATTACGCTAAC | | GTGSSSQVQIATLS | |
| | | | TTTAGGCGACCACTTA | | NKPVINATNISVI | |
| | | | GAAAATCTCATCTTGA | | | |
| | | | TCGGTAATAGCCCAAT | | | |
| | | | TGATGGTACTGGTAAT | | | |
| | | | GCTTTAAGAAATAATA | | | |
| | | | TTACTGGTAATGTCGC | | | |
| | | | AAACAACATCTTATCT | | | |
| | | | GGTGGTGCTGATAATG | | | |
| | | | ACACCATAATCAGTGG | | | |
| | | | AGATGGAGATGATAC | | | |
| | | | GCTTTATGGCGATAGT | | | |
| | | | GGTAATGATACTTTAA | | | |
| | | | CTGGCGGGAACGGCA | | | |
| | | | ACGATATACTCGTGGG | | | |
| | | | TGGTATGGGTAGTGAT | | | |
| | | | CGCTTGACTGGCGGTA | | | |
| | | | ATGGTAAAGATACTTT | | | |
| | | | TGCTTTCTCTGCTCCA | | | |
| | | | ATTACCGATGGCATCG | | | |
| | | | ACACGATTACAGACTT | | | |
| | | | TAATCCCCTTGACGAT | | | |
| | | | CTCCTTCGTGTTGACG | | | |
| | | | CTGCTGGATTTGGTGG | | | |
| | | | TGGGCTTGTAGCTGGT | | | |
| | | | ACTCTGCTTGCAAGTC | | | |
| | | | AGTTTGTTTTGGGTAC | | | |
| | | | AGCAGCCAAGACTAC | | | |
| | | | AAGCGATCGCTTTATT | | | |
| | | | TATAATCAATCCACAG | | | |
| | | | GTGCGTTATTCTTTGA | | | |
| | | | TGTTGACGGCACAGGT | | | |
| | | | TCTAGCAGTCAAGTTC | | | |
| | | | AGATTGCTACTCTATC | | | |
| | | | GAATAAACCTGTGATT | | | |
| | | | AATGCGACGAATATCT | | | |
| | | | CGGTAATTTAA | | | |
| Synechocystis | s110654 | F4 | GTGGATTTGGTTCTGC | | MDLVLPADAPRTG | |
| sp. | | | CAGCGGATGCTCCCCG | | LATFAPDGSEQDVL | |
| PCC 6803 | | | CACCGGCCTGGCCACC | | AEYLAANFNSLETA | |
| | | | TTTGCCCCCGATGGTT | | FNQADTSPEFDVRI | |
| | | | CCGAGCAAGATGTCCT | | QNLAFRVDTVIDST | |
| | | | AGCGGAGTATTTAGCA | | GPVDPIANEIGVVA | |
| | | | GCCAACTTCAATAGCC | | ENGFFFVLLPGGDE | |
| | | | TGGAGACTGCATTTAA | | VQLKFNNQPFASGT | |
| | | | TCAGGCAGACACTTCC | | FGNWQILEAETVN | |
| | | | CCGGAATTTGATGTCC | | GINQVLWQNPNLG | |
| | | | GAATCCAAAATCTAGC | | QIGVWNADSNWN | |
| | | | CTTCCGTGTGGATACT | | WISSQTWPTNSFNT | |
| | | | GTTATTGATTCCACTG | | LEAEVTFQIDINND | |
| | | | GGCCCGTTGACCCAAT | | DLLGDRLTTVENQ | |
| | | | CGCCAATGAGATTGGA | | GNVSLLEGILGNYY | |
| | | | GTAGTGGCCGAAAAC | | VQSGDDLTTPIKYL | |
| | | | GGCTTCTTCTTTGTCCT | | GEAFDNNLGNWQA | |
| | | | ACTTCCTGGGGGCGAT | | LAAETVQGVNQVL | |
| | | | GAAGTACAGCTTAAAT | | WQNLDTNQIGVWN | |
| | | | TTAACAATCAACCCTT | | SSADWNWISSNVFE | |
| | | | TGCCAGTGGCACCTTT | | AGSPQAIAQAEIFGI | |
| | | | GGCAATTGGCAAATTT | | PTTVLTTADSVLV | |
| | | | TGGAAGCAGAAACGG | | | |
| | | | TCAACGGCATCAATCA | | | |
| | | | AGTGCTTTGGCAAAAT | | | |
| | | | CCCAACCTTGGTCAGA | | | |
| | | | TTGGTGTTTGGAATGC | | | |
| | | | CGACTCCAACTGGAAC | | | |
| | | | TGGATTTCTTCGCAAA | | | |
| | | | CTTGGCCTACCAATTC | | | |
| | | | CTTCAATACTCTGGAA | | | |
| | | | GCAGAGGTTACCTTCC | | | |
| | | | AGATTGACATCAACAA | | | |
| | | | CGATGACCTCCTTGGC | | | |
| | | | GATCGCCTGACGACCG | | | |
| | | | TGGAAAACCAGGGCA | | | |
| | | | ACGTCAGTCTGCTGGA | | | |
| | | | AGGCATCTTGGGTAAT | | | |
| | | | TACTACGTCCAATCTG | | | |
| | | | GGGATGATTTAACCAC | | | |
| | | | ACCAATCAAATACCTA | | | |
| | | | GGGGAGGCTTTTGACA | | | |
| | | | ACAACCTCGGTAACTG | | | |
| | | | GCAAGCCCTAGCGGC | | | |
| | | | GGAAACTGTACAAGG | | | |
| | | | GGTTAATCAAGTGCTG | | | |
| | | | TGGCAAAATCTCGACA | | | |
| | | | CCAACCAAATCGGTGT | | | |
| | | | TTGGAACTCTAGTGCT | | | |
| | | | GATTGGAACTGGATTT | | | |
| | | | CCTCCAATGTATTTGA | | | |
| | | | AGCTGGTTCTCCCCAG | | | |
| | | | GCGATCGCCCAAGCTG | | | |
| | | | AAATTTTTGGTATCCC | | | |
| | | | AACTACCGTCCTAACC | | | |
| | | | ACGGCTGACTCCGTTT | | | |
| | | | TAGTCTAA | | | |
| Synechocystis | s110656 | F6 | AATACGTCCTATGTCT | | NTSYVFDGQTGTL | |
| sp. | | | TTGATGGTCAAACCGG | | DYAFASASLAAQV | |
| PCC 6803 | | | TACCCTGGACTATGCC | | TGATEWGINADEA | |
| | | | TTTGCCAGTGCTAGCT | | DALDYNLDFGRDV | |
| | | | TGGCAGCACAGGTAA | | NIFDGTVPYRSSDH | |
| | | | CTGGCGCAACAGAAT | | DPIIVGLNLASPVEP | |
| | | | GGGGGATCAACGCCG | | IANEIGVMAENGFF | |
| | | | ATGAAGCAGATGCCCT | | FVLLPGGDEVQLKF | |
| | | | GGACTACAACCTCGAC | | NNQPFASGTFGNW | |
| | | | TTTGGGCGGGATGTCA | | QILEAETVNGINQV | |
| | | | ATATTTTTGATTGTAC | | LWQNPNLGQIGVW | |
| | | | GGTTCCCTATCGCTCC | | NADSNWNWISSQT | |
| | | | TCAGACCATGACCCCA | | WPTNSFNTLEAEVT | |
| | | | TAATTGTCGGCCTTAA | | FQIDINNDDLLGDR | |
| | | | CCTTGCTTCCCCCGTT | | LTTVENQGSTTLLE | |
| | | | GAGCCGATCGCCAAC | | GILGNYYVQSGDD | |
| | | | GAAATTGGCGTAATGG | | LTTPIKYLGEAFDN | |
| | | | CCGAAAATGGCTTCTT | | NLGNWQALAAETV | |
| | | | CTTTGTCCTACTTCCTG | | QGVNQVLWQNLN | |
| | | | GGGGTGATGAAGTAC | | TNQIGVWNSSADW | |
| | | | AGCTTAAATTTAACAA | | NWISSSVFEAGSPQ | |
| | | | TCAACCCTTTGCCAGT | | AIAQAGIFGVDLNA | |
| | | | GGCACCTTTGGCAATT | | VI | |
| | | | GGCAAATTTTGGAAGC | | | |
| | | | AGAAACGGTCAATGG | | | |
| | | | CATCAATCAAGTGCTT | | | |
| | | | TGGCAAAATCCCAACC | | | |
| | | | TTGGTCAGATTGGTGT | | | |
| | | | TTGGAATGCCGACTCC | | | |
| | | | AACTGGAACTGGATTT | | | |
| | | | CTTCGCAAACTTGGCC | | | |
| | | | TACCAATTCCTTCAAT | | | |
| | | | ACTCTGGAAGCAGAA | | | |
| | | | GTTACCTTCCAGATTG | | | |
| | | | ACATCAACAACGATG | | | |
| | | | ACCTCCTTGGCGATCG | | | |
| | | | CCTGACGACCGTGGAA | | | |
| | | | AACCAAGGTTCTACAA | | | |
| | | | CTCTCCTGGAAGGCAT | | | |
| | | | CTTGGGTAATTACTAC | | | |
| | | | GTCCAATCTGGGGATG | | | |
| | | | ATTTAACCACACCAAT | | | |
| | | | CAAATACCTTGGGGAA | | | |
| | | | GCCTTTGACAACAACC | | | |
| | | | TCGGTAACTGGCAAGC | | | |
| | | | CCTAGCGGCGGAAACT | | | |
| | | | GTACAAGGGGTTAACC | | | |
| | | | AAGTGCTGTGGCAAA | | | |
| | | | ACCTCAACACTAATCA | | | |
| | | | AATTGGTGTTTGGAAC | | | |
| | | | TCTAGTGCTGACTGGA | | | |
| | | | ACTGGATTTCCTCCAG | | | |
| | | | TGTGTTTGAAGCTGGT | | | |
| | | | TCTCCCCAGGCGATCG | | | |
| | | | CCCAGGCTGGCATTTT | | | |
| | | | TGGTGTTGATCTGAAT | | | |
| | | | GCTGTAATTTAA | | | |
| Synechocystis | s111951 | F9 | GATGGTGGTAAAGGA | | DGGKGFQLGKDGT | |
| sp. | | | TTCCAGCTTGGCAAAG | | TSFIGGDDSISGGD | |
| PCC 6803 | | | ACGGTACTACCAGTTT | | GNDFLAGDFVLVD | |
| | | | CATCGGTGGTGACGAT | | QLSAPFDPLDPND | |
| | | | TCTATTTCTGGTGGCG | | WTFVNPYATLQGQ | |
| | | | ACGGCAATGATTTCTT | | AGDSKAQAAQAAI | |
| | | | AGCCGGTGACTTTGTC | | NLAQLRLEFRAVG | |
| | | | CTGGTAGACCAATTGT | | GDDELVGGRGNDT | |
| | | | CAGCGCCATTTGATCC | | FYGGLGADTIDIGN | |
| | | | CTTGGATCCCAACGAT | | DVTVGGVGVNGA | |
| | | | TGGACATTTGTCAATC | | NEIWYMNGAFENA | |
| | | | CCTACGCCACTCTCCA | | AVNGANVDNITGF | |
| | | | AGGCCAGGCGGGTGA | | NVNNDKFVFAAGA | |
| | | | TAGTAAAGCTCAAGCT | | NNFLSGDATSGLA | |
| | | | GCTCAAGCTGCTATCA | | VQRVLNLQAGNTV | |
| | | | ATTTGGCTCAACTCCG | | FNLNDPILNASANN | |
| | | | CCTTGAGTTCCGTGCC | | INDVFLAVNADNS | |
| | | | GTTGGCGGCGATGACG | | VGASLSFSLLPGLPS | |
| | | | AGCTCGTGGGTGGTCG | | LVEMQQINVSSGAL | |
| | | | TGGCAACGATACTTTC | | AGREFLFINNGVAA | |
| | | | TATGGTGGTCTTGGTG | | VSSQDDFLVELTGI | |
| | | | CAGACACTATTGATAT | | SGTFGLDLTPNFEV | |
| | | | CGGTAATGATGTCACT | | REFYA | |
| | | | GTCGGCGGTGTTGGCG | | | |
| | | | TTAACGGTGCCAATGA | | | |
| | | | AATCTGGTACATGAAT | | | |
| | | | GGTGCCTTTGAAAACG | | | |
| | | | CAGCGGTCAATGGAG | | | |
| | | | CCAACGTCGATAACAT | | | |
| | | | TACTGGTTTCAACGTA | | | |
| | | | AACAACGACAAATTTG | | | |
| | | | TCTTCGCGGCTGGAGC | | | |
| | | | CAATAACTTCTTGTCT | | | |
| | | | GGTGATGCTACATCCG | | | |
| | | | GCCTTGCCGTCCAACG | | | |
| | | | TGTCCTTAATTTACAG | | | |
| | | | GCGGGGAATACGGTCT | | | |
| | | | TCAATCTAAACGATCC | | | |
| | | | GATCCTTAATGCCTCT | | | |
| | | | GCTAATAACATCAACG | | | |
| | | | ATGTGTTCTTAGCTGT | | | |
| | | | AAATGCAGACAACAG | | | |
| | | | TGTCGGTGCGTCTCTC | | | |
| | | | TCCTTCTCCTTGCTACC | | | |
| | | | CGGCTTGCCTTCTCTG | | | |
| | | | GTTGAGATGCAACAG | | | |
| | | | ATCAATGTCTCTTCTG | | | |
| | | | GTGCTCTGGCTGGTCG | | | |
| | | | CGAATTCCTGTTCATC | | | |
| | | | AACAACGGTGTTGCGG | | | |
| | | | CTGTCAGCTCCCAAGA | | | |
| | | | CGACTTCCTCGTAGAA | | | |
| | | | CTTACAGGTATTAGCG | | | |
| | | | GTACCTTTGGTCTGGA | | | |
| | | | CTTGACTCCTAACTTC | | | |
| | | | GAGGTTCGTGAGTTCT | | | |
| | | | ACGCCTAA | | | |
| Synechococcus | Synpcc | F7 | AGCTATGTGGTGTTTG | | SYVVFGNAAPVLD | |
| elongatus | 7942_1 | | GCAACGCAGCACCGG | | LDGTTSPELNFGAV | |
| PCC 7942 | 337 | | TGCTTGATTTGGATGG | | FTGTPVSVVGSGLT | |
| | | | CACCACATCACCAGAG | | ITDLNSPTLAAATV | |
| | | | CTGAACTTTGGCGCTG | | TLVNRPDGIAESLS | |
| | | | TCTTTACTGGTACGCC | | AITDGTAIKASYDS | |
| | | | AGTCTCAGTTGTGGGT | | NTGVLLLVGLATV | |
| | | | TCAGGACTCACCATTA | | ADYEKVLRTVTYT | |
| | | | CCGATCTCAACTCTCC | | NTSNAADLDVSRR | |
| | | | AACCCTCGCCGCAGCG | | TIEFVLDDGADFAN | |
| | | | ACCGTGACCTTGGTCA | | TSAVVTTTLSFKNE | |
| | | | ACCGGCCCGATGGCAT | | VNTITGTPRLDFLR | |
| | | | TGCTGAAAGTTTGAGT | | GSKGDDLITGLGGN | |
| | | | GCAATCACGGATGGC | | DFLFGRAGNDTLIG | |
| | | | ACTGCAATTAAGGCCA | | GLGSDVLSGGAGK | |
| | | | GCTATGACAGCAATAC | | DRFVYTAVTEARD | |
| | | | CGGGGTGCTGCTGCTC | | LIIDFNAKQDVLDL | |
| | | | GTGGGTCTGGCTACTG | | SGLLDSLGYQGSNP | |
| | | | TGGCGGATTATGAGAA | | VADQVLRLNSQSFL | |
| | | | AGTCCTGCGCACCGTC | | GTTVSVNVAGLGG | |
| | | | ACCTATACCAACACCT | | VPDFVSLVTLLGVS | |
| | | | CTAATGCAGCCGATCT | | SSALVIGENIII | |
| | | | GGATGTAAGCCGTCGC | | | |
| | | | ACGATTGAGTTTGTCC | | | |
| | | | TCGACGATGGAGCAG | | | |
| | | | ATTTTGCCAACACCAG | | | |
| | | | TGCGGTAGTCACTACC | | | |
| | | | ACGCTGAGCTTCAAGA | | | |
| | | | ATGAAGTCAATACAAT | | | |
| | | | CACTGGAACCCCCAGA | | | |
| | | | CTCGACTTCCTCCGAG | | | |
| | | | GCAGCAAGGGAGATG | | | |
| | | | ACTTGATTACGGGGCT | | | |
| | | | CGGGGGGAATGACTTC | | | |
| | | | CTGTTTGGCAGGGCTG | | | |
| | | | GTAATGACACCTTGAT | | | |
| | | | TGGCGGACTCGGCTCT | | | |
| | | | GACGTCCTTTCTGGTG | | | |
| | | | GAGCCGGCAAGGACC | | | |
| | | | GCTTTGTCTACACCGC | | | |
| | | | TGTTACTGAGGCTCGC | | | |
| | | | GACTTAATCATCGACT | | | |
| | | | TTAATGCCAAGCAGGA | | | |
| | | | TGTTCTGGATCTAAGC | | | |
| | | | GGGTTGTTGGATAGTC | | | |
| | | | TGGGCTATCAAGGCTC | | | |
| | | | TAATCCTGTTGCGGAT | | | |
| | | | CAGGTCCTGCGCTTGA | | | |
| | | | ACAGTCAGTCTTTCTT | | | |
| | | | GGGCACGACGGTCTCT | | | |
| | | | GTCAATGTAGCGGGAC | | | |
| | | | TCGGTGGAGTGCCCGA | | | |
| | | | CTTTGTCTCCCTAGTG | | | |
| | | | ACCCTGCTTGGTGTCT | | | |
| | | | CTTCTTCTGCCCTCGTC | | | |
| | | | ATTGGTGAAAACATCA | | | |
| | | | TCATTTAG | | | |
| Synechococcus | Synpcc | F5 | AAAGGTCCTGAGCCTG | | KGPEPEGVVIGQIN | |
| elongatus | 7942_1 | | AAGGTGTCGTGATTGG | | DRTYAFVGLERTG | |
| PCC 7942 | 392 | | CCAGATTAACGATCGC | | GVIVYDVTTPNNPT | |
| | | | ACCTATGCCTTTGTCG | | FVQYLNNRNFNAD | |
| | | | GTCTTGAGCGGACCGG | | VESAEAGDLGPEGL | |
| | | | TGGCGTCATAGTCTAC | | AFISAEDSPNGKPL | |
| | | | GACGTGACTACCCCTA | | LVVANEISGTTTLY | |
| | | | ACAATCCCACCTTTGT | | EINVGSNPDLIKLD | |
| | | | TCAGTACCTCAACAAT | | NSAQIAYITYLGRP | |
| | | | CGTAATTTCAACGCTG | | GDRGGLTFWNEVL | |
| | | | ATGTTGAAAGTGCCGA | | RDAEISYDPQTGDL | |
| | | | AGCGGGTGATTTAGGC | | ITGEEVLPFNAFING | |
| | | | CCTGAGGGTCTTGCTT | | FGDSSEADQIYGGK | |
| | | | TCATCTCTGCAGAGGA | | SAADQVNLIYNFAF | |
| | | | CAGCCCCAACGGCAA | | NRNAESAGQAFWV | |
| | | | ACCTCTGTTGGTTGTC | | NQLNSRQLSLAELA | |
| | | | GCCAACGAGATCAGT | | LEIGLNATGNDSVV | |
| | | | GGAACTACAACGCTCT | | LNNKIRSATLFTDSI | |
| | | | ATGAGATTAATGTCGG | | DTNVELAAYQGSK | |
| | | | TTCTAATCCTGACTTG | | GTSFGQTWLDQFD | |
| | | | ATCAAGTTAGACAACA | | FSQSSQALVDSALN | |
| | | | GCGCCCAGATTGCTTA | | ALVNDLPLG | |
| | | | CATCACTTATCTAGGA | | | |
| | | | CGGCCTGGCGATCGCG | | | |
| | | | GTGGACTGACCTTTTG | | | |
| | | | GAATGAGGTTCTGAGA | | | |
| | | | GATGCCGAAATCAGCT | | | |
| | | | ACGACCCTCAAACTGG | | | |
| | | | TGATTTAATTACTGGT | | | |
| | | | GAAGAAGTTCTTCCCT | | | |
| | | | TCAACGCCTTCATCAA | | | |
| | | | CGGGTTTGGAGATTCT | | | |
| | | | TCTGAAGCTGATCAAA | | | |
| | | | TCTACGGTGGTAAATC | | | |
| | | | TGCAGCCGATCAGGTG | | | |
| | | | AACTTAATTTATAACT | | | |
| | | | TTGCCTTCAATCGTAA | | | |
| | | | TGCTGAGAGTGCTGGC | | | |
| | | | CAAGCCTTCTGGGTCA | | | |
| | | | ACCAGCTGAATAGTCG | | | |
| | | | CCAGCTCAGCTTGGCG | | | |
| | | | GAACTGGCTCTAGAAA | | | |
| | | | TTGGTCTGAACGCGAC | | | |
| | | | AGGCAATGATTCAGTA | | | |
| | | | GTTCTTAACAACAAGA | | | |
| | | | TTAGAAGTGCCACTCT | | | |
| | | | GTTCACCGATTCGATT | | | |
| | | | GACACGAATGTTGAAC | | | |
| | | | TAGCTGCTTATCAAGG | | | |
| | | | TAGTAAGGGGACCAG | | | |
| | | | CTTTGGTCAGACCTGG | | | |
| | | | CTAGATCAGTTTGACT | | | |
| | | | TTAGCCAAAGTAGCCA | | | |
| | | | AGCTCTGGTTGATAGT | | | |
| | | | GCTCTTAACGCTTTAG | | | |
| | | | TCAATGACCTACCTCT | | | |
| | | | TGGATAG | | | |

TABLE 17
Type IV Leader Sequences

| | | SEQ |
|---|---|---|
| | | ID |
| Name | Sequence | NO |
| A1602 | MINQPCIVPAEKGFTLIELLTGMLIVGILASISAPSFLGLVNRGRVNEALNRTRGALQE | |
| | AQREVIKKSNTCNLTFSPSGQTVNITGGCLVTGPRVMSRVTYRHTLANNDPANVIEL | |
| | DFKGVPVEDNFNDGQEVFVFRGNGNYERCLVISRALGLIRVGTYNTSGTSDTSTDA | |
| | TKCITGQV | |
| A1602- | MINQPCIVPAEKGFTLIELLTGMLIVGILASISAPSFLGLVNRGRVNEALNRTRGALQE | |
| C222 | AQREVIKKSNTCNLTFSPSGQTVNITGGCLVTGPRVMSRVTYRHTLANNDPANVIEL | |
| | DFKGVPVEDNFNDGQEVFVFRGNGNYERCLVISRALGLIRVGTYNTSGTSDTSTDA | |
| | TKCITGQVDTKKKVDDDLGTIENLEEAKKKLLKDVEVLSQRLEEKALAYDKLEKTK | |
| | TRLQQELDDLLVDLDHQ | |
| A1604 | MKIANFISRKNINLNYGFTLFELLAGLVIVGILAGISVPSFLAFVERGRVNEAANILRG | |
| | VIQSSQREAIKKSTDCTIQLPAKQTKNPTISSTCSIDGPRRLKNVVIQYNQTDQISIDY | |
| | QGRFNRKRTIVLYSENTNYKRCLVVSSFIGMTRTGIYTDQDLNTVSADYCQKTNVG | |
| A1604- | MKIANFISRKNINLNYGFTLFELLAGLVIVGILAGISVPSFLAFVERGRVNEAANILRG | |
| C222 | VIQSSQREAIKKSTDCTIQLPAKQTKNPTISSTCSIDGPRRLKNVVIQYNQTDQISIDY | |
| | QGRFNRKRTIVLYSENTNYKRCLVVSSFIGMTRTGIYTDQDLNTVSADYCQKTNVG | |
| | DTKKKVDDDLGTIENLEEAKKKLLKDVEVLSQRLEEKALAYDKLEKTKTRLQQEL | |
| | DDLLVDLDHQ | |
| A2335 | MLRLLFLHRKKAAQDFQGFTVIELMIVMIITGILTAIALPAFLNQVDKSRYAKARLQ | |
| | MRCMLQELKVYRLNHGSYPPDQNRNVPYYPGSECFKVHTGYVRDRPDINRNNNTD | |
| | IPFHSVYDYERWDYNSGCYIAVTFFGKNGLRRFTQAAINEISTTGFHFYDGTDDDLV | |
| | LVVDITDSPCD | |
| A2335- | MLRLLFLHRKKAAQDFQGFTVIELMIVMIITGILTAIALPAFLNQVDKSRYAKARLQ | |
| C222 | MRCMLQELKVYRLNHGSYPPDQNRNVPYYPGSECFKVHTGYVRDRPDINRNNNTD | |
| | IPFHSVYDYERWDYNSGCYIAVTFFGKNGLRRFTQAAINEISTTGFHFYDGTDDDLV | |
| | LVVDITDSPCDDTKKKVDDDLGTIENLEEAKKKLLKDVEVLSQRLEEKALAYDKLE | |
| | KTKTRLQQELDDLLVDLDHQ | |
| A2803 | MSSYKAICVWLIHYSKRNNQGFTLIELLVVMIIIGILSAISLPVMFSMAAKARQSEAK | |
| | TTLSVLNRGQQAYYAEKSTFSPDILNLGVTTIIETNNFSYGNAGSLVNYQTGAAYGA | |
| | TPKDPATVKDYSAGVTSLAIARVPLIICEEEDPTVVGPFPPLLDSGAGTLSCPVGYIKL | |
| | R | |
| A2803- | MSSYKAICVWLIHYSKRNNQGFTLIELLVVMIIIGILSAISLPVMFSMAAKARQSEAK | |
| C222 | TTLSVLNRGQQAYYAEKSTFSPDILNLGVTTIIETNNFSYGNAGSLVNYQTGAAYGA | |
| | TPKDPATVKDYSAGVTSLAIARVPLIICEEEDPTVVGPFPPLLDSGAGTLSCPVGYIKL | |
| | RDTKKKVDDDLGTIENLEEAKKKLLKDVEVLSQRLEEKALAYDKLEKTKTRLQQE | |
| | LDDLLVDLDHQ | |
| A2804 | MKNFTFKLLQQLNKKKADKGFTLIELLVVIIIIGILSAIALPAFLNQAAKAKQSEAKQ | |
| | TLGALNRGQQAYRLESPEFAPEVDLLALGVEIDTTNYAYGDDGSATTGNGEFAFNF | |
| | NNLEGTDFTETAGIGARAKDTAAVRDYDGATGATEDSEGNATTVTVICEETAPQDD | |
| | DQDMSYSFADGLGCDAGNQL | |
| A2804- | MKNFTFKLLQQLNKKKADKGFTLIELLVVIIIIGILSAIALPAFLNQAAKAKQSEAKQ | |
| C222 | TLGALNRGQQAYRLESPEFAPEVDLLALGVEIDTTNYAYGDDGSATTGNGEFAFNF | |
| | NNLEGTDFTETAGIGARAKDTAAVRDYDGATGATEDSEGNATTVTVICEETAPQDD | |
| | DQDMSYSFADGLGCDAGNQLDTKKKVDDDLGTIENLEEAKKKLLKDVEVLSQRLE | |
| | EKALAYDKLEKTKTRLQQELDDLLVDLDHQ | |

TABLE 18
Sec Leader Sequences

| | | Cyanobase | | | Alignment | | SEQ |
|---|---|---|---|---|---|---|---|
| | | Gene | NCBI | D | E- | | ID |
| | Leader# | Locus | ID | Value | Score | Leader sequence | NO |
| 1 | L1 | A2177 | 1.7E+ | 0.5 | 1.20E− | MKKFSFALAAASALSLSLASTAQAGQGGIAAGAA | |
| | | | 08 | | 10 | | |
| 2 | L2 | G0103 | 1.7E+ | 0.42 | 3.10E− | MHYWYRLLGFTGGVALFWAAQELSAVAASPQP | |
| | | | 08 | | 165 | SDATA | |
| 3 | L3 | A2679 | 1.7E+ | 0.49 | 2.00E− | MTTFTFSRPQSLKLATAGAFLALGVLSIAQPAKAD | |
| | | | 08 | | 02 | NVSSSTMIS | |
| 4 | L4 | A0482 | 1.7E+ | 0.46 | 3.50E− | MLSRFLILCLALCLWAVSPLPSFAASPFAGERPT | |
| | | | 08 | | 03 | | |
| 5 | L5 | A2579 | 1.7E+ | 0.48 | 1.30E− | MNFAKIAAVAAGAAALSLGFASSAKAEFAASVSF | |
| | | | 08 | | 05 | VD | |
| 6 | L6 | A1471 | 1.7E+ | 0.48 | 1.20E− | MTTFAFSRPQSLKLATAGAFLALGVLSIAQPAKAD | |
| | | | 08 | | 02 | NVSSSTMIS | |
| 7 | L7 | A0795 | 1.7E+ | 0.46 | 0.00E+ | MRKSNLSLKTLAIATLLSSSLFACGSPNQSITS | |
| | | | 08 | | 00 | | |
| 8 | L8 | A2284 | 1.7E+ | 0.44 | 5.30E− | MKQSATRLRTLSLGLAGLTLTAALAACNTTQTPT | |
| | | | 08 | | 15 | E | |
| 9 | L9 | G0161 | 1.7E+ | 0.44 | 7.40E− | MTMKIRYAATLVSISLLSLGAIAGCSGVKNPCA | |
| | | | 08 | | 02 | | |
| 10 | L10 | A2596 | 1.7E+ | 0.43 | 4.00E− | MAYSVVSWRKNLSWALCSLALLLPLPLNAQVQV | |
| | | | 08 | | 05 | SPMVIK | |
| 11 | L11 | A1490 | 1.7E+ | 0.46 | 4.60E− | MKNLSVKLLSGTATMTAVSLMAINPATADTVSG | |
| | | | 08 | | 12 | SVTFT | |
| 12 | L12 | A2870 | 1.7E+ | 0.42 | 2.80E− | MKITRHTIGKGLMLGTMILMGSSFSANAAPLSST | |
| | | | 08 | | 04 | GPLP | |
| 13 | L13 | A2813 | 1.7E+ | 0.45 | 7.70E− | MKTSLSLWKSLSIASAAVGVSVATAGTAQAQAN | |
| | | | 08 | | 03 | NS | |
| 14 | L14 | A0782 | 1.7E+ | 0.43 | 3.30E− | MTSLKTVSLAATAFVTMASQAIAADNQGLLEQI | |
| | | | 08 | | 05 | | |
| 15 | L15 | A2686 | 1.7E+ | 0.43 | 6.20E− | MFKPITLLNVALLGLLGFTPLLQASTPNASQIAS | |
| | | | 08 | | 08 | | |
| 16 | L16 | A2294 | 1.7E+ | 0.43 | 1.30E− | MTKFLNYCLSVALAIAVCFGVTQPASALPQPSFTL | |
| | | | 08 | | 05 | AS | |
| 17 | L17 | A0344 | 1.7E+ | 0.47 | 3.00E− | MTLKRKHLLALSAVFTTFAPLSLTTAPTLANTDTSP | |
| | | | 08 | | 05 | | |
| 18 | L18 | A1370 | 1.7E+ | 0.42 | 7.30E− | MKFKLPHFVLGLSIALVISLHGCTFGNSGQTLVVAI | |
| | | | 08 | | 79 | AA | |
| 19 | L19 | A0568 | 1.7E+ | 0.41 | 2.00E− | MKSQTKRLKRACSYLVLALSAMVPSVALAGHTNT | |
| | | | 08 | | 04 | ILHTM | |
| 20 | L20 | A1582 | 1.7E+ | 0.41 | 4.10E− | MKKIIALLSLGSVMLTAGAAQAQITPTNQYSY | |
| | | | 08 | | 07 | | |
| 21 | L21 | A0393 | 1.7E+ | 0.41 | 6.80E− | MSKSTMIHSRQFYSAAAIALCFGSLLVSC | |
| | | | 08 | | 76 | | |
| 22 | L22 | A2507 | 1.7E+ | 0.37 | 1.60E− | MKSRSLSLCGLFLGLAIATGCTPATNNN | |
| | | | 08 | | 119 | | |
| 23 | L23 | A2685 | 1.7E+ | 0.42 | 1.00E− | MKPPKIALLSSLCCLGFTSLAVATLPQASQIVS | |
| | | | 08 | | 05 | | |
| 24 | L24 | A0816 | 1.7E+ | 0.37 | 8.50E− | MGKTQFQPVSQILALASLATLAFSSQSLAQ | |
| | | | 08 | | 90 | | |
| 25 | L25 | G0006 | 1.7E+ | 0.36 | 6.30E− | MKQQARDSFALAVGSMMPVLIATQPAQAQTSA | |
| | | | 08 | | 142 | | |
| 26 | L26 | A1127 | 1.7E+ | 0.4 | 4.50E− | MLSPSKKFLILVLASLLILPMPAAIATPIDPCLLRE | |
| | | | 08 | | 03 | | |
| 27 | L27 | A2426 | 1.7E+ | 0.39 | 3.40E− | MTNCYRKLLLFLSLSLMMGAGQVSAASLVGPIQ | |
| | | | 08 | | 08 | DPL | |
| 28 | L28 | A2052 | 1.58E+ | 0.44 | 5.10E− | MSTTSISPGKTGTITCLSALLLSTAIAPFAALNPAQ | |
| | | | 08 | | 02 | A | |
| 29 | L29 | A2201 | 1.7E+ | 0.38 | 1.90E− | MQTSKFNLAIALSLAAIATFTGACQDTT | |
| | | | 08 | | 59 | | |
| 30 | L30 | A1096 | 1.7E+ | 0.43 | 8.30E− | MLRRVILAIAIALWWGLWVVWAAPQSQFLTIA | |
| | | | 08 | | 132 | | |
| 31 | L31 | A2531 | 1.7E+ | 0.42 | 2.20E− | MKSGLKLSLTLAFAAGIVVPAGSVNAQVCSDVG | |
| | | | 08 | | 18 | GGA | |
| 32 | L32 | A2439 | 1.7E+ | 0.42 | 6.60E− | MAFYKQISAFCSATSLLTIPLAIAPAQAQQSYPL | |
| | | | 08 | | 02 | | |
| 33 | L33 | A1469 | 1.7E+ | 0.39 | 8.30E− | MRFTKTLALSLALGSTLGFSTVAQAGDYGSYGDK | |
| | | | 08 | | 06 | T | |
| 34 | L34 | G0010 | 1.7E+ | 0.43 | 2.10E− | MFKTLIKNSAAIAFVLLGSIAVIPGASSQIS | |
| | | | 08 | | 02 | | |
| 35 | L35 | A0381 | 1.7E+ | 0.4 | 1.00E− | MNHFLPRPLLRSLFAVCLAVMTWAIAPAAFAVN | |
| | | | 08 | | 04 | NPE | |
| 36 | L36 | A1473 | 1.7E+ | 0.4 | 1.60E− | MKALILALGISCLAIPVAAQGTCLRISDF | |
| | | | 08 | | 05 | | |
| 37 | L37 | A1034 | 1.7E+ | 0.41 | 2.00E− | MSKTVRTFLSGASVALGATVAFSGTAQANTELLD | |
| | | | 08 | | 04 | QINS | |
| 38 | L38 | A1488 | 1.7E+ | 0.39 | 1.40E− | MKTLTFLMIPAMALSLMPQSVLAWNAYHLYNK | |
| | | | 08 | | 07 | D | |
| 39 | L39 | D0005 | 1.7E+ | 0.4 | 3.90E− | MKSTPHFSRTRMLVMGGFMSLSSVALAAPALAH | |
| | | | 08 | | 09 | HPFG | |
| 40 | L40 | A0934 | 1.7E+ | 0.37 | 6.10E− | MKRMLGLAMALFIASPASAGNLLQGEPYY | |
| | | | 08 | | 03 | | |
| 41 | L41 | A1795 | 48579 | 0.4 | 1.40E− | MNLKFLKSLWATAAIAFAISVNPSLVFAETEPPSE | |
| | | | 7 | | 01 | TKT | |
| 42 | L42 | A2616 | 1.7E+ | 0.41 | 8.60E− | MKFNLFNPYLLAASAIISACFILPKPTQAASWLEC | |
| | | | 08 | | 09 | NGDS | |
| 43 | L43 | A2016 | 1.7E+ | 0.44 | 1.30E− | MTRFFLVIAPILAGLAVAAGAFASHGLKETLDA | |
| | | | 08 | | 04 | | |
| 44 | L44 | F0093 | 1.7E+ | 0.44 | 1.70E− | MKLPLLWVSLVLILLLSFGWGSRSAATSAPTVDLE | |
| | | | 08 | | 05 | T | |
| 45 | L45 | A2859 | 1.7E+ | 0.41 | 3.10E− | MVKMFQFKRTLSVGAIATSLTMITGGVWAAEKP | |
| | | | 08 | | 05 | TIQIAI | |
| 46 | L46 | A0029 | 1.7E+ | 0.47 | 9.30E− | MDYLNFVYFFTTMIALAALPSTSVALVVTRSATA | |
| | | | 08 | | 02 | G | |
| 47 | L47 | A2751 | 1.7E+ | 0.38 | 5.80E− | MGIKKAIATFFISTALFPLGFSNSAQAEVATLEFDY | |
| | | | 08 | | 06 | E | |
| 48 | L48 | A1255 | 1.7E+ | 0.47 | 8.30E− | MNRLKTAATYLLLGAIALVMLFPLLWLLSTALKSP | |
| | | | 08 | | 10 | TENVFS | |

TABLE 19
Phosphatases

| | Protein sequence | DNA sequence |
|---|---|---|
| SYNPCC700 | MIHDDGRSNYSNNRPFQDI | ATGATTCACGACGACGGCAGAAGTAATTATTCAAATAATCGTC |
| 2_A0893 | FKARFSRRSMLQKSMMLSA | CTTTCCAAGATATTTTCAAGGCGCGATTCTCCCGCCGGAGTAT |
| | AGFIGAIAGNSVLKPSTAA | GCTCCAAAAAAGCATGATGCTCTCCGCCGCTGGTTTTATCGGG |
| | TQVAQRRTSPLLGFNAVTL | GCGATCGCCGGCAATAGCGTCCTCAAACCCAGCACCGCCGCCA |
| | AQGNGPVPSISSDYQYQVL | CCCAAGTTGCCCAACGGCGCACCAGTCCCCTTTTGGGATTCAA |
| | IPWGTPIQPGGPEYNGDPN | TGCTGTAACCCTAGCCCAAGGCAATGGCCCCGTCCCCAGTATT |
| | TRPTADEQAQQIGIGHDGM | TCCAGTGACTACCAATACCAAGTGTTGATCCCCTGGGGTACCC |
| | WFFPLGNNNDHGLLAINHE | CCATCCAACCCGGTGGCCCCGAATACAATGGCGACCCCAACAC |
| | FGINEHVLGKADPASLEDV | CCGACCCACCGCCGACGAACAGGCCCAGCAGATTGGCATCGGC |
| | RLSQHAHGASVVEIKKNNR | CACGATGGGATGTGGTTTTTCCCCCTCGGCAACAACAATGACC |
| | GVWEVVRSNYARRIHANTP | ATGGTTTGTTGGCAATTAACCACGAATTTGGCATCAACGAACA |
| | MAFSGPAANHPLLKTAAGN | CGTCCTGGGTAAAGCAGATCCCGCCAGCCTTGAGGATGTGCGA |
| | APKGTINNCSNGHTPWGTY | TTGTCTCAACATGCCCATGGTGCCTCCGTCGTTGAAATTAAGA |
| | LTCEENFNTYFGATGEWTP | AAAATAATCGTGGCGTTTGGGAAGTGGTTCGCAGTAACTATGC |
| | TEAQTRYGLASSSRYGWAN | CCGCCGGATCCATGCCAATACCCCCATGGCCTTCAGTGGCCCT |
| | YDERFDLSKAAYKNEENRF | GCAGCAAATCATCCTCTCCTAAAAACGGCAGCGGGCAATGCGC |
| | GWVVEIDPMDPNQTPVKRT | CGAAAGGGACTATCAATAACTGTTCTAACGGTCACACTCCCTG |
| | ALGRFKHEGAEIVVGRGGR | GGGCACCTACCTCACCTGTGAGGAAAACTTCAACACCTACTTT |
| | VVCYMGDDERFDYIYKFVS | GGGGCAACCGGAGAATGGACGCCCACCGAAGCCCAGACCCGCT |
| | ANNWQSMRARGISPFDEGQ | ATGGACTCGCCAGCAGTTCTCGCTATGGTTGGGCAAACTATGA |
| | LYVAKFNDDGSGEWLPLSM | CGAGCGATTCGACTTGTCAAAGGCGGCCTACAAAAATGAAGAA |
| | DNPALQGKFQDQAEILVYT | AACCGCTTTGGTTGGGTCGTCGAAATTGATCCGATGGATCCCA |
| | RLAADAAGATPMDRPEWIT | ACCAGACCCCTGTGAAGCGCACAGCCCTTGGTCGTTTTAAGCA |
| | VGTEENVYCALTNNSRRTE | TGAAGGGGCAGAAATTGTCGTTGGTCGTGGCGGTCGTGTGGTC |
| | ADAANPLAPNPDGHIIRWQ | TGCTATATGGGTGACGATGAACGCTTTGACTACATTTACAAGT |
| | DSDRHVGTTFTWDIFAIAQ | TCGTTTCGGCAAACAATTGGCAGTCAATGCGGGCGCGGGGGAT |
| | DTHGTEESFASPDGLWADP | CAGTCCCTTCGATGAAGGCCAGTTGTATGTTGCCAAGTTCAAC |
| | DGRLFIQTDGAQKDGLNDQ | GATGATGGCTCTGGAGAGTGGTTACCCCTCAGCATGGATAACC |
| | LLVADTNTKEIRRLFTGVT | CAGCCTTACAAGGAAAATTCCAAGACCAGGCTGAAATCCTTGT |
| | DCEVTGITVTPERRTMFIN | GTATACTCGCTTAGCGGCAGATGCGGCTGGGGCAACGCCGATG |
| | VQHPGDGNPATTNFPAPQG | GATCGTCCGGAATGGATCACTGTCGGCACCGAGGAAAACGTTT |
| | SGMVPRDSTVVITRKDGGI | ATTGTGCCCTCACTAACAATAGCCGTCGCACGGAAGCTGATGC |
| | VGS | GGCGAACCCCCTGGCACCGAATCCTGATGGCCACATTATTCGC |
| | | TGGCAGGATAGCGATCGCCACGTGGGGACAACCTTCACCTGGG |
| | | ATATTTTTGCGATCGCCCAAGATACCCATGGCACCGAAGAATC |
| | | TTTTGCCTCTCCCGATGGACTATGGGCTGACCCCGATGGCCGT |
| | | CTCTTTATCCAAACCGACGGTGCCCAGAAGGACGGCTTGAATG |
| | | ACCAACTGCTCGTAGCGGATACCAATACCAAGGAAATTCGGCG |
| | | TCTCTTTACTGGGGTGACAGATTGCGAAGTAACGGGGATTACG |
| | | GTGACCCCAGAGCGTCGCACGATGTTTATTAACGTGCAGCACC |
| | | CAGGCGATGGCAACCCAGCCACCACCAATTTCCCGGCTCCCCA |
| | | GGGGAGTGGGATGGTGCCCCGGGATAGCACCGTGGTCATCACC |
| | | CGTAAAGATGGCGGCATCGTTGGCTCATAG |
| SYNPCC700 | MNLNSGVKSLVASMVKPKL | ATGAACTTAAATAGTGGTGTGAAAAGCTTAGTGGCATCAATGG |
| 2_A2352 | KASFKLALLSTLAGLPLGT | TGAAGCCCAAGCTAAAAGCTAGTTTCAAGTTAGCTCTCTTATC |
| | LIFPPQAIAQNATIRGEVV | GACTCTTGCCGGCCTTCCATTGGGCACGCTAATCTTTCCGCCC |
| | FTLTDLAGAEMLAVTKDGR | CAAGCGATCGCCCAAAACGCAACTATTCGAGGTGAAGTTGTTT |
| | HALVVGAKTATLVAIEDNA | TCACATTAACGGATCTCGCCGGCGCAGAAATGCTCGCTGTCAC |
| | LTVEGTWTLTDEFLPAGSA | AAAAGATGGTCGCCACGCCCTTGTGGTCGGCGCAAAAACAGCG |
| | DAELTGVSISPDGAFALIG | ACCTTAGTGGCGATCGAAGATAATGCCTTAACCGTCGAAGGGA |
| | VKDADDANLDTFDEMPGKV | CTTGGACCCTAACGGATGAATTTTTGCCCGCAGGTTCTGCGGA |
| | VALSLPDLEPLGHVTVGRG | CGCTGAACTCACTGGAGTTTCCATTAGCCCAGACGGGGCCTTC |
| | PDSVAIAPNGQFAAVANED | GCACTCATCGGGGTCAAAGACGCAGATGACGCAAATCTGGATA |
| | EENEEDLTNLENGAGTVSI | CCTTTGACGAAATGCCAGGCAAGGTCGTGGCCCTCTCTCTCCC |
| | IDLRRGPNRMTQVEVPIPP | CGATCTAGAACCCCTTGGGCACGTAACTGTAGGTCGCGGCCCA |
| | DNIPFFPHDPQPETVRIAA | GACTCCGTGGCGATCGCCCCGAATGGTCAGTTTGCTGCCGTCG |
| | DSSFIVATLQENNAVARIE | CCAATGAAGATGAAGAAAACGAAGAAGATCTGACGAACCTAGA |
| | IPSPLPKRLTPDIFSVQNF | AAACGGCGCTGGAACCGTTTCGATCATTGATCTCCGACGTGGC |
| | DVGVRTGFGLVQDKVGEGS | CCCAATCGCATGACCCAGGTCGAGGTGCCCATTCCCCCCGACA |
| | CRSGSYDLSLRQEFTSARE | ATATTCCCTTTTTCCCCCACGACCCACAGCCTGAGACGGTTCG |
| | PDGIAITPDGRYFVTADED | CATCGCGGCTGATAGCTCTTTTATTGTCGCCACACTACAAGAA |
| | NLTNVNNQSYEGILLSPHG | AATAATGCTGTCGCTCGCATTGAAATTCCCTCTCCTTTGCCCA |
| | TRSISVFDATTGELLGDSG | AACGTCTAACCCCTGATATCTTTTCGGTGCAAAACTTTGATGT |
| | NSIEESIIALGLPQRCNSK | CGGCGTTCGTACGGGTTTCGGTTTAGTTCAAGATAAAGTTGGA |
| | GPEPEVVSVGVVNGRTLAF | GAAGGAAGCTGTCGTTCTGGCAGCTATGACCTATCCCTCAGAC |
| | VAIERSDAITIHDISNPRN | AAGAATTCACCTCTGCCCGTGAACCCGATGGCATTGCCATTAC |
| | VQLLDTVVLNPDVVRANQE | CCCAGATGGTCGCTACTTTGTCACCGCCGATGAAGATAATTTG |
| | AGFEPEGIEFIPATNQVIV | ACCAATGTCAATAACCAGTCCTACGAAGGAATTCTCTTAAGTC |
| | SNPEGNAMSLVNINVMPR | CCCATGGTACCCGCAGTATTAGTGTCTTTGACGCAACCACGGG |
| | | TGAACTTTTGGGAGATAGCGGCAATTCCATCGAAGAAAGCATC |
| | | ATCGCCCTCGCCTTGCCCCAGCGCTGTAACAGCAAAGGCCCAG |
| | | AACCTGAGGTTGTTTCCGTTGGTGTTGTAAATGGTCGTACCCT |
| | | AGCATTCGTGGCGATCGAGCGTTCAGATGCGATCACAATCCAT |
| | | GACATTTCCAACCCTAGAAATGTTCAGCTGCTCGATACTGTCG |
| | | TTCTCAACCCTGATGTTGTTCGGGCCAATCAAGAGGCTGGGTT |
| | | TGAGCCAGAAGGGATTGAATTTATTCCTGCAACGAATCAAGTG |
| | | ATTGTCTCCAACCCAGAAGGCAACGCCATGAGCTTGGTAAACA |
| | | TCAATGTGATGCCACGCTAG |
| SYNPCC700 | MVSLAIAPLSLWAETVELQ | ATGGTCAGTTTGGCAATCGCCCCCCTATCTCTCTGGGCTGAAA |
| 2_A0064 | LLHLNDVYEITPLGGGATG | CGGTAGAATTGCAACTGCTTCACCTCAATGATGTCTATGAAAT |
| Ser/Thr | GLARLATLRKELLAENPHT | TACGCCCCTGGGTGGTGGGGCAACGGGGGGCCTGGCGCGGTTG |
| protein | FTVLAGDLFSPSALGTAVV | GCGACCCTACGCAAGGAACTGCTCGCCGAAAATCCCCACACTT |
| phosphatase | DGDRLAGKQIVAVMNQVGL | TCACCGTTTTAGCTGGGGATTTATTTAGTCCGTCGGCCTTGGG |
| family | DLATFGNHEFDISESQFKQ | GACTGCGGTGGTTGATGGCGATCGCCTCGCAGGAAAACAAATT |
| protein | RLAESDFQWFSGNVLTAAG | GTGGCGGTGATGAACCAAGTGGGCTTGGATCTTGCCACCTTCG |
| family | EPWDNVPPYVIETIYGEAG | GTAACCACGAATTTGACATCAGCGAATCCCAGTTCAAGCAACG |
| | TPVRVGFVGVVIPSNPVDY | CTTAGCAGAATCAGATTTCCAGTGGTTTTCGGGGAATGTCCTG |
| | VTYLDPLEQMEILVAELEA | ACGGCGGCGGGGGAACCCTGGGATAATGTACCTCCCTACGTGA |
| | QTDIIVAVTHLAMQDDHHL | TTGAAACCATTTATGGTGAGGCGGGCACCCCGGTGCGTGTTGG |
| | AENIPEIDLILGGHDHENI | TTTTGTGGGGGTGGTAATTCCGAGCAATCCCGTAGATTACGTC |
| | QQWRGADFTPIFKADANAR | ACCTATCTCGACCCGCTAGAACAGATGGAAATCCTCGTCGCAG |
| | TVYLHNLSYDTETEQLTVQ | AATTAGAGGCACAAACGGATATTATTGTGGCGGTCACTCACCT |
| | SHLQPITGAIAADPETEQE | GGCGATGCAGGATGACCATCATCTTGCTGAAAATATCCCGGAA |
| | VNYWQQLAFDGFRADGFEP | ATTGACCTAATCCTGGGGGGCCACGACCATGAAAATATTCAAC |
| | EQIITESPIALDGLESSVR | AGTGGCGTGGTGCGGATTTTACGCCGATTTTCAAGGCCGATGC |
| | NQATALTDIIAQSMLTATP | CAATGCTCGCACGGTTTATCTCCATAATCTCAGCTACGACACA |
| | AAELAIFNGGSIRVDDVLP | GAAACGGAGCAGCTTACAGTTCAATCACATTTGCAACCGATTA |
| | PGPLSQYDVIRILPFGGNL | CCGGGGCGATCGCCGCAGATCCAGAAACAGAACAGGAGGTTAA |
| | ATVEIKGTTLERILNQGLA | TTATTGGCAGCAACTGGCCTTTGATGGTTTTCGGGCTGATGGT |
| | NRGTGGYLQTARVTFVPES | TTTGAACCAGAGCAAATCATTACCGAAAGTCCAATCGCCCTAG |
| | QTWQIGDRPLDPERIYRVA | ATGGTTTGGAAAGTTCCGTGCGCAACCAAGCCACAGCGTTAAC |
| | ATEFLISGRETGLDFFTPD | GGACATCATTGCCCAGTCGATGTTAACGGCGACACCCGCTGCC |
| | HPDVTLLETGEDVRFAFIQ | GAATTAGCCATTTTTAATGGCGGCTCGATCCGTGTTGATGATG |
| | QLQQEWID | TGCTGCCTCCCGGCCCGTTGTCCCAGTATGATGTGATTCGGAT |
| | | TTTGCCCTTCGGCGGAAATTTGGCCACCGTCGAGATCAAGGGC |
| | | ACAACCTTGGAACGCATTCTCAATCAAGGTTTAGCCAATCGCG |
| | | GCACCGGGGGATATTTGCAAACGGCGAGGGTGACCTTTGTCCC |
| | | GGAAAGTCAAACCTGGCAAATTGGCGATCGCCCTTTAGATCCC |
| | | GAACGCATTTATCGGGTCGCAGCGACGGAATTTCTCATCTCCG |
| | | GGCGAGAAACGGGCCTCGATTTCTTCACGCCTGACCATCCCGA |
| | | TGTGACCTTGCTCGAAACGGGAGAAGATGTACGTTTTGCCTTT |
| | | ATTCAACAGCTCCAACAGGAATGGATCGATTAG |
| SYNPCC700 | MHGNRRQFLTYGGLALGSV | ATGCACGGGAATCGACGACAGTTTTTAACCTATGGGGGCTTGG |
| 2_A2155 | LISRGIIAKSQAIANSAPT | CCCTAGGGAGTGTACTTATTTCGCGTGGGATTATTGCAAAATC |
| Ser/Thr | ALNAPAPGETRLVVISDLN | TCAGGCGATCGCTAATTCTGCACCGACTGCACTTAATGCCCCA |
| protein | SAYGSTDYLSQVKRAIALI | GCCCCAGGGGAGACGCGCCTGGTTGTGATTAGCGACCTGAACA |
| phosphatase | PDWQPDLVLCAGDMVAGQK | GTGCCTATGGTTCCACGGATTATCTGTCCCAAGTGAAACGGGC |
| family | SSLTPAQLTSMWQAFERYI | GATCGCCTTGATTCCCGATTGGCAACCGGATCTAGTGCTCTGT |
| protein | AQPLRQANIPFAFTLGNHD | GCGGGCGATATGGTCGCAGGCCAAAAAAGCAGCCTCACCCCAG |
| | ASGSLRNGQYAFAADRQAA | CCCAGCTCACCTCCATGTGGCAAGCCTTTGAACGATACATTGC |
| | SQYWRNPAHTPTLDFVDRR | CCAACCCCTGCGCCAAGCAAACATTCCCTTCGCCTTCACCCTC |
| | HFPFYYSFTQDNIFYSVWD | GGGAACCACGATGCTTCCGGCTCCCTGCGCAATGGACAATACG |
| | ASTARISPAQLAWIEASLA | CCTTTGCCGCAGATCGTCAGGCGGCCAGTCAATATTGGCGCAA |
| | SDQAQRSRLRFALGHLSLY | CCCTGCCCATACCCCGACCCTAGACTTTGTTGACCGTCGTCAT |
| | PVASGSRSEPGNYLHDGDR | TTTCCTTTCTATTACAGCTTTACCCAAGACAATATTTTTTACT |
| | LQALLEKYNVHTYISGHQH | CTGTGTGGGATGCTTCCACCGCCCGCATTAGTCCAGCACAGTT |
| | AYYPAHRGQLELLHTGALG | GGCTTGGATCGAAGCCAGTCTCGCCAGTGACCAAGCTCAACGG |
| | DGPRSLVQGNLSPYRSLTM | AGTCGTTTACGGTTCGCCCTAGGGCATTTATCCCTCTATCCTG |
| | IDIPRGGTNLRYTTYNMDR | TCGCTTCGGGCAGCCGCTCAGAGCCAGGAAATTATCTCCATGA |
| | LTVVDHGTLPGSLNTPRGY | TGGCGATCGCCTCCAGGCTCTGCTCGAAAAATACAACGTCCAC |
| | LQRRDLRAT | ACCTACATCAGCGGTCACCAACACGCTTACTATCCCGCTCACC |
| | | GGGGGCAATTGGAACTGCTCCATACAGGTGCTTTAGGAGATGG |
| | | GCCGCGTTCTCTAGTTCAAGGCAATCTTTCCCCTTACCGGAGC |
| | | CTCACGATGATTGATATTCCCAGGGGCGGCACAAACTTGCGCT |
| | | ACACCACCTACAACATGGATCGCCTGACTGTGGTTGATCACGG |
| | | CACTTTACCCGGCAGTTTGAATACTCCGAGGGGATATTTGCAA |
| | | CGCCGCGATCTGCGGGCCACTTGA |
| SYNPCC700 | MAYKLLFVCLGNICRSPSA | ATGGCCTATAAATTATTATTCGTTTGCCTCGGTAACATCTGCC |
| 2_A0973 | ENIMRHLLEQEGLSNKILC | GTTCCCCCTCCGCCGAAAATATTATGCGGCATCTTTTGGAGCA |
| low | DSAGTSSYHIGAAPDRRMQ | AGAAGGTTTAAGCAATAAAATTCTCTGCGATTCGGCCGGGACT |
| molecular | AAAQKRDIRLMGSARQFSR | TCTAGCTATCACATAGGAGCCGCCCCAGACCGACGGATGCAGG |
| weight | ADFEAFDLILAMDRANYRD | CAGCGGCCCAAAAGCGCGATATTCGTCTGATGGGTAGCGCCCG |
| phospho- | ILSLDRADIYGEKVKMMCD | GCAATTTTCCCGCGCTGATTTTGAAGCATTTGACCTGATCCTG |
| tyrosine | YATNFPDSEVPDPYYGGQS | GCAATGGATCGCGCTAATTATCGTGACATTTTGTCCCTAGACC |
| protein | GFDYVIDLLLDACQGLLTE | GGGCGGATATCTATGGCGAAAAAGTTAAAATGATGTGTGACTA |
| phosphatase | IKQEM | CGCCACGAATTTTCCCGATAGCGAAGTGCCAGATCCCTACTAC |
| | | GGCGGCCAATCGGGTTTTGACTATGTGATTGATTTGCTCCTCG |
| | | ATGCCTGCCAAGGACTCCTCACAGAAATTAAACAGGAAATGTG |
| | | A |
| SYNPCC700 | MSITLPYLRASGSLALTFQ | ATGTCTATTACTCTTCCTTATCTCCGAGCATCGGGTTCCTTGG |
| 2_A2585 | AADLVGDRYWVVAPQIWQD | CGTTAACCTTTCAGGCAGCGGATCTTGTTGGCGATCGCTACTG |
| protein | TKPEAPPDCTAPNDLAQRY | GGTGGTTGCACCGCAAATTTGGCAAGACACCAAGCCCGAAGCA |
| phosphatase | GKLYSRQLHLPRIYDILSL | CCACCGGACTGCACAGCCCCCAATGACCTGGCCCAACGCTATG |
| 2C | PEGEILLLDNIPINNQGEL | GCAAATTATATTCCCGTCAACTGCACTTGCCCCGCATTTACGA |
| domain | LPALGSVWADASPLQQLNW | TATTTTGTCTCTCCCGGAAGGGGAAATTTTACTCCTCGACAAC |
| protein | LWQMLDLWEDLAAVAMGTS | ATCCCAATTAACAATCAAGGGGAACTGCTGCCTGCCCTAGGAT |
| | LLPLENIRVDGWRLRLMEL | CGGTCTGGGCCGATGCTTCTCCCCTGCAACAGTTAAATTGGCT |
| | LADPPGAPVTLGALVTPWR | GTGGCAAATGCTCGATCTTTGGGAAGATTTGGCAGCCGTGGCC |
| | SLLAESTPPVQAMLTELIE | ATGGGCACCAGTCTTTTGCCGTTAGAAAATATCCGGGTCGATG |
| | SFSEPDADLEIILPRLNQL | GTTGGCGACTCCGACTGATGGAATTATTGGCCGATCCCCCTGG |
| | LLEQSSQQHLQMAIASATD | TGCCCCTGTCACCTTAGGGGCCTTAGTAACGCCCTGGCGATCG |
| | QGKLPTSNQDAHYPTTQDL | CTCCTGGCCGAAAGCACGCCGCCAGTCCAAGCAATGCTGACGG |
| | AAPPTATLALSDHLLMVCD | AACTAATTGAAAGCTTTAGCGAACCGGATGCAGATCTAGAGAT |
| | GVEGHGQGDVASQLAIQSL | TATTTTGCCCCGGTTAAATCAGCTCCTTTTAGAGCAGTCTAGC |
| | KLQLTGFFQGLFDTDEVVP | CAGCAACATCTGCAAATGGCGATCGCCAGTGCCACCGACCAGG |
| | PAVIEQQLAAYIRITNNLI | GAAAACTCCCCACAAGCAACCAAGATGCCCACTACCCCACGAC |
| | AERNDQEGRTGGDRMATTL | CCAGGATTTAGCCGCTCCCCCTACGGCAACCCTAGCCTTGAGT |
| | TLALQVPQRPKADKLQDS | GATCATTTACTAATGGTGTGTGACGGCGTTGAAGGCCATGGCC |
| | HSHELYIAQVGDSRAYWIT | AGGGGGATGTGGCGAGTCAGTTGGCAATTCAATCCCTCAAGTT |
| | KDQCVCLTVDDDLLSREVQ | GCAATTGACAGGTTTCTTCCAAGGGCTATTTGATACCGATGAA |
| | AGRAIYRQGLQRPDHMALT | GTGGTTCCCCCGGCGGTCATCGAACAACAGTTGGCGGCCTACA |
| | QALGIKGGDRLHPVIRRFV | TTCGCATTACAAATAACTTGATCGCCGAACGTAACGATCAAGA |
| | FAEDGVLVVCSDGLSDQQF | AGGACGCACGGGGGGCGATCGCATGGCCACTACTCTAACCCTG |
| | LESHWQTFAPVIIQGHLPP | GCCCTCCAGGTACCCCAAAGACCCAAGGCCGACAAACTCCAGG |
| | AALLQGLIEKAIAKNPEDN | ATAGCCACAGCCACGAACTCTACATTGCCCAGGTGGGGGACAG |
| | ITAAIAFYRFTTDTFTQAP | CCGTGCCTATTGGATCACTAAAGATCAATGCGTTTGCTTAACG |
| | DIETAPAPEDFEPEFVPPD | GTGGATGATGATCTGCTCAGTCGGGAAGTCCAGGCGGGCCGGG |
| | LALDTTLEAELESEPETEN | CTATTTATCGTCAAGGGTTACAGCGTCCTGATCACATGGCCCT |
| | SLSQFTLILVSLVAILLML | CACCCAAGCCCTAGGGATTAAAGGGGGCGATCGCCTCCATCCT |
| | VLAAFGLNWLLNRGPEPTQ | GTGATTCGCCGCTTCGTGTTTGCTGAAGATGGGGTGTTGGTGG |
| | PGEPNLETPTNA | TCTGTTCCGATGGCCTGAGTGACCAGCAATTTTTAGAGTCCCA |
| | | TTGGCAGACCTTCGCCCCGGTGATTATCCAGGGTCATTTGCCC |
| | | CCGGCGGCCCTGCTCCAGGGCTTAATCGAGAAGGCGATCGCCA |
| | | AAAATCCTGAAGATAACATTACGGCGGCGATCGCCTTCTACCG |
| | | CTTCACAACGGATACCTTCACCCAGGCCCCGGACATTGAAACG |
| | | GCCCCCGCCCCGGAAGACTTTGAGCCGGAATTTGTCCCCCCAG |
| | | ATCTCGCCCTAGACACAACCCTTGAGGCGGAACTGGAGTCGGA |
| | | ACCAGAAACAGAAAACAGTCTATCCCAGTTCACCTTAATTCTG |
| | | GTGAGTTTAGTGGCGATTCTTTTGATGTTAGTCCTGGCGGCCT |
| | | TTGGCTTGAACTGGCTGTTAAACCGTGGGCCTGAGCCGACGCA |
| | | ACCGGGGGAGCCAAATCTTGAAACCCCTACAAACGCAGAGTAG |
| SYNPCC700 | MATSVYQLKTNSTQFANVT | ATGGCTACTTCCGTCTATCAGCTTAAAACGAATTCCACTCAAT |
| 2_A1401 | QGEDCTLAAIDIGTNSIHM | TTGCGAATGTCACCCAAGGGGAGGACTGTACCCTAGCAGCGAT |
| ppx | VIVKIQPSLPAFTIVAREK | TGATATCGGCACCAACTCAATTCACATGGTGATTGTCAAAATT |
| exopoly- | DTVRLGHRDRLTGNLTEAA | CAACCCAGCCTGCCCGCATTTACAATTGTGGCCCGGGAAAAAG |
| phosphatase | MDRSLNALRRCQDLATSFQ | ATACGGTGCGCCTCGGTCATCGCGATCGCCTCACAGGAAACCT |
| | VDSLVAVATSAVREAPNGR | GACGGAAGCCGCCATGGATCGTTCTTTAAATGCCCTCCGTCGT |
| | EFLQPIEAELGLEVDLISG | TGTCAGGATCTAGCGACGAGTTTTCAGGTGGATTCTTTAGTAG |
| | QEEARRIYLGVLSAVDFNQ | CAGTGGCAACCAGTGCCGTGCGAGAAGCCCCCAACGGTCGAGA |
| | QPHVLIDIGGGSTEISLVE | ATTTTTACAACGGATTGAAGCAGAATTAGGGTTAGAAGTTGAT |
| | SHEARFLSS | CTAATCTCCGGCCAAGAAGAAGCGCGCCGTATCTACCTCGGTG |
| | TKVGAVRLTQDFVNTDPIS | TTTTATCAGCCGTTGACTTTAACCAACAACCCCATGTTTTGAT |
| | NREFAALQAYIRGMLERPI | TGATATTGGGGGCGGTTCGACAGAAATTAGCTTGGTGGAAAGC |
| | EELQEHLFPEEQVQMIGTS | CATGAAGCACGCTTTCTTAGCAGCACAAAGGTGGGAGCGGTGC |
| | GTIETLAAMHAMANLGNVP | GGTTAACCCAGGACTTTGTGAATACTGATCCGATTAGTAACCG |
| | SPLHGYTFSRQDLSKLIQQ | AGAATTTGCGGCCCTACAAGCTTATATTCGGGGGATGTTAGAG |
| | MRELNCRERSNLPGMSDKR | CGTCCCATTGAAGAACTACAAGAGCATCTTTTCCCGGAAGAAC |
| | AEIILAGAIILQEAMDLLQ | AGGTACAAATGATCGGGACCTCTGGCACCATTGAAACCTTGGC |
| | LKKITLCERALREGVIVDW | AGCAATGCACGCGATGGCCAATTTAGGAAATGTGCCGAGTCCC |
| | MLSHGLIESRLQYQSSIRE | CTCCATGGCTATACGTTTTCGCGTCAGGATTTGAGCAAACTGA |
| | RSVMAIAKK | TTCAACAGATGCGGGAGCTTAATTGTCGGGAGCGCTCAAATTT |
| | YRVDLVASKRTAVFSLSLF | ACCAGGAATGTCCGATAAGCGCGCAGAAATTATTCTGGCAGGG |
| | DQLQGGLHQWDTEAREMLW | GCAATCATCCTCCAAGAAGCGATGGATCTATTGCAGCTGAAAA |
| | AAAILHNCGIYISHAAHHK | AAATTACCCTCTGTGAACGGGCGTTGCGGGAAGGGGTGATCGT |
| | HSYYLIRNAELLGFNETQL | CGACTGGATGCTTTCCCATGGTTTGATTGAAAGTCGCCTGCAA |
| | EIVANLARYHRKSKPKKKH | TACCAAAGTTCGATTCGGGAACGGAGTGTGATGGCGATCGCCA |
| | ENYQNLIHKEHRQMVSELS | AAAAATATCGCGTTGATTTGGTCGCCAGTAAACGCACTGCCGT |
| | AIMRLAVALDRRQVGAIAE | ATTTTCCCTGAGTCTCTTTGATCAGCTCCAGGGGGGGCTGCAC |
| | IQCDFDAKQRLLTLKLIPT | CAATGGGACACCGAAGCGAGGGAGATGCTCTGGGCGGCGGCGA |
| | HRDDACELELWSLNYNKEI | TTCTCCATAACTGTGGCCTTTACATTAGCCATGCGGCTCACCA |
| | FEEEFAVTV | TAAACATTCCTACTATCTGATTCGTAATGCAGAGCTCCTCGGC |
| | AAHLCP | TTTAATGAAACCCAATTAGAAATCGTCGCGAACCTCGCCCGCT |
| | | ACCACCGCAAAAGCAAGCCGAAGAAAAAACACGAAAATTATCA |
| | | AAATCTCATCCACAAAGAACACCGACAGATGGTGAGTGAGTTG |
| | | AGTGCGATCATGCGGCTTGCGGTGGCCCTTGACCGACGCCAGG |
| | | TAGGGGCGATCGCCGAAATTCAGTGTGACTTTGATGCGAAACA |
| | | ACGCCTACTCACCCTCAAGCTAATCCCAACCCATAGGGATGAT |
| | | GCCTGCGAACTAGAGCTCTGGAGTTTAAACTATAACAAGGAGA |
| | | TCTTTGAAGAAGAATTTGCAGTGACCGTGGCCGCCCATCTATG |
| | | CCCCTAA |
| SYNPCC700 | MKLFVYHTPEATPTDQLPD | GTGAAACTTTTTGTGTATCACACGCCTGAGGCGACGCCAACGG |
| 2_A1835 | CAVVIDVLRATTTIATALH | ATCAACTCCCCGATTGTGCTGTGGTTATTGACGTACTGCGGGC |
| comB 2- | AGAEAVQTFADLDELFQFS | CACCACAACCATCGCTACGGCGCTCCACGCTGGAGCAGAAGCA |
| phospho- | ETWQQTPFLRAGERGGQQV | GTGCAAACCTTTGCTGACCTCGATGAACTGTTTCAATTTAGTG |
| sulpho- | EGCELGNSPRSCTPEMVAG | AAACTTGGCAGCAAACCCCCTTTCTCCGGGCTGGGGAACGGGG |
| lactate | KRLFLTTTNGTRALKRVEQ | CGGGCAACAGGTAGAAGGCTGTGAGCTTGGCAATTCTCCCCGC |
| phosphatase | APTVITAAQVNRQSVVKFL | AGTTGTACTCCAGAAATGGTGGCTGGGAAGCGCCTCTTCTTAA |
| | QTEQPDTVWFVGSGWQGDY | CAACCACCAACGGCACGAGGGCCCTCAAGCGCGTTGAGCAAGC |
| | SLEDTVCAGAIAKSLWNGD | ACCCACAGTGATTACCGCAGCCCAAGTGAATCGCCAGAGCGTG |
| | SDQLGNDEV | GTGAAGTTTCTCCAGACAGAACAGCCAGACACCGTTTGGTTCG |
| | IGAISLYQQWQQDLFGLFK | TTGGTTCCGGTTGGCAGGGGGATTATTCCCTCGAAGATACCGT |
| | LASHGQRLLRLDNEIDIRY | CTGTGCTGGGGCGATCGCCAAGTCCCTGTGGAATGGGGACAGT |
| | CAQSDTLAVLPIQTEPGVL | GACCAGTTAGGGAATGACGAAGTGATTGGGGCAATTTCCCTTT |
| | KAYRH | ACCAACAGTGGCAGCAAGATTTATTTGGCCTCTTCAAGCTCGC |
| | | AAGCCACGGCCAGCGTCTCCTGCGCTTAGACAATGAAATCGAT |
| | | ATTCGTTACTGTGCCCAAAGCGATACCCTGGCGGTTTTACCGA |
| | | TCCAAACAGAGCCGGGTGTCCTCAAAGCCTATCGCCACTAA |
| SYNPCC700 | MDQQKLTEVLAIARQIGWG | ATGGATCAGCAAAAGTTAACGGAAGTTTTGGCGATCGCCCGAC |
| 2_A0034 | AGDVLQSYYKGDIKNISDK | AAATCGGTTGGGGTGCAGGGGATGTTCTCCAAAGTTATTACAA |
| inositol | KDGPVTKADLAANHYILEA | AGGAGATATTAAAAATATTTCTGATAAAAAAGATGGCCCTGTC |
| monophos- | FQEKLGTEDFAYLSEETYD | ACCAAGGCAGATTTAGCAGCAAATCACTATATTCTGGAAGCGT |
| phatase | GNKVEHPWVWIIDPLDGTR | TTCAGGAAAAGTTAGGCACTGAAGATTTTGCCTATCTCAGCGA |
| family | DFIDQTGEYAVHICLVHEG | AGAAACCTACGACGGCAATAAAGTTGAACATCCTTGGGTGTGG |
| protein | RPVIAVVVVPEAEKLYFAS | ATTATTGATCCCCTCGATGGCACCCGTGATTTTATTGACCAAA |
| | KGNGTFVETRDGTVTPIKV | CGGGAGAATATGCCGTTCACATTTGCCTTGTTCATGAAGGTCG |
| | SERNQPEDLYLVASRTHRD | CCCGGTCATTGCGGTAGTGGTCGTCCCCGAAGCAGAAAAGCTT |
| | QRFQDLLDR | TATTTCGCGTCGAAAGGGAATGGCACTTTTGTGGAAACTCGTG |
| | LPFKDRNYVGSVGCKIAHI | ATGGCACCGTCACCCCAATTAAAGTTTCTGAGCGCAATCAACC |
| | LEQKSDVYISLSGKSAAKD | AGAAGATTTATATTTAGTCGCCAGCCGTACCCACCGGGATCAA |
| | WDFAAPELILTEAGGKFSY | CGCTTCCAGGATTTGTTAGATCGCCTACCCTTTAAAGATAGAA |
| | FAGNEVLYNQGDVVKWGGI | ATTATGTGGGGAGTGTCGGCTGTAAAATTGCCCATATTCTCGA |
| | MASNGPCHAELCQQAIAIL | ACAAAAATCCGATGTTTATATTTCTCTATCGGGGAAATCTGCA |
| | AELDRT | GCAAAAGATTGGGATTTTGCGGCCCCGGAACTAATCCTCACGG |
| | | AAGCAGGTGGAAAATTTAGTTATTTTGCAGGCAATGAAGTGCT |
| | | CTATAACCAAGGCGATGTGGTGAAGTGGGGCGGCATTATGGCG |
| | | TCTAATGGGCCGTGTCATGCAGAACTTTGTCAGCAGGCGATCG |
| | | CCATCCTTGCAGAACTAGATCGTACATAG |
| Predicted | MIHDDGRSNYSNNRPFQDI | |
| SYNPCC700 | FKARFSRRSMLQKSMMLSA | |
| 2_A0893 | AGFIGAIA | |
| leader | | |
| Predicted | MIHDDGRSNYSNNRPFQDI | |
| SYNPCC700 | FKARFSRRSMLQKSMMLSA | |
| 2_A0893 | AGFIGAIAGNSVLKPSTA | |
| leader | | |
| Predicted | MNLNSGVKSLVAS | |
| SYNPCC700 | MVKPKLKASFKLA | |
| 2_A2352 | LLSTLAGLPLGTL | |
| Leader | IFPPQAIA | |
| Predicted | MVSLAIAPLSLWA | |
| SYNPCC700 | | |
| 2_A0064 | | |
| Ser/Thr | | |
| protein | | |
| phosphatase | | |
| family | | |
| protein | | |
| family | | |
| leader | | |
| Predicted | MHGNRRQFLTYGGLALGSV | |
| SYNPCC700 | LISRGIIA | |
| 2_A2155 | | |
| Ser/Thr | | |
| protein | | |
| phosphatase | | |
| family | | |
| protein | | |
| leader | | |
| Predicted | MAYKLLFVCLGNICRSPSA | |
| SYNPCC700 | ENIMRHLLEQEGLSNKILC | |
| 2_A0973 | DSAGTSSYHIGAAP | |
| low | | |
| molecular | | |
| weight | | |
| phospho- | | |
| tyrosine | | |
| protein | | |
| phosphatase | | |
| leader | | |
| Predicted | MAYKLLFVCLGNICRSPSA | |
| SYNPCC700 | | |
| 2_A0973 | | |
| low | | |
| molecular | | |
| weight | | |
| phospho- | | |
| tyrosine | | |
| protein | | |
| phosphatase | | |
| leader | | |
| Predicted | MSITLPYLRASGS | |
| SYNPCC700 | LALTFQA | |
| 2_A2585 | | |
| protein | | |
| phosphatase | | |
| 2C | | |
| domain | | |
| protein | | |
| leader | | |
| Predicted | MATSVYQLKTNST | |
| SYNPCC700 | QFANVTQGEDCTL | |
| 2_A1401 | AAID | |
| ppx | | |
| exopoly- | | |
| phosphatase | | |
| leader | | |
| Predicted | MKLFVYHTPEATP | |
| SYNPCC700 | TDQLPDCAVVIDV | |
| 2_A1835 | LRATTTIATALHA | |
| comB 2- | | |
| phospho- | | |
| sulpho- | | |
| lactate | | |
| phosphatase | | |
| leader | | |
| Predicted | MDQQKLTEVLAIA | |
| SYNPCC700 | RQIGWGAGDVLQS | |
| 2_A0034 | YYKGDIKNISDKK | |
| inositol | DGPVTKADL | |
| monophos- | | |
| phatase | | |
| family | | |
| protein | | |
| leader | | |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1-96. (canceled)

97. A recombinant photosynthetic microorganism, comprising one or more recombinant nucleic acid sequences comprising a first nucleic acid sequence operatively linked to a second nucleic sequence, wherein the first nucleic acid sequence encodes a nutritive protein heterologous to the photosynthetic microorganism, wherein the second nucleic acid sequence encodes a signal peptide, and wherein the recombinant photosynthetic microorganism secretes the nutritive protein.

98. The photosynthetic microorganism of claim 97, wherein the nutritive protein is an abundant protein in food, and wherein the photosynthetic microorganism is a cyanobacterium.

99. The photosynthetic microorganism of claim 98, wherein the nutritive protein is an abundant protein in a food selected from a chicken egg, a cereal, a meat or a muscle.

100. The photosynthetic microorganism of claim 97, wherein the nutritive protein comprises a dairy enzyme, a food processing enzyme, a brewing industry enzyme, or a food enzyme.

101. The photosynthetic microorganism of claim 97, wherein the nutritive protein comprises an amylase or a protease.

102. The photosynthetic microorganism of claim 97, wherein the first nucleic acid sequence encodes a first polypeptide sequence comprising a fragment of a naturally-occurring nutritive protein from about 10 to about 200 amino acids in length.

103. The photosynthetic microorganism of claim 97, wherein the nutritive protein comprises a non-enzymatically active protein.

104. A liquid culture comprising a culture medium and photosynthetic microorganisms comprising one or more recombinant nucleic acid sequences comprising a first nucleic acid sequence operatively linked to a second nucleic sequence, wherein the first nucleic acid sequence encodes a nutritive protein heterologous to the photosynthetic microorganism, wherein the second nucleic acid sequence encodes a signal peptide, and wherein the recombinant photosynthetic microorganism secretes the nutritive protein.

105. The liquid culture of claim 104, wherein the photosynthetic microorganisms are cyanobacteria, and wherein the nutritive protein is secreted at a level of at least 1 mg/L/OD per hour.

106. The liquid culture of claim 104 wherein the signal peptide comprises an amino acid sequence selected from SEQ ID NOS: 1-12 or an amino acid sequence shown in Tables 16, 17, 18, and/or 19 or an amino acid sequence at least 90, 91, 92, 93, 94, 95, 96, 97, 98, or 99% identical to a sequence shown in SEQ ID NOS: 1-12 or an amino acid sequence shown in Tables 16, 17, 18, and/or 19.

107. The liquid culture of claim 104, wherein the second nucleic acid sequence i) encodes a signal peptide selected from SEQ ID NOS: 13-24 or ii) comprises a nucleotide sequence shown in Tables 16, 17, 18, and/or 19 or a nucleotide sequence at least 90, 91, 92, 93, 94, 95, 96, 97, 98, or 99% identical to a sequence shown in SEQ ID NOS: 13-24 or nucleotide sequence shown in Tables 16, 17, 18, and/or 19.

108. The liquid culture of claim 104, wherein the nutritive protein comprises a native structure.

109. The liquid culture of claim 108, wherein the nutritive protein comprises a characteristic functional property associated with the native structure.

110. A nutritive composition comprising a nutritive protein, wherein the nutritive composition comprises at least one of the following features: 1) at least a portion of the carbon used as raw material of the nutritive protein is inorganic carbon; 2) at least a portion of the carbon in the nutritive protein is inorganic carbon; or 3) the nutritive composition has a higher δp than a comparable nutritive composition made from fixed atmospheric carbon or plant-derived biomass.

111. A method for producing a nutritive composition, comprising: i) providing the liquid culture of claim 104; and ii) isolating the secreted nutritive protein to produce a nutritive composition, wherein the nutritive composition comprises at least 5% of the nutritive protein.

112. The method of claim 111, wherein the nutritive protein is an abundant protein in food, and wherein the photosynthetic microorganism is a cyanobacterium.

113. The method of claim 111, further comprising the step of allowing the nutritive protein to accumulate in the culture medium.

114. The method of claim 111, comprising the step of exposing the liquid culture to light and inorganic carbon.

115. The method of claim 111, comprising the step of separating at least one amino acid of the signal peptide from the nutritive protein.

116. A nutritive composition produced by the method of claim 111.
